(19) United States
(12) Patent Application Publication
(10) Pub. No.: US 2024/0299575 A1
(43) Pub. Date: Sep. 12, 2024

(54) OLIGONUCLEOTIDE CONJUGATES TARGETED TO THE TRANSFERRIN RECEPTOR

(71) Applicant: Denali Therapeutics Inc., South San Francisco, CA (US)

(72) Inventors: Scarlett Barker, San Francisco, CA (US); Mark S. Dennis, San Carlos, CA (US); Sarah L. Devos, San Francisco, CA (US); Anthony A. Estrada, Danville, CA (US); Mihalis S. Kariolis, San Mateo, CA (US); Cathal S. Mahon, San Francisco, CA (US); Lizanne G. Nilewski, Pacifica, CA (US); Joshua I. Park, Castro Valley, CA (US); Lu Shan, Belmont, CA (US); Mai B. Thayer, South San Francisco, CA (US); Raymond Ka Hang Tong, South San Francisco, CA (US); Hai L. Tran, San Francisco, CA (US); Robert C. Wells, Foster City, CA (US); Joy Yu Zuchero, San Mateo, CA (US)

(21) Appl. No.: 18/568,076

(22) PCT Filed: Jul. 1, 2022
(86) PCT No.: PCT/US2022/073377

§ 371 Date: Dec. 7, 2023

Related U.S. Application Data
(60) Provisional application No. 63/217,743, filed on Jul. 1, 2021.
(60) Provisional application No. 63/298,193, filed on Jan. 10, 2022.
(60) Provisional application No. 63/333,449, filed on Apr. 21, 2022.

(51) Int. Cl. A61K 47/68; A61K 9/00; A61K 39/00; C07K 16/28; C12N 15/113

(52) U.S. Cl. CPC A61K 47/6807; A61K 9/0019; A61K 47/6849; C07K 16/2881; C12N 15/1138; A61K 2039/505; C07K 2317/24; C07K 2317/526; C07K 2317/55; C07K 2317/92; C12N 2310/113; C12N 2310/14

(57) ABSTRACT

Provided herein are conjugates comprising proteins that bind to a transferrin receptor and oligonucleotides that are capable of modulating the expression of a target gene or sequence, as well as methods of use thereof.

### CROSS-REFERENCE TO RELATED APPLICATIONS

This application claims the benefit of U.S. Provisional Application No. 63/217,743, filed Jul. 1, 2021, U.S. Provisional Application No. 63/298,193, filed Jan. 10, 2022, and U.S. Provisional Application No. 63/333,449, filed Apr. 21, 2022, each of which is incorporated herein by reference.

### SEQUENCE LISTING

The Sequence Listing written in file 582256_SeqListing.xml is 574 kilobytes in size, was created Jun. 29, 2022, and is hereby incorporated by reference.

### BACKGROUND

In vivo delivery of nucleic acid-based molecules, such as antisense oligonucleotides or RNAi agents, often requires specific targeting to reach certain tissues or cell types. In particular, delivery to non-hepatic tissues remains an obstacle and has limited the use of such therapies. Delivery of oligonucleotides to the central nervous system (CNS) poses a distinct problem due to the blood brain barrier (BBB). One means to deliver oligonucleotides into the CNS is by intrathecal delivery. However, intrathecal delivery is invasive, has a higher risk of side-effects, and often leads to uneven distribution.

Thus, there is a continuing need for new and improved methods for delivering nucleic acid-based therapeutics in vivo, particularly to CNS tissues.

### BRIEF SUMMARY

Accordingly, certain embodiments described herein provide conjugates comprising at least one oligonucleotide (e.g., an antisense oligonucleotide (ASO) or an RNA interference (RNAi) agent) and at least one protein engineered to bind to the transferrin receptor (TfR). In certain aspects, a protein comprised within such a conjugate comprises a modified constant domain with substitutions that generate a TfR binding site. TfR is highly expressed on the blood-brain barrier (BBB), and TfR naturally moves transferrin from the blood into the brain. Because these proteins bind TfR, they too can be transported across the BBB and further can be used to transport attached oligonucleotides across the BBB. This approach can substantially improve brain uptake and/or biodistribution of these agents and is therefore highly useful for treating disorders and diseases where brain delivery is advantageous. The conjugates described herein may also be used to enhance the delivery of oligonucleotides, such as ASO and RNAi agents, to certain peripheral tissues, such as peripheral tissues that express TfR (e.g., muscle). Also disclosed are polypeptides and proteins containing modifications that add conjugation sites or facilitate conjugation, including cysteine substitutions, such as those described herein.

Thus, in certain embodiments, the subject matter described herein is directed to a conjugate of formula I:

P-(L-(X)_{y})ₙ (I)
wherein,
   each X is independently an oligonucleotide;
   each L is independently a linking group;
   P is a protein comprising an engineered binding site that specifically binds to a transferrin receptor (TfR) with an affinity of about 3 nM to about 600 nM;
   each y is independently at least 1 (e.g., 1, 2, 3, or 4); and
   n is at least 1 (e.g., 1, 2, 3, 4, 5, 6, 7 or 8).

In certain embodiments, the subject matter described herein is directed to an Fc polypeptide dimer, or a Fab-Fc dimer fusion thereof, comprising:
(a) a first Fc polypeptide comprising a modified constant domain that specifically binds to a transferrin receptor; and
(b) a second Fc polypeptide dimerized to the first Fc polypeptide of (a);
wherein the Fab-Fc dimer fusion further comprises a first Fab and a second Fab; and, wherein the Fc polypeptide dimer, or a Fab-Fc dimer fusion comprises one or more cysteine substitutions.

In certain embodiments, the subject matter described herein is directed to an Fc polypeptide dimer comprising:
(a) a first Fc polypeptide comprising a modified constant domain that specifically binds to a transferrin receptor; and
(b) a second Fc polypeptide dimerized to the first Fc polypeptide of (a); wherein the first and/or the second Fc polypeptide comprises one or more substitutions selected from the group consisting of N297A and N297G.

In certain embodiments, provide herein is a conjugate of formula I:

P-(L-(X)_{y})ₙ (I)
wherein,
each X is independently an oligonucleotide;
each L is independently a linking group;
P is a protein comprising a transferrin receptor (TfR) binding polypeptide as described herein;
each y is independently at least 1 (e.g., 1, 2, 3, or 4); and
n is at least 1 (e.g., 1, 2, 3, 4, 5, 6, 7 or 8).

In one aspect, a protein described herein comprises a modified constant domain that binds (e.g., specifically binds) to a TfR.

In one aspect, a protein described herein further comprises one or more modified sites, which facilitate the attachment of P to each L. Accordingly, certain embodiments provide a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1 (e.g., 1, 2, 3, or 4); and
n is at least 1 (e.g., 1, 2, 3, 4, 5, 6, 7 or 8).

In certain aspects, more than one oligonucleotide is attached to the protein, for example, through the attachment of more than one linking group. Thus, certain embodiments provide a conjugate of formula I that comprises more than one oligonucleotide:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1; and
n is 2 or more (e.g., 2 or 4).

In certain other aspects, more than one oligonucleotide is attached to a single linking group. Thus, certain embodiments provide a conjugate of formula I that comprises more than one oligonucleotide:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1, wherein at least one y is 2 or more; and
n is at least one.

As described herein, the TfR binding affinity of the protein (P) may be varied. Accordingly, certain embodiments provide a low affinity (e.g., about 3 nM to about 600 nM) transferrin receptor binding conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor with a low affinity (e.g., from about 40 nM to 1200 nM);
each y is independently at least 1; and
n is at least 1.

Certain embodiments provide a conjugate of formula I:

P-(L-(X)_{y})ₙ (I)

wherein,
each X is independently an oligonucleotide;
each L is a linking group independently selected from the group consisting of:

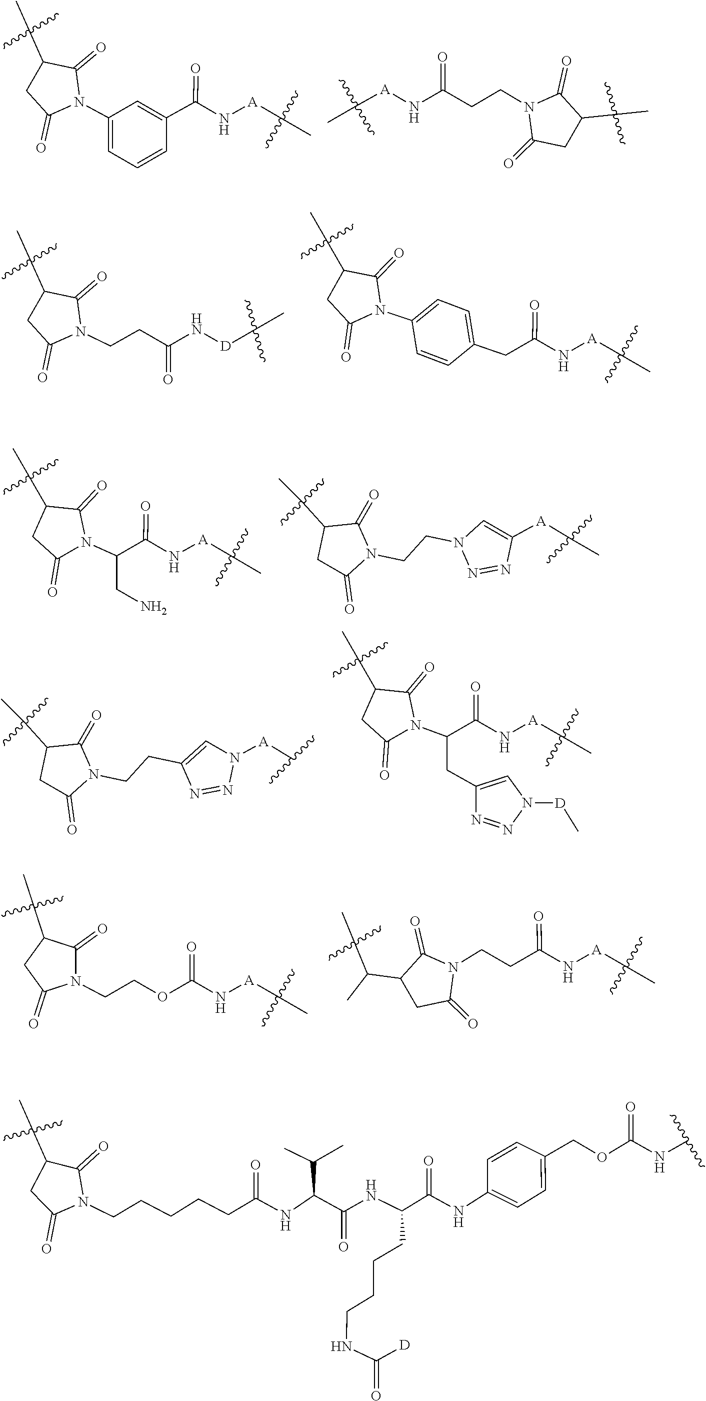

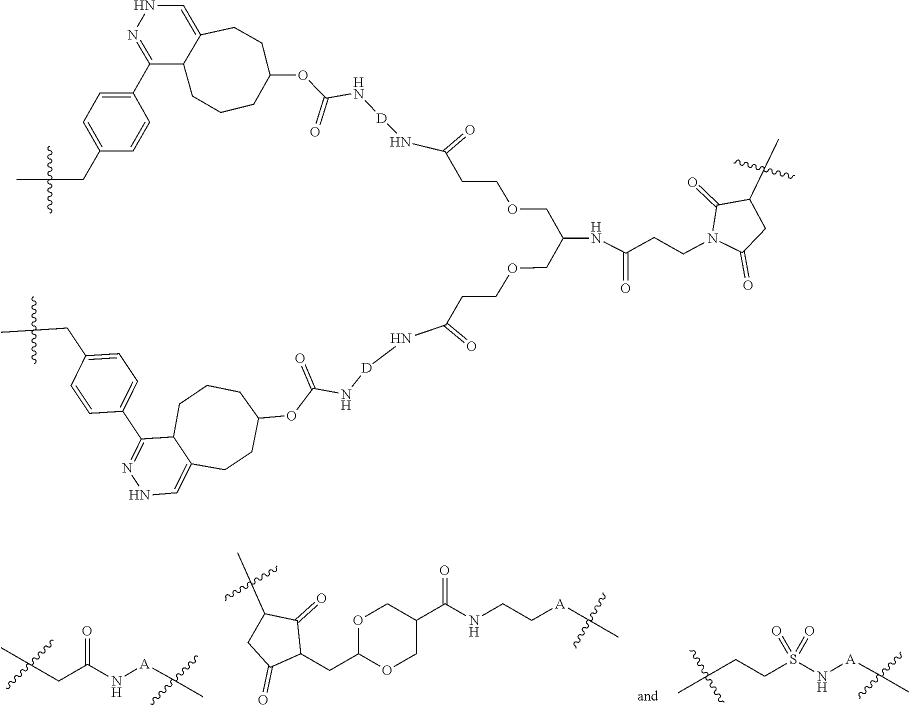

wherein
each A is independently (C₁-C₁₅)alkyl;
each D is —(CH₂—CH₂—O)ₘ—; and
each m is 1, 2, 3, 4, 5, 6, 7, 8, 9, 10, 11, 12, 13, 14, 15, 16, 17, 18, 19, 20, 21, 22, 23, or 24;
P is a protein comprising a modified constant domain that specifically binds to a transferrin receptor;
each y is independently at least 1; and
n is at least 1.

As described herein, the protein (P) may or may not comprise a Fab fragment or a portion thereof. For example, in certain embodiments, the protein does not comprise a Fab fragment or a portion thereof. In other embodiments, the protein comprises one or more non-targeting Fab fragments or portions thereof. Thus, certain embodiments provide a conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor, wherein P does not comprise a Fab fragment or a portion thereof;
each y is independently 1 or more; and
n is 1 or more.
   In certain embodiments, such a conjugate is greater than 50 kDa in size. In some embodiments, P does not comprise antigen-binding portion or variable domain portion of an Fab fragment. In some embodiments, P comprises an Fc dimer fusion but does not comprise an antibody antigen-binding domain or antibody variable domain.

Other aspects provide a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more non-targeting Fab fragments or a portion thereof,
each y is independently 1 or more; and
n is 1 or more.

Certain embodiments also provide a conjugate of formula I:

P-(L-(X)_{y})ₙ (I)

wherein,
each X is independently an oligonucleotide;
each L is independently

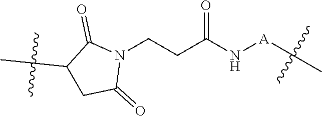

wherein A is (C₁-C₁₅)alkyl;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, selected from the group consisting of S239C, S442C, A330C, K149C, and T289C, according to EU numbering or A114C by Kabat numbering;
each y is independently at least 1; and
n is at least 1.

In certain aspects, the modified constant domain is a modified CL domain. In some embodiments, the modified constant domain is a modified CH1 domain. In some embodiments, the modified constant domain is a modified CH2 domain. In some embodiments the modified constant domain is a modified CH3 domain. In one aspect, a protein described herein comprises a modified CH3 domain that specifically binds to a transferrin receptor, wherein the modified CH3 domain comprises four, five, six, seven, eight, or nine substitutions in a set of amino acid positions comprising 157, 159, 160, 161, 162, 163, 186, 189, and 194, and wherein the substitutions and the positions are determined with reference to amino acids 114-220 of SEQ ID NO:1. In some embodiments, the modified CH3 domain further comprises one, two, three, or four substitutions at positions comprising 153, 164, 165, and 188. In one aspect, the modified CH3 domain further comprises one, two, three, four, five, six or seven substitutions at positions comprising 153, 164, 165, 187, 188, 197 and 199.

In certain embodiments, the modified constant domain is a modified CH3 domain comprising two, three, four, five, six, seven, eight, nine, ten, eleven, twelve, thirteen, fourteen, fifteen or sixteen positions selected from the following: position 153 is Trp, Tyr, Leu, Gln, or Glu; position 157 is Leu, Tyr, Met, Val, Phe or Trp; position 159 is Leu, Thr, His, Pro or Phe; position 160 is Val, Pro, or an acidic amino acid; position 161 is Trp; position 162 is Val, Ser, Ala or Gly; position 163 is Asn, Gly, His, Gln, Leu, Lys, Val, Phe, Ser, Ala, Asp, Thr or Glu; position 164 is Ser, Thr, Gln, Phe, Tyr or Val; position 165 is Gln, Phe, or His; position 186 is Glu, Ala, Ser, Leu, Thr, Pro or Asp; position 187 is Lys, Arg, Gly, or Pro; position 188 is Glu or Ser; position 189 is Thr, Asn or an acidic amino acid; position 194 is Trp, Tyr, His, or Phe; position 197 is Ser, Thr, Glu, Lys or Trp; and position 199 is Ser, Trp, Gly, Cys, Pro or Met, as numbered with reference to SEQ ID NO:1.

In certain embodiments, the protein comprises a first Fc polypeptide or Fab-Fc fusion comprising a sequence having at least 85%, 90%, 95%, 96%, 97%, 98% or 99% sequence identity to the sequence of any one of SEQ ID NOS:279, 281, 361-366, 491-494, 702-718, 632, 645-649, 738-746, 804; and a second Fc polypeptide or Fab-Fc fusion comprising a sequence having at least 85%, 90%, 95%, 96%, 97%, 98% or 99% sequence identity to the sequence of any one of SEQ ID NOS: 557-561, 627-631, 635-644, 719-723, 724-731, 733-737, and 810, wherein the first Fc polypeptide or Fab-Fc fusion and/or the second Fc polypeptide or Fab-Fc fusion comprises one or more modified sites, which facilitate the attachment of P to each L. In certain embodiments, the first Fc polypeptide or Fab-Fc fusion comprises a Glu at position 153, Tyr at position 157, Thr at position 159, Glu at position 160, Trp at position 161, Ser, or Ala at position 162, Asn at position 163, Thr or Ser at position 186, Glu at position 188, Glu at position 189, and Phe at position 194, as numbered with reference to SEQ ID NO:1.

In certain embodiments, the protein is a Fc polypeptide dimer or a Fab-Fc dimer fusion comprising a first Fc polypeptide or Fab-Fc fusion and a second Fc polypeptide or Fab-Fc fusion, wherein: the first Fc polypeptide or Fab-Fc fusion comprises the amino acid sequence of SEQ ID NO:362 and the second Fc polypeptide or Fab-Fc fusion comprises the amino acid sequence of SEQ ID NO:559; the first Fc polypeptide or Fab-Fc fusion comprises the amino acid sequence of SEQ ID NO:702 and the second Fc polypeptide or Fab-Fc fusion comprises the amino acid sequence of SEQ ID NO:559; the first Fc polypeptide or Fab-Fc fusion comprises the amino acid sequence of SEQ ID NO:708 and the second Fc polypeptide or Fab-Fc fusion comprises the amino acid sequence of SEQ ID NO:722; the first Fc polypeptide or Fab-Fc fusion comprises the amino acid sequence of SEQ ID NO:718 and the second Fc polypeptide or Fab-Fc fusion comprises the amino acid sequence of SEQ ID NO:559; the first Fc polypeptide or Fab-Fc fusion comprises the amino acid sequence of SEQ ID NO:362 and the second Fc polypeptide or Fab-Fc fusion comprises the amino acid sequence of SEQ ID NO:560; the first Fc polypeptide or Fab-Fc fusion comprises the amino acid sequence of SEQ ID NO:362 and the second Fc polypeptide or Fab-Fc fusion comprises the amino acid sequence of SEQ ID NO:561; or the first Fc polypeptide comprises the amino acid sequence of SEQ ID NO:804 and the second Fc polypeptide comprises the amino acid sequence of SEQ ID NO:810.

Certain embodiments also provide a first Fc polypeptide or a Fab-Fc fusion comprising:
1) modified constant domain that specifically binds to a transferrin receptor; and 2) one or more cysteine, alanine or glycine substitutions (e.g., S239C, S442C, A330C, K149C (light chain), T289C, N297A and/or N297G, according to EU numbering and/or A114C by Kabat numbering).

Certain embodiments also provide an Fc polypeptide dimer, or a Fab-Fc dimer fusion thereof, comprising:
(a) a first Fc polypeptide comprising a modified constant domain that specifically binds to a transferrin receptor; and
(b) a second Fc polypeptide capable of dimerizing with the first Fc polypeptide of (a);
wherein the first and/or the second Fc polypeptide comprises one or more cysteine substitutions; or wherein the first and/or second Fab-Fc fusion comprises one or more cysteine substitutions (e.g., S239C, S442C, A330C, K149C, A114C, and T289C).

Other aspects provide a Fc polypeptide dimer comprising:
(a) a first Fc polypeptide comprising a modified constant domain that specifically binds to a transferrin receptor; and
(b) a second Fc polypeptide capable of dimerizing with the first Fc polypeptide of (a);
wherein the first and/or the second Fc polypeptide comprises one or more substitutions selected from the group consisting of N297A and N297G.

In certain embodiments, the first and the second Fc polypeptides are each joined to a non-targeting Fab fragment or a portion thereof, to produce a Fab-Fc dimer fusion.

A non-targeting Fab (NTF) does not specifically bind to a naturally occurring epitope in a subject. In some embodiments, a NTF comprises a non-binding variable region (NBVR). A NBVR comprises a light chain variable domain and a heavy chain variable domain and does not specifically bind to a naturally occurring epitope in a subject. The NBVR can be, but is not limited to, an scFv.

In some embodiments, a NBVR comprises three Kabat heavy chain CDRs and three Kabat light chain CDRs from NBVR1 or NBVR2. In some embodiments, a NBVR comprises a CDR-H1, a CDR-H3, a CDR-L1, a CDlR-L2, and a CDR-L3 from NBVR1 or NB3VR2 and a CDlR-H2 comprising SEQ ID NO: 869.

In some embodiments, a NBVR, does not specifically bind to a naturally occurring epitope in a subject and comprises three light chain CDRs and three heavy chain CDRs, wherein each CDR has at least 90% sequence identity or 100% sequence identity to a corresponding CDR from the heavy and light chain variable regions of NBVR1 or NBVR2. The heavy chain CDRs, CDR-H1, CDR-H2, and CDR-H1, from NBVR1 comprise: SEQ ID NOs: 825, 827 or 869, and 829, respectively. The light chain CDRs, CDR-L1, CDR-L2, and CDR-L1 from NBVR1 comprise: SEQ ID NOs: 819, 821, and 823, respectively. The heavy chain CDRs, CDR-H1, CDR-H2, and CDR-H1, from NBVR2 comprise: SEQ ID NOs: 826, 828 or 869, and 829, respectively. The light chain CDRs, CDR-L1, CDR-L2, and CDR-L1 from NBVR2 comprise: SEQ ID NOs: 820, 822, and 824, respectively. In some embodiments, the CDR-H2 of NBVR1 or NBVR2 is replaced with SEQ ID NO: 869.

In some embodiments, an NBVR comprises a heavy chain variable region comprising an amino acid sequence having at least 70%, at least 75%, at least 80%, at least 85%, at least 90%, or at least 95% identity to the amino acid sequence of SEQ ID NO: 837, and a light chain variable region comprising an amino acid sequence having at least 70%, at least 75%, at least 80%, at least 85%, at least 90%, or at least 95% identity to the amino acid sequence of SEQ ID NO: 832. In some embodiments, an NBVR comprises a heavy chain variable region comprising an amino acid sequence having at least 70%, at least 75%, at least 80%, at least 85%, at least 90%, or at least 95% identity to the amino acid sequence of SEQ ID NO: 853, and a light chain variable region comprising an amino acid sequence having at least 70%, at least 75%, at least 80%, at least 85%, at least 90%, or at least 95% identity to the amino acid sequence of SEQ ID NO: 851.

In some embodiments, an NBVR comprises a heavy chain variable region comprising the amino acid sequence of SEQ ID NO: 837; and a light chain variable region comprising the amino acid sequence of SEQ ID NO: 832; or a heavy chain variable region comprising the amino acid sequence of SEQ ID NO: 853; and a light chain variable region comprising the amino acid sequence of SEQ ID NO: 851.

In some embodiments, a NTF comprises a heavy chain comprising an amino acid sequence having at least 70%, at least 75%, at least 80%, at least 85%, at least 90%, or at least 95%, or 100% identity to the amino acid sequence of any one of SEQ ID NO: 838, 839, 840, 841, 844, 845, 846, 847, 854, 855, 856, 857, 859, 860, 861, and 862; and a light chain comprising an amino acid sequence having at least 70%, at least 75%, at least 80%, at least 85%, at least 90%, or at least 95%, or 100% identity to the amino acid sequence of any one of SEQ ID NO: 833, 835, 850, and 852.

If the NTF light chain comprises the amino acid sequence of SEQ ID NO: 833, then the heavy chain can comprise the amino acid sequence of SEQ ID NO: 838, 839, 840, or 841.

If the NTF light chain comprises the amino acid sequence of SEQ ID NO: 833, then the heavy chain can comprise the amino acid sequence of SEQ ID NO: 844, 845, 846, or 847.

If the NTF light chain comprises the amino acid sequence of SEQ ID NO: 835, then the heavy chain can comprise the amino acid sequence of SEQ ID NO: 838, 839, 840, or 841.

If the NTF light chain comprises the amino acid sequence of SEQ ID NO: 835, then the heavy chain can comprise the amino acid sequence of SEQ ID NO: 844, 845, 846, or 847.

If the NTF light chain comprises the amino acid sequence of SEQ ID NO: 850, then the heavy chain can comprise the amino acid sequence of SEQ ID NO: 854, 855, 856, or 857.

If the NTF light chain comprises the amino acid sequence of SEQ ID NO: 850, then the heavy chain can comprise the amino acid sequence of SEQ ID NO: 859, 860, 861, or 862.

If the NTF light chain comprises the amino acid sequence of SEQ ID NO: 852, then the heavy chain can comprise the amino acid sequence of SEQ ID NO: 854, 855, 856, or 857.

If the NTF light chain comprises the amino acid sequence of SEQ ID NO: 852, then the heavy chain can comprise the amino acid sequence of SEQ ID NO: 859, 860, 861, or 862.

Also described are nucleic acids encoding the heavy chains and/or light chains of the any of the described NBVR. In some embodiments, a nucleic acid encoding heavy chain of a NBVR comprising a sequence having at least 70%, at least 75%, at least 80%, at least 85%, at least 90%, or at least 95%, or 100% identity to SEQ ID NO: 865 or 867. In some embodiments, a nucleic acid encoding light chain of a NBVR comprising a sequence having at least 70%, at least 75%, at least 80%, at least 85%, at least 90%, or at least 95%, or 100% identity to SEQ ID NO: 864 or 866.

In certain embodiments, an Fc polypeptide, Fc polypeptide dimer, Fab-Fc fusion or Fab-Fc fusion dimer is incorporated into a conjugate of formula (I), as described herein.

Certain embodiments also provide a pharmaceutical composition comprising a conjugate as described herein and a pharmaceutically acceptable carrier.

In certain embodiments, the subject matter described herein is directed to a method of targeting delivery of an oligonucleotide to muscle tissue and/or brain tissue in a patient comprising, administering to the subject any of the conjugates or the pharmaceutical compositions comprising a conjugate as described herein.

In certain embodiments, the subject matter described herein is directed to a method of modulating gene expression in a brain cell or a plurality of brain cells comprising administering to a patient a conjugate comprising:
(i) a protein that specifically binds TfR and;
(ii) an oligonucleotide,
wherein the protein binds TfR with a low affinity and the conjugate is transported across the BBB, and wherein in the cell, the oligonucleotide modulates the expression of a target gene.

In certain embodiments, the subject matter described herein is directed to a method of delivering an oligonucleotide to deep brain regions, comprising administering to a patient a conjugate comprising:
(i) a protein that specifically binds TfR and;
(ii) an oligonucleotide,
wherein the protein binds TfR with a low affinity and the conjugate is transported across the BBB, and wherein in a brain cell in the deep brain regions, the oligonucleotide modulates the expression of a target gene.

In certain embodiments, the subject matter described herein is directed to a method of delivering an antisense oligonucleotide across brain regions, comprising administering to a patient a conjugate comprising:
(i) a protein that specifically binds TfR and;
(ii) an oligonucleotide,
wherein the protein binds TfR with a low affinity and the conjugate is transported across the BBB, and wherein in a cell in a brain region, the oligonucleotide modulates the expression of a target gene.

In certain embodiments, the subject matter described herein is directed to a method of generating a neuron cell with decreased target gene expression, comprising delivering to the neuron cell an oligonucleotide, wherein the oligonucleotide is transported across the BBB as part of a conjugate with a protein that binds TfR with a low affinity, and wherein said oligonucleotide decreases the expression level of the target gene.

In certain embodiments, the subject matter described herein is directed to a method of modifying a neuron cell to decrease target gene expression, comprising delivering to the neuron cell an oligonucleotide, wherein the oligonucleotide is transported across the BBB as part of a conjugate with a protein that binds TfR with a low affinity, and wherein said oligonucleotide decreases the expression level of the target gene.

Certain embodiments provide a method for transcytosis of an oligonucleotide (e.g., ASO or an RNAi agent) across an endothelium, the method comprising contacting the endothelium (e.g., blood-brain barrier) with a conjugate as described herein.

Certain embodiments provide a method for transporting an oligonucleotide (e.g., ASO or an RNAi agent) across the blood brain barrier (BBB) of a patient, comprising administering a conjugate as described herein to the patient. For example, certain embodiments provide a method of transporting an oligonucleotide across the BBB of a patient, comprising administering to the patient a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1.

Certain embodiments also provide a method of transporting an oligonucleotide across the BBB of a patient, comprising administering to the patient a conjugate of formula I:

P-(L-(X)_{y})ₙ (I)

wherein,
each X is independently an oligonucleotide;
each L is independently

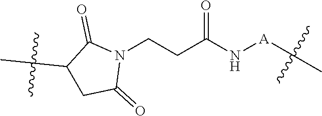

wherein A is (C₁-C₁₅)alkyl;
P is a protein comprising a Fab-Fc dimer fusion, comprising 1) a modified constant domain that specifically binds to a transferrin receptor with an affinity of about 100 nM; and 2) one or more modified sites, which facilitate the attachment of P to each L, selected from the group consisting of S239C, S442C, A330C, K149C, and T289C, according to EU numbering and A114C according to Kabat numbering;
each y is independently at least 1; and
n is at least 1. In certain embodiments, the conjugate is administered to the patient intravenously.
   In certain embodiments, the oligonucleotide is distributed throughout the central nervous system.
   In certain embodiments, the oligonucleotide is distributed across brain regions. In certain embodiments, the oligonucleotide is distributed to a deep brain region. In certain embodiments, the oligonucleotide is distributed to the spinal cord. In certain embodiments, the conjugate is administered to the patient intravenously. In certain embodiments, the oligonucleotide modulates the expression of a target gene. In certain embodiments, the modulation of target gene expression is gene knockdown or gene knockout.

Certain embodiments provide a conjugate as described herein for use transporting an oligonucleotide (e.g., ASO or an RNAi agent) across the blood brain barrier (BBB) of a patient.

Certain embodiments provide the use of a conjugate as described herein in the preparation of a medicament for transporting an oligonucleotide (e.g., ASO or an RNAi agent) across the blood brain barrier (BBB) of a patient.

Certain embodiments provide a method of modulating the expression of a target gene in a patient comprising administering an effective amount of a conjugate as described herein to the patient. In particular, certain embodiments provide a method of modulating the expression of a target gene in a cell within the brain of a patient, comprising administering to the patient a conjugate as described herein to the patient. For example, certain embodiments provide a method of modulating the expression of a target gene in a cell within the brain of a patient, comprising administering to the patient a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L; each y is independently at least 1; and
n is at least 1. Certain embodiments also provide a method of modulating the expression of a target gene in a cell within the brain of a patient, comprising administering to the patient a conjugate of formula I:

P-(L-(X)_{y})ₙ (I)

wherein,
each X is independently an oligonucleotide;
each L is independently

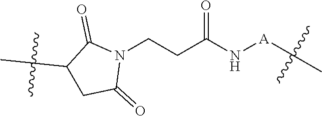

wherein A is (C₁-C₁₅)alkyl;
P is a protein comprising a Fab-Fc dimer fusion, comprising 1) a modified constant domain that specifically binds to a transferrin receptor with an affinity of about 100 nM; and 2) one or more modified sites, which facilitate the attachment of P to each L, selected from the group consisting of S239C, S442C, A330C, K149C, and T289C, according to EU numbering and A114C according to Kabat numbering;
each y is independently at least 1; and
n is at least 1.

In certain embodiments, the conjugate is administered to the patient intravenously.

In certain embodiments, the cell within the brain is a neuron, an endothelial cell, an oligodendrocyte, an astrocyte, or a microglia.

In certain embodiments, such a method modulates the expression of a target gene in a plurality of cells within the brain of a patient. In certain embodiments, the cells within the plurality are a single type of cell. In other embodiments, the plurality of cells comprise cells of different types. For example, in certain embodiments, the plurality of cells comprise at least two, three, four, or five cell types selected from the group consisting of: a neuron, an endothelial cell, an oligodendrocyte, an astrocyte, and a microglia. In certain embodiments, the neuron is an excitatory neuron. In certain embodiments, the neuron is an inhibitory neuron.

In certain embodiments, the modulation of target gene expression is gene knockdown or gene knockout.

Certain embodiments provide a conjugate as described herein for use in modulating the expression of a target gene a patient.

Certain embodiments provide the use of a conjugate as described herein in the preparation of a medicament for modulating the expression of a target gene in a patient.

Certain embodiments also provide a method of administering an effective amount of an oligonucleotide to the CNS of a patient, comprising administering to the patient a conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1; and
n is at least 1,
wherein the administered oligonucleotide modulates gene expression throughout the CNS.

Certain embodiments also provide a method of delivering an effective amount of an oligonucleotide to the CNS of a patient, comprising administering to the patient a conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1; and
n is at least 1,
   wherein the administered oligonucleotide modulates gene expression throughout the CNS.

In certain embodiments, the administered oligonucleotide modulates gene expression across brain regions and the spinal cord of the patient.

In certain embodiments, the brain regions comprise deep brain regions.

In certain embodiments, the brain regions comprise the frontal lobe, parietal lobe, temporal lobe, occipital lobe and cerebellum.

In certain embodiments, the brain regions comprise endothelial cells, neurons, astrocytes, oligodendrocytes, and microglia.

In certain embodiments, the oligonucleotide modulates gene expression in the endothelial cells, neurons, astrocytes, oligodendrocytes, and microglia.

In certain embodiments, the effective amount reduces gene expression by at least about 10%, 20%, 30%, 40%, 50%, 60%, 70%, 80%, 90%, 95%, 99% or 100%, as compared to the expression without administering the oligonucleotide.

In certain embodiments, the gene expression is reduced by at least about 50%.

In certain embodiments, the gene expression is reduced by at least about 70%.

In certain embodiments, the conjugate is administered to the subject intravenously.

Certain embodiments provide a conjugate as described herein for use in a method of administering an effective amount of an oligonucleotide to the CNS of a patient, the method comprising administering a conjugate as described herein to the patient, wherein the administered oligonucleotide modulates gene expression throughout the CNS.

Certain embodiments provide a conjugate as described herein for use in a method of delivering an effective amount of an oligonucleotide to the CNS of a patient, the method comprising administering a conjugate as described herein to the patient, wherein the administered oligonucleotide modulates gene expression throughout the CNS.

Certain embodiments provide the use of a conjugate as described herein in the preparation of a medicament for administering an effective amount of an oligonucleotide to the CNS of a patient, by administering the medicament to the patient, wherein the administered oligonucleotide modulates gene expression throughout the CNS.

### BRIEF DESCRIPTION OF THE FIGURES

FIG. 1 is an illustrative embodiment of a conjugate described herein, which comprises a Fab-Fc dimer fusion. The Fc polypeptide on the left comprises a modified CH3 domain that specifically binds to a transferrin receptor; and a “knob” substitution for heterodimerization. The Fc polypeptide on the right comprises “hole” substitutions for heterodimerization and is attached to an oligonucleotide via a linking group.

FIG. 2 illustrates plasma time course in Hu Tfr Mice across OTV variants Day 1 to Day 7 of multi-dose study.

FIG. 3 illustrates huIgG in plasma of Hu Tfr Mice at 24 hour timepoint of multi-dose study across OTV variants.

FIG. 4 illustrates intact drug and total ASO across OTV variants Day 1 to Day 7 of multi-dose study.

FIG. 5 illustrates intact drug and total ASO plasma time course in wild-type mice across OTV variants.

FIG. 6 illustrates huIgG and % intact drug plasma time course in wild-type mice across OTV variants.

FIG. 7 illustrates tissue PK overview 24 Hours Post Single Dose. For each tissue type for “HuIgG”, the bars represent the following from left to right: RSV-ASO; S239C; OTR2 (1.23); OTR4 (2.5); Low Affinity; A330C; S442C; and SansFab. For each tissue type for “Intact Drug”, the bars represent the following from left to right: RSV-ASO; S239C; OTR2 (1.23); OTR4 (2.5); Low Affinity; A330C; S442C; and SansFab. For each tissue type for “Total ASO”, the bars represent the following from left to right: RSV-ASO; S239C; OTR2 (1.23); OTR4 (2.5); Low Affinity; A330C; S442C; SansFab and Naked ASO.

FIG. 8 illustrates huIgG in tissue 24 hours after dosing in single dose study normalized to plasma AUC. For each tissue type, the bars represent the following from left to right: RSV-ASO; S239C; OTR2 (1.23); OTR4 (2.5); Low Affinity; A330C; S442C; and No Fab. Normalizing huIgG concentrations to plasma AUC results in more equivalence between the OTV variants, suggesting that much of the concentration disparity is due to plasma clearance kinetics.

FIG. 9 illustrates huIgG, intact drug, % intact drug, and total ASO in CNS, i.e., cortex, spinal cord, for increased OTR/ASO loading variants in single dose study.

FIG. 10 illustrates huIgG, intact drug, % intact drug, and total ASO in CNS, i.e., cortex, spinal cord, for low affinity variant in single dose study.

FIG. 11 illustrates huIgG, intact drug, % intact drug, and total ASO in CNS, i.e., cortex, spinal cord, for conjugation site variants in single dose study.

FIG. 12 illustrates huIgG, intact drug, % intact drug, and total ASO in CNS, i.e., cortex, spinal cord, for SansFab (i.e., No Fab) variant in single dose study.

FIG. 13 illustrates increased OTR/ASO loading variants tissue PK and knockdown in the CNS, i.e., cortex, spinal cord, 72 hours post final dose of multi-dose study.

FIG. 14 illustrates low affinity variant tissue PK and knockdown in the CNS, i.e., cortex, spinal cord, 72 hours post final dose of multi-dose study.

FIG. 15 illustrates conjugation site variants tissue PK and knockdown in the CNS, i.e., cortex, spinal cord, 72 hours post final dose of multi-dose study.

FIG. 16 illustrates SansFab (i.e., No Fab) variant tissue PK and knockdown in the CNS, i.e., cortex, spinal cord, 72 hours post final dose of multi-dose study.

FIG. 17 illustrates peripheral tissue PK at 72 hours post final dose for huIgG, intact drug and total ASO. For each tissue type, the bars represent the following from left to right: RSV-ASO; S239C; OTR2 (1.23); OTR4 (2.5); Low Affinity; A330C; S442C; and no Fab.

FIG. 18 illustrates knockdown in kidney, diaphragm, liver, and quadricep at 72 hours post final dose.

FIG. 19 illustrates knockdown in liver at 72 hours post final dose, with comparisons for OTR, TfR affinity, conjugation site differences, and Fab removal.

FIG. 20A illustrates ASO biodistribution in single dose non-human primate studies; and FIG. 20B illustrates Malat1 knockdown in brain frontal cortex.

FIG. 21 illustrates ASO biodistribution of intrathecal ASO v. OTV IV administration in non-human primates via ASO Immunofluorescence in hemibrain coronal section (neocortex inset).

FIG. 22A illustrates the brain dissection region for snRNAseq analysis; and FIG. 22B illustrates target knockdown across CNS cell types using single nuclei RNAseq analysis of multi-dose treated mouse brains.

FIG. 23 illustrates plasma PK of OTV linker variants in wildtype mice (A) total ASO; (B) total huIgG; (C) ASO to huIgG (linear); (D) ASO to huIgG (log).

FIG. 24 illustrates total ASO concentration in brain, spinal cord, and liver of OTV linker variants from multi-dose study with TfRms/hu knock-in mice.

FIG. 25 illustrates Malat1 knockdown in brain, spinal cord, and liver of OTV linker variants from multi-dose study with TfRms/hu knock-in mice.

FIG. 26 illustrates knockdown efficiency (knockdown normalized to ASO concentration) of OTV linker variants in multi-dose study with TfRms/hu knock-in mice.

FIG. 27 illustrates plasma PK profile for S239C, A114C, and T289C conjugation site variants.

FIG. 28 illustrates ASO uptake in brain for S239C, A114C, and T289C conjugation site variants in single and multi-dose studies.

FIG. 29 illustrates ASO uptake in liver for S239C, A114C, and T289C conjugation site variants in single and multi-dose studies.

FIG. 30 illustrates ASO uptake in kidney for S239C, A114C, and T289C conjugation site variants in single and multi-dose studies.

FIG. 31 illustrates brain Malat1 knockdown for S239C, A114C, and T289C conjugation site variants in single and multi-dose studies.

FIG. 32 illustrates liver and kidney Malat1 knockdown for S239C, A114C, and T289C conjugation site variants in single and multi-dose studies.

FIG. 33 illustrates huIgG, intact drug, % intact drug, and total ASO in CNS, i.e., cortex, spinal cord, for TfR affinity variants in 24 hours post single dose.

FIG. 34 illustrates huIgG, intact drug, total ASO, and Malat1 knockdown in CNS, i.e., cortex, spinal cord, for TfR affinity variants in 72 hours post final dose in multi-dose study.

FIG. 35 illustrates MCV measurement for TfR variants (A) 24 hours post single dose; (B) 72 hours post final dose in multi-dose study.

FIG. 36 illustrates (A) Tfr degradation (B) Malat1 knockdown (C) ASO concentration of TfR affinity variants.

FIG. 37 illustrates total ASO in brain and periphery in non-human primate studies

FIG. 38 illustrates ASO biodistribution of naked ASO IV v. OTV IV single and multi-dose administration in non-human primates via ASO Immunofluorescence in hemibrain coronal section (neocortex inset).

FIG. 39 illustrates Malat1 knockdown in the CNS and periphery in multi-dose non-human primate studies (A) IV delivered naked ASO; (B) delivered via OTV.

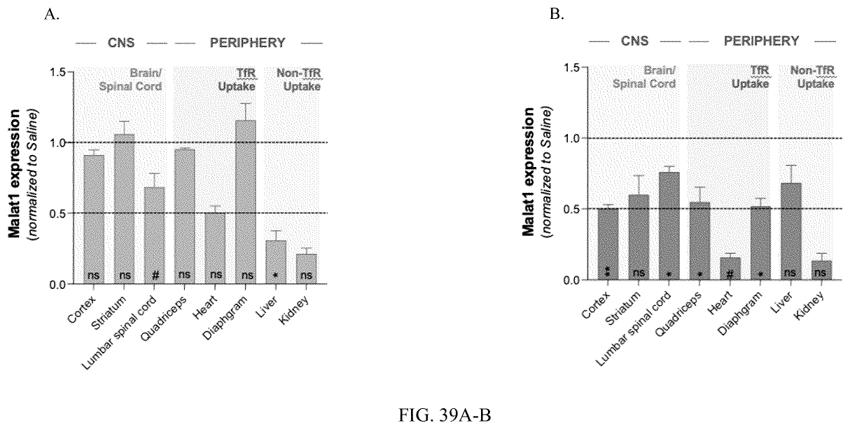

FIG. 40 illustrates ASO biodistribution in brain and lumbar spinal cord ASO Immunofluorescence and ASO level relative to lumbar spinal cord (A) via intrathecal ASO administration; (B) via IV OTV administration.

FIG. 41 illustrates Malat1 knockdown in CNS (frontal cortex, striatum, lumbar spinal cord) and periphery (liver and kidney) in non-human primate studies.

FIG. 42 illustrates Malat1 knockdown across CNS and periphery in mouse single-dose study via (A) IV naked ASO administration; (B) IV OTV administration.

FIG. 43 illustrates ASO PK/PD (A) Total ASO concentration after single dose administration; (B) Total RNA knockdown.

FIG. 44 illustrates ASO PK/PD (A) Total ASO concentration after multi-dose loading window; (B) Total RNA knockdown.

FIG. 45 illustrates (a) huIgG in brain of mice dosed with affinity variants; (B) huIgG ELISA results after brain capillary depletion.

### DETAILED DESCRIPTION

### V. Introduction

Oligonucleotide therapies for CNS disorders caused by genetic abnormalities or increased protein accumulation are becoming an increasingly popular approach to modulate gene expression of such neurological disorders. The blood brain barrier (BBB) represents a challenge to the delivery of systemically administered oligonucleotides to the relevant sites of action within the CNS. Intrathecal (IT) delivery, in which drugs are administered directly into the cerebrospinal fluid (CSF) space, enables the bypass of the BBB. However, one limitation of this approach is that delivery of these oligo therapies directly to the CSF via the IT approach does not achieve uniform distribution throughout the CNS.

Here, we use a human transferrin receptor binding molecule as a way to transport an oligonucleotide across the BBB, termed oligonucleotide transport vehicle (OTV). Intravenously delivered OTV is capable of crossing the BBB, reaching CNS cells, and ultimately providing target knockdown in the brain and spinal cord. Not only is the OTV molecule capable of providing knockdown across CNS regions including across brain regions that include the deep brain regions, as well as the frontal lobe, parietal lobe, temporal lobe, occipital lobe and cerebellum, it is also capable of providing target knockdown across all CNS cell types, including endothelial cells, neurons, astrocytes, oligodendrocytes, and microglia. OTV not only provided equivalent muscle knockdown levels in the periphery as a bivalent molecule, such as an antibody, it resulted in significantly greater levels of knockdown across the brain, spinal cord, and diaphragm. In sum, OTV provides an exciting new potential therapeutic path forward to transport oligonucleotides across the BBB with systemic administration. Additionally, OTV can be used for targeted delivery of oligo therapies to skeletal and cardiac muscle in neuromuscular disorders. This targeted delivery allows for the use of a lower dose of oligo therapies compared with administration of oligo therapies alone, i.e., non-targeted delivery to the periphery.

Described herein are conjugates comprising proteins that bind a transferrin receptor (TfR) and at least one oligonucleotide (e.g., an ASO or an RNAi agent). In particular, provided herein are conjugates of formula I:

P-(L-(X)_{y})ₙ (I)

wherein,
each X is independently an oligonucleotide;
each L is independently a linking group;
P is a protein comprising a TfR binding polypeptide as described herein;
each y is independently at least 1; and
n is at least 1.

In certain embodiments, y is 1 or more, 2 or more, 3 or more, or 4 or more. In certain embodiments, y is 1 or 2. In certain embodiments, y is 1 to 3. In certain embodiments, y is 1 to 4.

In certain embodiments, y is 2 to 4. In certain embodiments, y is 3 or 4. In certain embodiments, y is 1. In certain embodiments y is 2. In certain embodiments, y is 3. In certain embodiments, y is 4.

In certain embodiments, at least one y is 2 or more (e.g., 2 or 4, such as 2). In certain embodiments, at least one L is attached separately to two oligonucleotides. In certain other embodiments, more than oligonucleotide is attached to a single L. For example, the oligonucleotides may be linked to each other in tandem. In certain embodiments, an L is attached at the 5′ end of a first oligonucleotide and a second oligonucleotide is linked to the 3′end of the first oligonucleotide. In certain embodiments, the oligonucleotides may be linked via a nucleic acid linker or a non-oligonucleotide cleavable linker.

In certain embodiments, n is 1 or more; 2 or more; 3 or more; 4 or more; 5 or more; 6 or more; 7 or more; or 8 or more. In certain embodiments, n is 1 to 8, 1 to 7, 1 to 6, 1 to 5, 1 to 4, 1 to 3, or 1 to 2. In certain embodiments, n is 2 to 8, 2 to 7, 2 to 6, 2 to 5, 2 to 4, or 2 to 3. In certain embodiments, n is 1 to 4. In certain embodiments, n is 2 to 4. In certain embodiments, n is 2 to 3. In certain embodiments, n is 1. In certain embodiments, n is 2. In certain embodiments, n is 2. In certain embodiments, n is 3. In certain embodiments, n is 4. In certain embodiments, n is 5. In certain embodiments, n is 6. In certain embodiments, n is 7. In certain embodiments n is 8.

In certain embodiments, the TfR binding protein comprises a constant domain or an Fc polypeptide, wherein certain amino acids have been modified to generate a binding site specific for TfR. Taking advantage of the fact that TfR is highly-expressed on the blood-brain barrier (BBB) and that TfR naturally moves transferrin from the blood into the brain, these proteins can be used to transport oligonucleotides, such as ASOs and RNAi agents, across the BBB. This approach can substantially improve brain uptake and/or biodistribution of these therapeutic agents and is therefore highly useful for treating disorders and diseases where brain delivery is advantageous. TfR is also expressed in certain tissues of the periphery, such as skeletal and cardiac muscle. Therefore, the conjugates described herein may also be used to enhance the delivery of an oligonucleotide to certain peripheral tissues, such as peripheral tissues that express TfR (e.g., skeletal muscle and cardiac muscle).

In some embodiments, the TfR binding protein comprises a CL domain, a CH1 domain, a CH2 domain, and/or a CH3 domain having substitutions in certain sets of amino acids to generate a binding site specific for TfR. In some embodiments, the TfR binding protein comprises a CL domain with one or more substitutions to generate a binding site specific for TfR. In some embodiments, the TfR binding protein comprises a CH1 domain with one or more substitutions to generate a binding site specific for TfR. In some embodiments, the TfR binding protein comprises a CH2 domain with one or more substitutions to generate a binding site specific for TfR. In some embodiments, the TfR binding protein comprises a CH3 domain with one or more substitutions to generate a binding site specific for TfR. Exemplary CH2 and CH3 domains that bind specifically to TfR are described, e.g., in WO 2018/152326, which is incorporated herein by reference in its entirety.

In one aspect, the TfR binding protein comprises a CH3 domain having substitutions in certain sets of amino acids. Thus, in one aspect, the transferrin binding protein comprises multiple substitutions at a set of amino acids (i) 157, 159, 160, 161, 162, 163, 186, 189, and 194; or (ii) 118, 119, 120, 122, 210, 211, 212, and 213 as numbered with reference to SEQ ID NO:1. Anywhere from four to all of the amino acid positions of a set may be substituted. For purposes of this disclosure, a substitution is determined with reference to SEQ ID NO:1, unless stated otherwise. Thus, an amino acid is considered to be a substitution if it differs from the corresponding amino acid in position SEQ ID NO:1 even if the amino acid is present at that position in a naturally occurring CH3 domain protein.

In a further aspect, provided herein are treatment methods and methods of using a conjugate as described herein to target an oligonucleotide (e.g., ASO or an RNAi agent) to transferrin receptor-expressing cells, e.g., to deliver the oligonucleotide to that cell, or to deliver the molecule across an endothelium such as the blood-brain barrier.

### VI. Definitions

As used herein, the singular forms “a,” “an,” and “the” include plural referents unless the content clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “halo” is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₆ means one to six carbons). Examples include (C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkoxy” refers to an alkyl groups attached to the remainder of the molecule via an oxygen atom (“oxy”).

The term “alkylthio” refers to an alkyl groups attached to the remainder of the molecule via a thio group.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

The term “aryloxy” refers to an aryl group attached to the remainder of the molecule via an oxygen atom (Aryl-O—).

The term “heteroaryloxy” refers to a heteroaryl group attached to the remainder of the molecule via an oxygen atom (Heteroaryl-O—).

As used herein, the term “heteroatom” includes oxygen (O), nitrogen (N), sulfur (S) and silicon (Si).

The term “cycloalkyl” refers to a saturated or partially unsaturated (non-aromatic) all carbon ring having 3 to 6 carbon atoms (i.e., (C₃-C₆)carbocycle). Non-limiting examples of cycloalkyls include cyclopropyl, cyclobutyl, cyclopentyl, and cyclohexyl.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-12 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-12 membered monocyclic or bicyclic heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, 1,4-dioxane and

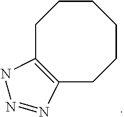 [SMILES: C1CCCc2[nH]nnc2CC1]

In one embodiment the heterocycle can be di-valent, i.e., attached to the remainder of the molecule or the linking group at two positions of the heterocycle (-heterocycle-). In one embodiment, the heterocycle is substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), and carboxy.

As used herein a wavy line “ [symbol] ” that intersects a bond in a chemical structure indicates the point of attachment of the bond that the wavy bond intersects in the chemical structure to the remainder of a molecule.

A “transferrin receptor” or “TfR” as used herein refers to transferrin receptor protein 1. The human transferrin receptor 1 polypeptide sequence is set forth in SEQ ID NO:235. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:107.

A “new binding site” or “non-native binding site” refers to the site on the variant protein that specifically recognizes and binds an antigen, such as transferrin receptor protein, wherein the unmodified or native protein does not specifically bind to the antigen. For example, a non-native antigen binding site may be introduced by substitution, deletion and/or insertion of amino acids into the native protein sequence resulting in the specific recognition and binding at the site of mutation.

As used herein, the term “constant domain” refers to a light chain constant region domain polypeptide (CL) and CH1, CH2 and CH3 domain polypeptides from the heavy chain.

The terms “CH1 domain”, “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, a CH2 domain polypeptide refers to the segment of amino acids from about position 231 to about position 340 as numbered according to the EU numbering scheme, and a CH1 domain polypeptide refers to the segment of amino acids from about position 118 to about position 215 according to the EU numbering scheme. CH1, CH2 and CH3 domain polypeptides may also be numbered by the IMGT (ImMunoGeneTics) numbering scheme in which the CH1 domain numbering is 1-98, the CH2 domain numbering is 1-110 and the CH3 domain numbering is 1-107, according to the IMGT Scientific chart numbering (IMGT website). CH2 and CH3 domains are part of the Fc polypeptide of an immunoglobulin. In the context of IgG antibodies, an Fc polypeptide refers to the segment of amino acids from about position 231 to about position 447 as numbered according to the EU numbering scheme.

As used herein, the term “Fc polypeptide” refers to the C-terminal region of a naturally occurring immunoglobulin heavy chain polypeptide that is characterized by an Ig fold as a structural domain. An Fc polypeptide typically contains constant region sequences including at least the CH2 domain and/or the CH3 domain and may contain at least part of the hinge region. Illustrative hinge region sequences, or portions thereof, are set forth in SEQ ID NOS:232-234.

As used herein, the term “CL domain” refers to the immunoglobulin constant domain of the light chain. In the context of IgG antibodies, a kappa CL domain polypeptide refers to the segment of amino acids from about position 108 to about position 214 as numbered according to the EU numbering scheme. Alternatively, the kappa and lambda CL domains may be numbered by the IMGT (ImMunoGeneTics) numbering scheme in which the kappa CL domain numbering is 1-107, and the lambda CL domain numbering is 1-106, according to the IMGT Scientific chart numbering (IMGT website).

As used herein, the term “Fab” or “Fab fragment” refers to a monovalent fragment consisting of a VL, VH, CL and CH1 domain. A Fab or Fab fragment may or may not contain all or part of an antibody hinge region.

As used herein, the term “non-targeting Fab fragment” or “NTF” refers to a Fab fragment that does not specifically bind to an antigen via its heavy or light chain variable domains or does not specifically bind to an antigen expressed in a given mammal, such as a primates, e.g., human and non-human primates, or rodents, e.g., mouse, or in a particular tissue within such a mammal via its heavy or light chain variable domains. In certain embodiments, a Fab for use in a Fab-Fc fusion or Fab-Fc dimer fusion as described herein does not specifically bind to transferrin via its heavy or light chain variable domains. Non-limiting examples of non-targeting Fab fragments include (a) RSV (palivizumab) Fab fragments, which are non-targeting in mice and non-human primates, and (b) Fab fragments to dinitrophenyl hapten (DNP) (See Leahy, PNAS 3661-3665, 1988).

The terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

As used herein, the term “modified site” refers to a particular position within a polypeptide that comprises a mutation or variant, with respect to a corresponding wild-type polypeptide (e.g., a wild-type CL, CH1, CH2 or CH3 domain). In certain embodiments, the mutation or variant is non-naturally occurring. A modified site may include, e.g., an insertion or a substitution. As used herein, the term “substitution” refers to an alteration that replaces an amino acid with another amino acid. For example, a “cysteine substitution” refers to the replacement of an amino acid with a cysteine.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln.

In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide or protein sequence, refers to the position of the residue of a specified reference sequence when the given amino acid sequence is maximally aligned and compared to the reference sequence. Thus, for example, an amino acid residue in a polypeptide “corresponds to” an amino acid in the region of SEQ ID NO:1 from amino acids 114-220 when the residue aligns with the amino acid in SEQ ID NO:1 when optimally aligned to SEQ ID NO:1. The polypeptide that is aligned to the reference sequence need not be the same length as the reference sequence.

A “binding affinity” as used herein refers to the strength of the non-covalent interaction between two molecules, e.g., a single binding site on a polypeptide/protein and a target, e.g., transferrin receptor, to which it binds. Thus, for example, the term may refer to 1:1 interactions between a polypeptide/protein and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide/protein and its target, but also apparent affinities for which K_{D}'s are calculated that may reflect avid binding. The term “low affinity” refers to an affinity that is sufficient for targeting as well as release. In embodiments, a low affinity has value of about 600 nM to about 3 nM, about 500 nM to about 3 nM, or about 400 nM to about 20 nM, or about 300 nM to about 30 nM, or about 200 nM to about 40 nM, or about 150 nM to about 50 nM or about 3 nM, 4 nM, 5 nM, 5 nM, 7 nM, 8 nM, 9 nM, 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, 400 nM, 410 nM, 420 nM, 430 nM, 440 nM, 450 nM, 460 nM, 470 nM, 480 nM, 490 nM, 500 nM.

The phrase “specifically binds” or “selectively binds” to a target, e.g., transferrin receptor, when referring to a protein comprising a modified constant domain as described herein, refers to a binding reaction whereby the protein binds to the target with greater affinity, greater avidity, and/or greater duration than it binds to a structurally different target, e.g., a target not in the transferrin receptor family. In typical embodiments, the protein has at least 5-fold, 10-fold, 100-fold, 1000-fold, 10,000-fold or greater affinity for a transferrin receptor compared to an unrelated target when assayed under the same affinity assay conditions. In some embodiments, a modified CH3 domain specifically binds to an epitope on a transferrin receptor that is conserved among species, e.g., conserved between non-human primate and human species. In some embodiments, a protein may bind exclusively to a human transferrin receptor.

As used herein, the term “nucleic acid” and “polynucleotide” refers to deoxyribonucleotides or ribonucleotides and polymers thereof in either single- or double-stranded form, composed of monomers (nucleotides) containing a sugar moiety, phosphate and a nucleobase. Unless specifically limited, the term encompasses both modified and unmodified nucleic acids.

As used herein, the term “nucleobase” refers to nitrogen-containing compounds that can be linked to a sugar moiety to form nucleosides, which in turn are components of nucleotides. The ability of nucleobases to form base pairs and to stack one upon another leads directly to long-chain helical structures such as ribonucleic acid (RNA) and deoxyribonucleic acid (DNA). Nucleobases may be naturally occurring (i.e., adenine (A), cytosine (C), guanine (G), thymine (T), and uracil (U)) or modified.

As used herein, the term “nucleoside” refers to a compound comprising a nucleobase and sugar moiety (e.g., deoxyribose or ribose, or a modified variant thereof). The term nucleoside includes both modified and unmodified nucleosides.

As used herein, the term “nucleotide” refers to a compound comprising a nucleobase, a sugar moiety, and one or more phosphate groups. The term nucleotide includes both modified and unmodified nucleotides.

As used herein, the term “internucleoside linkage” means the covalent linkages between two nucleosides in an oligonucleotide. Nucleosides may be linked via natural (i.e., a phophodiester (PO) linkage) or modified linkages.

The terms “chemical modification”, “modification” or “modified” may refer to a chemical change in a compound when compared to its naturally occurring counterpart. For example, a nucleobase, a sugar moiety or an internucleoside linkage may be chemically modified.

The terms “nucleotide sequence” and “nucleic acid sequence” and “nucleic acid strand” refer to a sequence of bases (purines and/or pyrimidines, or synthetic derivatives thereof) in a polymer of DNA or RNA, which can be single-stranded or double-stranded, optionally containing synthetic, non-natural or altered nucleotides capable of incorporation into DNA or RNA polymers, and/or backbone modifications (e.g., a modified oligomer). The terms “oligo”, “oligonucleotide” and “oligomer” may be used interchangeably and refer to such sequences of purines and/or pyrimidines. For example, the oligonucleotide may comprise chemically modified or unmodified nucleic acid molecules (RNA or DNA) having a length of less than about, e.g., about 200 nucleotides (for example, less than about 100 or 50 nucleotides). The oligonucleotide can, e.g., be single stranded DNA or RNA (e.g., an ASO); double stranded DNA or RNA (e.g., small interfering RNA (siRNA)), including double stranded DNA or RNA having a hairpin loop; or DNA/RNA hybrids. In one embodiment, the oligonucleotide has a length ranging from about 5 to about 60 nucleotides, or about 10 to about 50 nucleotides. In another embodiment, the oligonucleotide has a length ranging from about 5 to about 30 nucleotides or from about 15 to about 30 nucleotides. In yet another embodiment, the oligonucleotide has a length ranging from about 18 to about 24 nucleotides.

The terms “modified oligos”, “modified oligonucleotides” or “modified oligomers” may be similarly used interchangeably, and refer to such sequences that contain synthetic, non-natural or altered bases, sugars and/or backbone modifications.

The oligonucleotides described herein may be synthesized using standard solid or solution phase synthesis techniques that are known in the art. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

The terms “identical” or percent “identity,” in the context of two or more nucleotide sequences, refer to two or more sequences or subsequences that are the same or have a specified percentage of nucleotides, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of oligonucleotides (e.g., to determine identity or complementarity), typically one nucleotide sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art.

As used herein, “hybridize” or “hybridization” means the pairing of complementary nucleotide sequences (e.g., an antisense compound and its target nucleic acid; or between antisense and sense strands). As used herein, “specifically hybridizes” means the ability of a reference nucleic acid to hybridize to one nucleic acid molecule with greater affinity than it hybridizes to another.

“Expression” refers to the transcription and/or translation of an endogenous gene, heterologous gene or nucleic acid segment, or a transgene in cells. For example, expression may refer to the transcription and stable accumulation of sense (mRNA) or functional RNA. Expression may also refer to the production of protein.

The term “gene” refers to a nucleic acid (e.g., DNA or RNA) sequence that comprises coding sequences necessary for the production of a polypeptide or precursor.

The phrase “modulating the expression of a target gene or sequence” means a change (e.g., an increase or decrease) in expression of the target gene or sequence (e.g., via degradation of the target or translation inhibition). For example, it includes inhibiting, reducing or decreasing the expression of a target gene or sequence. This also includes a change in alternative splicing, which may result in a change in the absolute or relative amount of a particular splice variant.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of an injury, disease, or condition, including any objective or subjective parameter such as abatement, remission, improvement in patient survival, increase in survival time or rate, diminishing of symptoms or making the injury, disease, or condition more tolerable to the patient, slowing in the rate of degeneration or decline, or improving a patient's physical or mental well-being. Additionally, “treating” or “treatment” may refer to the modulation of the target gene expression such as gene knockdown or gene knockout. For instance, the expression of the target gene or sequence is inhibited or reduced, e.g., by at least about 10%, 20%, 30%, 40%, 50%, 60%, 70%, 80%, 90%, 95%, 99% or 100%, as compared to the expression in a control. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount” or “therapeutically effective amount” of an agent is an amount of the agent that treats, alleviates, abates, or reduces the severity of symptoms of a disease in a subject. A “therapeutic amount” or “therapeutically effective amount” of an agent may improve patient survival, increase survival time or rate, diminish symptoms, make an injury, disease, or condition more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the proteins described herein are administered intravenously.

The term “MCV volume” is a standard measure and refers herein to an indicator of conjugate tolerance.

### VII. Variable “X”: Oligonucleotides

As described herein, one or more oligonucleotides (e.g., ASOs or RNAi agents) may be linked through “L” to a transferrin receptor binding protein as described herein to form a conjugate.

While the length of the oligonucleotide may vary, in certain embodiments, the oligonucleotide is from about 10 to about 60 nucleotides in length, or from about 10 to about 30 nucleotides in length, or from about 18 to about 30 nucleotides in length or from about 15 to about 25 nucleotides in length, or from about 16 to about 20 nucleotides in length. Additionally, as described below, an oligonucleotide may comprise certain chemical modifications, such as a modified internucleoside linkage, a modified nucleobase, a modified sugar, or a combination thereof. In certain embodiments, one or more oligonucleotides are linked (i.e., through a linking group “L”) to the transferrin receptor binding protein. In certain embodiments, two or more oligonucleotides are linked to the transferrin receptor binding protein (e.g., 1, 2, 3, 4, 5, 6, 7 or 8 or more). In certain embodiments, one oligonucleotide is linked to the transferrin receptor binding protein. In certain embodiments, two oligonucleotides are linked to the transferrin receptor binding protein. In certain embodiments, four oligonucleotides are linked to the transferrin receptor binding protein.

In certain embodiments, 1 oligonucleotide is attached to a single linking group (L). In certain embodiments, 2 oligonucleotides are attached to a single linking group (L). For example, the oligonucleotides may be linked to each other in tandem. In certain embodiments, an L is attached at the 5′ end of a first oligonucleotide and a second oligonucleotide is linked to the 3′end of the first oligonucleotide. In certain embodiments, the oligonucleotides may be linked via a nucleic acid linker or a non-oligonucleotide cleavable linker.

In other embodiments, the linking group is a branched linking group and 2 or more oligonucleotides are attached separately to a single linking group (L) (i.e., y is 2 or more).

When two or more oligonucleotides are attached to the TfR binding protein, the oligonucleotides may be the same or different. In certain embodiments, the oligonucleotides are the same.

### ASOs

In one embodiment, each oligonucleotide is independently an ASO. The term “antisense oligonucleotide (ASO)” refers to single strands of DNA-like or RNA-like molecules (e.g., modified nucleotides such as those described herein) that are complementary or partially complementary to a chosen target polynucleotide sequence, e.g., an mRNA. By binding to a complementary target sequence ASOs can alter or modulate gene expression through a number of mechanisms, including, e.g., by altering splicing (exon exclusion or exon inclusion); by recruiting RNase H leading to target degradation; through translation inhibition; and by small RNA inhibition.

Typically, ASOs range from about 10 to 30 base pairs (bp) in length, but may be longer or shorter. For example, in certain embodiments, the ASO is about 10 to about 60 nucleotides in length, or about 10 to about 50 nucleotides in length, or about 10 to about 40 nucleotides in length. In certain embodiments, the ASO is about 10 to 30 nucleotides in length, or about 12 to 30 nucleotides in length, or about 14 to about 30 nucleotides in length, or about 15 to about 30 nucleotides in length, or about 16 to about 30 nucleotides in length, or about 17 to about 30 nucleotides in length, or about 18 to about 30 nucleotides in length, or about 18 to about 28 nucleotides in length or about 18 to 26 nucleotides in length, or about 18 to about 24 nucleotides in length, or about 15 to about 25 nucleotides in length, or about 16 to about 20 nucleotides in length. In certain embodiments, the ASO is about 15, 16, 17, 18, 19, 20, 21, 22, 23, 24, 25, 26, 27, 28, 29 or 30 nucleotides in length.

Selection of antisense oligonucleotide sequences specific for a given target sequence is based upon analysis of the chosen target sequence and determination of a number of factors, including secondary structure, Tm, binding energy, and relative stability. Additionally, antisense oligonucleotides may be selected based upon their relative inability to form dimers, hairpins, or other secondary structures that would reduce or prohibit specific binding to the target mRNA in a host cell. Target regions of the mRNA include those regions at or near the AUG translation initiation codon and those sequences that are substantially complementary to 5′ regions of the mRNA. Secondary structure analyses and target site selection considerations can be performed using software and algorithms known in the art, for example, using v.4 of the OLIGO primer analysis software (Molecular Biology Insights) and/or the BLASTN 2.0.5 algorithm software (Altschul et al, Nucleic Acids Res. 1997, 25(17):3389-402).

### RNAi Agents

In certain other embodiments, each oligonucleotide is independently an RNAi agent (e.g., a siRNA or shRNA). The term “RNA interference (RNAi) agent” refers to an RNA agent, or a molecule that can be cleaved into an RNA agent, that can inhibit the expression of a target gene or sequence (e.g., an mRNA, tRNA or viral RNA), in a sequence specific manner (e.g., via Dicer/RISC). RNAi agents may be single or double stranded. If the RNAi agent is a single strand it can include a 5′ modification, such as one or more phosphate groups or one or more analogs of a phosphate group. In one embodiment, the RNAi agent is double stranded and comprises a sense and an antisense strand (e.g., a short interfering RNA (siRNA)).

The RNAi agent typically includes a region of sufficient homology to the target gene, and is of sufficient length, such that the RNAi agent can mediate down regulation of the target gene. Complementarity between the RNAi agent and the target sequence should be sufficient to enable the RNAi agent, or a cleavage product thereof, to direct sequence specific silencing. In certain embodiments, the RNAi agent is, or comprises a region which is, at least partially complementary to the target RNA. In certain other embodiments, the RNAi agent is, or comprises a region which is, fully complementary to the target RNA.

In some embodiments, the RNAi agent comprises an unpaired region at one or both ends of the molecule. For example, a double stranded RNAi agent may have its strands paired with an overhang, e.g., 5′ and/or 3′ overhangs, such as an overhang of 1-3 nucleotides. In certain embodiments, an RNAi agent will comprise an unpaired overhang of 1, 2, 3 or 4 nucleotides in length at each end. The overhangs can be the result of one strand being longer than the other, or the result of two strands of the same length being staggered.

Duplexed regions within an RNAi agent may vary in length, but typically range between about 5 to about 30 nucleotides in length. In certain embodiments, the duplexed regions are between about 15-60, or about 15-50, or about 15-40, or about 15-30, or about 15-25, or about 19-25 nucleotides in length. In certain embodiments, the duplexed regions are between about 20-24, or about 21-23 nucleotides in length. In certain embodiments, the duplexed regions are about 5, 6, 7, 8, 9, 10, 11, 12, 13, 14, 15, 16, 17, 18, 19, 20, 21, 22, 23, 24 or more nucleotides in length.

A “single strand RNAi agent” or “ssRNAi agent” as used herein is made up of a single molecule. It may include a duplexed region, formed by intra-strand pairing, e.g., it may be, or include, a hairpin or pan-handle structure. Single strand RNAi agents may be antisense with regard to the target molecule. A single strand RNAi agent may be sufficiently long that it can enter RISC and participate in RISC mediated cleavage of a target mRNA. In certain embodiments, a single strand RNAi agent is at least 10, 15, 20, 25, 30, 35, 40, or 50 nucleotides in length. In certain embodiments, it is less than 200, 100, 80 or 60 nucleotides in length.

Small hairpin RNA (shRNA) agents typically have a duplex region less than 200, 100, or 50, in length. In certain embodiments, the duplex region ranges in length from about 15-60, or about 15-50, or about 15-40, or about 15-30, or about 15-25, or about 19-25 nucleotides in length. In certain embodiments, the duplexed regions are between about 17-23, or from about 19-23, or from about 20-23, or about 21-23, or about 19 to 21 nucleotides in length. In certain embodiments, the duplex region is at least about 17, 18, 19, 29, 21, 22, 23, 24, or 25 nucleotide pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded RNAi agent” or “dsRNAi agent” as used herein, includes more than one strand in which interchain hybridization can form a duplex region within the molecule (e.g., hybridization between a sense strand and an antisense strand). In certain embodiments, the RNAi agent is sufficiently large that it can be cleaved by an endogenous molecule, such as Dicer, to produce smaller molecules.

In certain embodiments, the RNAi agent is an siRNA molecule comprising sense and an antisense strands.

As used herein, term “antisense strand” refers to the strand of an RNAi agent that is sufficiently complementary to a target polynucleotide, e.g. a target mRNA. In certain embodiments, the antisense strand of a double stranded RNAi agent is at least about 10, 11, 12, 13, 14, 15, 16 17, 18, 19, 20, 21, 22, 23, 24, 25, 30, 40, 50 or 60 nucleotides in length. In certain embodiments, the antisense strand of a double stranded RNAi agent is less than about 200, 100, or 50, nucleotides in length. In certain embodiments, the antisense strand ranges in length from about 17 to 25, or about 19 to 23, or about 19 to 21 nucleotides in length.

As used herein, term “sense strand” refers to the strand of an RNAi agent that is sufficiently complementary to the antisense strand. In certain embodiments, the sense strand of a double stranded RNAi agent is at least about 10, 11, 12, 13, 14, 15, 16 17, 18, 19, 20, 21, 22, 23, 24, 25, 30, 40, 50 or 60 nucleotides in length. In certain embodiments, the sense strand of a double stranded RNAi agent is less than about 200, 100, or 50, nucleotides in length. In certain embodiments, the sense strand ranges in length from about 17 to 25, or about 19 to 23, or about 19 to 21 nucleotides in length.

In certain embodiments, the double strand portion of a double stranded RNAi agent is at least about 10, 11, 12, 13, 14, 15, 16, 17, 18, 19, 20, 21, 22, 23, 24, 25, 30, 40, 50, or 60 nucleotides in length. In certain embodiments, the sense strand of a double stranded RNAi agent is less than about 200, 100, or 50, nucleotides in length. In certain embodiments, the sense strand ranges in length from about 17 to 25, or about 19 to 23, or about 19 to 21 nucleotides in length.

In certain embodiments, the sense and antisense strands may be chosen such that the dsRNAi agent includes an unpaired region at one or both ends of the molecule. Thus, a dsRNAi agent may contain sense and antisense strands, paired to contain an overhang, e.g., 5′ and/or 3′ overhangs of between 1, 2, 3 or 4 nucleotides in length. The overhangs can be the result of one strand being longer than the other, or the result of two strands of the same length being staggered. In certain embodiments, the dsRNAi agent comprises at least one 3′ overhang. In certain embodiments, both ends of the dsRNAi agent comprise a 3′ overhang (e.g., of 2 nucleotides in length).

Duplexed regions within a dsRNAi agent may vary in length, but typically range between about 5 to about 30 nucleotides in length. In certain embodiments, the duplex region ranges in length from about 5-60, or about 15-60 or about 15-50, or about 15-40, or about 15-30, or about 15-25, or about 19-25 nucleotides in length. In certain embodiments, the duplexed regions are between about 17-23, or from about 19-23, or from about 20-23, or about 21-23, or about 19 to 21 nucleotides in length. In certain embodiments, the duplexed regions are about 5, 6, 7, 8, 9, 10, 11, 12, 13, 14, 15, 16, 17, 18, 19, 20, 21, 22, 23, 24 or more nucleotides in length.

Methods of producing RNAi agents, such as siRNA and shRNA, are known in the art and can be readily adapted to produce an RNAi agent that targets any polynucleotide sequence. In certain embodiments, an RNAi agent is chemically synthesized. For example, oligonucleotides can be synthesized using a variety of techniques, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997).

### Illustrative Oligonucleotide Modifications

In certain embodiments, an oligonucleotide described herein may comprise at least one nucleic acid modification, such as those selected from the group consisting of a modified internucleoside linkage, a modified nucleobase, a modified sugar, and combinations thereof. Such modifications may be used to alter pharmacokinetics (improved nuclease resistance resulting in a longer half-life), pharmacodynamics (superior affinity for the target RNA), or endocytic uptake. However, many modifications preclude cleavage by RNase H, which is the desired mechanism of action for many ASOs. Thus, certain RNase H ASOs may be designed as chimeras, where different bases are a mix of different chemistries, or as gapmers, where some modifications are placed on the “wings” and not the central bases. In contrast, for RNAi agents and ASOs intended to alter mRNA splicing or translation, considerations regarding RNase H are not necessary.

Accordingly, an oligonucleotide described herein may comprise one or more nucleic acid modifications. In certain embodiments, an oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 or more modifications.

In certain embodiments, an oligonucleotide described herein comprises one or more nucleotide modifications (e.g., to the nucleobase or sugar moiety). In certain embodiments, 25% or more of the nucleotides present in the oligonucleotide are modified. In certain embodiments, 50% or more of the nucleotides present in the oligonucleotide are modified. In certain embodiments, 75% or more of the nucleotides present in the oligonucleotide are modified. In certain embodiments, 100% of the nucleotides present in the oligonucleotide are modified.

In certain embodiments, the oligonucleotide comprises one or more nucleobase modifications. In certain embodiments, the oligonucleotide comprises one or more modifications to the sugar moiety (e.g., furanosyls comprising substituents at the 2′-position, the 3′-position, the 4′-position and/or the 5′-position). In certain embodiments, substituted sugar moieties include bicyclic sugar moieties.

In certain embodiments, the nucleic acid modifications with the oligonucleotide are included in a pattern. In certain embodiments, the oligonucleotide is a gapmer. The modification pattern of a gapmer oligonucleotide generally has the formula 5′-Xa-Ya-Za-3′, with Xa and Za as flanking regions around a gap region Ya. In certain embodiments, the Ya region is a contiguous stretch of nucleotides, e.g., a region of at least 6 DNA nucleotides, which are capable of recruiting an RNAse, such as RNAse H. In certain embodiments, the Ya region is at least 8 DNA nucleotides. In certain embodiments, the gapmer binds to the target nucleic acid, at which point an RNAse is recruited and can then cleave the target nucleic acid. In certain embodiments, the Ya region is flanked both 5′ and 3′ by regions Xa and Za, which comprise high-affinity modified nucleotides, e.g., one to six modified nucleotides in each of Xa and Za. In certain embodiments, the modified nucleotides are present in the 5′ and 3′ regions of the oligonucleotide, while certain modified nucleotides and/or modified linkages may or may not present in the central portion of the molecule. In certain embodiments, the modified nucleotides are present in the 5′ and 3′ regions of the oligonucleotide and certain modified nucleotides are not present in the central portion of the molecule (e.g., LNA residues are not present in the central portion; however, the central region may contain modified linkages, such as PS linkages. In certain embodiments, Xa and Za each comprise 3 modified nucleotides. In certain embodiments, the 3 modified nucleotides are arranged in tandem in each of Xa and Za.

Modified nucleosides/nucleotides are known in the art and include, but are not limited to, 2′-O methyl (2′OMe) residues, 2′ O-methoxyethyl (MOE) residues, constrained nucleic acid residues (e.g., S-cEt, R-cEt, S-cMOE, and R-cMOE), peptide nucleic acid (PNA) residues, locked nucleic acid (LNA) residues, and 5′-methylcytidine residues (methylated cytosine residues) (see, also, Scoles, et al., Neurol Genet April 2019, 5 (2) e323). In certain embodiments, the oligonucleotide comprises one or more MOE residues. In certain embodiments, the oligonucleotide comprises one or more OMe residues or F residues (e.g., 2′-F or 2′OMe). In certain embodiments, the oligonucleotide comprises one or more constrained (e.g., S-cEt, R-cEt, S-cMOE, and R-cMOE) and/or LNA residues. Nucleic acids are considered “locked” when they have a methylene bridge connection made between 2′-oxygen and the 4′-carbon of the ribose sugar molecule. In certain embodiments, the oligonucleotide is a morpholino (i.e., comprises certain modifications to the sugar moiety). In certain embodiments, an oligonucleotide described herein comprises one or more LNA residues and one or more 5′-methylcytidine residues.

In certain embodiments, the oligonucleotide comprises one or more modifications to the internucleoside backbone (i.e., the natural phosphodiester (PO) linkage is modified). In certain embodiments, such modifications are made to, e.g., reduce nuclease activity. Thus, in certain embodiments, an oligonucleotide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more modified internucleoside linkages. In certain embodiments, 25% or more of the internucleoside linkages are modified. In certain embodiments, 50% or more of the internucleoside linkages are modified. In certain embodiments, 75% or more of the internucleoside linkages are modified. In certain embodiments, 100% of the internucleoside linkages present in the oligonucleotide are modified.

Backbone modifications are known in the art and include, but are not limited, to, e.g., phosphorothioate linkages, phosphoroamidate linkages, and phosphorodiamidate linkages. For example, in certain embodiments, one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) of the internucleoside linkages in the oligonucleotide are replaced with a phosphorothioate (PS) linkage. In certain embodiments, the oligonucleotide comprises a mix of PO and PS linkages. In certain embodiments, the oligonucleotide comprises only PS linkages. In certain other embodiments, one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) of the internucleoside linkages in the oligonucleotide are replaced with a phosphorodiamidate linkage. In certain embodiments, the oligonucleotide is a phosphorodiamidate morpholino (PMO).

In certain embodiments, the internucleoside linkages are stereorandom with regard to the chiral centers (Rp and Sp). In certain other embodiments, the Rp and Sp configurations in the oligonucleotide are optimized in particular configurations.

In certain embodiments, the oligonucleotide is a gapmer comprising LNA and PS modifications. For example, in certain embodiments, the oligonucleotide is a gapmer having a modification pattern of the formula 5′-Xa-Ya-Za-3′, with Xa and Za as flanking regions around a gap region Ya, wherein Xa and Za each comprise 3 LNA modified nucleotides (e.g., 3 consecutive LNA modified nucleotides), and wherein the gap region Ya comprises PS linkages. In certain embodiments, the oligonucleotide further comprises one or more 5′-methylcytidine residues. In certain embodiments, the gap region Ya does not comprise LNA residues.

### VIII. Variable “L”: Linking Group

In certain embodiments, L is a linking group that joins each oligonucleotide (X) to a TfR binding protein (P). The linking group may be any group suitable for joining an oligonucleotide to the TfR binding protein.

The linking group may be attached to any region of the TfR binding protein comprising the TfR binding polypeptide, (e.g., to the N-terminal region, to the C-terminal region, or to an amino acid within the protein, such as a cysteine residue or a glutamine residue), so long as the oligonucleotide does not prevent binding of the TfR binding protein to the transferrin receptor. Similarly, the linking group may be attached to any region of the oligonucleotide (e.g., the 5′ end, the 3′end or to a nucleic acid residue within the molecule), so long as the TfR binding protein does not interfere with the functionality of the oligonucleotide (e.g., complementary binding to a target nucleic acid). For example, the linker may be attached to the oligonucleotide through any number of synthetically feasible points located throughout the oligo, such as at the 3′ or 5′ terminal residues of the oligo; at a sugar moiety; at a base moiety; or at a residue located within the backbone.

In certain embodiments, the linker is attached to the oligonucleotide at the 5′ terminal residue of the oligonucleotide. In certain embodiments, the linker is attached to the oligonucleotide at the 3′ terminal residue of the oligonucleotide. In certain embodiments, the linker is attached to the oligonucleotide at a residue within the oligonucleotide. In certain embodiments, the oligonucleotide is a double stranded RNAi molecule, wherein the linker is attached to the sense strand (e.g., at the 5′ or 3′ terminal residue). In certain embodiments, the oligonucleotide is a double stranded RNAi molecule, wherein the linker is attached to the antisense strand (e.g., at the 5′ or 3′ terminal residue). In certain embodiments, the oligonucleotide is siRNA, wherein the linker is attached to the 3′ end of the sense strand. In certain embodiments, the 3′ end of the sense strand of the siRNA is modified with a C6 amine.

In certain embodiments, the linking group comprises spacers. In certain embodiments, the spacers are hydrophilic spacers. In certain embodiments, the hydrophilic spacers are polyethylene glycol (PEG).

In certain embodiments, the linking group is a homobifuctional linker or a heterobifunctional linker.

In some embodiments, the linking group is cleavable (e.g., a nuclease-cleavable linker, an acid-labile linker, peptidase-sensitive linker, photolabile linker, dimethyl linker or disulfide-containing linker (Chari et al., Cancer Res. 52:127-131 (1992); U.S. Pat. No. 5,208,020). In certain embodiments, the linking group comprises one or more nucleotides (e.g., 1, 2, 3, or more) or one or more nucleosides (e.g., 1, 2, 3, or more). In certain embodiments, the one or more nucleotides or one or more nucleosides are unmodified. In certain embodiments, the linking group comprises one or more nucleotides having unmodified bases, unmodified sugar groups and/or unmodified phosphate groups. In certain embodiments, the linking group comprises one or more nucleosides having unmodified bases and/or unmodified sugar groups. In certain embodiments, the linking group comprises TCA, a nuclease linker. In certain embodiments, TCA is modified with a C6 amine at T position. In certain embodiments, the linking group does not comprise TCA. In certain embodiments, the linking group is enzymatically cleavable. In certain embodiments, the linking group is cleavable by an enzyme present in the central nervous system or muscle. In certain embodiments, a cleavable linking group is selected for conjugates comprising ASOs (e.g., to enable the ASO to dissociate from the remainder of the conjugate for transport into the nucleus). In certain embodiments, the cleavable linking group is a cleavable dipeptide linker. In certain embodiments, the cleavable dipeptide linker is a val-cit cleavable linking group or val-ala cleavable linker. In certain embodiments, the cleavable linking group is an acid cleavable linker. In certain embodiments, the acid cleavable linker is a carbonate linker or a hydrazone linker. In certain embodiments, the cleavable linking group comprises PEG spacers. In certain embodiments, the cleavable linking group is a disulfide such as SPDP (succinimidyl 3-(2-pyridyldithio)propionate) or lys-conjugated acid-cleavable hydrazide.

In certain embodiments, the linking group is a non-cleavable linking group. In certain embodiments, the linking group is a covalent linking group. In certain embodiments, the covalent linking group is derivable from an APN or an acrylamide. In certain embodiments, the covalent linking group comprises a group —CH₂CH₂C(═O)—. In certain embodiments, the covalent linking group comprises a group:

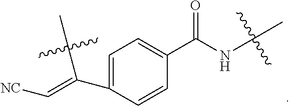 [SMILES: *NC(=O)c1ccc(/C(*)=C/C#N)cc1]

In certain embodiments, the covalent linking group is derivable from a haloacetamide, e.g., bromoacetamide, chloroacetamide, iodoacetamide.

In certain embodiments, the linking group comprises a C₆ amine group having the formula —(CH₂)₆—NH—.

In certain embodiments, the linking group is derivable from a maleimide. For example, in certain embodiments, the linking group comprises a group:

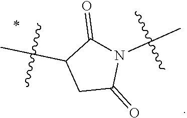 [SMILES: *C1CC(=O)N(*)C1=O]

In certain embodiments, the linking group may be attached to P at the valence marked * (e.g., to a sulfur atom of a modified site within P).

In certain embodiments, the maleimide is a modified maleimide. In certain embodiments, the modified maleimide is an alkyl-, aryl-, cycloalkyl-, or exocyclic-maleimide. In certain embodiments, the linking group comprises a protected maleimide. For example, in certain embodiments, the linking group comprises a protected maleimide of formula:

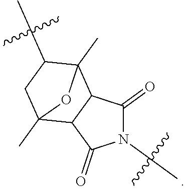 [SMILES: *C1CC2(C)OC1(C)C1C(=O)N(*)C(=O)C12]

In certain embodiments, the protected maleimide is removed after bioconjugation.

In certain embodiments, the linking group is a self-hydrolyzing linking group.

Certain specific, non-limiting embodiments (abbreviated as Linker Embodiments LE1-LE42) are described below.

In Linker Embodiments LE1, the linking group has a molecular weight of from about 20 daltons to about 5,000 daltons. In In Linker Embodiments LE2, the linking group has a molecular weight of from about 20 daltons to about 1,000 daltons. In Linker Embodiments LE3, the linking group has a molecular weight of from about 20 daltons to about 200 daltons. In Linker Embodiments LE4, the linking group has a length of about 5 angstroms to about 60 angstroms.

In Linker Embodiments LE5, the linking group separates the peptide from the remainder of the conjugate of formula I by about 5 angstroms to about 40 angstroms, inclusive, in length.

In Linker Embodiments LE6, the linking group is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), (—S), an amino acid, a hydrazone (—C(R′)═N═N(R′)—), a nucleotide, or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), a hydrazone (═N═N(R′)—) carboxy, aryl, aryloxy, heteroaryl, and heteroaryloxy; wherein each R′ is independently H or (C₁-C₆)alkyl.

In Linker Embodiments LE7, the linking group is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiments LE8, the linking group is a divalent, branched or unbranched, saturated or unsaturated, hydrocarbon chain, having from 2 to 10 carbon atoms, wherein one or more (e.g., 1, 2, 3, or 4) of the carbon atoms is optionally replaced by (—O—), (—NH—), (—S), an amino acid, a hydrazone (—C(R′)═N═N(R′)—), a nucleotide, or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), a hydrazone (═N═N(R′)—) carboxy, aryl, aryloxy, heteroaryl, and heteroaryloxy; wherein each R′ is independently H or (C₁-C₆)alkyl.

In Linker Embodiments LE9, the linking group is a divalent, branched or unbranched, saturated or unsaturated, hydrocarbon chain, having from 2 to 10 carbon atoms, wherein one or more (e.g., 1, 2, 3, or 4) of the carbon atoms is optionally replaced by (—O—), (—NH—), or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiments LE10, the linking group is a divalent, branched or unbranched, saturated or unsaturated, hydrocarbon chain, having from 2 to 25 carbon atoms, wherein the chain is optionally substituted on carbon with one or more (e.g., 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiments LE11, the linking group is a divalent, branched or unbranched, saturated or unsaturated, hydrocarbon chain, having from 2 to 10 carbon atoms, wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In Linker Embodiments LE12, the linking group is a divalent, branched or unbranched, saturated or unsaturated, hydrocarbon chain, having from 2 to 10 carbon atoms.

In Linker Embodiments LE13, the linking group is a divalent, branched or unbranched, saturated hydrocarbon chain, having from 2 to 10 carbon atoms.

In Linker Embodiments LE14, the linking group is a divalent, unbranched, saturated hydrocarbon chain, having from 2 to 10 carbon atoms.

In Linker Embodiments LE15, the linking group is a divalent, branched or unbranched, saturated or unsaturated, chain having from 2 to 25 atoms selected from carbon, oxygen, nitrogen and sulfur, where in the chain comprises one or more disulfide linkages.

In Linker Embodiments LE16, the linking group is a divalent, branched or unbranched, saturated or unsaturated, chain having from 2 to 25 atoms selected from carbon, oxygen, nitrogen and sulfur, where in the chain comprises one or more hydrazone groups in the chain or appended to a carbon atom of the chain.

In Linker Embodiments LE17, the linking group is a divalent, branched or unbranched, saturated or unsaturated, chain having from 2 to 35 atoms selected from carbon, oxygen, nitrogen and sulfur, where in the chain comprises one or more amino acids in the chain.

In Linker Embodiments LE18, the linking group is a divalent, branched or unbranched, saturated or unsaturated, chain having from 2 to 35 atoms selected from carbon, oxygen, nitrogen and sulfur, where in the chain comprises a dipeptide in the chain.

In Linker Embodiments LE19, the linking group is a divalent, branched or unbranched, saturated or unsaturated, chain having from 2 to 35 atoms selected from carbon, oxygen, nitrogen and sulfur, where in the chain comprises the dipeptide val-cit in the chain.

In Linker Embodiments LE20, the linking group comprises one or more nucleotides in the chain.

In Linker Embodiments LE21, the linking group comprises two or more nucleotides in the chain.

In Linker Embodiments LE22, the linking group comprises a tri-nucleotide group in the chain.

In Linker Embodiments LE23, a linking group is attached to two or more oligonucleotides (e.g., for a compound of formula (I) at least one “y” is greater than 1).

In Linker Embodiments LE24, only one linking group is attached to two or more oligonucleotides (e.g., for a compound of formula (I) one “y” is greater than 1).

In Linker Embodiments LE25, at least two linking groups are attached to two or more oligonucleotides (e.g., for a compound of formula (I) at least two “y” are greater than 1).

In Linker Embodiments LE26, at least two linking groups are attached to two oligonucleotides (e.g., for a compound of formula (I), “y” is 2 and n is greater than 1).

In Linker Embodiments LE27, the linking group is attached to the oligonucleotide through a phosphate of the oligonucleotide (e.g., associated with the 5′ terminal residue).

In Linker Embodiments LE28, the linking group is attached to the oligonucleotide through a phosphorothioate of the oligonucleotide (e.g., associated with the 5′ terminal residue).

In Linker Embodiments LE29, the linking group comprises a polyethyleneoxy chain.

In another embodiment of the invention the polyethyleneoxy chain comprises 2, 3, 4, 5, 6, 7, 8, 9, or 10 repeating ethyleneoxy units.

In Linker Embodiments LE30, the linking group comprises a 5-membered di-valent heterocycle.

In Linker Embodiments LE31, the linking group has the following structure:

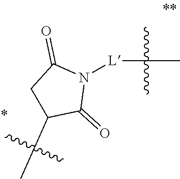

wherein L′ is a divalent, branched or unbranched, saturated or unsaturated, hydrocarbon chain, having from 2 to 25 carbon atoms, wherein one or more (e.g., 1, 2, 3, or 4) of the carbon atoms is optionally replaced by (—O—), (—NH—), (—S), an amino acid, a hydrazone (—C(R′)═N═N(R′)—), a nucleotide, or a 3-12 membered di-valent heterocycle, wherein the chain and any 3-12 membered di-valent heterocycle is optionally substituted with one or more (e.g., 1, 2, 3, or 4) substituents independently selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), a hydrazone (═N═N(R′)—) carboxy, aryl, aryloxy, heteroaryl, and heteroaryloxy; wherein each R′ is independently H or (C₁-C₆)alkyl; and wherein the valence marked * is attached to P and the valence marked ** is attached to X in formula (I). In another embodiment, L′ is a divalent, branched or unbranched, saturated or unsaturated, chain having from 2 to 25 atoms selected from carbon, oxygen, nitrogen and sulfur, where in the chain comprises one or more disulfide linkages. In another embodiment, L′ is a divalent, branched or unbranched, saturated or unsaturated, chain having from 2 to 25 atoms selected from carbon, oxygen, nitrogen and sulfur, where in the chain comprises one or more hydrazone groups in the chain or appended to a carbon atom of the chain. In another embodiment, L′ is a divalent, branched or unbranched, saturated or unsaturated, chain having from 2 to 35 atoms selected from carbon, oxygen, nitrogen and sulfur, where in the chain comprises one or more amino acids in the chain. In another embodiment, L′ is a divalent, branched or unbranched, saturated or unsaturated, chain having from 2 to 35 atoms selected from carbon, oxygen, nitrogen and sulfur, where in the chain comprises a dipeptide in the chain. In another embodiment, L′ is a divalent, branched or unbranched, saturated or unsaturated, chain having from 2 to 35 atoms selected from carbon, oxygen, nitrogen and sulfur, where in the chain comprises the dipeptide val-cit in the chain. In another embodiment, L′ comprises one or more nucleotides. In another embodiment, L′ comprises two or more nucleotides. In another embodiment, L′ comprises a tri-nucleotide group. In another embodiment, L′ comprises one or more nucleotides having unmodified bases, unmodified sugar groups and/or unmodified phosphate groups.

In Linker Embodiments LE32, L′ has the following structure:

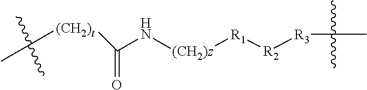

wherein t is 1, 2, 3, 4, 5, 6, 7, or 8; z is 0, 1, 2, 3, 4, 5, 6, 7, or 8; and each of R₁, R₂, and R₃ is independently a nucleotide.

In Linker Embodiments LE33, L′ has the following structure:

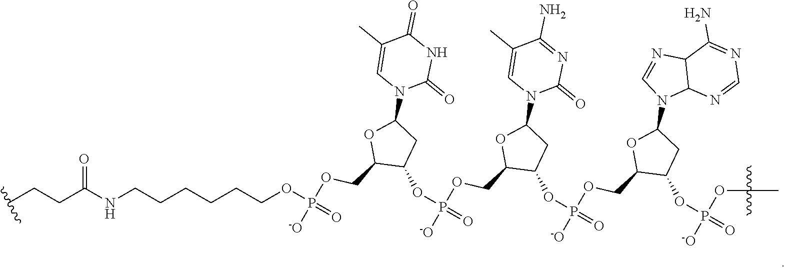

In Linker Embodiments LE34 the linking group has the following structure:

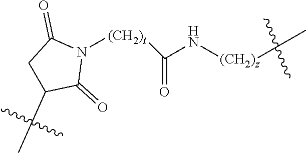

wherein t is 1, 2, 3, 4, 5, 6, 7, or 8; and z is 0, 1, 2, 3, 4, 5, 6, 7, or 8.

In Linker Embodiments LE35, the linking group has the following structure:

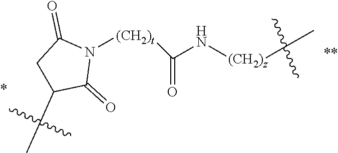

wherein t is 1, 2, 3, 4, 5, 6, 7, or 8; and z is 0, 1, 2, 3, 4, 5, 6, 7, or 8, wherein the valence marked * is attached to P and the valence marked ** is attached to X in formula (I). In certain embodiments, the valence marked ** is attached to X through a phosphate of the oligonucleotide (e.g., associated with the 5′ terminal residue).

In Linker Embodiments LE36, the linking group has the following structure:

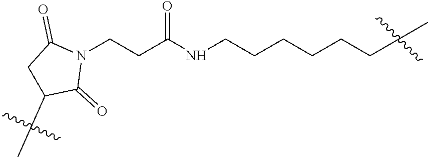 [SMILES: *CCCCCCNC(=O)CCN1C(=O)CC(*)C1=O]

In Linker Embodiments LE37, the linking group has the following structure:

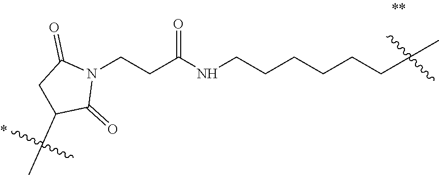 [SMILES: *CCCCCCNC(=O)CCN1C(=O)CC(*)C1=O]

wherein the valence marked * is attached to P and the valence marked ** is attached to X in formula (I). In certain embodiments, the valence marked ** is attached to X through a phosphate of the oligonucleotide (e.g., associated with the 5′ terminal residue). As such, the A group in the linker structures can, in embodiments, be covalently bound to —O—PO₃ at [symbol] which is itself covalently bound to the oligonucleotide.

In Linker Embodiments LE38, the linking group has the following structure:

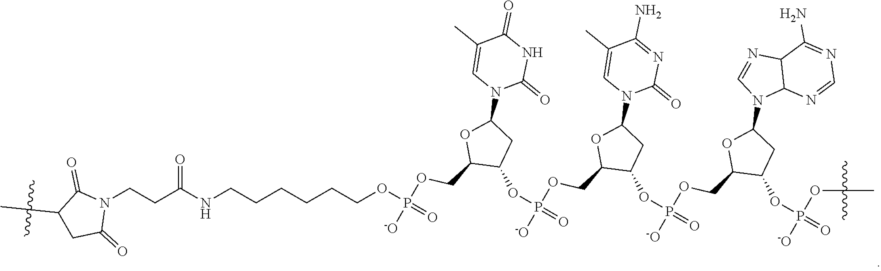

In Linker Embodiments LE39, the linker is a peptide linker or formed from a protein, peptide or amino acid. For example, in certain embodiments, the linking group is a divalent radical formed from a protein. In another embodiment, the linking group is a divalent radical formed from a peptide. In another embodiment, the linking group is a divalent radical formed from an amino acid.

In Linker Embodiments LE40, the linking group may be configured such that it allows for the rotation of the oligonucleotide and the TfR binding protein relative to each other; and/or is resistant to digestion by proteases. In some embodiments, the linking group may be a flexible linker, e.g., containing amino acids such as Gly, Asn, Ser, Thr, Ala, and the like. Such linking groups are designed using known parameters. For example, the linking groups may have repeats, such as Gly-Ser repeats.

In Linker Embodiments LE41, the linking group has or comprises a formula selected from the group consisting of:

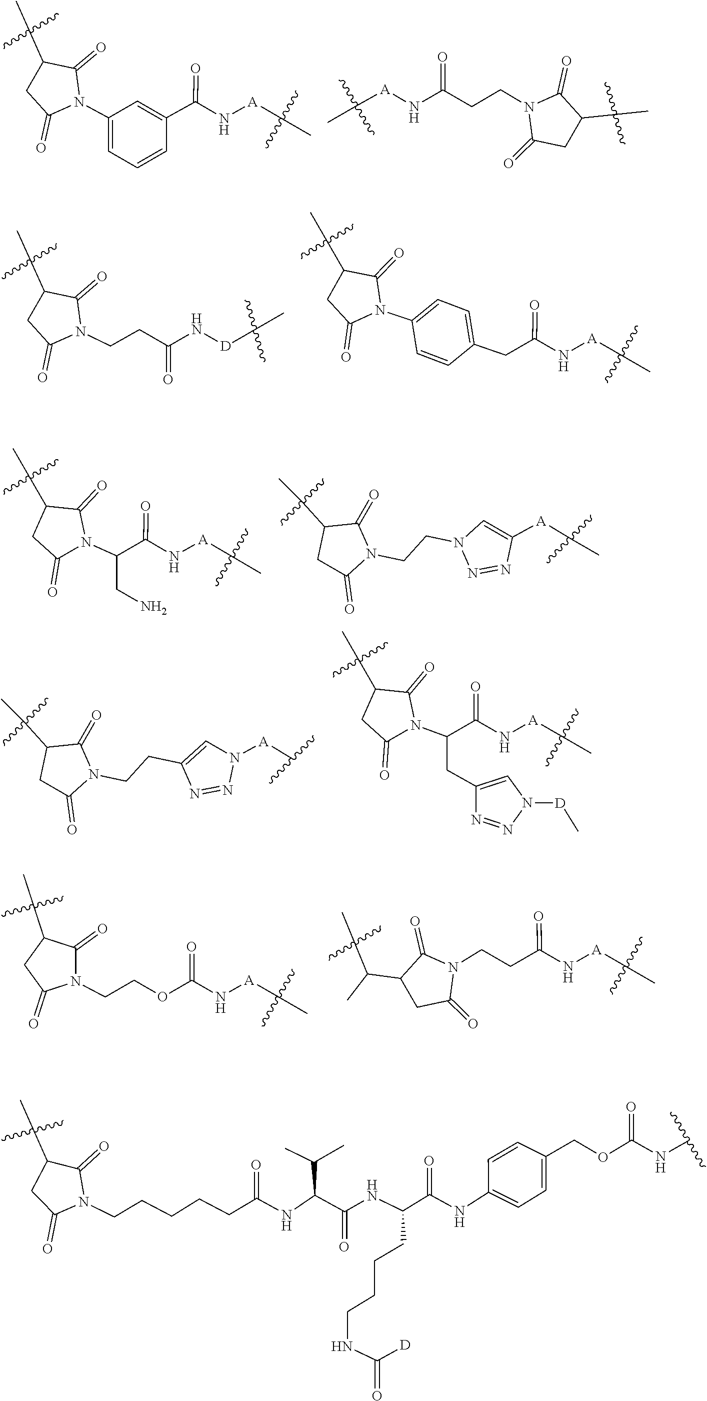

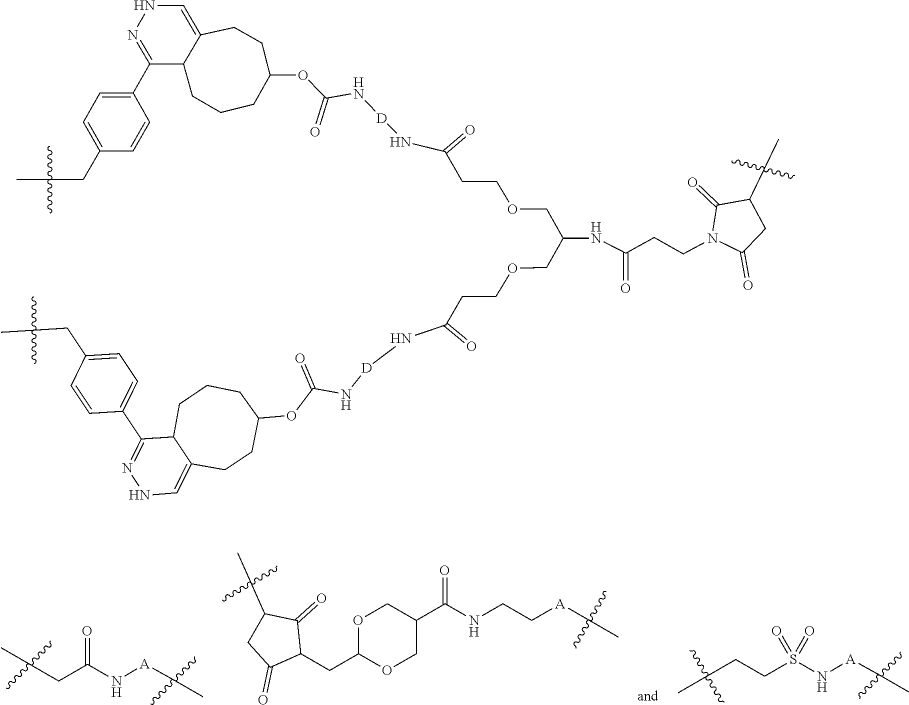

wherein
each A is independently (C₁-C₁₅)alkyl;
each D is —(CH₂—CH₂—O)ₘ—; and
each m is 1, 2, 3, 4, 5, 6, 7, 8, 9, 10, 11, 12, 13, 14, 15, 16, 17, 18, 19, 20, 21, 22, 23, or 24.

In Linker Embodiments LE42, the linking group has or comprises a formula selected from the group consisting of:

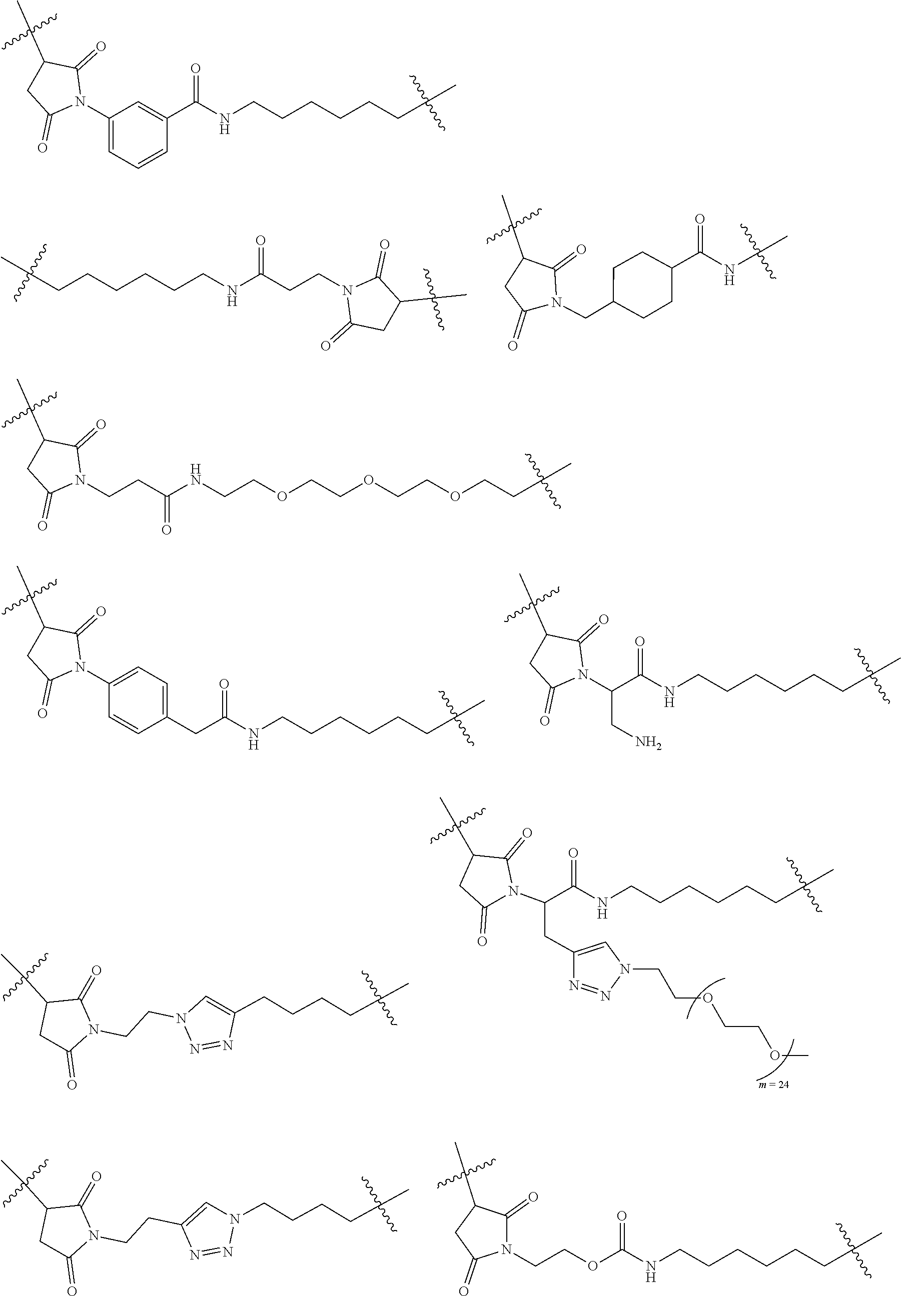

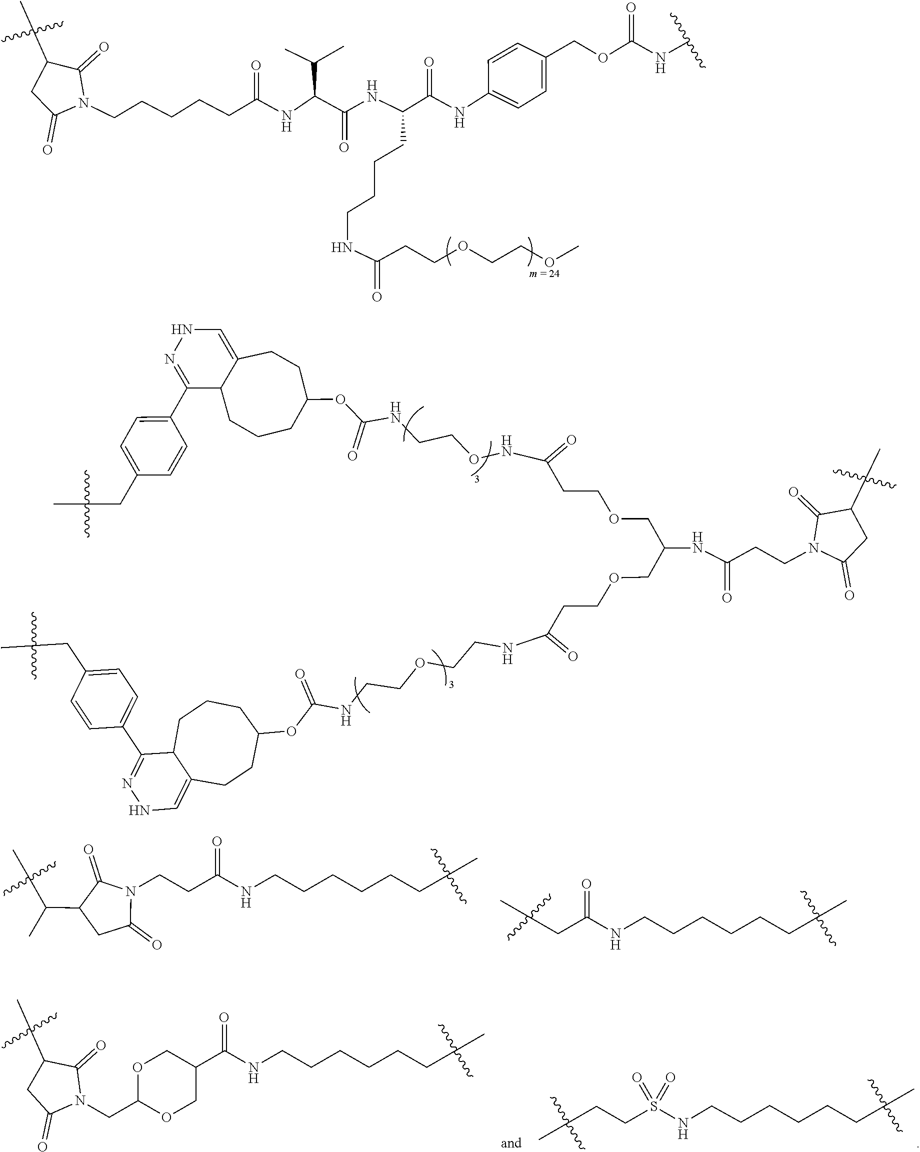

In various embodiments, the conjugates can be generated using well-known chemical cross-linking reagents and protocols. For example, there are a large number of chemical cross-linking agents that are known to those skilled in the art and useful for cross-linking a protein with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxy-succinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydro-chloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)-propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate·2HCl (DMP) are examples of useful homobifunctional cross-linking agents, and bis-[B-(4-azidosalicylamido)-ethyl]disulfide (BASED) and N-succinimidyl-6(4′-azido-2′-nitrophenylamino)hexanoate (SANPAH) are examples of useful photoreactive cross-linkers.

In embodiments, wherein the linking group L comprises a moiety having the structure:

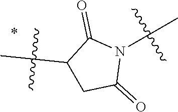

wherein, * denotes the attachment point to a sulfur atom of a modified site within P, refers specifically to any such linkers described in this section and linker embodiments described herein.

In embodiments comprising a conjugate of Formula (I) as described here, each L refers specifically to any of the linkers described in this section and linker embodiments described herein.

### IX. Variable “P”: Transferrin Receptor-Binding Proteins

This section describes proteins that bind to a transferrin receptor and are capable of being transported across the blood-brain barrier (BBB). Certain proteins that bind to a transferrin receptor are described herein, as well as in WO2018/152326. Certain methods of making TfR binding proteins are described in WO2018/152326, which is incorporated by reference herein for all purposes.

A TfR binding protein as described herein may have a range of binding affinities. For example, in some embodiments, a protein has an affinity for TfR, ranging anywhere from 1 pM to 10 μM. In some embodiments, the affinity for TfR ranges from 1 nM to 5 μM, or from 10 nM to 1 μM. In some embodiments, the protein binds to TfR with low affinity weaker than 3 nM, such as 3 nM, 4 nM, 5 nM, 5 nM, 7 nM, 8 nM, 9 nM, 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, 400 nM, 410 nM, 420 nM, 430 nM, 440 nM, 450 nM, 460 nM, 470 nM, 480 nM, 490 nM, or 500 nM. In some embodiments, the affinity for TfR ranges from about 40 nM to about 1200 nM, 50 nM to about 500 nM, or from about 75 nM to about 300 nM, or from about 100 nM to about 250 nM. In some embodiments, the protein binds (e.g., specifically binds) to a TfR with an affinity of about 40, 50, 80, 100, 130, 150, 200, 250, 300, 350, 400, 450, 500, 550, 600, 650, 700, 750, 800, 850, 900, 950, 1000, 1050, 1100, 1150, or 1200 nM. In some embodiments, the protein binds to a TfR with an affinity of from about 50 nM to about 300 nM, about 80 nM to about 300 nM, 100 nM to about 300 nM, or from about 150 nM to about 250 nM, or from about 200 nM to about 250 nM. In some embodiments, the protein binds to a TfR with an affinity of from about 40 nM to about 500 nM, about 50 nM to about 500 nM, about 50 nM to about 400 nM, about 50 nM to about 300 nM, about 50 nM to about 200 nM, about 50 nM to about 100 nM, about 100 nM to about 500 nM, about 100 nM to about 400 nM, about 100 nM to about 300 nM, about 100 nM to about 200 nM. In some embodiments, the protein binds to a TfR with an affinity of from about 5 nM to about 500 nM, about 5 nM to about 400 nM, about 5 nM to about 300 nM, about 5 nM to about 200 nM, about 5 nM to about 100 nM, about 10 nM to about 500 nM, about 10 nM to about 400 nM, about 10 nM to about 300 nM, or about 10 nM to about 200 nM. In some embodiments, the protein binds to a TfR with an affinity of from about 3 nM to about 600 nM In some embodiments, the protein binds to a TfR with an affinity of from about 80 nM to about 180 nM or about 50 nM to about 250 nM. In some embodiments, the protein binds to a TfR with an affinity of about 10 nM, about 100 nM or about 500 nM.

In one aspect, a TfR binding protein as described herein may have a new or non-native TfR binding site.

In one aspect, proteins are provided that comprise constant domains that have modifications that allow the proteins to specifically bind to a transferrin receptor. In some embodiments, the protein has modifications in the CL domain. In some embodiments, the protein has modifications in the CH1 domain. In some embodiments, the protein has modifications in the CH2 domain. In some embodiments, the protein has modifications in the CH3 domain. Exemplary CH2 and CH3 domains with modifications that allow specific binding to TfR are described, e.g., in WO 2018/152326, which is incorporated herein by reference in its entirety.

In one aspect, proteins are provided that comprise constant domains that have modifications that allow the proteins to specifically bind to a transferrin receptor. The modifications are introduced into specified sets of amino acids that are present at the surface of the constant (e.g., CH3) domain. In some embodiments, proteins comprising a modified constant domain specifically bind to an epitope in the apical domain of the transferrin receptor. In some embodiments, the protein binds to the transferrin receptor without inhibiting binding of transferrin to the transferrin receptor. In some embodiments, the protein binds to an epitope that comprises amino acid 208 of the transferrin receptor sequence.

One of skill understands that constant (e.g., CH3) domains of other immunoglobulin isotypes, e.g., IgM, IgA, IgE, IgD, etc. may be similarly modified by identifying the amino acids in those domains that correspond to sets (i)-(ii) described herein. Modifications may also be made to corresponding domains from immunoglobulins from other species, e.g., non-human primates, monkey, mouse, rat, rabbit, dog, pig, chicken, and the like.

### CH3 Transferrin Receptor-Binding Proteins

In one aspect, proteins are provided that comprise CH3 domains that have modifications that allow the proteins to specifically bind to a transferrin receptor. In some embodiments, the domain that is modified is a human Ig CH3 domain. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme. The positions in the CH3 domain for purposes of identifying the corresponding set of amino acid positions for transferrin receptor binding are determined with reference to SEQ ID NO:3 or determined with reference to amino acids 114-220 of SEQ ID NO:1 unless otherwise specified. Substitutions are also determined with reference to SEQ ID NO:1, i.e., an amino acid is considered to be a substitution relative to the amino acid at the corresponding position in SEQ ID NO:1. SEQ ID NO:1 includes a partial hinge region sequence, PCP, as amino acids 1-3. The numbering of the positions in the CH3 domain with reference to SEQ ID NO:1 includes the first three amino acids.

As indicated above, sets of residues of a CH3 domain that can be modified as described herein are numbered herein with reference to SEQ ID NO:1; however, any CH3 domain, e.g., an IgG1, IgG2, IgG3, or IgG4 CH3 domain, may have modifications, e.g., amino acid substitutions, in one or more sets of residues that correspond to residues at the noted positions in SEQ ID NO:1. The positions of each of the IgG2, IgG3, and IgG4 sequences that correspond to any given position of SEQ ID NO:1 can be readily determined.

In one embodiment, a modified CH3 domain protein that specifically binds transferrin receptor binds to the apical domain of the transferrin receptor at an epitope that comprises position 208 of the full-length human transferrin receptor sequence (SEQ ID NO:235), which corresponds to position 11 of the human transferrin receptor apical domain sequence set forth in SEQ ID NO:107. SEQ ID NO:107 corresponds to amino acids 198-378 of the human transferrin receptor-1 uniprotein sequence P02786 (SEQ ID NO:235). In some embodiments, the modified CH3 domain protein binds to the apical domain of the transferrin receptor at an epitope that comprises positions 158, 188, 199, 207, 208, 209, 210, 211, 212, 213, 214, 215, and/or 294 of the full length human transferrin receptor sequence (SEQ ID NO:235). The modified CH3 domain protein may bind to the transferrin receptor without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7% 6%, 5% 4%, 3%, 2%, or 1%). Illustrative CH3 domain proteins that exhibit this binding specificity include proteins having amino acid substitutions at positions 157, 159, 160, 161, 162, 163, 186, 189, and 194 as determined with reference to amino acids 114-220 of SEQ ID NO:1.

### CH3 Transferrin Receptor Binding Set (i): 157, 159, 160, 161, 162, 163, 186, 189, and 194

In some embodiments, a modified CH3 domain protein as described herein comprises at least three or at least four, and typically five, six, seven, eight, or nine substitutions in a set of amino acid positions comprising 157, 159, 160, 161, 162, 163, 186, 189, and 194 (set i). Illustrative substitutions that may be introduced at these positions are shown in Tables B and C. In some embodiments, the amino acid at position 161 and/or 194 is an aromatic amino acid, e.g., Trp, Phe, or Tyr. In some embodiments, the amino acid at position 161 is Trp. In some embodiments, the amino acid at position 161 is Gly. In some embodiments, the aromatic amino acid at position 194 is Trp or Phe.

In some embodiments, a modified CH3 domain protein that specifically binds a transferrin receptor comprises at least one position having a substitution, relative to SEQ ID NO:1, as follows: Leu, Tyr, Met, or Val at position 157; Leu, Thr, His, or Pro at position 159; Val, Pro, or an acidic amino acid at position 160; an aromatic amino acid, e.g., Trp or Gly (e.g., Trp) at position 161; Val, Ser, or Ala at position 162; an acidic amino acid, Ala, Ser, Leu, Thr, or Pro at position 186; Thr or an acidic amino acid at position 189; or Trp, Tyr, His, or Phe at position 194. In some embodiments, the modified CH3 domain comprises two, three, four, five, six, seven, or eight positions selected from the following: position 157 is Leu, Tyr, Met, or Val; position 159 is Leu, Thr, His, or Pro; position 160 is Val, Pro, or an acidic amino acid; position 161 is Trp or Gly; position 162 is Val, Ser, or Ala; position 186 is Glu, Ala, Ser, Leu, Thr, or Pro; position 189 is Thr or an acidic amino acid; and position 194 is Trp, Tyr, His, or Phe. In some embodiments, the modified CH3 domain comprises Leu or Met at position 157; Leu, His, or Pro at position 159; Val at position 160; Trp or Gly at position 161; Val or Ala at position 162; Pro at position 186; Thr at position 189; and/or Trp at position 194. In some embodiments, a modified CH3 domain protein may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 157, 159, and/or position 186. In some embodiments, the acidic amino acid at position one, two, or each of positions 160, 186, and 189 is Glu. In other embodiments, the acidic amino acid at one, two or each of positions 160, 186, and 189 is Asp. In some embodiments, two, three, four five, six, seven, or all eight of positions 157, 159, 160, 161, 162, 186, 189, and 194 have an amino acid substitution as specified in this paragraph.

In some embodiments, a CH3 domain protein having modifications in set (i) comprises a native Asn at position 163. In some embodiments, the modified CH3 domain protein comprises Gly, His, Gln, Leu, Lys, Val, Phe, Ser, Ala, or Asp at position 163. In some embodiments, the modified CH3 domain protein further comprises one, two, three, or four substitutions at positions comprising 153, 164, 165, and 188. In some embodiments, Trp, Tyr, Leu, or Gln may be present at position 153. In some embodiments, Ser, Thr, Gln, or Phe may be present at position 164. In some embodiments, Gln, Phe, or His may be present at position 165. In some embodiments, Glu may be present at position 188.

In certain embodiments, the modified CH3 domain protein comprises two, three, four, five, six, seven, eight nine, or ten positions selected from the following: Trp, Leu, or Glu at position 153; Tyr or Phe at position 157; Thr at position 159; Glu at position 160; Trp at position 161; Ser, Ala, Val, or Asn at position 162; Ser or Asn at position 163; Thr or Ser at position 186; Glu or Ser at position 188; Glu at position 189; and/or Phe at position 194. In some embodiments, the modified CH3 domain protein comprises all eleven positions as follows: Trp, Leu, or Glu at position 153; Tyr or Phe at position 157; Thr at position 159; Glu at position 160; Trp at position 161; Ser, Ala, Val, or Asn at position 162; Ser or Asn at position 163; Thr or Ser at position 186; Glu or Ser at position 188; Glu at position 189; and/or Phe at position 194.

In certain embodiments, the modified CH3 domain protein comprises Leu or Met at position 157; Leu, His, or Pro at position 159; Val at position 160; Trp at position 161; Val or Ala at position 162; Pro at position 186; Thr at position 189; and/or Trp at position 194. In some embodiments, the modified CH3 domain protein further comprises Ser, Thr, Gln, or Phe at position 164. In some embodiments, a modified CH3 domain protein further comprises Trp, Tyr, Leu, or Gln at position 153 and/or Gln, Phe, or His at position 165. In some embodiments, Trp is present at position 153 and/or Gln is present at position 165. In some embodiments, a modified CH3 domain protein does not have a Trp at position 153.

In other embodiments, a modified CH3 domain protein comprises Tyr at position 157; Thr at position 159; Glu or Val and position 160; Trp at position 161; Ser at position 162; Ser or Thr at position 186; Glu at position 189; and/or Phe at position 194. In some embodiments, the modified CH3 domain protein comprises a native Asn at position 163. In certain embodiments, the modified CH3 domain protein further comprises Trp, Tyr, Leu, or Gln at position 153; and/or Glu at position 188. In some embodiments, the modified CH3 domain protein further comprises Trp at position 153 and/or Glu at position 188.

In some embodiments, the modified CH3 domain comprises one or more of the following substitutions: Trp at position 153; Thr at position 159; Trp at position 161; Val at position 162; Ser or Thr at position 186; Glu at position 188; and/or Phe at position 194.

In additional embodiments, the modified CH3 domain further comprises one, two, or three positions selected from the following: position 187 is Lys, Arg, Gly, or Pro; position 197 is Ser, Thr, Glu, or Lys; and position 199 is Ser, Trp, or Gly.

In some embodiments, a modified CH3 domain protein that specifically binds transferrin receptor has at least 70% identity, at least 75% identity, at least 80% identity, at least 85% identity, at least 90% identity, at least 95% identity, at least 96% identity, at least 97% identity, at least 98% identity or at least 99% identity to amino acids 114-220 of any one of SEQ ID NOS:4-29, 236-299, 422-435, 645, 650 and 746. In some embodiments, such a modified CH3 domain protein comprises amino acids 157-163 and/or 186-194 of any one of SEQ ID NOS:4-29, 236-299, 422-435, 645, 650 and 746. In some embodiments, such a modified CH3 domain protein comprises amino acids 153-163 and/or 186-194 of any one of SEQ ID NOS:4-29, 236-299, 422-435, 645, 650 and 746. In some embodiments, a modified CH3 domain protein comprises amino acids 153-163 and/or 186-199 of any one of SEQ ID NOS:4-29, 236-299, 422-435, 645, 650 and 746. In certain embodiments, the residues of at least 5, 6, 7, 8, 9, 10, 11, 12, 13, 14, 15, or 16 (e.g., 11 to 16) of the positions corresponding to positions 153, 157, 159, 160, 161, 162, 163, 164, 165, 186, 187, 188, 189, 194, 197 and 199, determined with reference to SEQ ID NO:1, are not deleted or substituted in SEQ ID NOS:4-29, 236-299 645, 650 or 746.

In some embodiments, a modified CH3 domain protein that specifically binds transferrin receptor has at least 70% identity, at least 75% identity, at least 80% identity, at least 85% identity, at least 90% identity, at least 95% identity, at least 96% identity, at least 97% identity, at least 98% identity or at least 99% identity to amino acids 114-220 of SEQ ID NO:1 or 634, with the proviso that the percent identity does not include the set of positions 157, 159, 160, 161, 162, 163, 186, 189, and 194. In some embodiments, the modified CH3 domain protein comprises amino acids 157-163 and/or amino acids 186-194 as set forth in any one of SEQ ID NOS:4-29, 236-299, 422-435, 645, 650 and 746.

In some embodiments, a modified CH3 domain protein has at least 70% identity, at least 75% identity, at least 80% identity, at least 85% identity, at least 90% identity, at least 95% identity, at least 96% identity, at least 97% identity, at least 98% identity or at least 99% identity to any one of SEQ ID NOS:4-29, 236-299, 422-435, 645, 650 and 746 with the proviso that at least five, six, seven, eight, nine, ten, eleven, twelve, thirteen, fourteen, fifteen, or sixteen of the positions that correspond to positions 153, 157, 159, 160, 161, 162, 163, 164, 165, 186, 187, 188, 189, 194, 197, and 199 of any one of SEQ ID NOS:4-29, 236-299, 422-435, 645, 650 and 746 are not deleted or substituted.

In some embodiments, the modified CH3 domain protein has at least 75% identity, at least 80% identity, at least 85% identity, at least 90% identity, at least 95% identity, at least 96% identity, at least 97% identity, at least 98% identity or at least 99% identity to any one of SEQ ID NOS:4-29, 236-299, 422-435, 645, 650 and 746 and also comprises at at least five, six, seven, eight, nine, ten, eleven, twelve, thirteen, fourteen, fifteen or sixteen of the positions as follows: Trp, Tyr, Leu, Gln, or Glu at position 153; Leu, Tyr, Met, or Val at position 157; Leu, Thr, His, or Pro at position 159; Val, Pro, or an acidic amino acid at position 160; an aromatic amino acid, e.g., Trp, at position 161; Val, Ser, or Ala at position 162; Ser or Asn at position 163; Ser, Thr, Gln, or Phe at position 164; Gln, Phe, or His at position 165; an acidic amino acid, Ala, Ser, Leu, Thr, or Pro at position 186; Lys, Arg, Gly or Pro at position 187; Glu or Ser at position 188; Thr or an acidic amino acid at position 189; Trp, Tyr, His or Phe at position 194; Ser, Thr, Glu or Lys at position 197; and Ser, Trp, or Gly at position 199.

In some embodiments, a modified CH3 domain protein as described herein comprises one or more substitutions in a set of amino acid positions comprising 153, 157, 159, 160, 162, 163, 186, 188, 189, 194, 197, and 199; and wherein the substitutions and the positions are determined with reference to the sequence of SEQ ID NO:13. In some embodiments, the modified CH3 domain comprises Glu, Leu, Ser, Val, Trp, or Tyr at position 153; an aromatic amino acid (e.g., Tyr, Phe, or Trp), Met, Pro, or Val at position 157; Thr, Asn, or Val at position 159; Glu, Ile, Pro, or Val at position 160; an aliphatic amino acid (e.g., Ala, Ile, or Val), Ser, or Thr at position 162; Ser, Asn, Arg, or Thr at position 163; Thr, His, or Ser at position 186; Glu, Ser, Asp, Gly, Thr, Pro, Gln, or Arg at position 188; Glu or Arg at position 189; Phe, His, Lys, Tyr, or Trp at position 194; Ser, Thr, or Trp at position 197; and Ser, Cys, Pro, Met, or Trp at position 199. A modified CH3 domain protein may have the sequence: GQPREPQVYTLPPS-RDELTKNQVSLTCLVKGFYPSDIAVX₁WESX₂GX₃X₄WX₅X₆YKTTPPVLDSDGSFFLYS-KLTVX₇KX₈X₉WQQGX₁₀VFX₁₁CX₁₂VMHEALHNHYTQKSLSLSPGK (SEQ ID NO:556), in which X₁ is E, L, S, V, W, or Y; X₂ is an aromatic amino acid (e.g., Y, F, or W), M, P, or V; X₃ is T, N, or V; X₄ is E, I, P, or V; X₅ is an aliphatic amino acid (e.g., A, I, or V), S, or T; X₆ is S, N, R, or T; X₇ is T, H, or S; X₈ is E, S, D, G, T, P, Q, or R; X₉ is E or R; X₁₀ is F, H, K, Y, or W; X₁₁ is S, T, or W; and X₁₂ is S, C, P, M, or W. In certain embodiments, a modified CH3 domain protein may comprise the sequence: X₁WESX₂GX₃X4WX₅X₆(SEQ ID NO:554), in which X₁ is E, L, S, V, W, or Y; X₂ is an aromatic amino acid (e.g., Y, F, or W), M, P, or V; X₃ is T, N, or V; X₄ is E, I, P, or V; X₅ is an aliphatic amino acid (e.g., A, I, or V), S, or T; and X₆ is S, N, R, or T. In certain embodiments, a modified CH3 domain protein may comprise the sequence: X₁KX₂X₃WQQGX₄VFX₅CX₆ (SEQ ID NO:555), in which X₁ is T, H, or S; X₂ is E, S, D, G, T, P, Q, or R; X₃ is E or R; X₄ is F, H, K, Y, or W; X₅ is S, T, or W; and X₆ is S, C, P, M, or W.

In some embodiments, the modified CH3 domain protein comprises Glu, Leu, or Trp at position 153; an aromatic amino acid at position 157; Thr at position 159; Glu at position 160; an aliphatic amino acid or Ser at position 162; Ser or Asn at position 163; Thr or Ser at position 186; Glu or Ser at position 188; Glu at position 189; Phe, His, Tyr, or Trp at position 194; Ser at position 197; and Ser at position 199, wherein the substitutions and the positions are determined with reference to the sequence of SEQ ID NO:13. In particular embodiments, the aromatic amino acid at position 157 is Tyr or Phe and the aliphatic amino acid at position 162 is Ala or Val. In further embodiments, the modified CH3 domain protein may comprise Glu, Leu, or Trp at position 153; Tyr or Phe at position 157; Thr at position 159; Glu at position 160; Ala, Val, or Ser at position 162; Ser or Asn at position 163; Thr or Ser at position 186; Glu or Ser at position 188; Glu at position 189; Phe at position 194; Ser at position 197; and Ser at position 199, wherein the substitutions and the positions are determined with reference to the sequence of SEQ ID NO:13.

In some embodiments, a modified CH3 domain protein as described herein comprises only one substitution in a set of amino acid positions comprising 153, 157, 159, 160, 162, 163, 186, 188, 189, 194, 197, and 199; and wherein the substitutions and the positions are determined with reference to the sequence of SEQ ID NO:238. In some embodiments, the modified CH3 domain protein comprises Glu, Leu, Ser, Val, Trp, or Tyr at position 153. The modified CH3 domain protein may comprise Glu at position 153. The modified CH3 domain protein may comprise Leu at position 153. The modified CH3 domain protein may comprise Ser at position 153. The modified CH3 domain protein may comprise Val at position 153. The modified CH3 domain protein may comprise Trp at position 153. The modified CH3 domain protein may comprise Tyr at position 153. In some embodiments, the modified CH3 domain protein comprises Tyr, Phe, Trp, Met, Pro, or Val at position 157. The modified CH3 domain protein may comprise Tyr at position 157. The modified CH3 domain protein may comprise Phe at position 157. The modified CH3 domain protein may comprise Trp at position 157. The modified CH3 domain protein may comprise Met at position 157. The modified CH3 domain protein may comprise Pro at position 157. The modified CH3 domain protein may comprise Val at position 157. In some embodiments, the modified CH3 domain protein comprises Thr, Asn, or Val at position 159. The modified CH3 domain protein may comprise Thr at position 159. The modified CH3 domain protein may comprise Asn at position 159. The modified CH3 domain protein may comprise Val at position 159. In some embodiments, the modified CH3 domain protein comprises Glu, Ile, Pro, or Val at position 160. The modified CH3 domain protein may comprise Glu at position 160. The modified CH3 domain protein may comprise Ile at position 160. The modified CH3 domain protein may comprise Pro at position 160. The modified CH3 domain protein may comprise Val at position 160. In some embodiments, the modified CH3 domain protein comprises Ala, Ile, Val, Ser, or Thr at position 162. The modified CH3 domain protein may comprise Ala at position 162. The modified CH3 domain protein may comprise Ile at position 162. The modified CH3 domain protein may comprise Val at position 162. The modified CH3 domain protein may comprise Ser at position 162. The modified CH3 domain protein may comprise Thr at position 162. In some embodiments, the modified CH3 domain protein comprises Ser, Asn, Arg, or Thr at position 163. The modified CH3 domain protein may comprise Ser at position 163. The modified CH3 domain protein may comprise Asn at position 163. The modified CH3 domain protein may comprise Arg at position 163. The modified CH3 domain protein may comprise Thr at position 163. In some embodiments, the modified CH3 domain protein comprises Thr, His, or Ser at position 186. The modified CH3 domain protein may comprise Thr at position 186. The modified CH3 domain protein may comprise His at position 186. The modified CH3 domain protein may comprise Ser at position 186. In some embodiments, the modified CH3 domain protein comprises Glu, Ser, Asp, Gly, Thr, Pro, Gln, or Arg at position 188. The modified CH3 domain protein may comprise Glu at position 188. The modified CH3 domain protein may comprise Ser at position 188. The modified CH3 domain protein may comprise Asp at position 188. The modified CH3 domain protein may comprise Gly at position 188. The modified CH3 domain protein may comprise Thr at position 188. The modified CH3 domain protein may comprise Pro at position 188. The modified CH3 domain protein may comprise Gln at position 188. The modified CH3 domain protein may comprise Arg at position 188. In some embodiments, the modified CH3 domain protein comprises Glu or Arg at position 189. The modified CH3 domain protein may comprise Glu at position 189. The modified CH3 domain protein may comprise Arg at position 189. In some embodiments, the modified CH3 domain protein comprises Phe, His, Lys, Tyr, or Trp at position 194. The modified CH3 domain protein may comprise Phe at position 194. The modified CH3 domain protein may comprise His at position 194. The modified CH3 domain protein may comprise Lys at position 194. The modified CH3 domain protein may comprise Tyr at position 194. The modified CH3 domain protein may comprise Trp at position 194. In some embodiments, the modified CH3 domain protein comprises Ser, Thr, or Trp at position 197. The modified CH3 domain protein may comprise Ser at position 197. The modified CH3 domain protein may comprise Thr at position 197. The modified CH3 domain protein may comprise Trp at position 197. In some embodiments, the modified CH3 domain protein comprises Ser, Cys, Pro, Met, or Trp at position 199. The modified CH3 domain protein may comprise Ser at position 199. The modified CH3 domain protein may comprise Cys at position 199. The modified CH3 domain protein may comprise Pro at position 199. The modified CH3 domain protein may comprise Met at position 199. The modified CH3 domain protein may comprise Trp at position 199.

In some embodiments, a modified CH3 domain protein as described herein comprises one or more substitutions in a set of amino acid positions comprising 153, 157, 159, 160, 162, 163, 164, 186, 189, and 194; and wherein the substitutions and the positions are determined with reference to the sequence of SEQ ID NO:9. In some embodiments, the modified CH3 domain comprises Glu or Trp at position 153; Val, Trp, Leu, or Tyr at position 157; Leu, Pro, Phe, Thr, or His at position 159; Pro, Val, or Glu at position 160; Ala, Ser, Val, or Gly at position 162; Leu, His, Gln, Gly, Val, Ala, Asn, Asp, Thr, or Glu at position 163; Thr, Phe, Gln, Val, or Tyr at position 164; Leu, Ser, Glu, Ala, or Pro at position 186; Glu, Asp, Thr, or Asn at position 189; and Trp, Tyr, Phe, or His at position 194.

In some embodiments, the modified CH3 domain protein comprises Glu or Trp at position 153; Trp, Leu, or Tyr at position 157; Thr or His at position 159; Val at position 160; Ala, Ser, or Val at position 162; Val, Asn, or Thr at position 163; Gln or Tyr at position 164; Pro at position 186; Thr or Asn at position 189; and Trp, Tyr, Phe, or His at position 194, wherein the substitutions and the positions are determined with reference to the sequence of SEQ ID NO:9.

In additional embodiments, a transferrin receptor-binding protein comprises amino acids 157-194, amino acids 153-194, or amino acids 153-199, of any one of SEQ ID NOS:4-29, 236-299, and 422-435. In further embodiments, the protein comprises an amino acid sequence having at least 75% identity, at least 80% identity, at least 85% identity, at least 90% identity, at least 95% identity, at least 96% identity, at least 97% identity, at least 98% identity or at least 99% identity to amino acids 157-194 of any one of SEQ ID NOS:4-29, 236-299, 422-435, 645, 650 and 746 or to amino acids 153-194, or to amino acids 153-199, of any one of SEQ ID NOS:4-29, 236-299, 422-435, 645, 650 and 746.

In some embodiments, the protein comprises any one of SEQ ID NOS:4-29, 236-299, 422-435, 645-650 and 746. In further embodiments, the protein comprises any one of SEQ ID NOS:4-29, 236-299, 422-435, 645-650 and 746 without the first three amino acids “PCP” at the amino-terminal end. In further embodiments, the protein may have at least 75% identity, at least 80% identity, at least 85% identity, at least 90% identity, at least 95% identity, at least 96% identity, at least 97% identity, at least 98% identity or at least 99% identity to any one of SEQ ID NOS:4-29, 236-299, 422-435, 645, 650 and 746 as determined without the first three amino acids “PCP” at the amino-terminal end.

### CH3 Transferrin Receptor Binding Set (ii): 118, 119, 120, 122, 210, 211, 212, and 213

In some embodiments, a modified CH3 domain protein as described herein comprises at least three or at least four, and typically five, six, seven, or eight substitutions in a set of amino acid positions comprising 118, 119, 120, 122, 210, 211, 212, and 213 (set ii). Illustrative substitutions that may be introduced at these positions are shown in Table A. In some embodiments, the modified CH3 domain protein comprises Gly at position 210; Phe at position 211; and/or Asp at position 213. In some embodiments, Glu is present at position 213. In certain embodiments, a modified CH3 domain protein comprises at least one substitution at a position as follows: Phe or Ile at position 118; Asp, Glu, Gly, Ala, or Lys at position 119; Tyr, Met, Leu, Ile, or Asp at position 120; Thr or Ala at position 122; Gly at position 210; Phe at position 211; His Tyr, Ser, or Phe at position 212; or Asp at position 213. In some embodiments, two, three, four, five, six, seven, or all eight of positions 118, 119, 120, 122, 210, 211, 212, and 213 have a substitution as specified in this paragraph. In some embodiments, a modified CH3 domain protein may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH3 domain protein of the has at least 70% identity, at least 75% identity, at least 80% identity, at least 85% identity, at least 90% identity, at least 95% identity, at least 96% identity, at least 97% identity, at least 98% identity or at least 99% identity to amino acids 114-220 of SEQ ID NO:1 or 634, with the proviso that the percent identity does not include the set of positions 118, 119, 120, 122, 210, 211, 212, and 213.

### Illustrative Proteins Comprising Modified CH2 or CH3 Domains

A modified constant (e.g., CH3) domain as described herein may be joined to another domain of an Fc polypeptide. In some embodiments, a modified CH3 domain as described herein is joined to a CH2 domain, which may be a naturally occurring CH2 domain or a variant CH2 domain, typically at the C-terminal end of the CH2 domain. In some embodiments, a modified CH2 domain as described herein is joined to a CH3 domain, which may be a naturally occurring CH3 domain or a variant CH3 domain, typically at the N-terminal end of the CH3 domain. In some embodiments, the protein comprising a modified CH3 domain joined to a CH2 domain or a modified CH2 domain joined to a CH3 domain, further comprises a partial or full hinge region of an antibody, thus resulting in a format in which the modified CH2 or CH3 domain is part of an Fc polypeptide having a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:234), or portions thereof as shown in SEQ ID NOS:232-233. In further embodiments, the protein, which may be in an Fc format containing a hinge or partial hinge region, is further joined to another moiety, for example, a Fab fragment or a portion thereof, thus generating a transferrin receptor-binding Fab-Fc fusion. In some embodiments, the transferrin receptor-binding Fab-Fc fusion comprises a modified CH2 or CH3 domain, a hinge region, and a Fab fragment or a portion thereof. The Fab fragment may be targeting or non-targeting. In certain embodiments, the Fab fragment is targeting. In certain embodiments, the Fab fragment is non-targeting. In certain embodiments, the Fab fragment does not specifically bind to transferrin via its heavy or light chain variable regions. In certain embodiments, a TfR binding protein as described herein does not comprise a Fab fragment or a portion thereof. In some embodiments, a TfR binding protein as described herein does not comprise antigen-binding portion or variable domain portion of an Fab fragment. In some embodiments, a TfR binding protein as described herein does not comprise an antibody antigen-binding domain or antibody variable domain.

In some embodiments, an Fc polypeptide comprising a modified CH2 or CH3 domain as described herein or a Fab-Fc fusion comprising a modified CH2 or CH3 domain as described herein is a subunit of a dimer. Thus, in certain embodiments, a protein as described herein may comprise a Fc polypeptide dimer or a Fab-Fc dimer fusion comprising a first Fc polypeptide comprising a modified constant domain; and a second Fc polypeptide capable of dimerizing to the first Fc polypeptide. In some embodiments, the dimer is a heterodimer. In some embodiments, the dimer is a homodimer. In some embodiments, the dimer comprises a single polypeptide that binds to the transferrin receptor, i.e., is monovalent for transferrin receptor binding. In some embodiments, the dimer comprises a second polypeptide that binds to the transferrin receptor. The second polypeptide may comprise the same modified CH2/CH3 domain present in the Fc or Fab-Fc fusion to provide a bivalent binding homodimer, or a second modified CH2/CH3 domain as described herein may provide a second transferrin receptor binding site. In some embodiments, the dimer comprises a first subunit comprising a modified CH2 or CH3 domain and a second subunit comprising CH2 and CH3 domains where neither binds a transferrin receptor.

Transferrin receptor-binding proteins as described herein may have a broad range of binding affinities, e.g., based on the format of the protein. For example, in some embodiments, a protein comprising a modified CH3 domain has an affinity for transferrin receptor binding ranging anywhere from 1 pM to 10 μM. In some embodiments, affinity may be measured in a monovalent format. In other embodiments, affinity may be measured in a bivalent format, e.g., as a dimer comprising a polypeptide-Fab fusion protein.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet® (ForteBio, Inc., Menlo Park, CA)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

### Additional Mutations in an Fc Polypeptide

As described herein, a constant domain modified to bind to TfR may be comprised within an Fc polypeptide or a Fab-Fc fusion. In certain embodiments, the polypeptide may comprise additional mutations, e.g., to increase serum stability, to modulate effector function, to influence glyscosylation, to reduce immunogenicity in humans, and/or to provide for knob and hole heterodimerization of the polypeptide. In certain embodiments, a polypeptide described herein may be further modified to remove the C-terminal Lys residue (i.e., the Lys residue at position 220, as numbered with reference to SEQ ID NO:1)).

In certain embodiments, the Fc polypeptide modified to bind to TfR may be dimerized to a second Fc polypeptide. Thus, in certain embodiments, a protein as described herein may comprise an Fc dimer comprising a modified CH2 or CH3 domain within a first polypeptide; and a second Fc polypeptide. Thus, in some aspects, a protein described herein comprises two Fc polypeptides, wherein one or both Fc polypeptides each comprise independently selected modifications (e.g., a modification or mutation described herein).

In some embodiments, a polypeptide as described herein has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, polypeptides present in an Fc dimer may include knob and hole mutations to promote heterodimer formation. Generally, the method involves introducing a protuberance (“knob”) at the interface of one polypeptide and a corresponding cavity (“hole”) in the interface of the other polypeptide. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). Such additional mutations are at a position in the polypeptide that does not have a negative effect on binding of the modified CH2 or CH3 domain to the transferrin receptor.

In one illustrative embodiment of a knob and hole approach for dimerization, a position corresponding to position 139 of SEQ ID NO:1 of a first Fc polypeptide subunit to be dimerized has a tryptophan in place of a native threonine and a second Fc polypeptide subunit of the dimer has a valine at a position corresponding to position 180 of SEQ ID NO:1 in place of the native tyrosine. The second subunit of the Fc polypeptide may further comprise a substitution in which the native threonine at the position corresponding to position 139 of SEQ ID NO:1 is substituted with a serine and a native leucine at the position corresponding to position 141 of SEQ ID NO:1 is substituted with an alanine.

A polypeptide as described herein may also be engineered to contain other modifications for heterodimerization, e.g., electrostatic engineering of contact residues within a CH3-CH3 interface that are naturally charged or hydrophobic patch modifications.

In some embodiments, modifications to enhance serum half-life may be introduced. For example, in some embodiments, an Fc polypeptide comprises a CH2 domain comprising a Tyr at a position corresponding to position 25 of SEQ ID NO:1, Thr at a position corresponding to 27 of SEQ ID NO:1, and Glu at a position corresponding to position 29 of SEQ ID NO:1.

In some embodiments, a mutation, e.g., a substitution, is introduced at one or more of positions 17-30, 52-57, 80-90, 156-163, and 201-208 as determined with reference to SEQ ID NO:1. In some embodiments, one or more mutations are introduced at positions 24, 25, 27, 28, 29, 80, 81, 82, 84, 85, 87, 158, 159, 160, 162, 201, 206, 207, or 209 as determined with reference to SEQ ID NO:1. In some embodiments, mutations are introduced into one or two of positions 201 and 207 as determined with reference to SEQ ID NO:1 (e.g., M201L and N207S). In some embodiments, a polypeptide as described herein further comprises mutation N207S or N207A, with or without M201L. In some embodiments, a polypeptide as described herein comprises a substitution at one, two or all three of positions T80, E153, and N207 as numbered with reference to SEQ ID NO:1 (e.g., T80Q and N207A or T80A, E153A, and N207A). In some embodiments, a polypeptide as described herein comprises substitutions at positions T23 and M201 as numbered with reference to SEQ ID NO:1 (e.g., T23Q and M201L).

In some embodiments, the C-terminal Lys residue is removed or is absent in an Fc polypeptide described herein (i.e., the Lys residue at position 220 is removed or is absent, as numbered with reference to SEQ ID NO:1) or in a protein comprising an Fc polypeptide described herein. For example, in certain embodiments, the C-terminal Lys residue may be removed or is absent from any one of SEQ ID NOs: 1, 4-29, 236-299, 302, 361-372, 397-408, 421-435, 491-497, 521-518, 557-561, 623-633, 655, 702-731, 743-744, 801-810, and 812-818. Illustrative truncated Fc polypeptide sequences or proteins comprising such truncated Fc polypeptide sequences include, e.g., SEQ ID NOs: 634-654, 733-742, and 745-746.

### Fc Effector Functions

In some embodiments, a first Fc polypeptide comprising a modified CH2 or CH3 domain and/or a second Fc polypeptide has an effector function, i.e., they have the ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, a polypeptide as described herein may include additional modifications that reduce effector function or eliminate effector function. Alternatively, in some embodiments, a first polypeptide comprising a modified CH2 or CH3 domain as described herein and/or a second polypeptide may include additional modifications that enhance effector function.

Illustrative Fc polypeptide mutations that modulate an effector function include, but are not limited to, substitutions in a CH2 domain, e.g., at positions corresponding to positions 7 and 8 of SEQ ID NO:1. In some embodiments, the substitutions in a modified CH2 domain comprise Ala at positions 7 and 8 of SEQ ID NO:1. In some embodiments, the substitutions in a modified CH2 domain comprise Ala at positions 7 and 8 and Gly at position 102 of SEQ ID NO:1. In some embodiments, the substitutions in a modified CH2 domain comprise Ala at positions 7 and 8 and Ser at position 102 of SEQ ID NO:1.

Additional Fc polypeptide mutations that modulate an effector function include, but are not limited to, one or more substitutions at positions 238, 265, 269, 270, 297, 327 and 329 (EU numbering scheme, which correspond to positions 11, 38, 42, 43, 70, 100, and 102 as numbered with reference to SEQ ID NO:1). Illustrative substitutions (as numbered with EU numbering scheme), include the following: position 329 may have a mutation in which proline is substituted with a glycine, alanine, serine, or arginine or an amino acid residue large enough to destroy the Fc/Fc□ receptor interface that is formed between proline 329 of the Fc and tryptophan residues Trp 87 and Trp 110 of Fc□RIII. Additional illustrative substitutions include S228P, E233P, L235E, N297A, N297D, and P331S. Multiple substitutions may also be present, e.g., L234A and L235A of a human IgG1 Fc polypeptide; L234A, L235A, and P329G of a human IgG1 Fc polypeptide; L234A, L235A, and P329S of a human IgG1 Fc polypeptide; S228P and L235E of a human IgG4 Fc polypeptide; L234A and G237A of a human IgG1 Fc polypeptide; L234A, L235A, and G237A of a human IgG1 Fc polypeptide; V234A and G237A of a human IgG2 Fc polypeptide; L235A, G237A, and E318A of a human IgG4 Fc polypeptide; and S228P and L236E of a human IgG4 Fc polypeptide. In some embodiments, a polypeptide as described herein may have one or more amino acid substitutions that modulate ADCC, e.g., substitutions at positions 298, 333, and/or 334 of the Fc polypeptide, according to the EU numbering scheme.

In some embodiments, a polypeptide as described herein may have one or more amino acid substitutions that increase or decrease ADCC or may have mutations that alter C1q binding and/or CDC.

### Illustrative Polypeptides Comprising Additional Mutations

A polypeptide as described herein, such as a polypeptide comprising a modified CH3 domain (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) and/or a second polypeptide, may comprise mutations including a knob mutation (e.g., T139W as numbered with reference to SEQ ID NO:1), hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and/or mutations that increase serum stability (e.g., (i) M25Y, S27T, and T29E as numbered with reference to SEQ ID NO:1, or (ii) N207S with or without M201L as numbered with reference to SEQ ID NO:1).

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have a knob mutation (e.g., T139W as numbered with reference to SEQ ID NO:1) and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have a knob mutation. In some embodiments, a polypeptide as described herein (e.g., a second Fc polypeptide) may have a knob mutation (e.g., T139W as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have a knob mutation.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO: 1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have a knob mutation and mutations that modulate effector function. In some embodiments, a polypeptide as described herein (e.g., a second Fc polypeptide) may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have a knob mutation and mutations that modulate effector function.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have a knob mutation (e.g., T139W as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have a knob mutation and mutations that increase serum stability. In some embodiments, a polypeptide as described herein (e.g., a second Fc polypeptide) may have a knob mutation (e.g., T139W as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have a knob mutation and mutations that increase serum stability.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have a knob mutation, mutations that modulate effector function, and mutations that increase serum stability. In some embodiments, a polypeptide as described herein (e.g., a second Fc polypeptide) may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have a knob mutation, mutations that modulate effector function, and mutations that increase serum stability.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have hole mutations (e.g., T139S, L141A, and Y180V as numbered with reference to SEQ ID NO:1) and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have hole mutations. In some embodiments, a polypeptide as described herein (e.g., a second Fc polypeptide) may have hole mutations (e.g., T139S, L141A, and Y180V as numbered with reference to SEQ ID NO: 1) and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have hole mutations.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have hole mutations (e.g., T139S, L141A, and Y180V as numbered with reference to SEQ ID NO:1), mutations that modulate effector function (e.g., L7A, L8A, and/or P102G or (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have hole mutations and mutations that modulate effector function. In some embodiments, a polypeptide as described herein (e.g., a second Fc polypeptide) may have hole mutations (e.g., T139S, L141A, and Y180V as numbered with reference to SEQ ID NO:1), mutations that modulate effector function (e.g., L7A, L8A, and/or P102G or (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NO:1 or 634. In some embodiments, a polypeptide having the sequence of SEQ ID NO:1 or 634 may be modified to have hole mutations and mutations that modulate effector function.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have hole mutations (e.g., T139S, L141A, and Y180V as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have hole mutations and mutations that increase serum stability. In some embodiments, a polypeptide as described herein (e.g., a second Fc polypeptide) may have hole mutations (e.g., T139S, L141A, and Y180V as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO: 1), and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have hole mutations and mutations that increase serum stability.

In some embodiments, a polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.23.2.1, and CH3C.35.23.1.1) may have hole mutations (e.g., T139S, L141A, and Y180V as numbered with reference to SEQ ID NO: 1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of any one of SEQ ID NOS:4-29, 236-299, 422-435, 645, 650 and 746. In some embodiments, a polypeptide having the sequence of any one of SEQ ID NOS:4-29, 236-299, 422-435, 645, 650 and 746 may be modified to have hole mutations, mutations that modulate effector function, and mutations that increase serum stability. In some embodiments, a polypeptide as described herein (e.g., a second Fc polypeptide) may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., (i) M25Y, S27T, and T29E as numbered with reference to SEQ ID NO:1, or (ii) N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:1 or 634. In some embodiments, a polypeptide having the sequence of SEQ ID NO:1 or 634 may be modified to have hole mutations, mutations that modulate effector function, and mutations that increase serum stability.

### Clone CH3C.35.23.2

In some embodiments, clone CH3C.35.23.2 may have a knob mutation (e.g., T139W as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:361 or 646. In some embodiments, clone CH3C.35.23.2 with the knob mutation has the sequence of SEQ ID NO:361. In some embodiments, clone CH3C.35.23.2 with the knob mutation has the sequence of SEQ ID NO:646.

In some embodiments, clone CH3C.35.23.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:362, 363, 632 and 647-649 (e.g., 362 or 363). In some embodiments, clone CH3C.35.23.2 with the knob mutation and the mutations that modulate effector function has the sequence of SEQ ID NO:362, 363 or 632. In some embodiments, clone CH3C.35.23.2 with the knob mutation and the mutations that modulate effector function has the sequence of SEQ ID NO:647-649. In some embodiments, the N-terminus of clone CH3C.35.23.2 with the knob mutation and the mutations that modulate effector function includes a hinge sequence or a portion of a hinge sequence (see, e.g., SEQ ID NO:804 or 741). In some embodiments, the N-terminus of clone CH3C.35.23.2 with the knob mutation and the mutations that modulate effector function is further joined to a CH1 region (see, e.g., SEQ ID NOS:743-745).

In some embodiments, clone CH3C.35.23.2 may have a knob mutation (e.g., T139W as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:364. In some embodiments, clone CH3C.35.23.2 with the knob mutation and the mutations that increase serum stability has the sequence of SEQ ID NO:364.

In some embodiments, clone CH3C.35.23.2 may have a knob mutation (e.g., T139W as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:492. In some embodiments, clone CH3C.35.23.2 with the knob mutation and the mutations that increase serum stability has the sequence of SEQ ID NO:492.

In some embodiments, clone CH3C.35.23.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:365 or 366. In some embodiments, clone CH3C.35.23.2 with the knob mutation, the mutations that modulate effector function, and the mutations that increase serum stability has the sequence of SEQ ID NO:365 or 366.

In some embodiments, clone CH3C.35.23.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:493 or 494. In some embodiments, clone CH3C.35.23.2 with the knob mutation, the mutations that modulate effector function, and the mutations that increase serum stability has the sequence of SEQ ID NO:493 or 494.

In some embodiments, clone CH3C.35.23.2 may have hole mutations (e.g., T139S, L141A, and Y180V as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:367. In some embodiments, clone CH3C.35.23.2 with the hole mutations has the sequence of SEQ ID NO:367.

In some embodiments, clone CH3C.35.23.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:368 or 369. In some embodiments, clone CH3C.35.23.2 with the hole mutations and the mutations that modulate effector function has the sequence of SEQ ID NO:368 or 369.

In some embodiments, clone CH3C.35.23.2 may have hole mutations (e.g., T139S, L141A, and Y180V as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:370. In some embodiments, clone CH3C.35.23.2 with the hole mutations and the mutations that increase serum stability has the sequence of SEQ ID NO:370.

In some embodiments, clone CH3C.35.23.2 may have hole mutations (e.g., T139S, L141A, and Y180V as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:495. In some embodiments, clone CH3C.35.23.2 with the hole mutations and the mutations that increase serum stability has the sequence of SEQ ID NO:495.

In some embodiments, clone CH3C.35.23.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:371 or 372. In some embodiments, clone CH3C.35.23.2 with the hole mutations, the mutations that modulate effector function, and the mutations that increase serum stability has the sequence of SEQ ID NO:371 or 372.

In some embodiments, clone CH3C.35.23.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:496 or 497. In some embodiments, clone CH3C.35.23.2 with the hole mutations, the mutations that modulate effector function, and the mutations that increase serum stability has the sequence of SEQ ID NO:496 or 497.

### Clone CH3C.35.23.3

In some embodiments, clone CH3C.35.23.3 may have a knob mutation (e.g., T139W as numbered with reference to SEQ ID NO:1. In some embodiments, clone CH3C.35.23.3 may have a knob mutation (e.g., T139W as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23.3 may have a knob mutation (e.g., T139W as numbered with reference to SEQ ID NO:1) and mutations that increase serum stability (e.g., M25Y, S27T, and T29E as numbered with reference to SEQ ID NO:1). In some embodiments, clone CH3C.35.23.3 may have a knob mutation (e.g., T139W as numbered with reference to SEQ ID NO:1) and mutations that increase serum stability (e.g., N207S with or without M201L as numbered with reference to SEQ ID NO:1). In some embodiments, clone CH3C.35.23.3 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.3 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

In some embodiments, clone CH3C.35.23.3 may have hole mutations (e.g., T139S, L141A, and Y180V as numbered with reference to SEQ ID NO:1). In some embodiments, clone CH3C.35.23.3 may have hole mutations (e.g., T139S, L141A, and Y180V as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23.3 may have hole mutations (e.g., T139S, L141A, and Y180V as numbered with reference to SEQ ID NO:1) and mutations that increase serum stability (e.g., M25Y, S27T, and T29E as numbered with reference to SEQ ID NO:1). In some embodiments, clone CH3C.35.23.3 may have hole mutations (e.g., T139S, L141A, and Y180V as numbered with reference to SEQ ID NO:1) and mutations that increase serum stability (e.g., N207S with or without M201L as numbered with reference to SEQ ID NO:1). In some embodiments, clone CH3C.35.23.3 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.3 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

### Clone CH3C.35.23.4

In some embodiments, clone CH3C.35.23.4 may have a knob mutation (e.g., T139W as numbered with reference to SEQ ID NO:1). In some embodiments, clone CH3C.35.23.4 may have a knob mutation (e.g., T139W as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). For example, in some embodiments, clone CH3C.35.23.4 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:718 or 742. In some embodiments, clone CH3C.35.23.4 may have a knob mutation (e.g., T139W as numbered with reference to SEQ ID NO:1) and mutations that increase serum stability (e.g., M25Y, S27T, and T29E as numbered with reference to SEQ ID NO:1). In some embodiments, clone CH3C.35.23.4 may have a knob mutation (e.g., T139W as numbered with reference to SEQ ID NO:1) and mutations that increase serum stability (e.g., N207S with or without M201L as numbered with reference to SEQ ID NO:1). In some embodiments, clone CH3C.35.23.4 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.4 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

In some embodiments, clone CH3C.35.23.4 may have hole mutations (e.g., T139S, L141A, and Y180V as numbered with reference to SEQ ID NO:1). In some embodiments, clone CH3C.35.23.4 may have hole mutations (e.g., T139S, L141A, and Y180V as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23.4 may have hole mutations (e.g., T139S, L141A, and Y180V as numbered with reference to SEQ ID NO:1) and mutations that increase serum stability (e.g., M25Y, S27T, and T29E as numbered with reference to SEQ ID NO:1). In some embodiments, clone CH3C.35.23.4 may have hole mutations (e.g., T139S, L141A, and Y180V as numbered with reference to SEQ ID NO:1) and mutations that increase serum stability (e.g., N207S with or without M201L as numbered with reference to SEQ ID NO:1). In some embodiments, clone CH3C.35.23.4 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.4 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

### Clone CH3C.35.21.17.2

In some embodiments, clone CH3C.35.21.17.2 may have a knob mutation (e.g., T139W as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:397 or 651. In some embodiments, clone CH3C.35.21.17.2 with the knob mutation has the sequence of SEQ ID NO:397. In some embodiments, clone CH3C.35.21.17.2 with the knob mutation has the sequence of SEQ ID NO:651.

In some embodiments, clone CH3C.35.21.17.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:398, 399, 633, or 652-654 (e.g., 398 or 399). In some embodiments, clone CH3C.35.21.17.2 with the knob mutation and the mutations that modulate effector function has the sequence of SEQ ID NO:398, 399 or 633. In some embodiments, clone CH3C.35.21.17.2 with the knob mutation and the mutations that modulate effector function has the sequence of SEQ ID NO:652-654.

In some embodiments, clone CH3C.35.21.17.2 may have a knob mutation (e.g., T139W as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:400. In some embodiments, clone CH3C.35.21.17.2 with the knob mutation and the mutations that increase serum stability has the sequence of SEQ ID NO:400.

In some embodiments, clone CH3C.35.21.17.2 may have a knob mutation (e.g., T139W as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:513. In some embodiments, clone CH3C.35.21.17.2 with the knob mutation and the mutations that increase serum stability has the sequence of SEQ ID NO:513.

In some embodiments, clone CH3C.35.21.17.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:401 or 402. In some embodiments, clone CH3C.35.21.17.2 with the knob mutation, the mutations that modulate effector function, and the mutations that increase serum stability has the sequence of SEQ ID NO:401 or 402.

In some embodiments, clone CH3C.35.21.17.2 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:514 or 515. In some embodiments, clone CH3C.35.21.17.2 with the knob mutation, the mutations that modulate effector function, and the mutations that increase serum stability has the sequence of SEQ ID NO:514 or 515.

In some embodiments, clone CH3C.35.21.17.2 may have hole mutations (e.g., T139S, L141A, and Y180V as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:403. In some embodiments, clone CH3C.35.21.17.2 with the hole mutations has the sequence of SEQ ID NO:403.

In some embodiments, clone CH3C.35.21.17.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:404 or 405. In some embodiments, clone CH3C.35.21.17.2 with the hole mutations and the mutations that modulate effector function has the sequence of SEQ ID NO:404 or 405.

In some embodiments, clone CH3C.35.21.17.2 may have hole mutations (e.g., T139S, L141A, and Y180V as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:406. In some embodiments, clone CH3C.35.21.17.2 with the hole mutations and the mutations that increase serum stability has the sequence of SEQ ID NO:406.

In some embodiments, clone CH3C.35.21.17.2 may have hole mutations (e.g., T139S, L141A, and Y180V as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:516. In some embodiments, clone CH3C.35.21.17.2 with the hole mutations and the mutations that increase serum stability has the sequence of SEQ ID NO:516.

In some embodiments, clone CH3C.35.21.17.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., M25Y, S27T, and T29E as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:407 or 408. In some embodiments, clone CH3C.35.21.17.2 with the hole mutations, the mutations that modulate effector function, and the mutations that increase serum stability has the sequence of SEQ ID NO:407 or 408.

In some embodiments, clone CH3C.35.21.17.2 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), mutations that increase serum stability (e.g., N207S with or without M201L as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:517 or 518. In some embodiments, clone CH3C.35.21.17.2 with the hole mutations, the mutations that modulate effector function, and the mutations that increase serum stability has the sequence of SEQ ID NO:517 or 518.

### Clone CH3C.35.23

In some embodiments, clone CH3C.35.23 may have a knob mutation (e.g., T139W as numbered with reference to SEQ ID NO:1). In some embodiments, clone CH3C.35.23 may have a knob mutation (e.g., T139W as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23 may have a knob mutation (e.g., T139W as numbered with reference to SEQ ID NO:1) and mutations that increase serum stability (e.g., M25Y, S27T, and T29E as numbered with reference to SEQ ID NO:1). In some embodiments, clone CH3C.35.23 may have a knob mutation (e.g., T139W as numbered with reference to SEQ ID NO:1) and mutations that increase serum stability (e.g., N207S with or without M201L as numbered with reference to SEQ ID NO:1). In some embodiments, clone CH3C.35.23 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

In some embodiments, clone CH3C.35.23 may have hole mutations (e.g., T139S, L141A, and Y180V as numbered with reference to SEQ ID NO:1). In some embodiments, clone CH3C.35.23 may have hole mutations (e.g., T139S, L141A, and Y180V as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23 may have hole mutations (e.g., T139S, L141A, and Y180V as numbered with reference to SEQ ID NO:1) and mutations that increase serum stability (e.g., M25Y, S27T, and T29E as numbered with reference to SEQ ID NO:1). In some embodiments, clone CH3C.35.23 may have hole mutations (e.g., T139S, L141A, and Y180V as numbered with reference to SEQ ID NO:1) and mutations that increase serum stability (e.g., N207S with or without M201L as numbered with reference to SEQ ID NO:1). In some embodiments, clone CH3C.35.23 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

### Clone CH3C.35.21

In some embodiments, clone CH3C.35.21 may have a knob mutation (e.g., T139W as numbered with reference to SEQ ID NO:1). In some embodiments, clone CH3C.35.21 may have a knob mutation (e.g., T139W as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.21 may have a knob mutation (e.g., T139W as numbered with reference to SEQ ID NO:1) and mutations that increase serum stability (e.g., M25Y, S27T, and T29E as numbered with reference to SEQ ID NO:1). In some embodiments, clone CH3C.35.21 may have a knob mutation (e.g., T139W as numbered with reference to SEQ ID NO:1) and mutations that increase serum stability (e.g., N207S with or without M201L as numbered with reference to SEQ ID NO:1). In some embodiments, clone CH3C.35.21 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.21 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

In some embodiments, clone CH3C.35.21 may have hole mutations (e.g., T139S, L141A, and Y180V as numbered with reference to SEQ ID NO:1). In some embodiments, clone CH3C.35.21 may have hole mutations (e.g., T139S, L141A, and Y180V as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.21 may have hole mutations (e.g., T139S, L141A, and Y180V as numbered with reference to SEQ ID NO:1) and mutations that increase serum stability (e.g., M25Y, S27T, and T29E as numbered with reference to SEQ ID NO:1. In some embodiments, clone CH3C.35.21 may have hole mutations (e.g., T139S, L141A, and Y180V as numbered with reference to SEQ ID NO:1) and mutations that increase serum stability (e.g., N207S with or without M201L as numbered with reference to SEQ ID NO:1). In some embodiments, clone CH3C.35.21 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.21 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

### Clone CH3C.35.23.1.1

In some embodiments, clone CH3C.35.23.1.1 may have a knob mutation (e.g., T139W as numbered with reference to SEQ ID NO:1). In some embodiments, clone CH3C.35.23.1.1 may have a knob mutation (e.g., T139W as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23.1.1 may have a knob mutation (e.g., T139W as numbered with reference to SEQ ID NO:1) and mutations that increase serum stability (e.g., M25Y, S27T, and T29E as numbered with reference to SEQ ID NO:1). In some embodiments, clone CH3C.35.23.1.1 may have a knob mutation (e.g., T139W as numbered with reference to SEQ ID NO:1) and mutations that increase serum stability (e.g., N207S with or without M201L as numbered with reference to SEQ ID NO:1). In some embodiments, clone CH3C.35.23.1.1 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.1.1 may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

In some embodiments, clone CH3C.35.23.1.1 may have hole mutations (e.g., T139S, L141A, and Y180V as numbered with reference to SEQ ID NO:1). In some embodiments, clone CH3C.35.23.1.1 may have hole mutations (e.g., T139S, L141A, and Y180V as numbered with reference to SEQ ID NO:1) and mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1). In some embodiments, clone CH3C.35.23.1.1 may have hole mutations (e.g., T139S, L141A, and Y180V as numbered with reference to SEQ ID NO:1) and mutations that increase serum stability (e.g., M25Y, S27T, and T29E as numbered with reference to SEQ ID NO:1). In some embodiments, clone CH3C.35.23.1.1 may have hole mutations (e.g., T139S, L141A, and Y180V as numbered with reference to SEQ ID NO:1) and mutations that increase serum stability (e.g., N207S with or without M201L as numbered with reference to SEQ ID NO:1). In some embodiments, clone CH3C.35.23.1.1 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., M25Y, S27T, and T29E as numbered with reference to SEQ ID NO:1). In some embodiments, clone CH3C.35.23.1.1 may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1) and mutations that increase serum stability (e.g., N207S with or without M201L as numbered with reference to SEQ ID NO:1).

### Fc Polypeptides

In certain embodiments, an Fc polypeptide comprising a modified CH2 or CH3 domain as described herein or an Fab-Fc fusion comprising a modified constant domain as described herein is a subunit of a dimer. Thus, in certain embodiments, a protein as described herein may comprise an Fc dimer comprising a constant domain modified to bind to TfR within a first polypeptide; and a second Fc polypeptide.

In some embodiments, the second Fc polypeptide may have hole mutations (e.g., T139S, L141A, and Y180V as numbered with reference to SEQ ID NO:1) and at least 85% identity, at least 90% identity, at least 95% identity, at least 96% identity, at least 97% identity, at least 98% identity or at least 99% identity to the sequence of SEQ ID NO:557 or 635. In some embodiments, the second Fc polypeptide with the hole mutations has the sequence of SEQ ID NO:557 or 635.

In some embodiments, the second Fc polypeptide may have hole mutations (e.g., T139S, L141A, and Y180V as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:558, 627, 628, 636, 637, or 638. In some embodiments, the second Fc polypeptide with the hole mutations and the mutations that modulate effector function has the sequence of SEQ ID NO: 558, 627, 628, 636, 637, or 638.

In some embodiments, the second Fc polypeptide may have a knob mutation (e.g., T139W as numbered with reference to SEQ ID NO:1). In some embodiments, the second Fc polypeptide may have a knob mutation (e.g., T139W as numbered with reference to SEQ ID NO:1), mutations that modulate effector function (e.g., L7A, L8A, and/or P102G or P102S (e.g., L7A and L8A; L7A, L8A, and P102G; or L7A, L8A, and P102S)) as numbered with reference to SEQ ID NO:1), and at least 85% identity, at least 90% identity, at least 95% identity, at least 96% identity, at least 97% identity, at least 98% identity or at least 99% identity to the sequence of SEQ ID NO:655. In some embodiments, the second Fc polypeptide with the knob mutation and the mutations that modulate effector function has the sequence of SEQ ID NO:655.

### Illustrative Proteins Comprising Non-Targeting Fabs Fragments

In some embodiments, the first and the second Fc polypeptides are each joined to a NTF or a portion thereof, to produce a Fab-Fc dimer fusion.

In some embodiments, a NTF or portion thereof comprises a non-binding variable region (NBVR). A NBVR contains a light chain variable region and a heavy chain variable region and does not specifically bind to a naturally occurring epitope in a subject. In some embodiments, a NBVR does not specifically bind to an antigen expressed in a given mammal, mammalian tissue, or mammalian cell type. The antigen can be a mammalian antigen or an antigen found in the mammal such as from an infectious organism such as a virus, bacteria, fungus, or parasite. The mammal can be, but is not limited to, a non-human primate, a human, or a rodent (e.g., a mouse). An NBVR can be, but is not limited to a scFv.

Specific binding of an antibody to an antigen means an affinity of at least 106 M−1. Specific binding is detectably higher in magnitude and distinguishable from non-specific binding occurring to at least one unrelated target. Nonspecific binding is often the result of van der Waals forces. Non-binding does not imply the NBVR does not bind any antigen with any affinity. Rather, in some embodiments, a NBVR does not exhibit specific binding to (a) any protein or epitope in mammalian cell, mammalian tissue, or mammal; (b) any surface accessible protein or epitope on a mammalian cell or mammalian tissue; or (c) any serum accessible protein or epitope in a mammalian tissue, or mammal.

In some embodiments, the NBVR is a humanized NBVR. Humanized Fabs can be humanized in one or more of: a light chain variable domain, a heavy chain variable domain, a light chain constant domain, and a heavy chain constant (CH1) domain. A humanized NBVR is a genetically engineered NBVR in which CDRs from a non-human “donor” antibody are grafted into human “acceptor” antibody heavy and/or light chain variably region, light chain constant region and/or heavy chain CH1 region sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having at least three, four, five or all CDRs entirely or substantially from a donor antibody and entirely or substantially human antibody variable region framework sequences and/or constant region sequences. Similarly, a humanized heavy chain has at least one, two and usually all three CDRs entirely or substantially from a donor antibody heavy chain, and a heavy chain variable region framework sequence and heavy chain constant region sequences, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region sequences, if present, substantially from human light chain variable region framework and constant region. A CDR in a humanized antibody is substantially from a corresponding CDR in a non-human antibody when at least 85%, 90%, 95% or 100% of corresponding residues (as defined by any conventional definition but preferably defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical.

In some embodiments, the NBVR is a chimeric NBVR. A chimeric NBVR comprises a non-human light and/or heavy chain variable region and a human heavy chain (CH1) and/or light chain constant region.

In some embodiments, the NBVR is a veneered NBVR. A veneered NBVR comprises a partially humanized light and/or heavy chain variable region and a human heavy chain (CH1) and/or light chain constant region.

Exemplary NBVRs include NBVR1 or NBVR2. Unless otherwise apparent from context, reference to NBVR1 or NBVR2 should be understood as referring to any of mouse, chimeric, veneered, humanized, and modified forms of the NBVR1 or NBVR2.

Exemplary NTFs include NBVR1 or NBVR2. Unless otherwise apparent from context, reference to NBVR1 or NBVR2 should be understood as referring to any of mouse, chimeric, veneered, humanized, and modified forms of the NBVR1 or NBVR2.

The sequences of the light and heavy chain variable regions of NBVR1 are designated SEQ ID NOs: 832 and 837, respectively. The sequences of the light and heavy chains of NBVR1 are designated SEQ ID NOs: 833 and 838, respectively

In some embodiments, a NBVR comprises the CDR sequences of NBVR1. The CDRs (L1, L2, and L3) of the light chain of NBVR1 are designated SEQ ID NOs: 819, 821, and 823, respectively. The CDRs (H1, H2, and H3) of the heavy chain of NBVR1 are designated SEQ ID NOs: 825, 827, and 829, respectively. In some embodiments, a NBVR comprises the CDR-L1, CDR-L2, CDR-L3, CDR-H1, and CDR-H3 sequences of NBVR1 and a CDR-H2 sequence comprising SEQ ID NO: 869.

In some embodiments, a NBVR comprises a light chain comprising the amino acid sequence of SEQ ID NO: 833 or 835, and a heavy chain comprising the amino acid sequence of SEQ ID NOs: 838, 839, 840, 841, 844, 845, 846, or 847.

In some embodiments, a NBVR comprises a light chain containing an amino acid sequence that is at least 70%, at least 80%, at least 90%, at least 95%, at least 96%, at least 97%, at least 98%, or at least 99% identical to the amino acid sequence of SEQ ID NOs: 832, 833, or 835, and a heavy chain containing an amino acid sequence that is at least 70%, at least 80%, at least 90%, at least 95%, at least 96%, at least 97%, at least 98%, or at least 99% identical to the amino acid sequence of SEQ ID NOs: 837, 838, 839, 840, 841, 844, 845, 846, or 847, and contains the CDR sequence of the NBVR1 and maintains the non-binding properties of NBVR1.

In some embodiments, a NBVR comprises light chain and heavy chain variably regions that differ from NBVR1 light chain and heavy chain variably regions by a small number of functionally inconsequential amino acid substitutions (e.g., conservative substitutions), deletions, or insertions. NBVRs having at 1, 2, 3, 4, 5, or 6 CDR(s) as defined by any conventional definition, but preferably Kabat, that are 90%, 95%, 99% or 100% identical to corresponding CDRs of NBVR1 or NBVR2 are also included.

The sequences of the light and heavy chain variable regions of NBVR2 are designated SEQ ID NOs: 851 and 853, respectively. The sequences of the light and heavy chains of NBVR2 are designated SEQ ID NOs: 851 and 854, respectively.

In some embodiments, a NBVR comprises the CDR sequences of NBVR2. The CDRs (L1, L2, and L3) of the light chain of NBVR2 are designated SEQ ID NOs: 820, 822, and 824, respectively. The CDRs (H1, H2, and H3) of the heavy chain of NBVR2 are designated SEQ ID NOs: 826, 828, and 829, respectively. In some embodiments, a NBVR comprises the CDR-L1, CDR-L2, CDR-L3, CDR-H1, and CDR-H3 sequences of NBVR2 and a CDR-H2 sequence comprising SEQ ID NO: 869.

In some embodiments, a NBVR comprises a light chain containing an amino acid sequence that is at least 70%, at least 80%, at least 90%, at least 95%, at least 96%, at least 97%, at least 98%, or at least 99% identical to the amino acid sequence of SEQ ID NOs: 850, 851, or 852, and a heavy chain containing an amino acid sequence that is at least 70%, at least 80%, at least 90%, at least 95%, at least 96%, at least 97%, at least 98%, or at least 99% identical to the amino acid sequence of SEQ ID NOs: 853, 854, 855, 856, 857, 859, 860, 861, or 862, and contains the CDR sequence of the NBVR2 and maintains the non-binding properties of NBVR2.

In some embodiments, a NBVR comprises light chain and heavy chain variably regions that differ from NBVR2 light chain and heavy chain variably regions by a small number of functionally inconsequential amino acid substitutions (e.g., conservative substitutions), deletions, or insertions. NBVRs having at 1, 2, 3, 4, 5, or 6 CDR(s) as defined by any conventional definition, but preferably Kabat, that are 90%, 95%, 99% or 100% identical to corresponding CDRs of NBVR1 or NBVR2 are also included.

In some embodiments, a NBVR comprises light and heavy chain variable regions having some or all (e.g., 3, 4, 5, and 6) CDRs entirely or substantially from NBVR1 or NBVR2. Such NBVRs can include a heavy chain variable region that has at least two, and usually all three, CDRs entirely or substantially from the heavy chain variable region of NBVR1 or NBVR2 and/or a light chain variable region having at least two, and usually all three, CDRs entirely or substantially from the light chain variable region of NBVR1 or NBVR2. A CDR is substantially from a corresponding NBVR1 or NBVR2 CDR when it contains no more than 4, 3, 2, or 1 substitutions, insertions, or deletions, except that CDR-H2 (when defined by Kabat) can have no more than 6, 5, 4, 3, 2, or 1 substitutions, insertions, or deletions. Such antibodies can have at least 70%, at least 80%, at least 90%, at least 95%, at least 96%, at least 97%, at least 98%, or at least 99% identity to any of the described NBVR1 or NBVR2 light chain and heavy chain amino acid sequences and maintain their functional properties, and/or differ from NBVR1 or NBVR2. In some embodiments, a NBVR does not exhibit specific binding to (a) any protein or epitope in naturally occurring in mammalian cell, mammalian tissue, or mammal; (b) any surface accessible protein or epitope on a naturally occurring mammalian cell or mammalian tissue; or (c) any serum accessible protein or epitope in a naturally occurring mammalian tissue, or mammal.

In some embodiments, a nucleic acid encoding a NBVR light chain comprises a nucleotide sequence encoding the amino acid sequence of SEQ ID NO: 832, 833, 835, 850, 851, or 852. In some embodiments, a nucleic acid encoding a NBVR heavy chain comprises a nucleotide sequence encoding the amino acid sequence of SEQ ID NO: 837, 838, 839, 840, 841, 844, 845, 846, 847, 853, 854, 855, 856, 857, 859, 860, 861, or 862.

In some embodiments, a nucleic acid encoding a NBVR light chain comprises a nucleotide sequence having at least 70%, at least 75%, at least 80%, at least 85%, at least 90%, at least 95%, at least 96%, at least 97%, at least 98%, or at least 99%, or 100% identity to the nucleotide sequence of SEQ ID NOs: 864 or 866. In some embodiments, a nucleic acid encoding a NBVR heavy chain comprises a nucleotide sequence having at least 70%, at least 75%, at least 80%, at least 85%, at least 90%, at least 95%, at least 96%, at least 97%, at least 98%, or at least 99%, or 100% identity to the nucleotide sequence of SEQ ID NO: 865 or 867.

Described are cells containing nucleic acids encoding the heavy and light chains of any of the described NBVRs. In some embodiments, the cell contains a nucleic acid encoding a NBVR light chain comprising the amino acid sequence of SEQ ID NO: 832, 833, or 835, and a nucleic acid encoding a NBVR heavy chain comprising the amino acid sequence of SEQ ID NO: 837, 838, 839, 840, 841, 844, 845, 846, or 847. In some embodiments, the cell contains a nucleic acid encoding a NBVR light chain comprising the amino acid sequence of SEQ ID NO: 850, 851, or 852, and a nucleic acid encoding a NBVR heavy chain comprising the amino acid sequence of SEQ ID NO: 853, 854, 855, 856, 857, 859, 860, 861, or 862. The cell can be a bacterial cell, a yeast cell, an insect cell or a mammalian cell.

### Engineered Protein Variants for Conjugation to “L”

As described herein, a TfR binding protein may be linked to an oligonucleotide(s) through a linking group “L”. In one aspect, the protein comprises one or more amino acid residues (e.g., amino acid residues that are present at accessible sites in the protein), which may be used to attach the protein to L. For example, in one aspect, the protein comprises one or more cysteine residues (e.g., cysteine residues that are present at accessible sites in the protein). In certain embodiments, the protein is attached to L through a cysteine residue of the protein (e.g., through a sulfur atom of a cysteine residue). In other embodiments, the protein comprises one or more glutamine residues. In certain embodiments, the protein is attached to L through a glutamine residue (e.g., through an amide bond in the side chain of a glutamine residue).

In other aspects, it may be desirable to create engineered proteins with one or more modified sites. These modified sites may be used to facilitate the attachment of P to each L. For example, P may be attached to each L at the modified site. In other embodiments, the modified site may enable the attachment of L to an amino acid residue located near the modified site (e.g., within 1, 2, 3, 4 5, 6, 7, 8, 9 or 10 amino acids of the modified site, such as within 2 or 3 amino acids of the modified site). In particular embodiments, such modified sites are substituted residues that occur at accessible sites of the protein. In certain embodiments, a protein described herein comprises one or more modified sites (e.g., one or more amino acid substitutions, such as a cysteine, alanine or glycine substitution). In certain embodiments, the protein comprises at least or exactly 1, 2, 3, 4, 5, 6, 7, or 8 modified sites. In certain embodiments, the protein comprises 1 to 8, 1 to 7, 1 to 6, 1 to 5, 1 to 4, 1 to 3, or 1 to 2 modified sites. In certain embodiments, the protein comprises 2 to 4 modified sites.

In certain embodiments, a modified site within the protein (P) is an amino acid substitution or insertion. In certain embodiments, the protein (P) is or comprises an Fc dimer, for example, where at least one of the Fc polypeptides has been modified to bind TfR. The modified site within the protein (P) may be in an Fc polypeptide that binds TfR and/or in an Fc polypeptide that has formed a dimer with an Fc polypeptide that binds TfR. In certain embodiments, the Fc polypeptide(s) may be part of a Fab-Fc fusion or Fab-Fc dimer fusion and the modified site may be in a Fab-Fc polypeptide that binds TfR and/or in a Fab-Fc polypeptide that has formed a dimer with a Fab-Fc polypeptide that binds TfR.

In certain embodiments, a modified site is present in a CL domain. In certain embodiments, a modified site is present in a CH1 domain. In certain embodiments, a modified site is present in a CH2 domain. In certain embodiments, a modified site is present in a CH3 domain.

In certain embodiments, the modified site is an amino acid substitution. In certain embodiments, the modified site is a cysteine, glycine or alanine substitution.

In certain embodiments, the modified site is a cysteine substitution. By substituting those residues with cysteine, reactive thiol groups are thereby positioned at accessible sites of the protein and may be used to conjugate the protein (P) to an oligonucleotide (X) via a linking group (L), to create a conjugate as described herein. In certain embodiments, protein contains an Fc polypeptide or Fc polypeptide dimer and includes a cysteine substitution selected from the group consisting of S239C, S442C, A330C, and T289C, wherein the positions and substitutions are according to EU numbering. In other embodiments, the Fc polypeptide is joined to CH1 domain and includes an A114C substitution. In other embodiments, the protein comprises a Fab-Fc fusion, and the light chain includes a K149C substitution.

In other aspects, the modified site is an alanine or glycine substitution. Such modified amino acids may facilitate enzymatic conjugation of L to the protein at a nearby amino acid, such as a glutamine residue (e.g., using bacterial transglutaminase (BTG). For example, in certain embodiments, the alanine/glycine substitution is N297A or N297G, wherein the positions and substitutions are according to EU numbering. These substitutions eliminate glycosylation at position 297, which would hinder enzymatic conjugation of the linker to the protein at position Q295 (i.e., the linker is attached to the protein through an amide bond in the side chain of the glutamine). Thus, in certain embodiments, the modified site is N297A or N297G and the protein (P) is attached to L at Q295 (e.g., by enzymatic conjugation).

Thus, certain embodiments provide a Fc polypeptide, an Fc dimer (e.g., comprising the Fc polypeptide (“a first Fc polypeptide”) and a second Fc polypeptide), or a Fab-Fc fusion (e.g., comprising an Fc dimer) comprising:
a) one or more cysteine, alanine or glycine substitutions (e.g., S239C, S442C, A330C, K149C (light chain), T289C, N297A and/or N297G, according to EU numbering and/or A114C according to Kabat numbering); and
b) a modified constant domain (e.g., a modified CH2 or CH3 domain) that specifically binds to a transferrin receptor described herein (e.g., the modified CH3 domain comprises five, six, seven, eight, or nine substitutions in a set of amino acid positions comprising 157, 159, 160, 161, 162, 163, 186, 189, and 194, wherein the substitutions and the positions are determined with reference to SEQ ID NO:1). In certain embodiments, the Fc polypeptide comprises a sequence having at least 85% identity, at least 90% identity, at least 95% identity, at least 96% identity, at least 97% identity, at least 98% identity or at least 99% identity any one of SEQ ID NOS:4-29, 236-299, 361-372, 397-408, 422-435, 491-497, 512-518, 632-633, 645-654, 702-708, 710-716, 718, 738-740, and 742. In certain embodiments, the N-terminus of the Fc polypeptide includes a portion of the hinge region (e.g., DKTHTCP (SEQ ID NO:232 or DKTHTCPPCP (SEQ ID NO:233)). Thus, in some embodiments, the Fc polypeptide comprises a sequence having at least 85% identity, at least 90% identity, at least 95% identity, at least 96% identity, at least 97% identity, at least 98% identity or at least 99% identity to any one of SEQ ID NOS:804 and 741. In certain embodiments, the N-terminus of the Fc polypeptide is linked via the hinge region to a CH1 domain sequence. Thus, in some embodiments, the protein comprises a sequence having at least 85% identity, at least 90% identity, at least 95% identity, at least 96% identity, at least 97% identity, at least 98% identity or at least 99% identity to any one of SEQ ID NOS: 709, 717, and 743-745. In certain embodiments, the Fc polypeptide has a tryptophan at a position corresponding to position 139 of SEQ ID NO:1. In certain embodiments, the second polypeptide or second Fab-Fc fusion may or may not comprise a modified constant domain. In certain embodiments, the second polypeptide or second Fab-Fc fusion may or may not comprise one or more modified sites (e.g., S239C, S442C, A330C, K149C (light chain), T289C, N297A and/or N297G, according to EU numbering and/or A114C according to Kabat numbering).

Certain embodiments also provide a protein as described herein, which is or comprises a Fc polypeptide dimer, or a Fab-Fc dimer fusion thereof, comprising a first and a second Fc polypeptide, wherein the first Fc polypeptide comprises a modified constant domain (e.g., a modified CH2 or CH3 domain) that specifically binds to a transferrin receptor; wherein the second Fc polypeptide is capable of dimerizing with the first Fc polypeptide; and wherein the first and/or the second Fc polypeptide comprise one or more modified sites; or wherein the first and/or second Fab-Fc fusion comprise one or more modified sites (e.g., one or more amino acid substitutions, such as cysteine substitutions). In certain embodiments, the first Fc polypeptide comprises a modified CH3 domain that specifically binds to a transferrin receptor, wherein the modified CH3 domain comprises five, six, seven, eight, or nine substitutions in a set of amino acid positions comprising 157, 159, 160, 161, 162, 163, 186, 189, and 194. In certain embodiments, the first and the second Fc polypeptides are each joined to a non-targeting Fab fragment or a portion thereof, to produce a Fab-Fc dimer fusion. In certain embodiments, the first Fc polypeptide and the second Fc polypeptide (or first and second Fab-Fc fusion) of the dimer contain substitutions that promote heterodimerization. For example, in certain embodiments, the first polypeptide or first Fab-Fc fusion has a tryptophan in place of a native threonine at a position corresponding to position 139 of SEQ ID NO:1 and a second Fc polypeptide or second Fab-Fc fusion has a valine at position 180 of SEQ ID NO:1, a serine at the position corresponding to position 139 of SEQ ID NO:1, and an alanine at the position corresponding to position 141 of SEQ ID NO:1.

In certain embodiments, the first Fc polypeptide or Fab-Fc fusion comprises one or more amino acid substitutions (e.g., 1 or more cysteine substitutions). In certain embodiments, the first Fc polypeptide or Fab-Fc fusion comprises one or more substitutions selected from the group consisting of S239C, S442C, A330C, K149C (light chain), T289C, N297A and N297G, according to EU numbering and A114C according to Kabat numbering. In certain embodiments, the first Fc polypeptide or first Fab-Fc fusion comprises S239C. In certain embodiments, the first Fc polypeptide or first Fab-Fc fusion comprises S442C. In certain embodiments, the first Fc polypeptide or first Fab-Fc fusion comprises A330C. In certain embodiments, the first Fc polypeptide or first Fab-Fc fusion comprises T289C. In certain embodiments, the first Fc polypeptide or first Fab-Fc fusion comprises N297A. In certain embodiments, the first Fc polypeptide or first Fab-Fc fusion comprises N297G. In certain embodiments, the first Fc polypeptide or first Fab-Fc fusion comprises S239C and A330C. In certain embodiments, the first Fab-Fc fusion comprises K149C on the light chain. In certain embodiments, the first Fab-Fc fusion comprises A114C. In certain embodiments, the first Fc polypeptide or first Fab-Fc fusion comprises a sequence having at least 85% identity, at least 90% identity, at least 95% identity, at least 96% identity, at least 97% identity, at least 98% identity or at least 99% identity any one of SEQ ID NOS:4-29, 236-299, 361-372, 397-408, 422-435, 491-497, 512-518, 632-633, 645-654, 702-708, 709-717, 718, 738-740, 741-746, and 804.

In certain embodiments, the second Fc polypeptide or Fab-Fc fusion comprises one or more amino substitutions (e.g., 1 or more cysteine substitutions). In certain embodiments, the second Fc polypeptide or Fab-Fc fusion comprises one or more substitutions selected from the group consisting of S239C, S442C, A330C, K149C (light chain), T289C, N297A and N297G, according to EU numbering and A114C according to Kabat numbering. In certain embodiments, the second Fc polypeptide or second Fab-Fc fusion comprises S239C. In certain embodiments, the second Fc polypeptide or second Fab-Fc fusion comprises S442C. In certain embodiments, the second Fc polypeptide or second Fab-Fc fusion comprises A330C. In certain embodiments, the second Fc polypeptide or second Fab-Fc fusion comprises T289C. In certain embodiments, the second Fc polypeptide or second Fab-Fc fusion comprises N297A. In certain embodiments, the second Fc polypeptide or second Fab-Fc fusion polypeptide comprises N297G. In certain embodiments, the second Fc polypeptide or second Fab-Fc fusion comprises S239C and A330C. In certain embodiments, the second Fab-Fc fusion comprises K149C on the light chain. In certain embodiments, the second Fab-Fc fusion comprises A114C. In certain embodiments, the second Fc polypeptide or Fab-Fc fusion comprises a sequence having at least 85% identity, at least 90% identity, at least 95% identity, at least 96% identity, at least 97% identity, at least 98% identity, or at least 99% identity any one of the sequence of any one of SEQ ID NOS:557-561, 627-631, 635-644, 719-723, and 724-731, 733-737, and 810.

In certain embodiments, the first Fc polypeptide and the second Fc polypeptide (or first and second Fab-Fc fusion) each comprise one or more amino acid substitutions (e.g., 1 or more cysteine substitutions). In certain embodiments, the one or more substitutions are: S239C, S442C, A330C, T289C, N297A and/or N297G, according to EU numbering and A114C according to Kabat numbering. In certain embodiments, the light chain contains a K149C substitution. In certain embodiments, the one or more substitutions are: S239C, S442C, A330C, K149C (light chain), A114C, and/or T289C. In certain embodiments, the one or more substitutions are: S239C, S442C, A114C, and/or T289C. In certain embodiments, the one or more substitutions are: N297A and/or N297G. In certain embodiments, the first Fc polypeptide and the second Fc polypeptide (or first and second Fab-Fc fusion) each comprise one amino acid substitution (e.g., 1 cysteine substitution). In certain embodiments, the first and second Fc polypeptides each comprise a cysteine substitution at S239C; or the first and second Fab-Fc fusions each comprise a cysteine substitution at S239C. In certain embodiments, the first and second Fc polypeptides each comprise two amino acid substitutions (e.g., 2 cysteine substitutions); or wherein the first and second Fab-Fc fusions each comprise two amino acid substitutions (e.g., cysteine substitutions). In certain embodiments, the first and second Fc polypeptides each comprise a cysteine substitution at S239C and A330C; or the first and second Fab-Fc fusions each comprise a cysteine substitution at S239C and A330C.

In certain embodiments, the first Fc polypeptide or first Fab-Fc fusion comprises a sequence having at least 85%, 90%, 95%, 96%, 97%, 98% or 99% sequence identity to SEQ ID NO:279, 361, 362, 363, 743-744 or 804, wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS:559-561, 719-731 or 810. In certain embodiments, the first polypeptide or first Fab-Fc fusion comprises the sequence of SEQ ID NO:362, and the second polypeptide or second Fab-Fc fusion comprises the sequence of SEQ ID NO:559. In certain embodiments, the first polypeptide or first Fab-Fc fusion comprises the sequence of SEQ ID NO:362, and the second polypeptide or second Fab-Fc fusion comprises the sequence of SEQ ID NO:560. In certain embodiments, the first polypeptide or first Fab-Fc fusion comprises the sequence of SEQ ID NO:362, and the second polypeptide or second Fab-Fc fusion comprises the sequence of SEQ ID NO:561. In certain embodiments, the first polypeptide or first Fab-Fc fusion comprises the sequence of SEQ ID NO:363, and the second polypeptide or second Fab-Fc fusion comprises the sequence of SEQ ID NO:726. In certain embodiments, the first polypeptide or first Fab-Fc fusion comprises the sequence of SEQ ID NO:744, and the second polypeptide or second Fab-Fc fusion comprises the sequence of SEQ ID NO:731. In certain embodiments, the first polypeptide or first Fab-Fc fusion comprises the sequence of SEQ ID NO:363, and the second polypeptide or second Fab-Fc fusion comprises the sequence of SEQ ID NO:725. In certain embodiments, the N-terminus of the first and the second Fc polypeptide includes a portion of the hinge region (e.g., DKTHTCP (SEQ ID NO:232 or DKTHTCPPCP (SEQ ID NO:233)). Thus, in certain embodiments, the first polypeptide comprises the sequence of SEQ ID NO:804, and the second polypeptide comprises the sequence of SEQ ID NO:810.

In certain embodiments, the first Fc polypeptide or first Fab-Fc fusion comprises a sequence having at least 85%, 90%, 95%, 96%, 97%, 98% or 99% sequence identity to any one of SEQ ID NOS:645-648, 741 and 745, wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS:639-641 and 733-737. In certain embodiments, the first polypeptide or first Fab-Fc fusion comprises the sequence of SEQ ID NO:647, and the second polypeptide or second Fab-Fc fusion comprises the sequence of SEQ ID NO:639. In certain embodiments, the first polypeptide or first Fab-Fc fusion comprises the sequence of SEQ ID NO:647, and the second polypeptide or second Fab-Fc fusion comprises the sequence of SEQ ID NO:640. In certain embodiments, the first polypeptide or first Fab-Fc fusion comprises the sequence of SEQ ID NO:647, and the second polypeptide or second Fab-Fc fusion comprises the sequence of SEQ ID NO:641. In certain embodiments, the first polypeptide or first Fab-Fc fusion comprises the sequence of SEQ ID NO:648, and the second polypeptide or second Fab-Fc fusion comprises the sequence of SEQ ID NO:735. In certain embodiments, the first polypeptide or first Fab-Fc fusion comprises the sequence of SEQ ID NO:745, and the second polypeptide or second Fab-Fc fusion comprises the sequence of SEQ ID NO:737. In certain embodiments, the first polypeptide or first Fab-Fc fusion comprises the sequence of SEQ ID NO:648, and the second polypeptide or second Fab-Fc fusion comprises the sequence of SEQ ID NO:734. In certain embodiments, the N-terminus of the first and second Fc polypeptide includes a portion of the hinge region (e.g., DKTHTCP (SEQ ID NO:232 or DKTHTCPPCP (SEQ ID NO:233)). Thus, in certain embodiments, the first polypeptide comprises the sequence of SEQ ID NO:741, and the second polypeptide comprises the sequence of SEQ ID NO:736.

In certain embodiments, the first Fc polypeptide or first Fab-Fc fusion comprises a sequence having at least 85%, 90%, 95%, 96%, 97%, 98% or 99% sequence identity to SEQ ID NO:702-717, wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS:557-561, 627, 719-731 and 810. In certain embodiments, the first polypeptide or first Fab-Fc fusion comprises the sequence of SEQ ID NO:702, and the second polypeptide or second Fab-Fc fusion comprises the sequence of SEQ ID NO:559. In certain embodiments, the first polypeptide or first Fab-Fc fusion comprises the sequence of SEQ ID NO:708, and the second polypeptide or second Fab-Fc fusion comprises the sequence of SEQ ID NO:722. In certain embodiments, the first polypeptide or first Fab-Fc fusion comprises the sequence of SEQ ID NO:713, and the second polypeptide or second Fab-Fc fusion comprises the sequence of SEQ ID NO:726. In certain embodiments, the first polypeptide or first Fab-Fc fusion comprises the sequence of SEQ ID NO:713, and the second polypeptide or second Fab-Fc fusion comprises the sequence of SEQ ID NO:627.

In certain embodiments, the first Fc polypeptide or first Fab-Fc fusion comprises a sequence having at least 85%, 90%, 95%, 96%, 97%, 98% or 99% sequence identity to SEQ ID NO:738-741, wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS:635-637, 639-641, and 733-737. In certain embodiments, the first polypeptide or first Fab-Fc fusion comprises the sequence of SEQ ID NO:738, and the second polypeptide or second Fab-Fc fusion comprises the sequence of SEQ ID NO:639. In certain embodiments, the first polypeptide or first Fab-Fc fusion comprises the sequence of SEQ ID NO:739, and the second polypeptide or second Fab-Fc fusion comprises the sequence of SEQ ID NO:733. In certain embodiments, the first polypeptide or first Fab-Fc fusion comprises the sequence of SEQ ID NO:740, and the second polypeptide or second Fab-Fc fusion comprises the sequence of SEQ ID NO:735. In certain embodiments, the first polypeptide or first Fab-Fc fusion comprises the sequence of SEQ ID NO:740, and the second polypeptide or second Fab-Fc fusion comprises the sequence of SEQ ID NO:637.

In certain embodiments, the first Fc polypeptide or first Fab-Fc fusion comprises a sequence having at least 85%, 90%, 95%, 96%, 97%, 98% or 99% sequence identity to SEQ ID NO:281 or 718, wherein the polypeptide comprises a Glu at position 153, Tyr at position 157, Thr at position 159, Glu at position 160, Trp at position 161, Ser at position 162, Asn at position 163, Ser at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS: 559-561, 719-731 or 810. In certain embodiments, the first polypeptide or first Fab-Fc fusion comprises the sequence of SEQ ID NO:718, and the second polypeptide or second Fab-Fc fusion comprises the sequence of SEQ ID NO:559.

In certain embodiments, the first Fc polypeptide or first Fab-Fc fusion comprises a sequence having at least 85%, 90%, 95%, 96%, 97%, 98% or 99% sequence identity to SEQ ID NO:742 or 746, wherein the polypeptide comprises a Glu at position 153, Tyr at position 157, Thr at position 159, Glu at position 160, Trp at position 161, Ser at position 162, Asn at position 163, Ser at position 186, Glu at position 188, Glu at position 189, and Phe at position 194, as numbered with reference to SEQ ID NO:1; and the second Fc polypeptide or second Fab-Fc fusion comprises the sequence of any one of SEQ ID NOS: 639-641 and 733-737. In certain embodiments, the first polypeptide or first Fab-Fc fusion comprises the sequence of SEQ ID NO:742, and the second polypeptide or second Fab-Fc fusion comprises the sequence of SEQ ID NO:639.

In certain embodiments, the first Fab-Fc fusion comprises a sequence having at least 85%, 90%, 95%, 96%, 97%, 98% or 99% sequence identity to SEQ ID NO:279, 361, 362, 363 743-744, or 804, wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fab-Fc fusion comprises the sequence of any one of SEQ ID NOS:557-558 and 627. In certain embodiments, the first and Fab-Fc fusions each further comprise a CL comprising the sequence of SEQ ID NO:732.

In certain embodiments, the first Fab-Fc fusion comprises a sequence having at least 85%, 90%, 95%, 96%, 97%, 98% or 99% sequence identity to SEQ ID NO: 645-648, 741, and 745 wherein the polypeptide comprises a Glu at position 153, Tyr at position 157, Thr at position 159, Glu at position 160, Trp at position 161, Ala at position 162, Asn at position 163, Thr at position 186, Glu at position 188, Glu at position 189, and Phe at position 194, as numbered with reference to SEQ ID NO:1; and the second Fab-Fc fusion comprises the sequence of any one of SEQ ID NOS:635-637. In certain embodiments, the first and Fab-Fc fusions each further comprise a CL comprising the sequence of SEQ ID NO:732.

Fc polypeptides or Fab-Fc fusions as described herein, or dimers thereof, that comprise one or more modified sites (e.g., cysteine substitutions) (P) may be used in a conjugate as described herein. Thus, certain embodiments also provide, a conjugate of formula (I):

P-(L-(X)_{y})ₙ (I)

wherein,
each X is independently an oligonucleotide (e.g., selected from an ASO and an RNAi agent);
each L is independently a linking group as described herein;
P is a protein comprising a first Fc polypeptide as described herein, or a dimer thereof, or a Fab-Fc fusion as described herein, or a dimer thereof, wherein the protein comprises one or more modified sites (e.g., cysteine substitutions), which facilitate the attachment of P to each L;
each y is independently 1 or more (e.g., 1, 2, 3 or 4); and
n is 1 or more (e.g., 1, 2, 3, 4, 5, 6, 7 or 8).

In certain embodiments, 1 or more oligonucleotides are attached to the linking group (L). In certain embodiments, 2 or more oligonucleotides are attached to the linking group (L). In certain embodiments, 1 oligonucleotide is attached to the linking group (L). In certain embodiments, 2 oligonucleotides are attached to the linking group (L).

In embodiments, useful values of y include integers from 1-50, 1-40, 1-30, 1-20, 1-10, or 1, 2, 3, 4, 5, 6, 7, 8, 9, 10, 11, 12, 13, 14, 15, 16, 17, 18, 19 or 20.

In embodiments, useful values of n include integers from 1-50, 1-40, 1-30, 1-20, 1-10, or 1, 2, 3, 4, 5, 6, 7, 8, 9, 10, 11, 12, 13, 14, 15, 16, 17, 18, 19 or 20.

### X. Methods of Use

A conjugate as described herein may be used for a variety of purposes, including therapeutic indications.

In some embodiments, the conjugate is used to deliver an oligonucleotide (e.g., an ASO or RNAi agent) to a target cell type that expresses the transferrin receptor. In some embodiments, a conjugate may be used to transport an oligonucleotide (e.g., an ASO or RNAi agent) across an endothelium, e.g., the blood-brain barrier, to be taken up by the brain.

For example, certain embodiments provide a method for transcytosis of an oligonucleotide (e.g., an ASO or an RNAi agent) across an endothelium, the method comprising contacting the endothelium (e.g., blood-brain barrier (BBB)) with a conjugate as described herein. Thus, certain embodiments provide a method of transporting an oligonucleotide across the BBB of a subject in need thereof, comprising administering a conjugate as described herein to the subject. In certain embodiments, a conjugate as described herein for use in transporting an oligonucleotide across the BBB of a subject in need thereof is provided. In some embodiments, provided herein are methods of delivering oligonucleotides to the CNS. In some embodiments, provided herein are methods of delivering an oligonucleotide to deep brain regions (e.g., cortex, brainstem, hippocampus, striatum, cerebellum, thalamus, caudate putamen, and substantia nigra). In some embodiments, provided herein are methods of delivering an oligonucleotide to deep brain regions and spinal cord (e.g., cervical spinal cord, lumbar spinal cord). In some embodiments, provided herein are methods of delivering oligonucleotides to the CNS and muscle (e.g., cardiac and skeletal). In some embodiments, provided herein are methods of delivering oligonucleotides to the CNS, peripheral nerves (e.g., retina, sciatic nerve), muscle (e.g., quadricep), and other peripheral organs (e.g., heart, diaphragm, spleen, intestine, lung, liver, and kidney).

Certain embodiments also provide a method of modulating the expression of a target gene or sequence in a subject in need thereof, comprising administering an effective amount of a conjugate as described herein to the subject. In some embodiments, a conjugate as described herein in for use modulating the expression of a target gene is provided.

In certain embodiments, the target gene or sequence is expressed in a cell in the brain of a subject. In certain embodiments, the target gene or sequence is expressed in a cell that expresses TfR. In certain embodiments, the target gene or sequence is expressed in a muscle cell, such as a skeletal muscle cell or a cardiac muscle cell.

In certain embodiments, the modulation of the target gene expression is gene knockdown or gene knockout. Thus, in certain embodiments, the expression of the target gene or sequence is inhibited or reduced, e.g., by at least about 10%, 20%, 30%, 40%, 50%, 60%, 70%, 80%, 90%, 95%, 99% or 100%, as compared to the expression in a control (e.g., a subject that was not administered the conjugate).

A conjugate as described herein is administered to a subject at a therapeutically effective amount or dose. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient.

In various embodiments, a conjugate as described herein is administered parenterally. In some embodiments, the conjugate is administered intravenously. Intravenous administration can be by infusion, e.g., over a period of from about 10 to about 30 minutes, or over a period of at least 1 hour, 2 hours, or 3 hours. In some embodiments, the conjugate is administered as an intravenous bolus. Combinations of infusion and bolus administration may also be used.

In some parenteral embodiments, a conjugate is administered intraperitoneally, subcutaneously, intradermally, or intramuscularly. In some embodiments, the conjugate is administered intradermally or intramuscularly. In some embodiments, the conjugate is administered intrathecally, such as by epidural administration, or intracerebroventricularly.

In other embodiments, a conjugate as described herein may be administered orally, by pulmonary administration, intranasal administration, intraocular administration, or by topical administration. Pulmonary administration can also be employed, e.g., by use of an inhaler or nebulizer, and formulation with an aerosolizing agent.

### XI. Pharmaceutical Compositions and Kits

In another aspect, pharmaceutical compositions and kits comprising a conjugate as described herein are provided.

### Pharmaceutical Compositions

Guidance for preparing formulations for use as described herein can be found in any number of handbooks for pharmaceutical preparation and formulation that are known to those of skill in the art.

In some embodiments, a pharmaceutical composition comprises a conjugate as described herein and further comprises one or more pharmaceutically acceptable carriers and/or excipients. In certain embodiments, the composition comprises a plurality of conjugates as described herein, which can be the same or different (e.g., a mixture of different conjugates). In certain embodiments, the ratio of oligonucleotide to protein in the composition is about 1:1 to about 4:1. In certain embodiments, the ratio of oligonucleotide to protein in the composition is about 1:1 to about 2:1. In certain embodiments, the ratio of oligonucleotide to protein in the composition is about 1.23. In certain embodiments, the ratio of oligonucleotide to protein in the composition is about 2:1 to about 3:1. In certain embodiments, the ratio of oligonucleotide to protein in the composition is about 2.5.

As used herein, the term pharmaceutically acceptable carrier includes any solvents, dispersion media, or coatings that are physiologically compatible and that preferably does not interfere with or otherwise inhibit the activity of the active agent. Various pharmaceutically acceptable excipients are well-known. In some embodiments, the carrier is suitable for intravenous, intrathecal, intracerebroventricular, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the conjugate. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes. The following methods and excipients are merely exemplary and are in no way limiting.

For oral administration, a conjugate as described herein can be formulated by combining it with pharmaceutically acceptable carriers that are well-known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the conjugates with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as a cross-linked polyvinyl pyrrolidone, agar, or alginic acid or a salt thereof such as sodium alginate.

As disclosed above, a conjugate as described herein can be formulated for parenteral administration by injection, e.g., by bolus injection or continuous infusion. For injection, the conjugates can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, conjugates can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions as described herein may vary depending on the particular use envisioned. The determination of the appropriate dosage or route of administration is well within the skill of one in the art. Suitable dosages are also described above.

### Kits

In some embodiments, kits comprising a conjugate as described herein are provided. In some embodiments, the kits are for use in modulating the expression of a target gene or sequence (e.g., a target gene expressed in the brain or central nervous system (CNS)). In some embodiments, the kits are for use in in modulating the expression of a target gene.

In some embodiments, the kit further comprises one or more additional therapeutic agents. For example, in some embodiments, the kit comprises a conjugate as described herein and further comprises one or more additional therapeutic agents. In some embodiments, the kit further comprises instructional materials containing directions (i.e., protocols) for the practice of the methods described herein (e.g., instructions for using the kit for administering a composition across the blood-brain barrier). While the instructional materials typically comprise written or printed materials, they are not limited to such. Any medium capable of storing such instructions and communicating them to an end user is contemplated herein. Such media include, but are not limited to, electronic storage media (e.g., magnetic discs, tapes, cartridges, chips), optical media (e.g., CD-ROM), and the like. Such media may include addresses to internet sites that provide such instructional materials.

The subject matter described herein includes the following non-limiting embodiments:

1. An Fc polypeptide dimer, or a Fab-Fc dimer fusion thereof, comprising:
(a) a first Fc polypeptide comprising a modified constant domain that specifically binds to a transferrin receptor; and
(b) a second Fc polypeptide capable of dimerizing with the first Fc polypeptide of (a); wherein the first and/or the second Fc polypeptide comprises one or more cysteine substitutions; or wherein the first and/or second Fab-Fc fusion comprises one or more cysteine substitutions.
   2. The Fc polypeptide dimer of embodiment 1, wherein the Fab-Fc dimer fusion is a non-targeting Fab-Fc dimer fusion.
   3. The Fc polypeptide dimer of embodiment 1 or 2, wherein the cysteine substitution(s) is selected from the group consisting of: S239C, S442C, A330C, K149C, A118C, and T289C.
   4. The Fc polypeptide dimer of any one of embodiments 1-3, wherein the first and second Fc polypeptides each comprise a cysteine substitution; or wherein the first and second Fab-Fc fusions each comprise a cysteine substitution.
   5. The Fc polypeptide dimer of embodiment 4, wherein the first and second Fc polypeptides each comprise a cysteine substitution at S239C; or wherein the first and second Fab-Fc fusions each comprise a cysteine substitution at S239C.
   6. The Fc polypeptide dimer of any one of embodiments 1-5, wherein the first and second Fc polypeptides each comprise two cysteine substitutions; or wherein the first and second Fab-Fc fusions each comprise two cysteine substitutions.
   7. The Fc polypeptide dimer of embodiment 6, wherein the first and second Fc polypeptides each comprise a cysteine substitution at S239C and A330C; or wherein the first and second Fab-Fc fusions each comprise a cysteine substitution at S239C and A330C.
   8. The Fc polypeptide dimer of any one of embodiments 1-3, wherein the second Fc polypeptide comprises a cysteine substitution; or wherein the second Fab-Fc fusion comprises a cysteine substitution.
   9. The Fc polypeptide dimer of embodiment 8, wherein the cysteine substitution is S239C.
   10. An Fc polypeptide dimer comprising:
(a) a first Fc polypeptide comprising a modified constant domain that specifically binds to a transferrin receptor; and
(b) a second Fc polypeptide capable of dimerizing with the first Fc polypeptide of (a); wherein the first and/or the second Fc polypeptide comprises one or more substitutions selected from the group consisting of N297A and N297G.
   11. The Fc polypeptide dimer of embodiment 10, wherein the first and the second Fc polypeptides are each joined to a non-targeting Fab fragment or a portion thereof, to produce a Fab-Fc dimer fusion.
   12. The Fc polypeptide dimer of any one of embodiments 1-11, wherein the modified constant domain is a modified CH3 domain comprising two, three, four, five, six, seven, eight, nine, ten, eleven, twelve, thirteen, fourteen, fifteen or sixteen positions selected from the following: position 153 is Trp, Tyr, Leu, Gln, or Glu; position 157 is Leu, Tyr, Met, Val, Phe or Trp; position 159 is Leu, Thr, His, Pro or Phe; position 160 is Val, Pro, or an acidic amino acid; position 161 is Trp; position 162 is Val, Ser, Ala or Gly; position 163 is Asn, Gly, His, Gln, Leu, Lys, Val, Phe, Ser, Ala, Asp, Thr or Glu; position 164 is Ser, Thr, Gln, Phe, Tyr or Val; position 165 is Gln, Phe, or His; position 186 is Glu, Ala, Ser, Leu, Thr, Pro or Asp; position 187 is Lys, Arg, Gly, or Pro; position 188 is Glu or Ser; position 189 is Thr, Asn or an acidic amino acid; position 194 is Trp, Tyr, His, or Phe; position 197 is Ser, Thr, Glu, Lys or Trp; and position 199 is Ser, Trp, Gly, Cys, Pro or Met, as numbered with reference to SEQ ID NO:1.
   13. The Fc polypeptide dimer of any one of embodiments 1-12,
wherein the first Fc polypeptide or Fab-Fc fusion comprises a sequence having at least 85%, 90%, 95%, 96%, 97%, 98% or 99% sequence identity to the sequence of any one of SEQ ID NOS:279, 281, 361-366, 491-494, 702-718, 632, 645-649, 738-746, 804, and
wherein the second Fc polypeptide or Fab-Fc fusion comprises a sequence having at least 85%, 90%, 95%, 96%, 97%, 98% or 99% sequence identity to the sequence of any one of SEQ ID NOS: 557-561, 627-631, 635-644, 719-723, 724-731, 733-737, and 810.
   14. The Fc polypeptide dimer of embodiment 13, wherein the first Fc polypeptide or Fab-Fc fusion comprises a Glu at position 153, Tyr at position 157, Thr at position 159, Glu at position 160, Trp at position 161, Ser, or Ala at position 162, Asn at position 163, Thr or Ser at position 186, Glu at position 188, Glu at position 189, and Phe at position 194, as numbered with reference to SEQ ID NO:1.
   15. The Fc polypeptide dimer of embodiment 13, wherein:
the first Fc polypeptide or Fab-Fc fusion comprises the amino acid sequence of SEQ ID NO:362 and the second Fc polypeptide or Fab-Fc fusion comprises the amino acid sequence of SEQ ID NO:559;
the first Fc polypeptide or Fab-Fc fusion comprises the amino acid sequence of SEQ ID NO:702 and the second Fc polypeptide or Fab-Fc fusion comprises the amino acid sequence of SEQ ID NO:559;
the first Fc polypeptide or Fab-Fc fusion comprises the amino acid sequence of SEQ ID NO:708 and the second Fc polypeptide or Fab-Fc fusion comprises the amino acid sequence of SEQ ID NO:722;
the first Fc polypeptide or Fab-Fc fusion comprises the amino acid sequence of SEQ ID NO:718 and the second Fc polypeptide or Fab-Fc fusion comprises the amino acid sequence of SEQ ID NO:559;
the first Fc polypeptide or Fab-Fc fusion comprises the amino acid sequence of SEQ ID NO:362 and the second Fc polypeptide or Fab-Fc fusion comprises the amino acid sequence of SEQ ID NO:560;
the first Fc polypeptide or Fab-Fc fusion comprises the amino acid sequence of SEQ ID NO:362 and the second Fc polypeptide or Fab-Fc fusion comprises the amino acid sequence of SEQ ID NO:561; or
the first Fc polypeptide comprises the amino acid sequence of SEQ ID NO:804 and the second Fc polypeptide comprises the amino acid sequence of SEQ ID NO:810.
   16. A conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1.
   17. The conjugate of embodiment 16, wherein the protein (P) comprises an Fc polypeptide comprising the modified constant domain.
   18. The conjugate of embodiment 16, wherein the protein comprises an Fc polypeptide dimer, comprising a first Fc polypeptide comprising the modified constant domain; and a second Fc polypeptide capable of dimerizing to the first Fc polypeptide.
   19. The conjugate of embodiment 17 or 18, wherein the Fc polypeptide is joined to a non-targeting Fab fragment or a portion thereof, to produce a Fab-Fc fusion; or the first and second Fc polypeptides of the Fc polypeptide dimer are each joined to a non-targeting Fab fragment or a portion thereof, to produce a Fab-Fc dimer fusion.
   20. The conjugate of any one of embodiments 16-19, wherein the modified site is an amino acid substitution or insertion.
   21. The conjugate of embodiment 20, wherein the modified site is a cysteine substitution.
   22. The conjugate of embodiment 21, wherein the cysteine substitution is selected from the group consisting of: S239C, S442C, A330C, K149C, A118C, and T289C, according to EU numbering.
   23. The conjugate of any one of embodiments 16-22, wherein the linking group L comprises a group of the following formula:

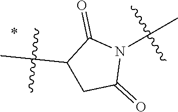 [SMILES: *C1CC(=O)N(*)C1=O]

that is attached at the position marked * to a sulfur atom of a modified site within P.
24. The conjugate of embodiment 20, wherein the modified site is an alanine or glycine substitution.
25. The conjugate of embodiment 24, wherein the alanine or glycine substitution is N297A or N297G, according to EU numbering.
6. The conjugate of any one of embodiments 16-20 and 24-25, that is prepared by conjugating L to P at Q295 by enzymatic conjugation.
27. The conjugate of embodiment 26, wherein the enzymatic conjugation uses a bacterial transglutaminase (BTG).
28. The conjugate of any one of embodiments 16-27, wherein the modified constant domain is a modified CH3 domain comprising two, three, four, five, six, seven, eight, nine, ten, eleven, twelve, thirteen, fourteen, fifteen or sixteen positions selected from the following: position 153 is Trp, Tyr, Leu, Gln, or Glu; position 157 is Leu, Tyr, Met, Val, Phe or Trp; position 159 is Leu, Thr, His, Pro or Phe; position 160 is Val, Pro, or an acidic amino acid; position 161 is Trp; position 162 is Val, Ser, Ala or Gly; position 163 is Asn, Gly, His, Gln, Leu, Lys, Val, Phe, Ser, Ala, Asp, Thr or Glu; position 164 is Ser, Thr, Gln, Phe, Tyr or Val; position 165 is Gln, Phe, or His; position 186 is Glu, Ala, Ser, Leu, Thr, Pro or Asp; position 187 is Lys, Arg, Gly, or Pro; position 188 is Glu or Ser; position 189 is Thr, Asn or an acidic amino acid; position 194 is Trp, Tyr, His, or Phe; position 197 is Ser, Thr, Glu, Lys or Trp; and position 199 is Ser, Trp, Gly, Cys, Pro or Met, as numbered with reference to SEQ ID NO:1.
29. The conjugate of any one of embodiments 16-28, wherein the first Fc polypeptide or Fab-Fc fusion comprises a sequence having at least 85%, 90%, 95%, 96%, 97%, 98% or 99% sequence identity to the sequence of any one of SEQ ID NOS:279, 281, 361-366, 491-494, 702-718, 632, 645-649, 738-746, 804; and wherein the second Fc polypeptide or Fab-Fc fusion comprises a sequence having at least 85%, 90%, 95%, 96%, 97%, 98% or 99% sequence identity to the sequence of any one of SEQ ID NOS: 557-561, 627-631, 635-644, 719-723, 724-731, 733-737, and 810.
30. The conjugate of embodiment 29, wherein the first Fc polypeptide or Fab-Fc fusion comprises a Glu at position 153, Tyr at position 157, Thr at position 159, Glu at position 160, Trp at position 161, Ser, or Ala at position 162, Asn at position 163, Thr or Ser at position 186, Glu at position 188, Glu at position 189, and Phe at position 194, as numbered with reference to SEQ ID NO:1.
31. The conjugate of embodiment 29, wherein:
the first Fc polypeptide or Fab-Fc fusion comprises the amino acid sequence of SEQ ID NO:362 and the second Fc polypeptide or Fab-Fc fusion comprises the amino acid sequence of SEQ ID NO:559;
the first Fc polypeptide or Fab-Fc fusion comprises the amino acid sequence of SEQ ID NO:702 and the second Fc polypeptide or Fab-Fc fusion comprises the amino acid sequence of SEQ ID NO:559;
the first Fc polypeptide or Fab-Fc fusion comprises the amino acid sequence of SEQ ID NO:708 and the second Fc polypeptide or Fab-Fc fusion comprises the amino acid sequence of SEQ ID NO:722;
the first Fc polypeptide or Fab-Fc fusion comprises the amino acid sequence of SEQ ID NO:718 and the second Fc polypeptide or Fab-Fc fusion comprises the amino acid sequence of SEQ ID NO:559;
the first Fc polypeptide or Fab-Fc fusion comprises the amino acid sequence of SEQ ID NO:362 and the second Fc polypeptide or Fab-Fc fusion comprises the amino acid sequence of SEQ ID NO:560;
the first Fc polypeptide or Fab-Fc fusion comprises the amino acid sequence of SEQ ID NO:362 and the second Fc polypeptide or Fab-Fc fusion comprises the amino acid sequence of SEQ ID NO:561; or
the first Fc polypeptide comprises the amino acid sequence of SEQ ID NO:804 and the second Fc polypeptide comprises the amino acid sequence of SEQ ID NO:810.
   32. The conjugate of any one of embodiments 16-31, wherein the oligonucleotide is an antisense oligonucleotide (ASO) or a siRNA.
   33. The conjugate of embodiment 32, wherein each oligonucleotide is independently an ASO.
   34. The conjugate of embodiment 33, wherein the ASO is a gapmer having a modification pattern of formula 5′-Xₐ-Yₐ-Zₐ-3′, wherein Xₐ and Zₐ each comprise 3 LNA modified nucleotides, and wherein the gap region Ya comprises PS linkages.
   35. The conjugate of embodiment 33 or 34, wherein each L is linked to the 5′ end of an ASO.
   36. The conjugate of embodiment 33 or 34, wherein each L is linked to the 3′ end of an ASO.
   37. The conjugate of any one of embodiments 16-36, wherein L comprises a C₆ amine group having the formula —(CH₂)₆—NH—.
   38. The conjugate of any one of embodiments 16-37, wherein P binds the transferrin receptor with an affinity of from 3 nM to 600 nM, or from 50 nM to 250 nM.
   39. The conjugate of embodiment 38, wherein P binds the transferrin receptor with an affinity of about 100 nM.
   40. The conjugate of any one of embodiments 16-18 and 20-39, wherein P does not comprise a Fab fragment or a portion thereof.
   41. A conjugate of formula I that comprises more than one oligonucleotide:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1; and
n is 2 or more.
   42. The conjugate of embodiment 41, wherein n is 2 or 4.
   43. The conjugate of embodiment 41 or 42, wherein the protein (P) comprises an Fc polypeptide comprising the modified constant domain.
   44. The conjugate of any one of embodiments 41-43, wherein the protein (P) comprises an Fc polypeptide dimer, comprising a first Fc polypeptide comprising the modified constant domain; and a second Fc polypeptide capable of dimerizing to the first Fc polypeptide.
   45. The conjugate of embodiment 43 or 44, wherein the Fc polypeptide is joined to a non-targeting Fab fragment or a portion thereof to produce a Fab-Fc fusion; or the first and second Fc polypeptides of the Fc polypeptide dimer are each joined to a non-targeting Fab fragment or a portion thereof to produce a Fab-Fc dimer fusion.
   46. The conjugate of any one of embodiments 41-45, wherein the protein (P) comprises one or more modified sites, which facilitate the attachment of P to each L.
   47. The conjugate of embodiment 46, wherein the modified site is an amino acid substitution or insertion.
   48. The conjugate of embodiment 47, wherein the modified site is a cysteine substitution.
   49. The conjugate of embodiment 48, wherein the cysteine substitution is selected from the group consisting of: S239C, S442C, A330C, K149C, A118C, and T289C, according to EU numbering.
   50. The conjugate of any one of embodiments 44-49, wherein n is 2, and wherein the first Fc polypeptide and the second Fc polypeptide are each attached to an L through a cysteine residue at position 239.
   51. The conjugate of any one of embodiments 44-49, wherein n is 4, and wherein the first Fc polypeptide and the second Fc polypeptide are each attached to two Ls through cysteine residues at positions 239 and 330.
   52. The conjugate of any one of embodiments 41-51, wherein the oligonucleotide is an antisense oligonucleotide (ASO) or a siRNA.
   53. A conjugate of formula I that comprises more than one oligonucleotide:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1, wherein at least one y is 2 or more; and
n is at least one.
   54. The conjugate of embodiment 53, wherein y is 2.
   55. The conjugate of any one of embodiment 53-54, wherein at least one L is attached separately to two oligonucleotides.
   56. The conjugate of any one of embodiments 53-55, wherein y is 2, and wherein at least one L is attached at the 5′ end of a first oligonucleotide and a second oligonucleotide is linked to the 3′end of the first oligonucleotide.
   57. The conjugate of embodiment 56, wherein the first and second oligonucleotide are linked by a nucleic acid linker or a non-oligonucleotide cleavable linker.
   58. The conjugate of any one of embodiments 53-57, wherein the protein (P) comprises an Fc polypeptide comprising the modified constant domain.
   59. The conjugate of any one of embodiments 53-57, wherein the protein (P) comprises an Fc polypeptide dimer, comprising a first Fc polypeptide comprising the modified constant domain; and a second Fc polypeptide capable of dimerizing to the first Fc polypeptide.
   60. The conjugate of embodiment 58 or 59, wherein the Fc polypeptide is joined to a non-targeting Fab fragment or a portion thereof to produce a Fab-Fc fusion; or the first and second Fc polypeptides of the Fc polypeptide dimer are each joined to a non-targeting Fab fragment or a portion thereof to produce a Fab-Fc dimer fusion.
   61. The conjugate of any one of embodiments 53-60, wherein P further comprises one or more modified sites, which facilitate the attachment of P to each L.
   62. The conjugate of embodiment 61, wherein the modified site is an amino acid substitution or insertion.
   63. The conjugate of embodiment 62, wherein the modified site is a cysteine substitution.
   64. The conjugate of embodiment 63, wherein the cysteine substitution is selected from the group consisting of: S239C, S442C, A330C, K149C, A118C, and T289C, according to EU numbering.
   65. The conjugate of any one of embodiments 53-64, wherein the oligonucleotide is an antisense oligonucleotide (ASO) or a siRNA.
   66. A low affinity transferrin receptor binding conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor with an affinity of about 3 nM to about 600 nM;
each y is independently at least 1; and
n is at least 1.
   67. The conjugate of embodiment 66, wherein P binds the transferrin receptor with an affinity of optionally about 3 nm to about 600 nM or about 40 nM to about 1200 nM.
   68. The conjugate of embodiment 67, wherein P binds the transferrin receptor with an affinity of from about 50 nM to 250 nM.
   69. The conjugate of embodiment 68, wherein P binds the transferrin receptor with an affinity of about 100 nM.
   70. The conjugate of any one of embodiments 66-69, wherein the oligonucleotide is an antisense oligonucleotide (ASO) or a siRNA.
   71. The conjugate of any one of embodiments 66-70, wherein the protein (P) comprises an Fc polypeptide comprising the modified constant domain.
   72. The conjugate of any one of embodiments 66-70, wherein the protein (P) comprises an Fc polypeptide dimer, comprising a first Fc polypeptide comprising the modified constant domain; and a second Fc polypeptide capable of dimerizing to the first Fc polypeptide.
   73. The conjugate of embodiment 71 or 72, wherein the Fc polypeptide is joined to a non-targeting Fab fragment or a portion thereof to produce a Fab-Fc fusion; or the first and second Fc polypeptides of the Fc polypeptide dimer are each joined to a non-targeting Fab fragment or a portion thereof to produce a Fab-Fc dimer fusion.
   74. The conjugate of any one of embodiments 66-73, wherein P further comprises one or more modified sites, which facilitate the attachment of P to each L.
   75. The conjugate of embodiment 74, wherein the modified site is an amino acid substitution or insertion.
   76. The conjugate of embodiment 75, wherein the modified site is a cysteine substitution.
   77. The conjugate of embodiment 76, wherein the cysteine substitution is selected from the group consisting of: S239C, S442C, A330C, K149C, A118C, and T289C, according to EU numbering.
   78. A conjugate of formula I:

P-(L-(X)_{y})ₙ (I)

wherein,
each X is independently an oligonucleotide;
each L is a linking group independently selected from the group consisting of:

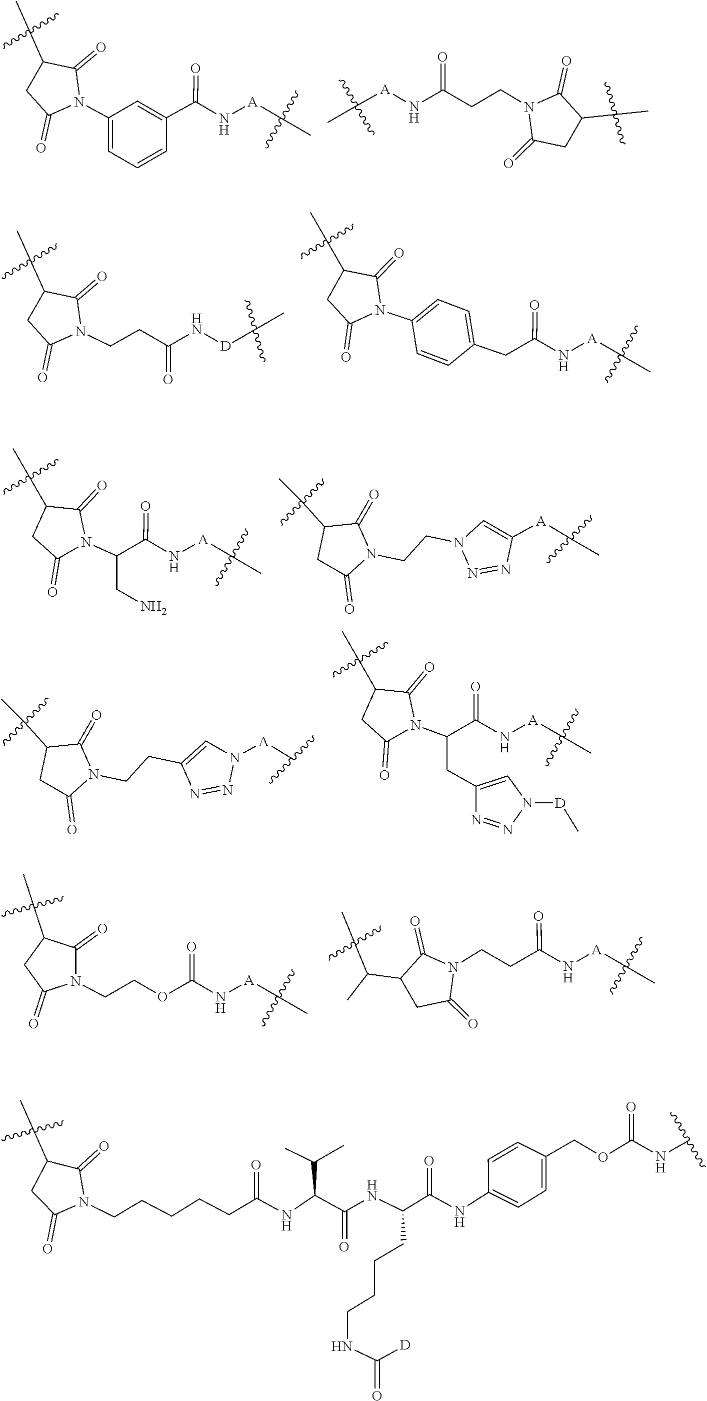

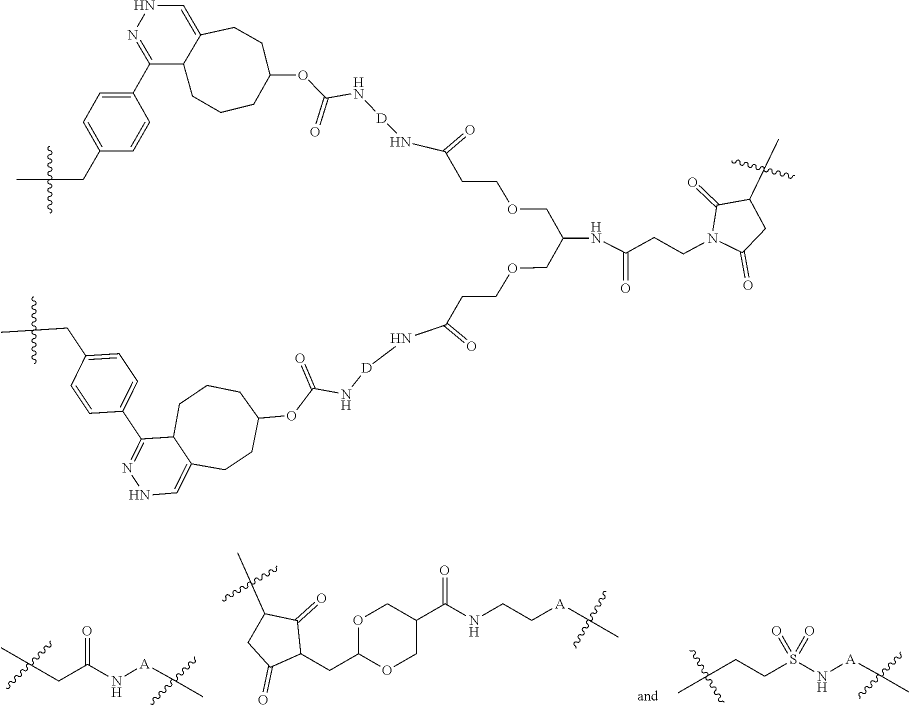

wherein
each A is independently (C₁-C₁₅)alkyl;
each D is —(CH₂—CH₂—O)ₘ—; and
each m is 1, 2, 3, 4, 5, 6, 7, 8, 9, 10, 11, 12, 13, 14, 15, 16, 17, 18, 19, 20, 21, 22, 23, or 24;
P is a protein comprising a modified constant domain that specifically binds to a transferrin receptor;
each y is independently at least 1; and
n is at least 1.
   79. The conjugate of embodiment 78, wherein each L is a linking group independently selected from the group consisting of:

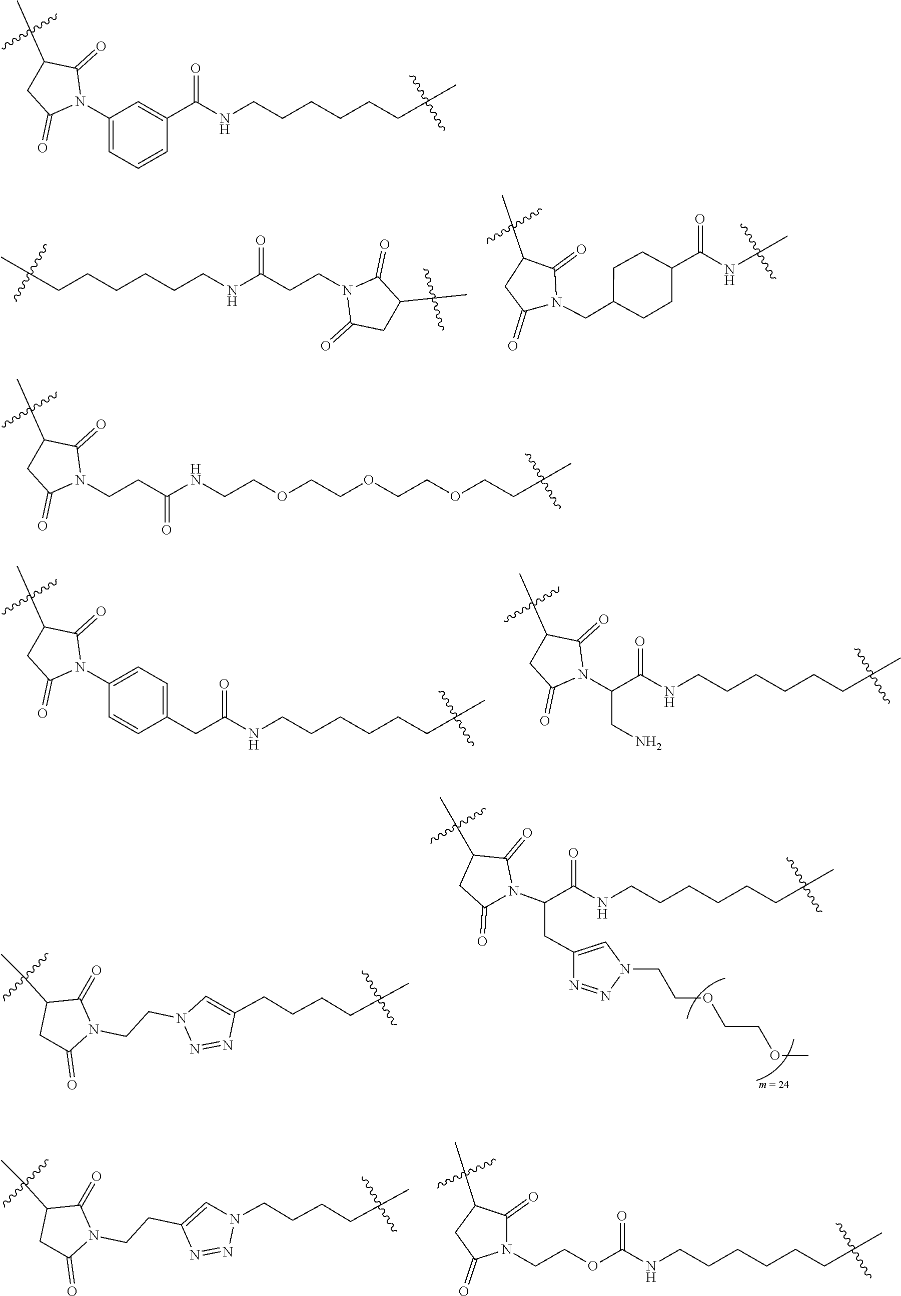

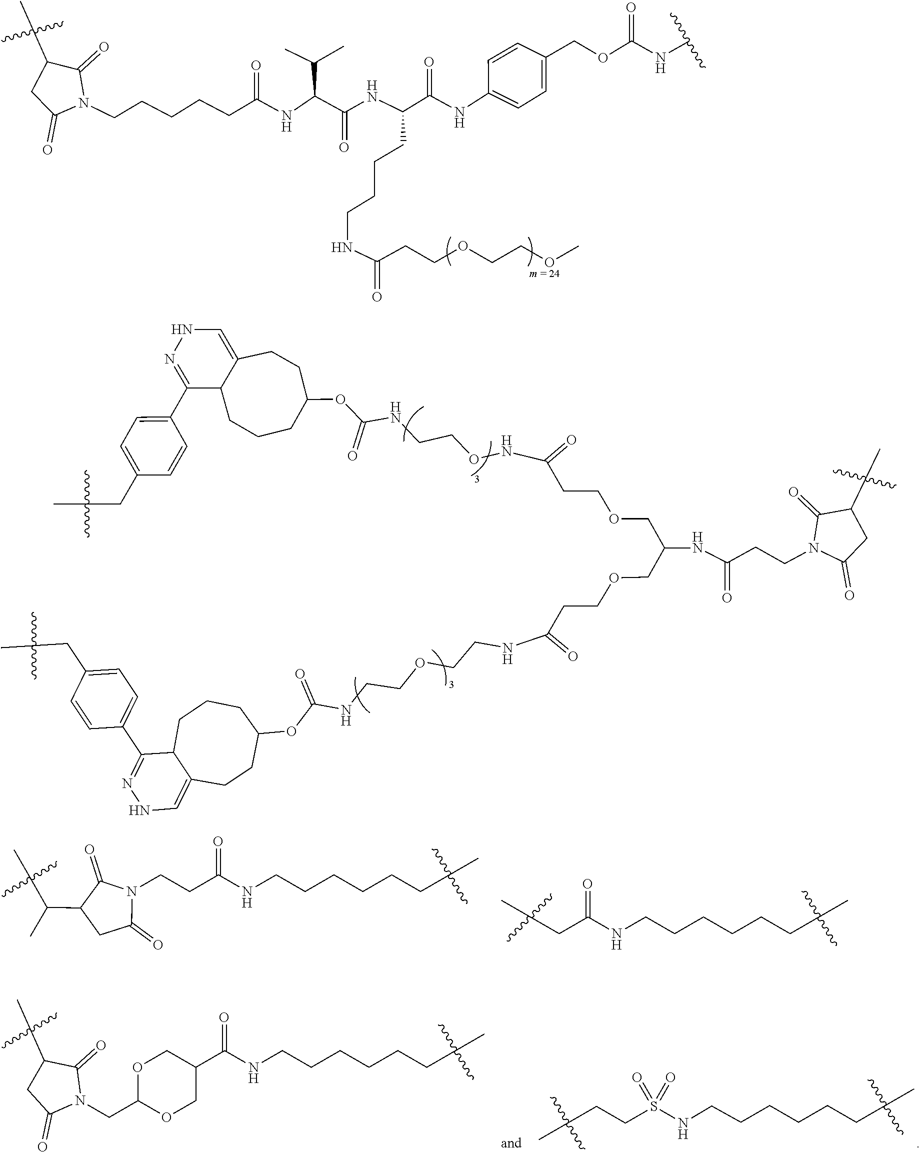

80. The conjugate of embodiment 78, wherein each L is a linking group that comprises a protected maleimide of formula:

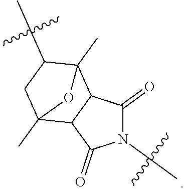 [SMILES: *C1CC2(C)OC1(C)C1C(=O)N(*)C(=O)C12]

81. The conjugate of any one of embodiments 78-80, wherein the protein (P) comprises an Fc polypeptide comprising the modified constant domain.
82. The conjugate of any one of embodiments 78-80, wherein the protein (P) comprises an Fc polypeptide dimer, comprising a first Fc polypeptide comprising the modified constant domain; and a second Fc polypeptide capable of dimerizing to the first Fc polypeptide.
83. The conjugate of embodiment 81 or 82, wherein the Fc polypeptide is joined to a non-targeting Fab fragment or a portion thereof to produce a Fab-Fc fusion; or the first and second Fc polypeptides of the Fc polypeptide dimer are each joined to a non-targeting Fab fragment or a portion thereof to produce a Fab-Fc dimer fusion.
84. The conjugate of any one of embodiments 78-83, wherein P further comprises one or more modified sites, which facilitate the attachment of P to each L.
85. The conjugate of embodiment 84, wherein the modified site is an amino acid substitution or insertion.
86. The conjugate of embodiment 85, wherein the modified site is a cysteine substitution.
87. The conjugate of embodiment 86, wherein the cysteine substitution is selected from the group consisting of: S239C, S442C, A330C, K149C, A118C, and T289C.
88. A conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor, wherein P does not comprise a Fab fragment or a portion thereof;
each y is independently 1 or more; and
n is 1 or more.
   89. The conjugate of embodiment 88, wherein the oligonucleotide is an antisense oligonucleotide (ASO) or a siRNA.
   90. The conjugate of embodiment 88, wherein the conjugate is greater than 50 kDa in size.
   91. The conjugate of any one of embodiments 88-90, wherein the protein (P) comprises an Fc polypeptide comprising the modified constant domain.
   92. The conjugate of any one of embodiments 88-90, wherein the protein (P) comprises an Fc polypeptide dimer, comprising a first Fc polypeptide comprising the modified constant domain; and a second Fc polypeptide capable of dimerizing to the first Fc polypeptide.
   93. The conjugate of any one of embodiments 88-92, wherein P further comprises one or more modified sites, which facilitate the attachment of P to each L.
   94. The conjugate of embodiment 93, wherein the modified site is an amino acid substitution or insertion.
   95. The conjugate of embodiment 94, wherein the modified site is a cysteine substitution.
   96. The conjugate of embodiment 95, wherein the cysteine substitution is selected from the group consisting of: S239C, S442C, A330C, and T289C.
   97. A conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more non-targeting Fab fragments or a portion thereof,
each y is independently 1 or more; and
n is 1 or more.
   98. The conjugate of embodiment 97, wherein the oligonucleotide is an antisense oligonucleotide (ASO) or a siRNA.
   99. The conjugate of any one of embodiments 97-98, wherein the protein (P) comprises an Fc polypeptide comprising the modified constant domain.
   100. The conjugate of any one of embodiments 97-98, wherein the protein (P) comprises an Fc polypeptide dimer, comprising a first Fc polypeptide comprising the modified constant domain; and a second Fc polypeptide capable of dimerizing to the first Fc polypeptide.
   101. The conjugate of embodiment 99 or 100, wherein the Fc polypeptide is joined to the non-targeting Fab fragment or a portion thereof to produce a Fab-Fc fusion; or the first and second Fc polypeptides of the Fc polypeptide dimer are each joined to a non-targeting Fab fragment or a portion thereof to produce a Fab-Fc dimer fusion.
   102. The conjugate of any one of embodiments 97-101, wherein P further comprises one or more modified sites, which facilitate the attachment of P to each L.
   103. The conjugate of embodiment 102, wherein the modified site is an amino acid substitution or insertion.
   104. The conjugate of embodiment 103, wherein the modified site is a cysteine substitution.
   105. The conjugate of embodiment 104, wherein the cysteine substitution is selected from the group consisting of: S239C, S442C, A330C, K149C, A118C, and T289C.
   106. A conjugate of formula I:

P-(L-(X)_{y})ₙ (I)

wherein,
each X is independently an oligonucleotide;
each L is independently

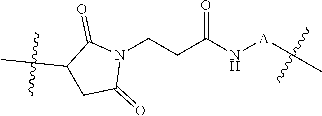

wherein A is (C₁-C₁₅)alkyl;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, and wherein the one or more modified sites are selected from the group consisting of S239C, S442C, A330C, K149C, A118C, and T289C, according to EU numbering;
each y is independently at least 1; and
n is at least 1.
   107. A pharmaceutical composition comprising a conjugate of formula (I) as described in any one of embodiments 16-106 and a pharmaceutically acceptable carrier.
   108. The composition of embodiment 107, comprising a plurality of conjugates of formula (I).
   109. The composition of embodiment 108, wherein the ratio of oligonucleotide to protein in the composition is about 1:1 to about 4:1.
   110. The composition of embodiment 108, wherein the ratio of oligonucleotide to protein in the composition is about 1:1 to about 2:1.
   111. The composition of embodiment 108, wherein the ratio of oligonucleotide to protein in the composition is about 1.23.
   112. The composition of embodiment 108, wherein the ratio of oligonucleotide to protein in the composition is about 2:1 to about 3:1.
   113. The composition of embodiment 108, wherein the ratio of oligonucleotide to protein in the composition is about 2.5.
   114. A method of transporting an oligonucleotide across the blood brain barrier (BBB) of a patient, comprising administering to the patient a conjugate of formula I:

P-(L-(X)_{y})ₙ (I)

wherein,
each X is independently an oligonucleotide;
each L is independently

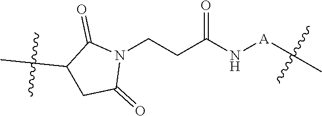

wherein A is (C₁-C₁₅)alkyl;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, and wherein the one or more modified sites are selected from the group consisting of S239C, S442C, A330C, K149C, A118C, and T289C, according to EU numbering;
each y is independently at least 1; and
n is at least 1.
   115. The method of embodiment 114, wherein the conjugate is administered to the patient intravenously.
   116. A method of modulating the expression of a target gene in a cell within the brain of a patient, comprising administering to the patient a conjugate of formula I:

P-(L-(X)_{y})ₙ (I)

wherein,
each X is independently an oligonucleotide;
each L is independently

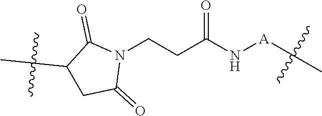

wherein A is (C₁-C₁₅)alkyl;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, selected from the group consisting of S239C, S442C, A330C, K149C, A118C, and T289C, according to EU numbering;
each y is independently at least 1; and
n is at least 1.
   117. The method of embodiment 116, wherein the modulation of target gene expression is gene knockdown or gene knockout.
   118. The method of any one of embodiments 116 or 117, wherein the conjugate is administered to the patient intravenously.
   119. A method of modulating the expression of a target gene in a cell within the brain of a patient, comprising administering to patient a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1.
   120. The method of embodiment 119, wherein the cell within the brain is a neuron, an endothelial cell, an oligodendrocyte, an astrocyte, or a microglia.
   121. The method of embodiment 119, which modulates the expression of a target gene in a plurality of cells within the brain of the patient.
   122. The method of embodiment 121, wherein the plurality of cells comprise at least two, three, four, or five cell types selected from the group consisting of: a neuron, an endothelial cell, an oligodendrocyte, an astrocyte, and a microglia.
   123. The method of any one of embodiments 120-122, wherein the neuron is an excitatory neuron or inhibitory neuron.
   124. The method of any one of embodiments 119-123, wherein the modulation of target gene expression is gene knockdown or gene knockout.
   125. The method of any one of embodiments 119-124, wherein the conjugate is administered to the patient intravenously.
   126. A method of transporting an oligonucleotide across the blood brain barrier (BBB) of a patient, comprising administering to the patient a conjugate of formula I:

P-(L-(X)_{y})ₙ (I)

wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1,
wherein the oligonucleotide is distributed throughout the central nervous system.
   127. The method of embodiment 126, wherein the oligonucleotide is distributed across brain regions.
   128. The method of embodiment 126 or 127, wherein the oligonucleotide is distributed to a deep brain region.
   129. The method of embodiment 126, wherein the oligonucleotide is distributed to the spinal cord.
   130. The method of any one of embodiments 126-129, wherein the conjugate is administered to the patient intravenously.
   131. The method of any one of embodiments 126-130, wherein the oligonucleotide modulates the expression of a target gene.
   132. The method of embodiment 131, wherein the modulation of target gene expression is gene knockdown or gene knockout.
   133. A method of delivering or administering an effective amount of an oligonucleotide to the CNS of a patient, comprising administering to the patient a conjugate of formula I:

P-(L-(X)_{y})ₙ (I)

wherein,
each X is independently an oligonucleotide;
each L is independently a linking group;
P is a protein comprising a modified constant domain that specifically binds to a transferrin receptor;
each y is independently at least 1; and
n is at least 1,
wherein the administered oligonucleotide modulates gene expression throughout the CNS.
   134. The method of embodiment 133, wherein the administered oligonucleotide modulates gene expression across brain regions and the spinal cord of the patient.
   135. The method of embodiment 134, wherein the brain regions comprise deep brain regions.
   136. The method of any one of embodiments 134-135, wherein the brain regions comprise the frontal lobe, parietal lobe, temporal lobe, occipital lobe and cerebellum.
   137. The method of any one of 134-136, wherein the brain regions comprise endothelial cells, neurons, astrocytes, oligodendrocytes, and microglia.
   138. The method of embodiment 137 wherein the oligonucleotide modulates gene expression in the endothelial cells, neurons, astrocytes, oligodendrocytes, and microglia.
   139. The method of any one of embodiments 133-138, wherein the effective amount reduces gene expression by at least about 10%, 20%, 30%, 40%, 50%, 60%, 70%, 80%, 90%, 95%, 99% or 100%, as compared to the expression without administering the oligonucleotide.
   140. The method of any one of embodiments 133-139, wherein the gene expression is reduced by at least about 50%.
   141. The method of any one of embodiments 133-140, wherein the gene expression is reduced by at least about 70%.
   142. The method of any one of embodiments 133-141, wherein the conjugate is administered to the patient intravenously.
   143. The Fab-Fc dimer of any one of embodiments 2-9 or 11-15 or the conjugate any one of embodiments 19-40, 45-52, 60-65, 73-77, 83-87, or 97-105, wherein each of the Fab of the Fab-Fc dimer and the non-targeting Fab (NTF) of the conjugate comprises a light chain variable region and a heavy change variable region, does not specifically bind to a naturally occurring epitope in a subject, and comprises three heavy chain CDRs and three light chain CDRs, wherein
CDR-H1 comprises an amino acid sequence having at least 90% sequence identity to SEQ ID NO: 825 or 826,
CDR-H2 comprises an amino acid sequence having at least 90% sequence identity to SEQ ID NO: 827, 828, or 869,
CDR-H3 comprises an amino acid sequence having at least 90% sequence identity to SEQ ID NO: 829,
CDR-L1 comprises an amino acid sequence having at least 90% sequence identity to SEQ ID NO: 819 or 820,
CDR-L2 comprises an amino acid sequence having at least 90% sequence identity to SEQ ID NO: 821 or 822, and
CDR-L3 comprises an amino acid sequence having at least 90% sequence identity to SEQ ID NO: 823 or 824.
   144. The Fab-Fc dimer or the conjugate of embodiment 143, wherein
(a) the heavy chain variable region comprises an amino acid sequence having at least 70%, at least 75%, at least 80%, at least 85%, at least 90%, or at least 95% identity to the amino acid sequence of SEQ ID NO: 837 or 853; and
(b) the light chain variable region comprises an amino acid sequence having at least 70%, at least 75%, at least 80%, at least 85%, at least 90%, or at least 95% identity to the amino acid sequence of SEQ ID NO: 832 or 851.
   145. The Fab-Fc dimer or the conjugate of embodiment 144, wherein
(a) the heavy chain variable region comprises an amino acid sequence having at least 70%, at least 75%, at least 80%, at least 85%, at least 90%, or at least 95% identity to the amino acid sequence of SEQ ID NO: 837 and the light chain variable region comprises an amino acid sequence having at least 70%, at least 75%, at least 80%, at least 85%, at least 90%, or at least 95% identity to the amino acid sequence of SEQ ID NO: 832; or
(a) the heavy chain variable region comprises an amino acid sequence having at least 70%, at least 75%, at least 80%, at least 85%, at least 90%, or at least 95% identity to the amino acid sequence of SEQ ID NO: 853 and the light chain variable region comprises an amino acid sequence having at least 70%, at least 75%, at least 80%, at least 85%, at least 90%, or at least 95% identity to the amino acid sequence of SEQ ID NO: 851.
   146. The Fab-Fc dimer or the conjugate of embodiment 144, wherein
(a) the heavy chain variable region comprises the amino acid sequence of SEQ ID NO: 837 or 853; and
(b) the light chain variable region comprises the amino acid sequence of SEQ ID NO: 832 or 851.
   147. The Fab-Fc dimer or the conjugate of embodiment 149, wherein
(a) the heavy chain variable region comprises the amino acid sequence of SEQ ID NO: 837 and the light chain variable region comprises to the amino acid sequence of SEQ ID NO: 832; or
(a) the heavy chain variable region comprises the amino acid sequence of SEQ ID NO: 853 and the light chain variable region comprises the amino acid sequence of SEQ ID NO: 851.
   148. The Fab-Fc dimer or the conjugate of embodiment 143, wherein the NTF comprises
(a) a heavy chain comprising an amino acid sequence having at least 70%, at least 75%, at least 80%, at least 85%, at least 90%, or at least 95% identity to the amino acid sequence of any one of SEQ ID NO: 838, 839, 840, 841, 844, 845, 846, 847, 854, 855, 856, 857, 859, 860, 861, and 862; and
(b) a light chain comprising an amino acid sequence having at least 70%, at least 75%, at least 80%, at least 85%, at least 90%, or at least 95% identity to the amino acid sequence of any one of SEQ ID NO: 833, 835, 850, and 852.
   149. The Fab-Fc dimer or the conjugate of embodiment 148, wherein
(a) the heavy chain comprises the amino acid sequence of any one of SEQ ID NO: 838, 839, 840, 841, 844, 845, 846, 847, 854, 855, 856, 857, 859, 860, 861, and 862; and
(b) the light chain comprises the amino acid sequence of any one of SEQ ID NO: 833, 835, 850, and 852.
   150. The Fab-Fc dimer or the conjugate of embodiment 149, wherein
(a) the heavy chain comprises the amino acid sequence of SEQ ID NO: 838, 839, 840, or 841, and the light chain comprises the amino acid sequence of SEQ ID NO: 833;
(b) the heavy chain comprises the amino acid sequence of SEQ ID NO: 844, 845, 846, or 847, and the light chain comprises the amino acid sequence of SEQ ID NO: 833;
(c) the heavy chain comprises the amino acid sequence of SEQ ID NO: 838, 839, 840, or 841, and the light chain comprises the amino acid sequence of SEQ ID NO: 835;
(d) the heavy chain comprises the amino acid sequence of SEQ ID NO: 844, 845, 846, or 847, and the light chain comprises the amino acid sequence of SEQ ID NO: 835;
(e) the heavy chain comprises the amino acid sequence of SEQ ID NO: 854, 855, 856, or 857, and the light chain comprises the amino acid sequence of SEQ ID NO: 850;
(f) the heavy chain comprises the amino acid sequence of SEQ ID NO: 859, 860, 861, or 862, and the light chain comprises the amino acid sequence of SEQ ID NO: 850;
(g) the heavy chain comprises the amino acid sequence of SEQ ID NO: 854, 855, 856, or 857, and the light chain comprises the amino acid sequence of SEQ ID NO: 852; and,
(h) the heavy chain comprises the amino acid sequence of SEQ ID NO: 859, 860, 861, or 862, and the light chain comprises the amino acid sequence of SEQ ID NO: 852.
   151. The Fab-Fc dimer of any one of embodiments 2-9 or 11-15 or the conjugate any one of embodiments 19-40, 45-52, 60-65, 73-77, 83-87, or 97-105, wherein each of the Fab of the Fab-Fc dimer and the non-targeting Fab (NTF) of the conjugate comprises a light chain variable region and a heavy change variable region, does not specifically bind to a naturally occurring epitope in a subject, and comprises three light chain CDRs and three heavy chain CDRs, wherein
CDR-H1 comprises SEQ ID NO: 825 or 826,
CDR-H2 comprises SEQ ID NO: 827, 828, or 869,
CDR-H3 comprises SEQ ID NO: 829,
CDR-L1 comprises SEQ ID NO: 819 or 820,
CDR-L2 comprises SEQ ID NO: 821 or 822, and
CDR-L3 comprises SEQ ID NO: 823 or 824.

### XII. EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present invention will employ, unless otherwise indicated, conventional methods of protein chemistry, biochemistry, recombinant DNA techniques and pharmacology, within the skill of the art. Such techniques are explained fully in the literature.

### Example 1. Construction of Exemplary TV Variants

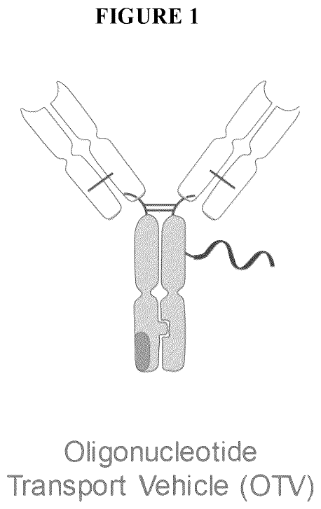

A Fab-Fc dimer fusion, wherein one Fc has been modified to bind to TfR as described herein, and wherein the Fab region is non-targeting, was generated using methods similar to those described in WO2018/152326. This modified fusion protein is hereinafter referenced as “TV” (see, e.g., FIG. 1). As described herein, these TV molecules may be attached to an oligonucleotide via a linking group; such conjugates may be referred to as “OTV”.

Construction of TVs with Cysteine Substitutions (S239C, S442C, A330C) for OTV

The following OTV genes were synthesized using a Gblock (IDT DNA) and cloned into a construct containing RSV (palivizumab) Fabs, LALA mutations, and TV35 23.2 (i.e, CH3C.35.23.2) on the knob chain, (wild-type on the hole chain) with an IgG backbone with Eu numbering with different conjugation mutations as follows:
“S239C” contains S239C on the hole chain only.
“S442C” contains S442C on the hole chain only.
“A330C” contains A330C on the hole chain only.
   The light chains are RSV (palivizumab). RSV (palivizumab) Fabs are non-targeting in mice and non-human primates.

### Construction of 500 nM TVs for OTV

Constructs were the same as above except TV35.23.4 (i.e., CH3C.35.23.4) was used on the knob chain instead, and S239C was included on the hole chain only.

### Construction of TVs for ASO Loading/Increasing Oligonucleotide to TV Ratio (Increased OTR Variants)

Constructs were same as above with TV35.23.2 (i.e, CH3C.35.23.2) on the knob chain except:
“OTR2” contains S239C on the knob chain and the hole chain.
“OTR4” contains S239C and A330C on the knob chain and the hole chain.

### Construction of SansFab TV for OTV

A modified constant domain was prepared without a fab region (“Sans Fab”), optionally with a portion or all of the IgG1 hinge region. A SansFab gene was synthesized using a Gblock (IDT DNA) containing “S239C” with a portion of the IgG1 hinge region, LALA mutations, in a knob and hole format with TV35.23.2 (i.e., CH3C.35.23.2) on the knob chain (WT Fe on the hole chain), and S239C on the hole chain. As used herein, SansFab may also be used interchangeably with No Fab.

TABLE 1
Exemplary TV Variant Sequences

| Variant | | Conjugation | | | |
|---|---|---|---|---|---|
| Name | Fab | Site(s) | Affinity | Chain Description | SEQ ID |
| S239C | RSV | S239C | 100 nM | Light chain (Knob) | SEQ ID NO: 800 |
| | | | | Heavy chain (Knob) | SEQ ID NO: 801 |
| | | | | Light chain (Hole) | SEQ ID NO: 800 |
| | | | | Heavy chain (Hole) | SEQ ID NO: 806 |
| OTR2 | RSV | S239C (on | 100 nM | Light chain (Knob) | SEQ ID NO: 800 |
| | | both sides) | | Heavy chain (Knob) | SEQ ID NO: 802 |
| | | | | Light chain (Hole) | SEQ ID NO: 800 |
| | | | | Heavy chain (Hole) | SEQ ID NO: 806 |
| OTR4 | RSV | S239C (on | 100 nM | Light chain (Knob) | SEQ ID NO: 800 |
| | | both sides) | | Heavy chain (Knob) | SEQ ID NO: 803 |
| | | and A330C (on | | Light chain (Hole) | SEQ ID NO: 800 |
| | | both sides) | | Heavy chain (Hole) | SEQ ID NO: 807 |
| 500 nM low | RSV | S239C | 500 nM | Light chain (Knob) | SEQ ID NO: 800 |
| affinity | | | | Heavy chain (Knob) | SEQ ID NO: 805 |
| | | | | Light chain (Hole) | SEQ ID NO: 800 |
| | | | | Heavy chain (Hole) | SEQ ID NO: 806 |
| A330C | RSV | A330C | 100 nM | Light chain (Knob) | SEQ ID NO: 800 |
| | | | | Heavy chain (Knob) | SEQ ID NO: 801 |
| | | | | Light chain (Hole) | SEQ ID NO: 800 |
| | | | | Heavy chain (Hole) | SEQ ID NO: 808 |
| S442C | RSV | S442C | 100 nM | Light chain (Knob) | SEQ ID NO: 800 |
| | | | | Heavy chain (Knob) | SEQ ID NO: 801 |
| | | | | Light chain (Hole) | SEQ ID NO: 800 |
| | | | | Heavy chain (Hole) | SEQ ID NO: 809 |
| SansFab | None | S239C | 100 nM | Heavy chain (Knob) | SEQ ID NO: 804 |
| | | | | Heavy chain (Hole) | SEQ ID NO: 810 |

The heavy chain may be further processed during cell culture production, such that the C-terminal lysine residue is removed. Thus, the variant names listed in Table 1 above may refer to protein molecules comprising heavy chains that are unprocessed (i.e., comprise the C-terminal lysine residue); protein molecules comprising one or more heavy chains that are processed (i.e., the C-terminal lysine residue is absent); or a mixture of protein molecules having processed and/or unprocessed heavy chains.

### Example 2. OTV Bioconjugation

TVs containing the cysteine(s) for conjugation were first reduced using a reducing reagent (e.g., TCEP). Post reduction, remaining reducing agent was removed (purification by e.g. dialysis) and the TVs are reoxidized with an oxidizing agent (e.g. dHAA). An ASO comprising a linking group was also generated, followed by a reduction and oxidation step. The reduced and oxidized linker-ASO was then conjugated to the free cysteine on the TV polypeptide and on a control antibody. The resulting conjugates are referred to as TV ASO or OTV. The resulting conjugate was purified to remove unwanted and unconjugated products and purity is determined by LC/MS and SEC.

An exemplary ASO sequence used herein that targets MALAT1 is: 5′-GksmCksAksTdsTdsmCdsTdsAdsAdsTdsAdsGdsmCdsAksGksmCk-3′ (SEQ ID NO: 656). The abbreviations refer to the components as follows: d: DNA; k: LNA; mC:5-methylcytidine (methylated cytosine); s: phosphorothioate backbone (PS). The ASO is modified with a 5′ C6 amine.

An exemplary linking group used herein is shown below, wherein the linking group is attached to a sulfur atom of a cysteine residue within the TV protein and is attached to the ASO through a phosphate associated with the 5′ terminal residue of the ASO:

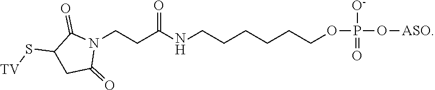

### Example 3. Construction of Exemplary OTVs with Non-Targeting Fabs, DNP02

Further Fab-Fc dimer fusions, wherein one Fc has been modified to bind to TfR as described herein, and wherein the Fab region is non-targeting, was generated with an alternative non-targeting Fab, DNP02.

Construction of TVs with N297G Substitution

The following OTV genes were synthesized using a Gblock (IDT DNA) and cloned into a construct containing DNP02 Fabs, LALA and PG mutations, and TV35.23.2 (i.e., CH3C.35.23.2) on the knob chain, (wild-type on the Hole chain) with an IgG backbone with Eu numbering except with mutations as follows:

The following molecules were clone for BTGase ASO conjugation:
“BTGase DAR2”: contains N297G on the TV dimer (contains Knob-in-hole)
“BTGase Knob” contains N297G on the knob only
“BTGase Hole” contains N297G on the hole only
   Construction of TVs with Cysteine Substitutions (A114C, T239C, K149C) for OTV

The following OTV genes were synthesized using a Gblock (IDT DNA) and cloned into a construct containing DNP02 Fabs, LALA and PG mutations, and TV35.23.2 (i.e., CH3C.35.23.2) on the knob chain, (wild-type on the Hole chain) with an IgG backbone with Eu numbering except with mutations as follows:

The following molecules were cloned for conjugation as follows:
“A114C” contains A114C on the hole (Kabat numbering).
“T289C” contains T289C on the hole (EU numbering).
“K149C” contains K149C on the DNP02 Light chain using EU numbering.

TABLE 2
Exemplary TV Variant Sequences

| Variant | | Conjugation | | | |
|---|---|---|---|---|---|
| Name | Fab | Site(s) | Affinity | Chain Description | SEQ ID NO. |
| N297G | DNP02 | N297G (on | 100 nM | Light chain (Knob) | SEQ ID NO: 811 |
| OTR2 | | both sides) | | Heavy chain (Knob) | SEQ ID NO: 814 |
| | | | | Light chain (Hole) | SEQ ID NO: 811 |
| | | | | Heavy chain (Hole) | SEQ ID NO: 816 |
| N297G | DNP02 | N297G | 100 nM | Light chain (Knob) | SEQ ID NO: 811 |
| knob | | | | Heavy chain (Knob) | SEQ ID NO: 814 |
| | | | | Light chain (Hole) | SEQ ID NO: 811 |
| | | | | Heavy chain (Hole) | SEQ ID NO: 815 |
| N297G | DNP02 | N297G | 100 nM | Light chain (Knob) | SEQ ID NO: 811 |
| hole | | | | Heavy chain (Knob) | SEQ ID NO: 813 |
| | | | | Light chain (Hole) | SEQ ID NO: 811 |
| | | | | Heavy chain (Hole) | SEQ ID NO: 816 |
| A114C | DNP02 | A114C | 100 nM | Light chain (Knob) | SEQ ID NO: 811 |
| | | | | Heavy chain (Knob) | SEQ ID NO: 813 |
| | | | | Light chain (Hole) | SEQ ID NO: 811 |
| | | | | Heavy chain (Hole) | SEQ ID NO: 817 |
| T289C | DNP02 | T289C | 100 nM | Light chain (Knob) | SEQ ID NO: 811 |
| | | | | Heavy chain (Knob) | SEQ ID NO: 813 |
| | | | | Light chain (Hole) | SEQ ID NO: 811 |
| | | | | Heavy chain (Hole) | SEQ ID NO: 818 |
| K149C | DNP02 | K149C | 100 nM | Light chain (Knob) | SEQ ID NO: 812 |
| | | | | Heavy chain (Knob) | SEQ ID NO: 813 |
| | | | | Light chain (Hole) | SEQ ID NO: 812 |
| | | | | Heavy chain (Hole) | SEQ ID NO: 815 |

The heavy chain may be further processed during cell culture production, such that the C-terminal lysine residue is removed. Thus, the variant names listed in Table 2 above may refer to protein molecules comprising heavy chains that are unprocessed (i.e., comprise the C-terminal lysine residue); protein molecules comprising one or more heavy chains that are processed (i.e., the C-terminal lysine residue is absent); or a mixture of protein molecules having processed and/or unprocessed heavy chains.

The resulting TVs were conjugated with ASOs for further testing via the bioconjugation method above for cysteine variants or will be conjugated via residue Q295 using enzymatic conjugation for the variants comprising the N297G modification.

### Example 4. Single Dose Study for OTV Variant Molecules

2 mo old TfR^{ms/hu} female mice were administered doses intravenously according to the groups (n=4) in Table 3. As controls, saline, unconjugated ASO, and RSV-ASO groups were included using the same treatment paradigm. As used herein, the term “RSV-ASO” refers to palivizumab linked to the ASO via a cysteine residue at position 239, wherein the position is according to EU numbering.

TABLE 3
Single Dose Study Groups

| | Group | Dose |
|---|---|---|
| 1 | Neg Control: Saline | |
| 2 | Neg Control: Naked ASO | 1 mpk |
| | (unconjugated ASO) | |
| 3 | Neg Control: RSV-ASO | 25 mpk |
| 4 | Conjugation site S239C | 25 mpk |
| 5 | OTR2 | molar equivalent (match for same |
| | | number of TV molecules) |
| 6 | OTR4 | molar equivalent (match for same |
| | | number of TV molecules) |
| 7 | Low affinity (500 nM) | 25 mpk |
| 8 | Conjugation site A330C | 25 mpk |
| 9 | Conjugation site S442C | 25 mpk |
| 10 | SansFab | molar equivalent (8.7 mpk) OR 25 mpk |

Tissue was collected 24 hours after the single dose. In particular, brain, spinal cord, and peripheral organs (kidney, lung, liver, and quadricep muscle) were harvested. Terminal blood was also collected 24 hours after the single dose.

huIgG Assay. Quantification of humanized antibodies in mouse plasma and tissue lysates were measured using a generic electrochemiluminescence immunoassay (ECLIA). Briefly, to the wells of an MSD GOLD 96-well streptavidin-coated microtiter plate (Meso Scale Discovery, Rockville, MD), a working concentration of biotinylated goat anti-human IgG polyclonal primary antibody (Southern Biotech, Birmingham, AL) prepared in assay diluent was incubated for approximately 1 hr. Following this incubation and a plate wash step, prepared test samples (with sample pre-dilution, where appropriate) and relevant standards were added to the assay plate and allowed to incubate for approximately 1 hr. Following test sample incubation and a plate wash step, secondary ruthenylated (SULFO-TAG) goat anti-human IgG antibody (Meso Scale Discovery, Rockville, MD) at a working concentration in assay diluent was added to the assay plate and incubated for approximately 1 hr. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

Intact OTV Assay. Quantification of Intact OTV (humanized anti-TfR antibody conjugated to an antisense oligonucleotide (ASO)) in mouse plasma and tissue lysates were measured using a hybridization-based electrochemiluminescence immunoassay (ECLIA). Briefly, custom biotinylated antisense probes (synthesized by Integrated DNA Technologies, Coralville, 1A) at a working concentration were incubated with prepared test samples (with sample pre-dilution, where appropriate) and relevant standards in TE Buffer (10 mM Tris-HCL containing 1 mM EDTA) and hybridized at an appropriate temperature for 45 mins. Following the incubation, hybridized product was added to the wells of an MSD GOLD 96-well streptavidin-coated microtiter plate (Meso Scale Discovery, Rockville, MD) and incubated for approximately 30 mins. Following hybrid product incubation and a plate wash step, secondary ruthenylated (SULFO-TAG) goat anti-human IgG antibody (Meso Scale Discovery, Rockville, MD) at a working concentration in assay diluent was added to the assay plate and incubated for approximately 1 hr. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

Total ASO Assay. Quantification of total ASO (in conjugated and free forms) in mouse plasma and tissue homogenates were measured using a hybridization-based electrochemiluminescence immunoassay (ECLIA). Briefly, custom biotinylated and digoxigenin-conjugated antisense probes (synthesized by integrated DNA Technologies, Coralville, IA) at working concentrations were combined with prepared test samples (with sample pre-dilution, where appropriate) and relevant standards in TE Buffer (10 mM Tris-HCL containing 1 mM EDTA). Prepared samples in TE buffer were added, in a 1:1 mix, into 1×SSC Buffer (Sigma-Aldrich, St. Louis, MO) containing a working concentration of recombinant proteinase K enzyme (ThermoFisher, Waltham, MA). Hybridization/Enzyme mixture was then digested, denatured, annealed, and cooled in a thermal cycler instrument. Following hybrid product incubation, samples were added to the wells of an MSD GOLD 96-well streptavidin-coated microtiter plate (Meso Scale Discovery, Rockville, MD) and incubated for approximately 30 mins. Following incubation and a plate wash step, secondary ruthenylated (SULFO-TAG) sheep anti-digoxigenin antibody (Novus Biologicals, Littleton, CO) at a working concentration in assay diluent was added to the plate and incubated for approximately 30 mins. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

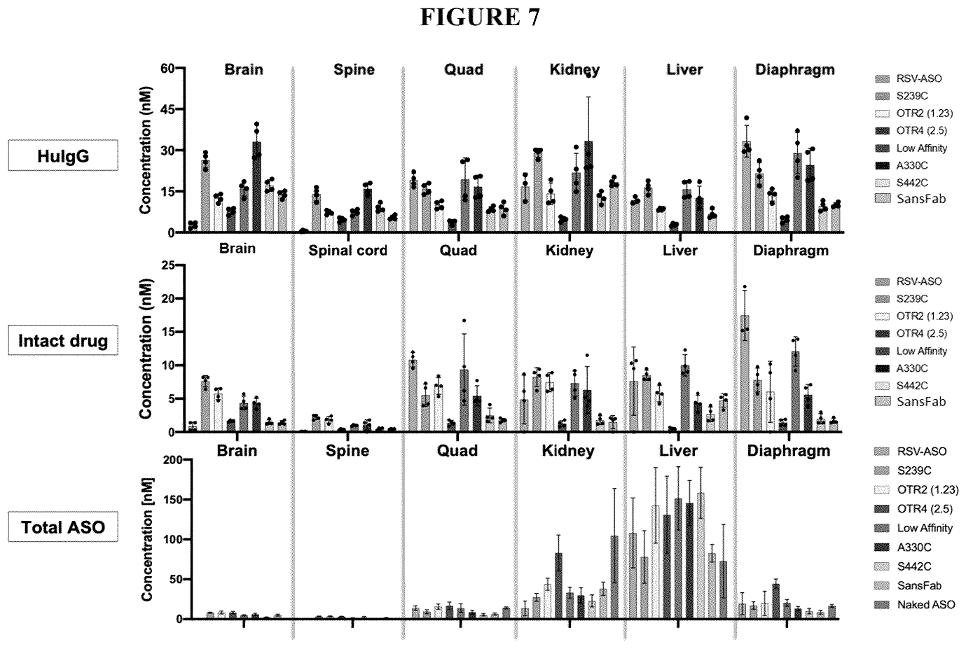

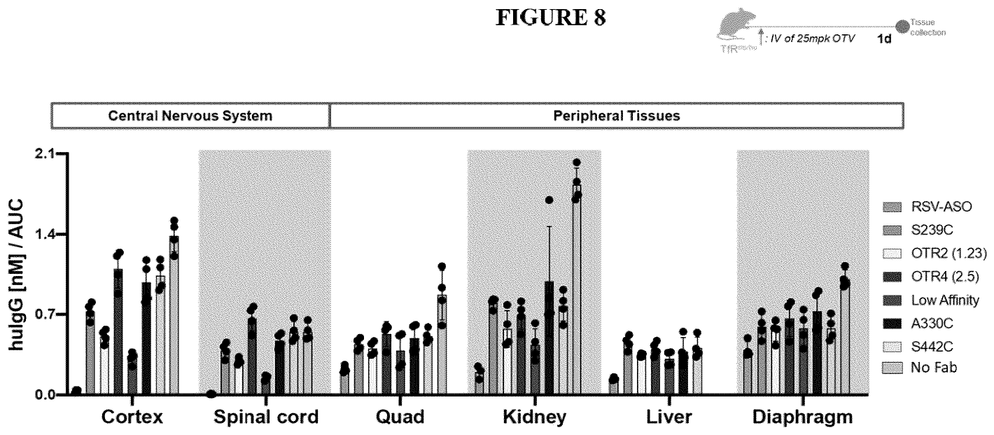

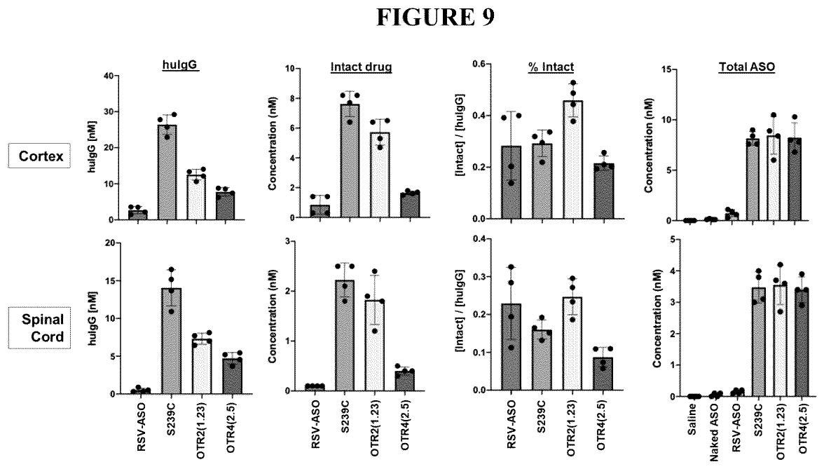

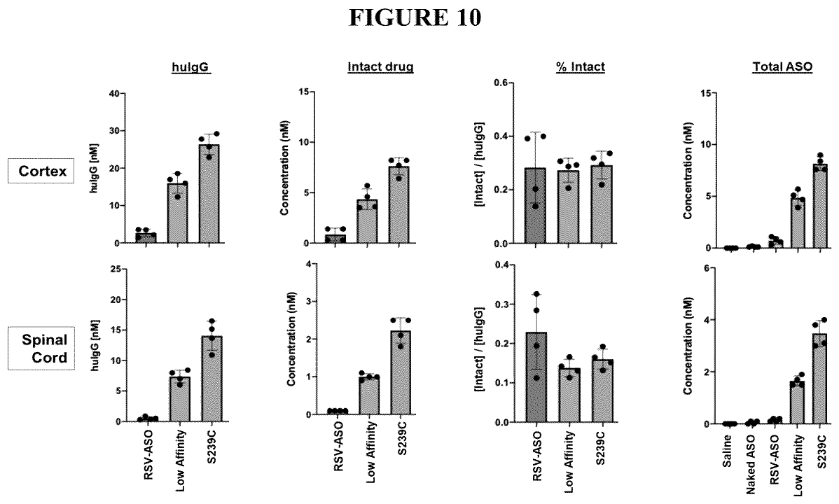

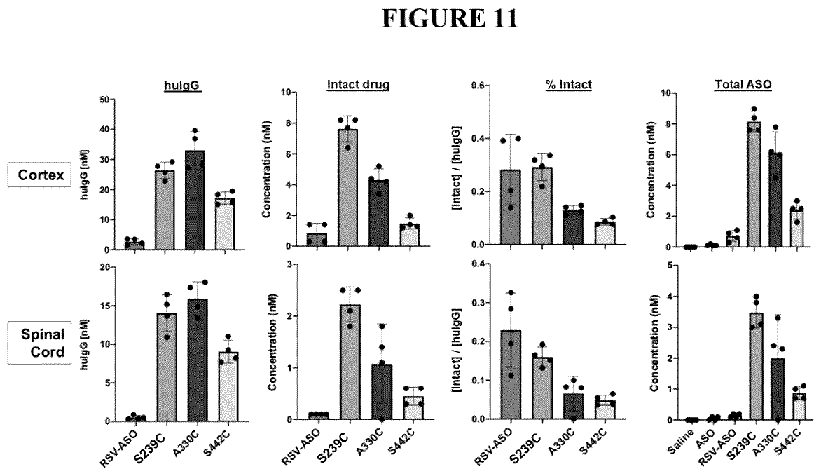

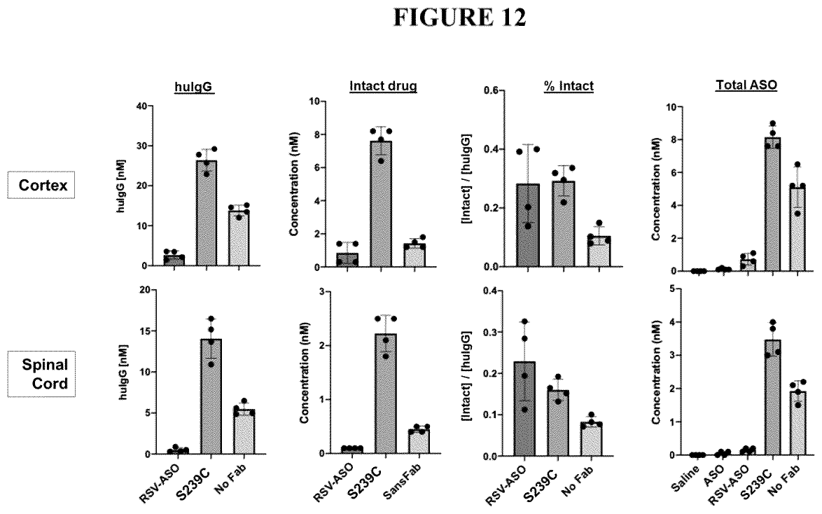

FIG. 7-8 shows tissue PK (both CNS and peripheral) 24 hours post single dose. The data shows that the liver is a sink for ASO 24 hours after dosing. FIG. 9 shows delivery of intact drug and total ASO to CNS by ASO loading variants. The data shows that increased drug loading delivers less intact drug but similar levels of total ASO to the CNS. FIG. 10 shows delivery of shows delivery of intact drug and total ASO to CNS by the 500 nM variant. The data shows a decreased delivery of intact drug and total ASO to the CNS when compared to the 100 nM variant. FIG. 11 shows delivery of intact drug and total ASO to CNS by conjugation site variants. The data shows decreased delivery of intact drug and total ASO to the CNS by A330C and S442C when compared to the S239C variant. FIG. 12 shows delivery of intact drug and total ASO to CNS by the SansFab variant. The data shows that despite the decreased intact drug delivery by the SansFab variant, the SansFab variant delivers a surprising amount of total ASO to the CNS.

### Example 5: Multi-Dose OTV Variant Molecules

2 mo old TfR^{ms/hu} female mice were administered doses intravenously according to the groups (n=6) in Table 4 on Day 1, Day 7, and Day 14. As controls, saline, unconjugated ASO, and RSV-ASO groups were included using the same treatment paradigm.

TABLE 4
Multi-Dose Study Groups

| | Group | Dose |
|---|---|---|
| 1 | Neg Control: Saline | |
| 2 | Neg Control: Naked ASO | 1 mpk |
| | (unconjugated ASO) | |
| 3 | Neg Control: RSV-ASO | 25 mpk |
| 4 | Conjugation site S239C | 25 mpk |
| 5 | OTR2 | molar equivalent (match for same |
| | | number of TV molecules) |
| 6 | OTR4 | molar equivalent (match for same |
| | | number of TV molecules) |
| 7 | Low affinity (500 nM) | 25 mpk |
| 8 | Conjugation site A330C | 25 mpk |
| 9 | Conjugation site S442C | 25 mpk |
| 10 | No Fab | molar equivalent (8.7 mpk) |

Plasma collections were taken at 30 min, 4 hours, 24 hours, 48 hours, 72 hours, and 1 week. Tissue was collected 72 hours after the last dose. In particular, brain, spinal cord, and peripheral organs (kidney, lung, liver, and quadricep muscle) were harvested. Terminal blood was also collected 72 hours after the last dose.

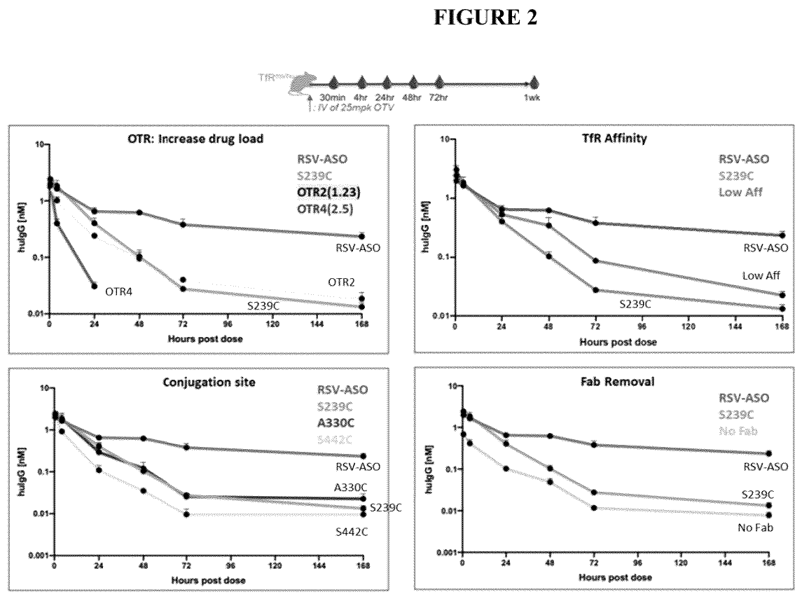

Plasma PK across the OTV variant molecules is shown in FIG. 2. The OTR4(2.5) variant and S442C conjugation site show significantly faster clearance than S239C conjugation site molecule, while the lower affinity 500 nM molecule clears more slowly.

TABLE 5
C₀, AUC, CL from FIG. 2

| | C₀ (uM) | AUC (uM*hr) | CL (mL/d/kg) |
|---|---|---|---|
| RSV-ASO | 2.04 | 84.7 | 35.5 |
| S239C | 2.53 | 36.2 | 113 |
| OTR2 (1.23) | 2.03 | 24.6 | 160 |
| OTR4 (2.5) | 2.17 | 7.07 | 569 |
| Low Affinity | 3.29 | 50.0 | 81.0 |
| A330C | 2.36 | 33.7 | 120 |
| S442 | 2.27 | 16.5 | 246 |
| SansFab | 0.73 | 9.97 | 1140 |

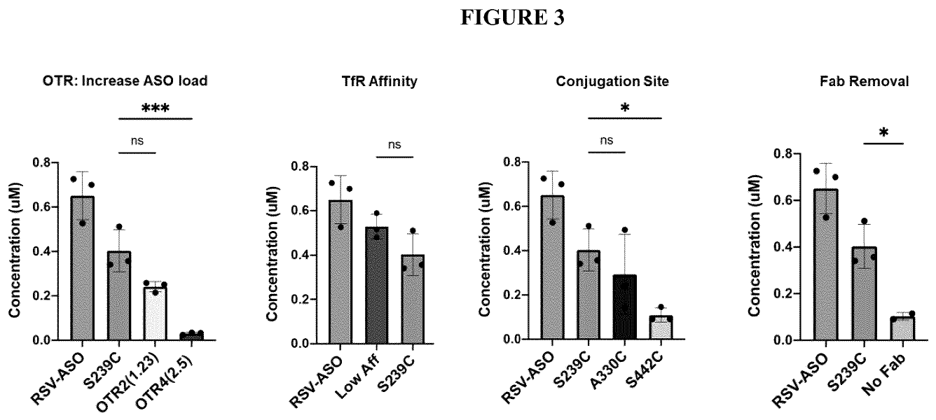

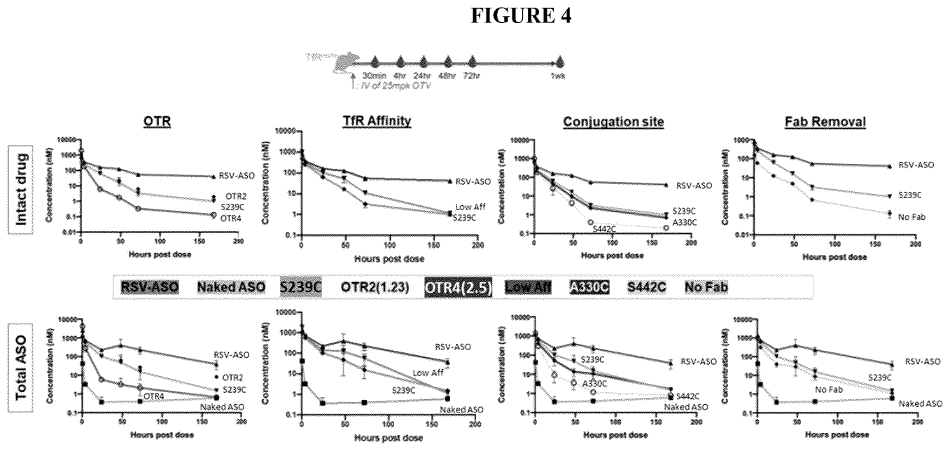

Plasma PK at the 24 hours timepoint is shown in FIG. 3. Variables that enhance plasma clearance appear to be, increased ASO drug load, S442C conjugation site, and SansFab molecules. Plasma concentration of intact drug and total ASO is shown in FIG. 4. The naked ASO control clears rapidly, as anticipated. Additionally, OTR4(2.5), S442C, and SansFab molecules clear more quickly than S239C molecules. Increased OTR variants deliver less intact drug but similar levels of total ASO to CNS (FIG. 9). However, increased OTR results in faster plasma clearance (FIGS. 2-3). OTR1 delivered the same amount of ASO to the CNS, while having decreased plasma clearance.

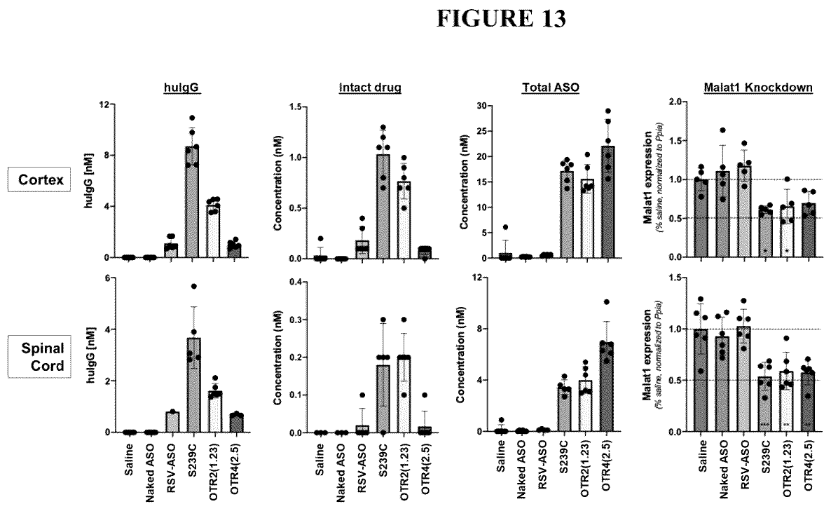

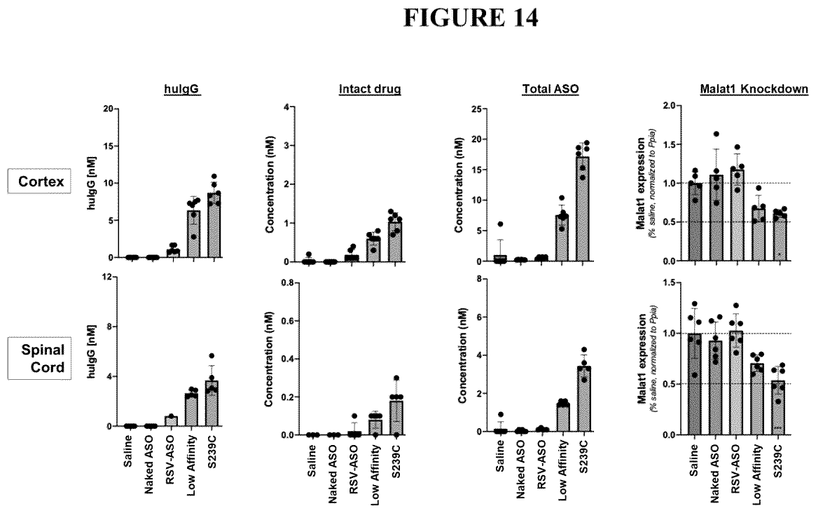

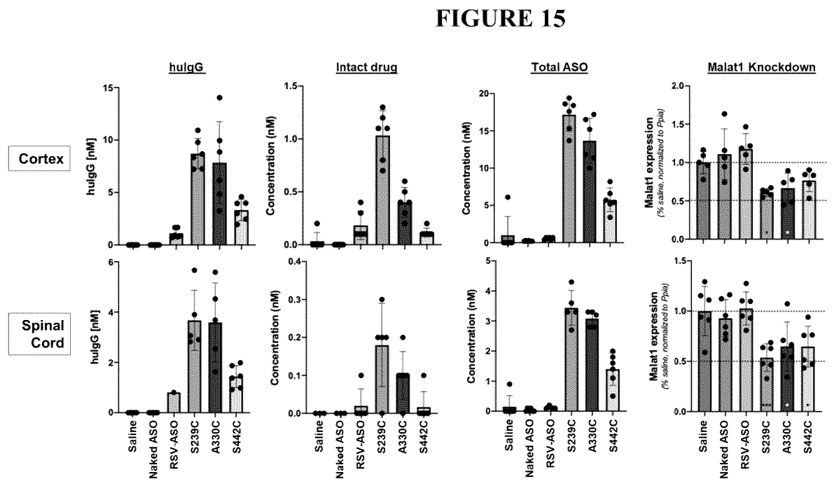

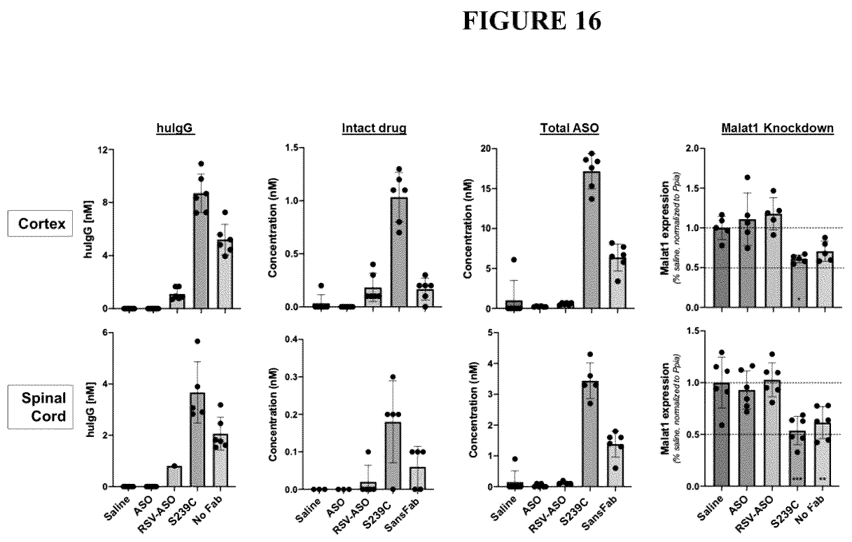

CNS PK and MALAT1 knockdown results in the cortex and spinal cord at 72 hours post final dose are shown in FIGS. 13-15. FIG. 13 shows that increasing ASO loading increased total ASO in the cortex and spinal cord and shows MALAT1 knockdown. Increased ASO loading delivers significant ASO in brain despite faster clearance. Faster clearance of the increased OTR variants may be compensated for by increased targeted cargo delivery per OTV molecule. FIG. 14 shows that decreasing affinity, i.e., 500 nM variant (“low affinity”), decreased total ASO compared to the 100 nM variant (“S239C”). Nonetheless MALAT1 knockdown in the cortex and spinal cord was still observed with the decreased affinity variant, albeit at a lower level compared to the 100 nM variant. FIG. 15 shows that A330C and S442C conjugation sites decreased total ASO in the cortex and spinal cord compared to S239C but was still able to achieve MALAT1 knockdown. FIG. 16 shows that removing the Fab arms decreased drug delivery in the cortex and spinal cord but nonetheless still achieved MALAT1 knockdown. The fast clearance of the SansFab molecule is likely due to the lower molecular weight of these molecules, approximately one-third of the molecular weight of the other molecules. The renal clearance threshold is thought to be a molecular weight of 30-50 kDa. Therefore, adding more weight to the SansFab molecule, for instance, by increasing drug loading, may slow down clearance.

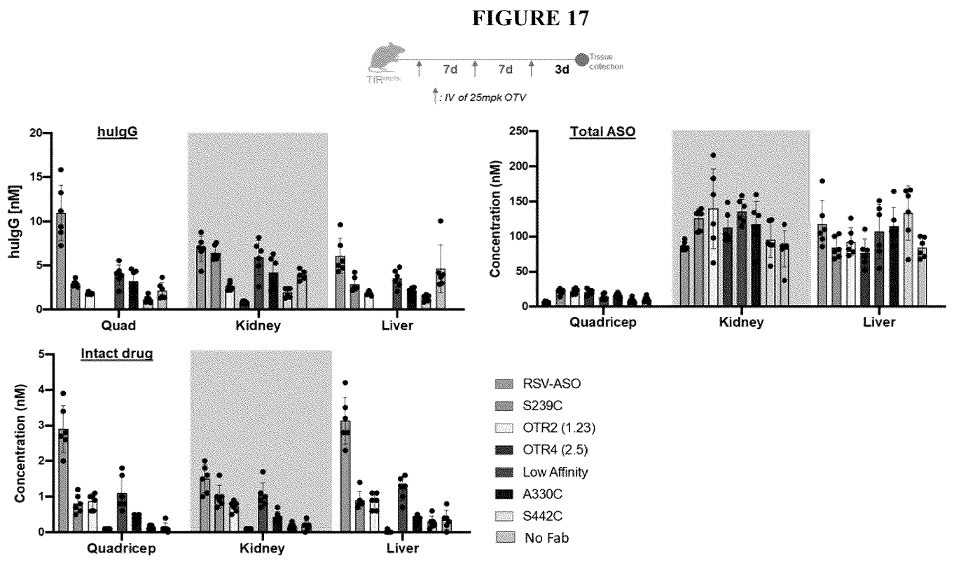

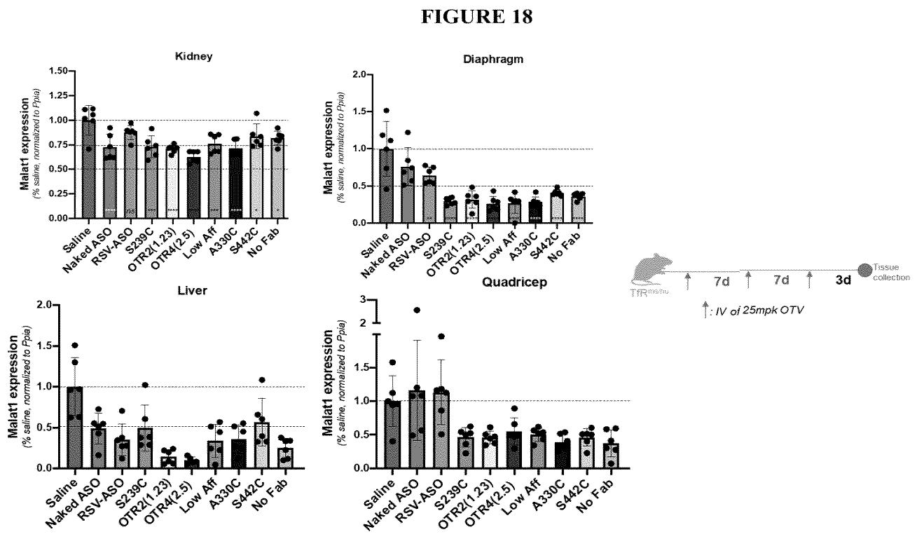

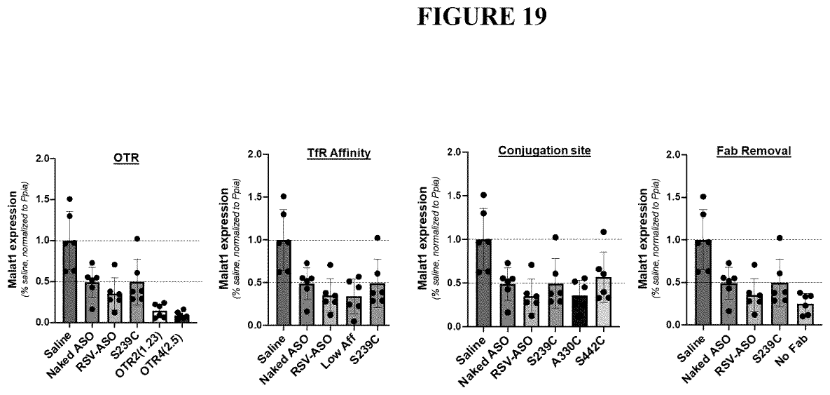

Peripheral tissue PK and knockdown at 72 hours post final dose is shown in FIGS. 17-19. ASO levels are roughly similar across variants in peripheral tissues 72 hours after the final dose. All variants induce target knockdown in peripheral tissues. Naked ASO significantly reduces MALAT1 levels in liver as anticipated, because of free ASO accumulation in the liver. OTR2(1.23), OTR4 (2.5), and SansFab variants result in significantly more target knockdown in the liver.

### Example 6: PK in Wildtype Mice

Wild-type mice, i.e., no human TfR, were administered a 10 mpk dose via IV. Plasma collections were taken at 4 hours, 1 day, 3 day, and 7 day post-dose.

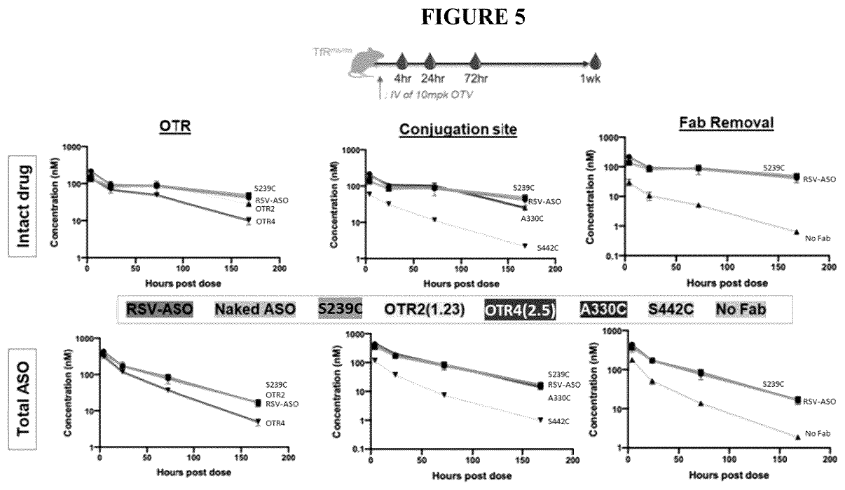

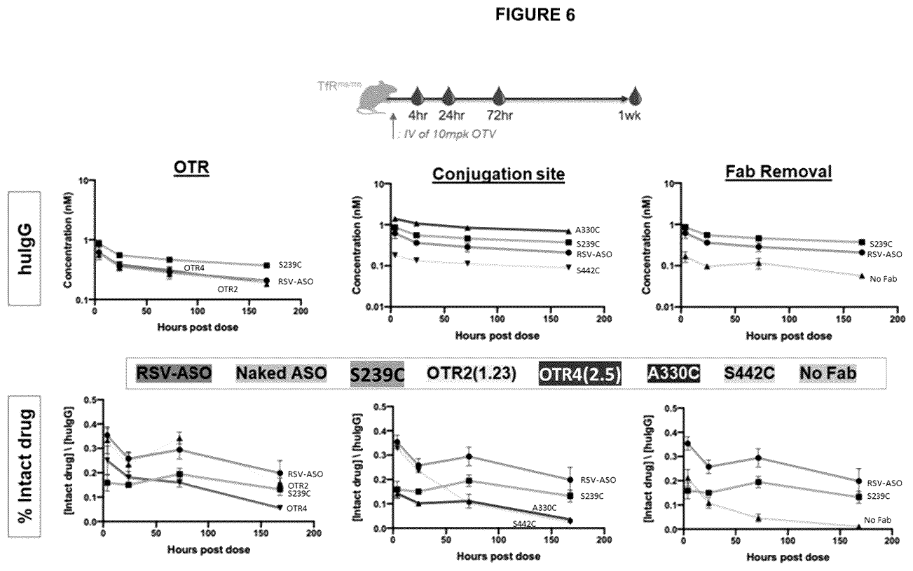

Plasma PK of intact drug and total ASO are shown in FIG. 5. huIgG concentration and % intact drug is shown in FIG. 6. These data show non-TfR-mediated clearance rates of the OTV variants. All OTV variant molecules clear more slowly in wildtype mice compared to huTfR-KI mice. The rate of deconjugation across all OTV variant molecules is at a much lower (if any) rate than in huTfR-KI mice, suggesting that deconjugation is receptor-pathway mediated. Data also suggests that the OTV variant molecules are stable in vivo when not subject to internalization. The S442C conjugation and the SansFab molecules cleared faster than both S239C and RSV-ASO. This suggests that a partial explanation for their faster clearance in huTfR-KI mice is non-TfR mediated and may be a result of renal clearance or molecular instability.

### Example 7: Brain Knockdown and ASO Biodistribution in Single and Multi-Dose Non-Human Primate Studies

Malat1 ASO was bioconjugated as described above via a maleimide linker to a TV with a S239C modification that binds cynomolgous monkey TfR with an affinity of 250 nM. The resulting molecule (“OTV:MALAT1”) was used to compare the biodistribution of intravenously delivered OTV, intravenously delivered ASO, and intrathecally delivered ASO in the central nervous system of cynomolgus monkeys, five cohorts of three cynomolgus monkeys each were dosed as outlined below:
1) Single dose of 4 mg MALAT1 ASO intrathecally (“ASO IT”);
2) Single dose of 30 mpk OTV:MALAT1 intravenously (“OTV IV”);
3) Four biweekly doses of saline intravenously (“Saline” or “Vehicle”);
4) Four biweekly doses of 30 mpk OTV:MALAT1 intravenously (“OTV IV”);
5) Four biweekly doses of 1.1 mpk MALAT1 ASO intravenously (i.e. the molar equivalent amount of ASO))(“ASO IV”).

One week after dosing, tissues were collected from group 2 above for pharmacokinetic modeling. For all other cohorts, tissues were collected two weeks after the final dose to allow sufficient time for target knockdown. Hemi-brains and spinal cords were dissected and either fixed for anti-ASO immunostaining or homogenized for pharmacokinetic and pharmacodynamic analysis.

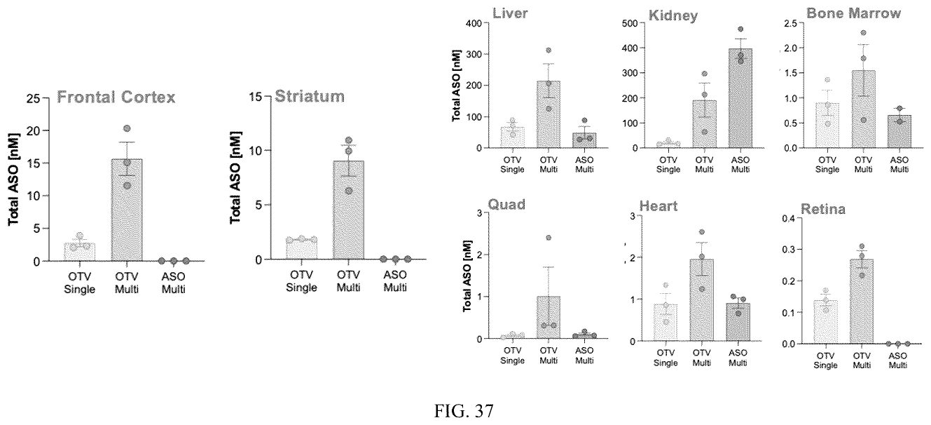

Total ASO in brain and periphery in non-human primate single and multi-dose studies is shown in FIG. 37. OTV drives ASO uptake via TfR to similar organs in NHP as observed in TfR KI mice studies, namely, CNS, muscle, and retina. Additionally, multi-dose OTV shows ASO accumulation compared to single dose OTV.

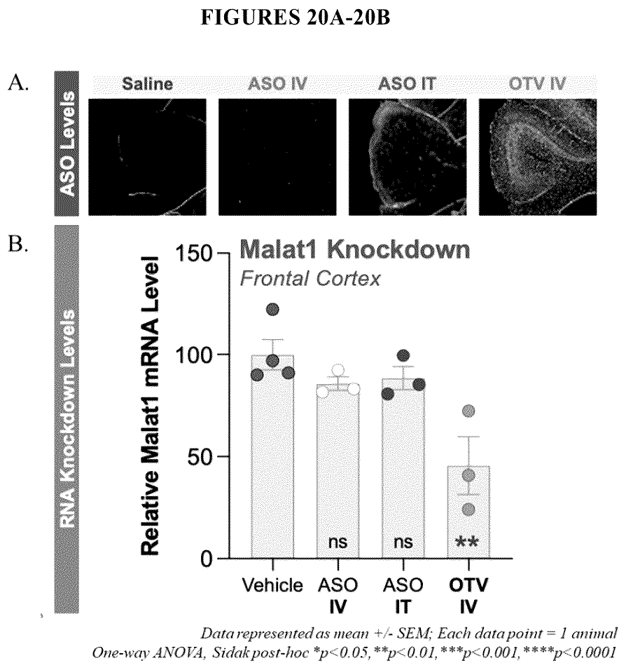

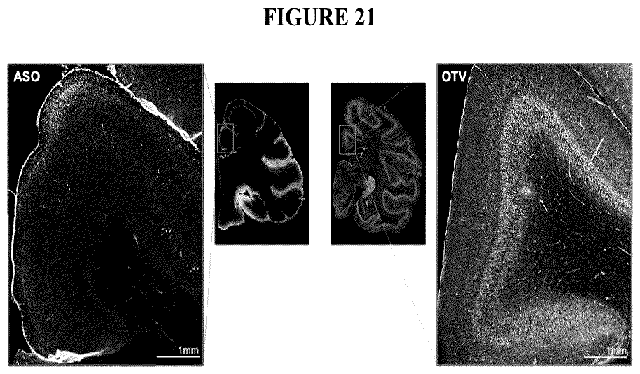

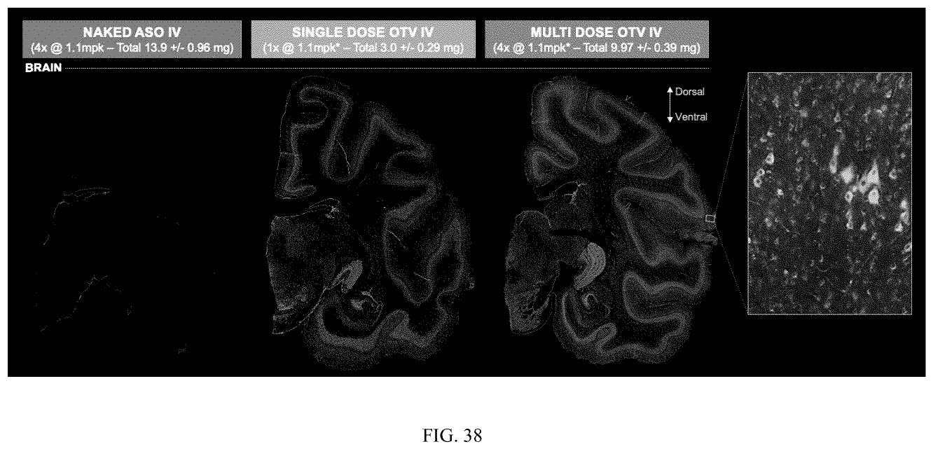

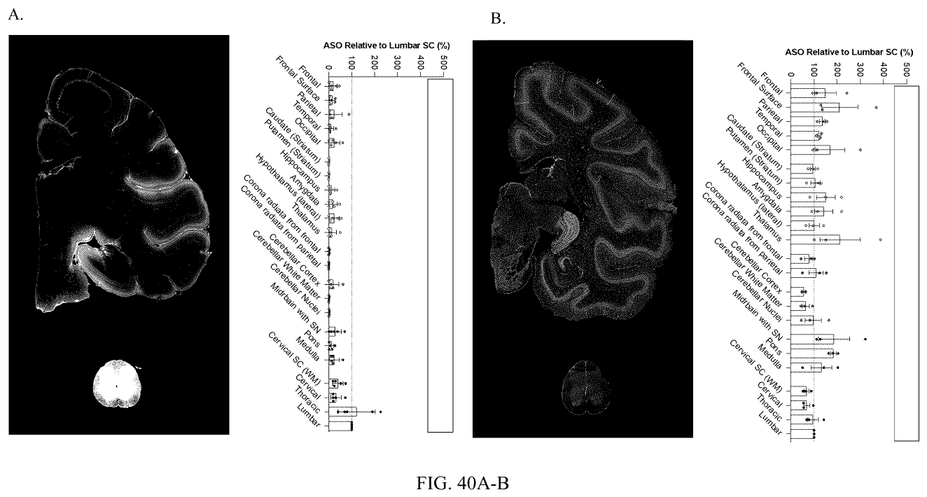

Unconjugated ASO delivered intravenously is absent from the central nervous system, as it clears quickly and does not effectively cross the blood brain barrier. Compared to an intrathecal injection of ASO, systemic administration of OTV results in significantly more widespread and homogenous biodistribution of ASO in the central nervous system. (See FIG. 20A, FIG. 21, and FIG. 38.) After intrathecal injection, ASO concentration is substantially higher in the spinal cord compared to the brain (>10 fold difference in many regions) Even within the brain, ASO concentration is much higher in surface areas adjacent to CSF and along cortical surfaces). Very little ASO is observed in the deep brain regions (FIG. 40A). However, after systemic OTV administration, superior biodistribution of ASO across brain regions is observed. ASO concentration is roughly equivalent in the spinal cord and many brain regions, including deep brain structures and deeper cortical layers (FIG. 40B). Additionally, FIG. 38 also shows cumulative ASO levels with multi-doses of OTV.

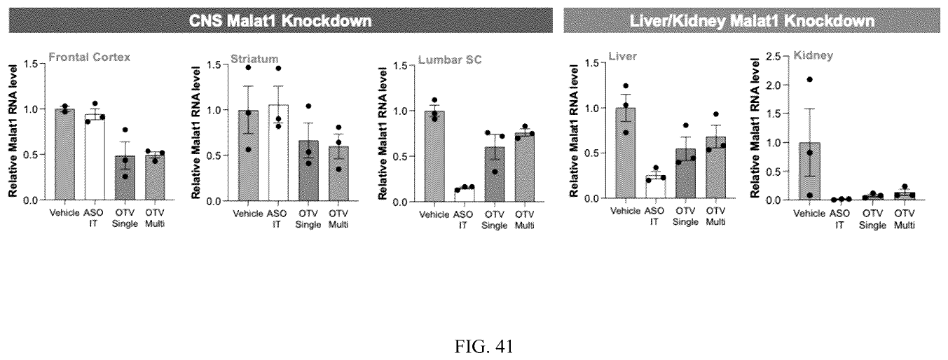

Additionally, relative MALAT1 RNA levels were measured via qPCR after a Trizol RNA extraction from brain samples. After a single 30 mpk dose of OTV, MALAT1 expression in the frontal cortex was reduced to 46% of control levels. MALAT1 expression was not significantly reduced in the frontal cortex after a single intrathecal or intravenous dose of naked ASO (FIG. 20B). In the multi-dose study, OTV provides target RNA knockdown across not only multiple regions of the CNS, but also in peripheral muscles in non-human primates compared to naked ASO (FIG. 41) This enables OTV for use in not only CNS disorders but also neuromuscular disorders, including disorders that have both a CNS and muscle phenotype.

Differences in ASO biodistribution throughout the central nervous system ultimately translate to differences in target knockdown. Following intrathecal administration, substantially greater MALAT1 knockdown is seen in the spinal cord vs. brain. In contrast, systemic OTV administration provides enhanced knockdown in brain compared to intrathecally delivered ASO and the degree of MALAT1 knockdown in the spinal cord and brain are more equal. Compared to a single intravenous dose of OTV, the multi-dose group (group 4) shows an accumulation of ASO across the brain and spinal cord with a similar biodistribution pattern that demonstrates uniform ASO accumulation across all CNS. In sum, OTV is a superior method by which to deliver ASO and to achieve more uniform target knockdown throughout the central nervous system.

### Example 8: Single Nuclei RNAseq Analysis for Multi-Dose Treated hTfR-KI Mice

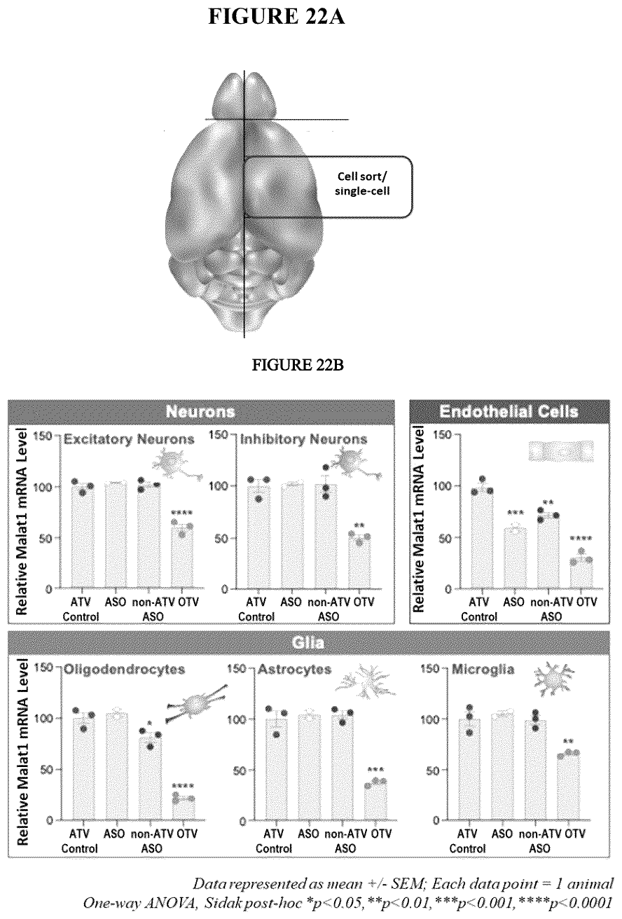

hTfR-KI mice were dosed four times weekly with either Malat1 ASO (“ASO”); TV with no ASO (“ATV control”); OTV conjugated to Malat1 ASO (“OTV”); or a non-TV Fab-Fc dimer fusion (i.e., does not have a Tfr binding site in a constant domain) conjugated to Malat1 ASO (“Non-ATV” ASO) at molar equivalence. Three days after the final dose mice were perfused with PBS and brains were dissected for single nuclei RNAseq analysis (“snRNAseq”) according to FIG. 22A.

snRNA-seq data generation. 16 single-nuclei suspensions were prepared and processed in four batches of four tissue samples, each containing samples from all four experimental groups. Nuclei were prepared in Nuclei EZ Prep buffer (Sigma Aldrich, #NUC101) containing 1× protease inhibitor (Complete, EDTA-free (Roche, cat. no. 11873580001) and 0.4 U/uL RNase inhibitor (Ambion, cat. no. AM2682). For each sample, 80 mg of tissue was dissociated with 25 strokes of pestle A and B using a 2 mL glass dounce homogenizer and the lysates were incubated on ice for 15 minutes. Afterward, the suspension was centrifuged at 500 g at 4° C. for 5 mins, the pellet was resuspended in Nuclei EZ Prep buffer and incubated for another 15 minutes on ice.

Finally, the nuclei were sedimented by centrifugation at 300 g at 4° C. for 5 mins, resuspended in 500 uL of 1% BSA in PBS, loaded onto a 2M sucrose cushion and centrifuged at 13000 g at 4° C. for 45 mins. The pellet was resuspended in 2% BSA in PBS and passed through a 30-um strainer. Concentrations of nuclei were determined using a Vi-CELL cell counter (Beckman Coulter).

Single nuclei were captured on a 10× Genomics Chromium instrument with the Chromium Next GEM Single Cell 3′ kit (version 3.1) targeting a recovery of 10,000 nuclei per sample. Next-generation sequencing library preparation was performed as per manufacturer's protocol CG000204 (10× Genomics, Revision B).

Next-generation sequencing was performed by SeqMatic (Fremont, CA) on an Illumina NovaSeq instrument with a S2 flow cell generating paired-end reads (28×10×10×90 bases). For each sample, a minimum of 200 million reads pairs were obtained.

snRNA-seq data analysis. Raw sequence files in FASTQ format were mapped to the mouse reference genome (10× Genomics, version mm10-2020-A) and quantified using Cell Ranger (10× Genomics, version 5.0.0). Raw count matrices were processed in R (version 4.0.3)/Bioconductor (version 3.12) using the DropletUtils package (version 1.10.2) to identify cell-containing droplets. Droplets with a high fraction of mitochondrial transcripts (>3 median-absolute deviations higher than the sample median) were discarded.

After removing putative doublets with the scDblFinder package (version 1.4.0), a total of 108,959 nuclei remained (7,378-13,521 per sample, median 9,724). Counts were normalized via library size factors calculated with the computeSumFactors function from the scran R package (version 1.18.3) and log transformed.

Coarse cell type labels were assigned to 107,261 nuclei (98%) using the SingleR algorithm (version 1.4.0) with default parameters and single-cell data published by Zeisel et al, Cell 174(4): 999-1014, 2018 as a reference.

For dimensionality reduction and visualization purposes, the top 1000 most variable genes were identified with the modelGeneVar and getTopHVGs functions from the scran R package. Principal component analysis (PCA) was performed on these features with the BiocSingular R package (version 1.6.0). Afterward TSNE, and UMAP dimensionality reductions were performed based on 50 principal components with the scater R package (version 1.18.3).

For differential expression analysis, gene expression counts were aggregated within each cell type for each biological replicate using the muscat R package (version 1.4.0). Only cell types observed with a median of ≥50 cells per sample were included in this analysis.

For each cell type, treatment conditions were compared using generalized linear regression adjusted for the sex of the animal with the edgeR R package (version 3.32.1). Statistical significance of differential expression was adjusted for both gene- and cell-type multiplicity using Bonferroni's method.

After cell-type clustering, Malat1 knockdown was analyzed in excitatory neurons, inhibitory neurons, endothelial cells, oligodendrocytes, astrocytes, and microglia. Results of cell type specific knockdown are shown in FIG. 22. OTV provides CNS knockdown across all major cell types compared to ASO, non-ATV ASO, and ATV control groups.

### Example 9: Plasma PK and ASO Concentration and Knockdown in Brain, Spinal Cord, and Liver of OTV Linker Variants

Seven distinct chemical linkers were used to conjugate a Malat1 ASO to Fab-Fc dimer fusions (TV35.23.2 (i.e., CH3C.35.23.2), DNP02 Fabs, S239C) described above. The resulting OTV conjugates were diluted in sterile saline and administered to mice as follows:

To determine plasma PK (rates of clearance and loss of intact OTV in circulation) in wildtype mice, 6 wildtype mice for each linking group in Table 6 were administered OTV conjugates intravenously at a dose of 25 mg/kg and plasma was collected 15 minutes, 30 minutes, 4 hours, 24 hours, 48 hours, 72 hours, and one week post dose.

To determine ASO biodistribution and target knockdown in the brain, spinal cord, and liver, 6 TfR^{ms/hu} knock-in mice for each linking group in Table 6 were administered OTV conjugates three times on Day 1, Day 4, Day 8 intravenously at a dose of 25 mg/kg. Plasma was collected at 4 hours, 24 hours, and 72 hours post-final dose. Tissue collection was conducted a week post-final dose. Mice dosed with OTV were compared to mice dosed with sterile saline as a control.

TABLE 6

| | |
|---|---|
| Linking group 1 | 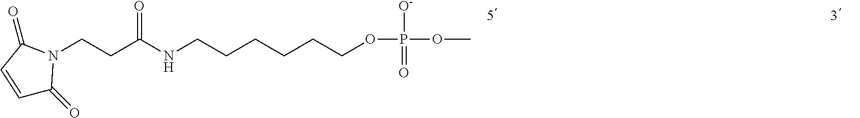 [SMILES: COP(=O)([O-])OCCCCCCNC(=O)CCN1C(=O)C=CC1=O] |
| Linking group 2 | 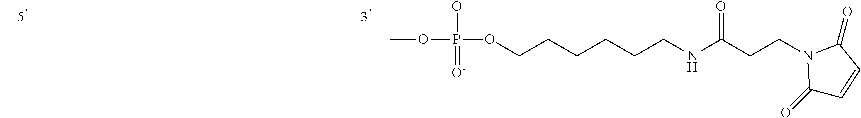 |
| Linking group 3 | 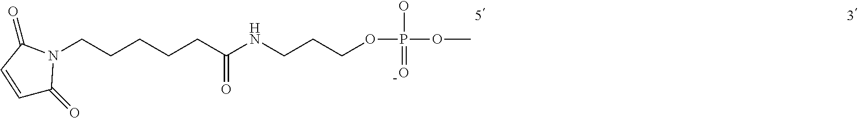 [SMILES: COP(=O)(O)OCCCNC(=O)CCCCCN1C(=O)C=CC1=O] |
| Linking group 4 | 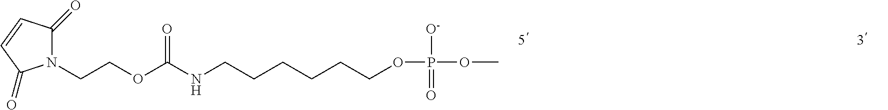 [SMILES: COP(=O)([O-])OCCCCCCNC(=O)OCCN1C(=O)C=CC1=O] |
| Linking group 5 | 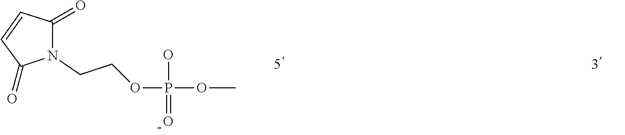 |
| Linking group 6 | 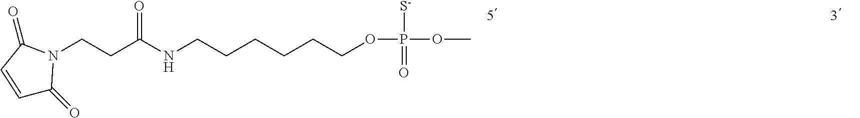 [SMILES: COP(=O)([S-])OCCCCCCNC(=O)CCN1C(=O)C=CC1=O] |
| Linking group 7 | 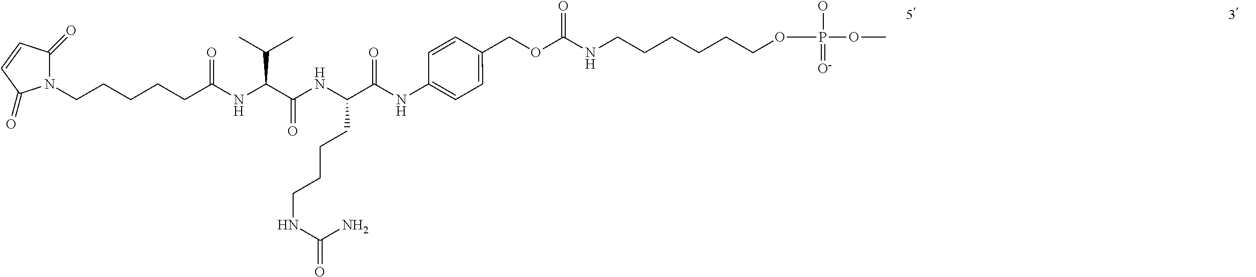 |

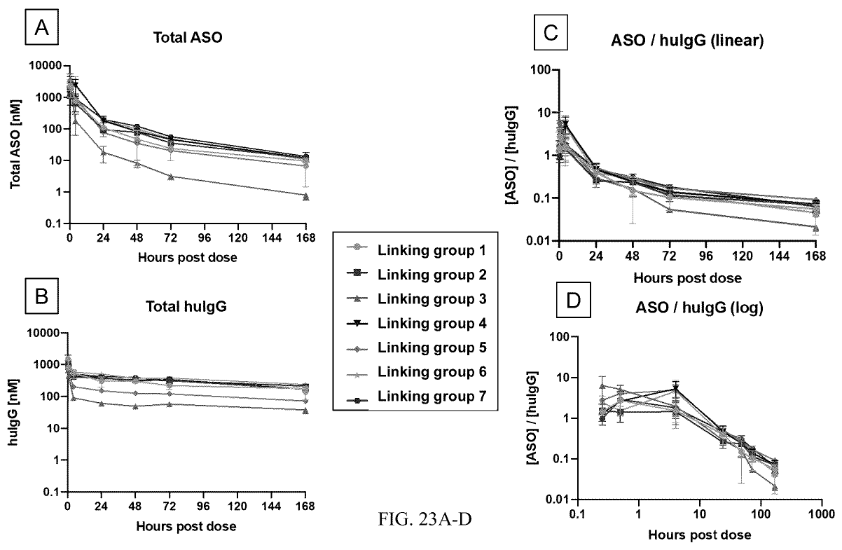

Plasma samples were analyzed for total ASO concentration and total huIgG concentration using the assays described above. Results are shown in FIG. 23. All linker variants showed similar rates of clearance and loss of intact OTV in wildtype mice.

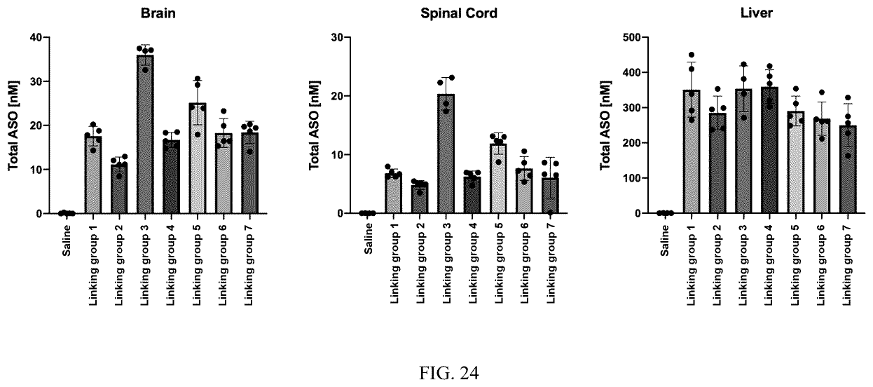

ASO concentration in the brain, spinal cord, and liver one week after three 25 mpk doses was measured using the methods described above. Results are shown in FIG. 24. All linker variants resulted in between 10 and 40 nM of ASO deposited in the brain; between 5 and 20 nM deposited in the spinal cord; and between 200 and 400 nM deposited in the liver.

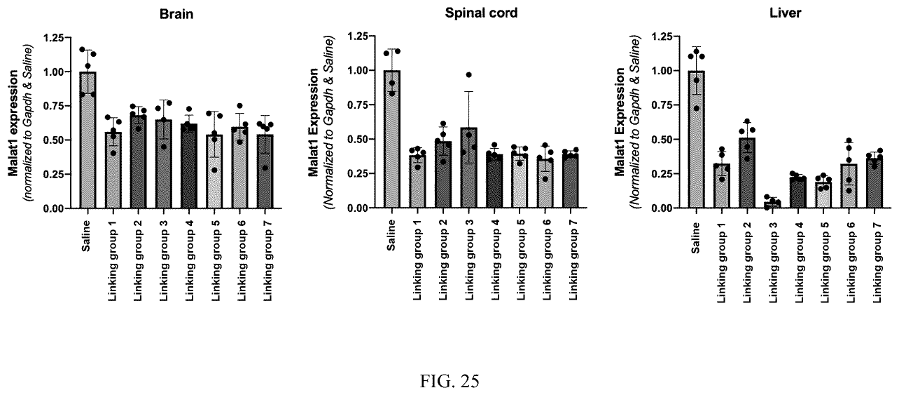

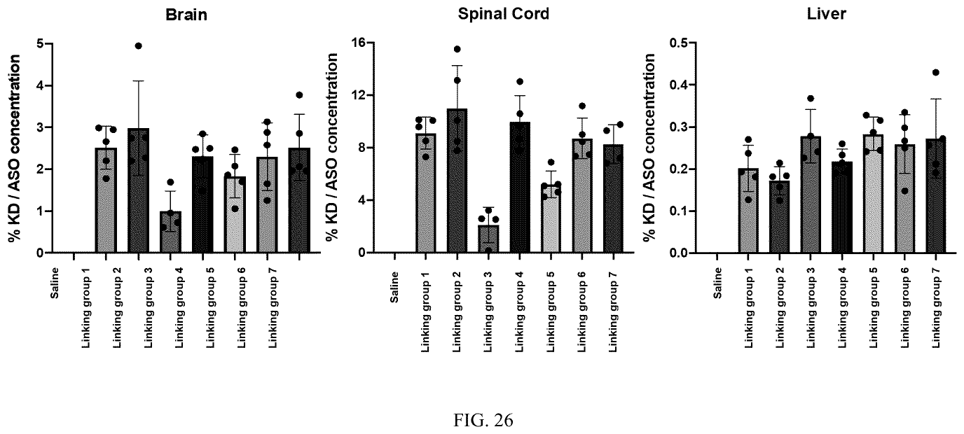

Malat1 expression was measured in the brain (frontal lobe), spinal cord (cervical), and liver (right lobe) as follows. A ˜50 mg piece of tissue was homogenized with a bead homogenizer in Trizol for bulk RNA isolation. Briefly, homogenized tissues were incubated with chloroform for 3-5 minutes to allow for phase separation after centrifugation. The aqueous phase was then incubated with isopropanol for 10 minutes to allow for RNA precipitation followed by a 75% ethanol wash and resuspension in nuclease-free water. Malat1 expression was then measured by qPCR using the Express One-Step Superscript Kit and normalized to expression of the housekeeping gene Gapdh. Results are shown in FIG. 25. All linker variants resulted in robust Malat1 knockdown in the brain, spinal cord, and liver. Interestingly, Malat1 knockdown normalized to total ASO concentration (FIG. 26) revealed less efficient knockdown from Linking Group 3 in both the brain and spinal cord (a larger value for % knockdown to ASO concentration equals more efficient knockdown.)

### Example 10: PK/PD Analysis of S239C, A114C, and T289C Conjugation Site Variants

The systemic stability, brain uptake, and brain knockdown of S239C, A114C, and T289C conjugation site variants was analyzed. Malat1 ASO was conjugated via Linking Group 1 in Table 6 to S239C, A114C, and T289C Fab-Fc dimer fusions described above. The resulting OTV conjugates were diluted in sterile saline and administered to 3-month-old mice expressing a humanized transferrin receptor (hTfR KI) via tail vein intravenous injection at a dose of 25 mg/kg as follows: One group of animals received a single dose, to assess plasma pharmacokinetics. In these mice, in-life plasma was collected at 0.5, 24 and 72 hours after dosing and terminal plasma and tissues were collected at 168 hours after injection. A second group of animals received 3 doses of the OTV variants to assess impact on tissue knockdown of the ASO target (Malat1). In life plasma was collected in these mice after the first dose at 4 and 48 hours to further profile single dose plasma pharmacokinetics. Additional doses were administered at 72 and 168 hours after the first dose, and terminal tissues were collected at 336 hours after the first injection. At study termination, brain, liver and kidney samples were collected for tissue pharmacokinetic and pharmacodynamic response.

### Methods

Mouse handling and tissue collection. Mice were peripherally administered therapeutic treatment via intravenous (IV) tail vein injection (˜200 uL total volume). For in-life plasma collection, blood was collected via submental puncture and transferred to EDTA coated tubes. then spun down at 12,700 rpm for 7 min at 4C before collecting the top plasma layer. For tissue collection, animals were anesthetized with tribromoethanol and whole blood was collected via cardiac puncture into EDTA coated tubes for plasma drug concentration assessment. Following transfer to EDTA coated tubes, whole blood was spun down at 12,700 rpm for 7 min at 4 C before collecting the top plasma layer. Mice were then perfused with ice-cold PBS transcardially at a rate of 5 mL/min for 5 min. For biochemical analysis, tissues were collected, weighed, snap frozen on dry ice, and then stored at −80° C.

Tissue homogenization for drug concentration measurement. Weighed frozen tissue samples were processed for biochemical assays by adding 5× or 10× volume chilled 1% NP40+ PBS homogenization buffer with added cOmplete Protease Inhibitor (Roche #04693132001) and PhosStop (Roche 04906837001) phosphatase inhibitors. Samples were homogenized using 3 mm tungsten carbide beads in 1.5 mL Eppendorf tubes, shaken using the Qiagen TIssueLyzer II (Cat No./ID: 85300) (2×3 min at 27 Hz). For those samples where lysate was used for analysis, samples were centrifuged at 14,000×g for 20 min at 4° C. Lysate supernatant or crude homogenate was then used for downstream analyses. For assays that require total protein normalization, BCA was performed.

Tissue homogenization for RNA measurements. Weighed frozen tissue samples were processed for RNA assays by adding 10× volume Qiazol reagent. Samples were homogenized using 5 mm tungsten carbide beads in 2 mL Eppendorf tubes, shaken using the Qiagen TIssueLyzer II (Cat No./ID: 85300) (2×3 min at 27 Hz). After lysis, samples were incubated for 5 min at room temperature, then chloroform was added. Samples were vortexed, incubated at room temperature for 3 min, then centrifuged for 15 min at 12000×g at 4° C. The aqueous phase was then isolated. RNA was then isolated by adding isopropanol, vortexing, incubating for 10 minutes at room temperature, then centrifuging for 10 min at 12000×g at 4° C. The resulting pellet was then resuspended in 75% ethanol, vortexed and centrifuged for 5 min at 7500×g at 4° C. The final pellet was resuspended in water.

huIgG ELISA protocol. Total therapeutic antibody concentrations in murine plasma and tissue homogenates were quantified using a generic anti-human IgG sandwich-format enzyme-linked immunoassay (ELISA). Briefly, plates were coated overnight at 4° C. with donkey anti-human IgG pAb (Jackson ImmunoResearch, #709-006-098) at 1 μg/mL in sodium bicarbonate solution (Sigma, #C3041-50CAP). Following incubation and plate wash with buffer (PBS+0.05% Tween20), prepared test samples (with sample pre-dilution, where appropriate, in PBS+0.05% Tween20+BSA (10 mg/mL)) and relevant standards were added to the assay plate and were allowed to incubate for 2 hrs. Following test sample incubation and wash step, secondary antibody, goat anti-human IgG (Jackson ImmunoResearch, #109-036-098), was diluted in blocking buffer (PBS+0.05% Tween20+5% BSA (50 mg/mL)) to a final concentration of 0.02 μg/mL. Following a 1 hr incubation and final wash step, plates were developed by adding TMB substrate (Thermo Fisher Scientific, #34028) and incubated for 5-10 minutes. Reaction was quenched by adding 4N H₂SO₄ (Life Technologies, #SS03) and read using 450 nM absorbance. All assay reaction steps were performed at ambient temperature with gentle agitation (where appropriate); and all test samples were pre-diluted at the assay minimum-required-dilution (MRD) if 1:20 prior to analysis. The assay standard curves were fitted with a weighted four-parameter (4PL) nonlinear logistic regression for use in calculating concentrations unknown/test samples.

Intact OTV assay and total ASO assay are as described above. qPCR analysis. To evaluate target mRNA levels, qRT-PCR was run on RNA extracted from either tissue lysates. Target mRNA levels were evaluated using the following Taqman probes: mMalat1, mGAPDH. Express One-Step Kit Taqman. For each sample, Malat1 mRNA levels were normalized to the housekeeping gene Gapdh. qRT-PCR was performed using a QuantStudio 6 Flex system (Applied Biosystems) and average CT values were measured for each probe using sample duplicates. Next, the delta delta CT values were calculated relative to the non-ASO treated group and plotted as relative expression levels.

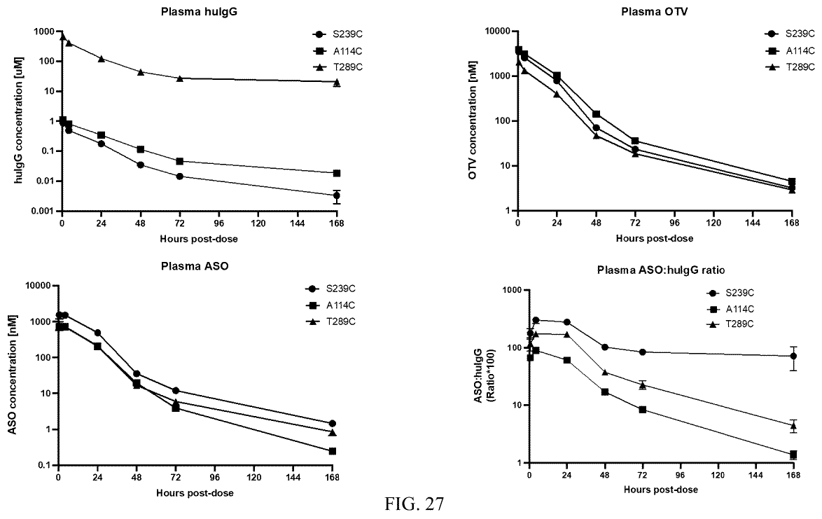

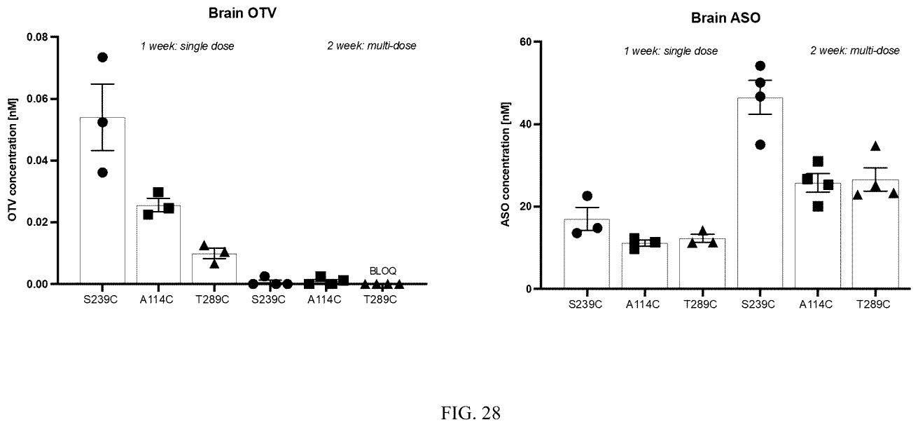

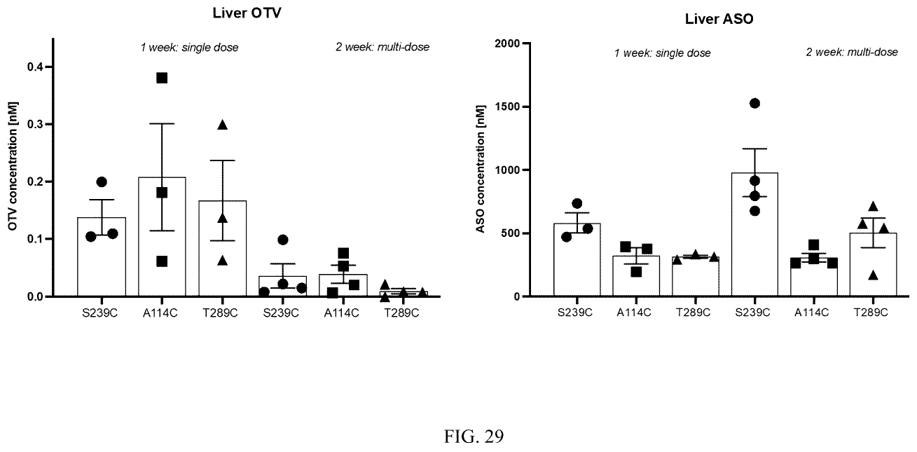

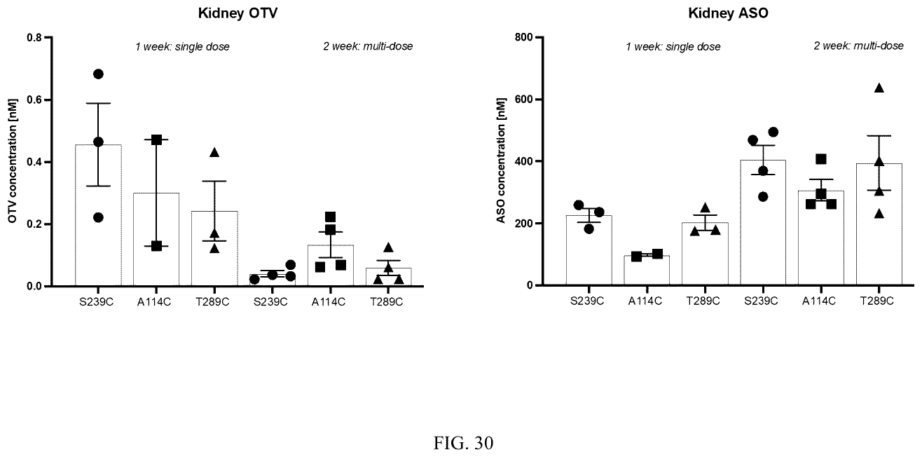

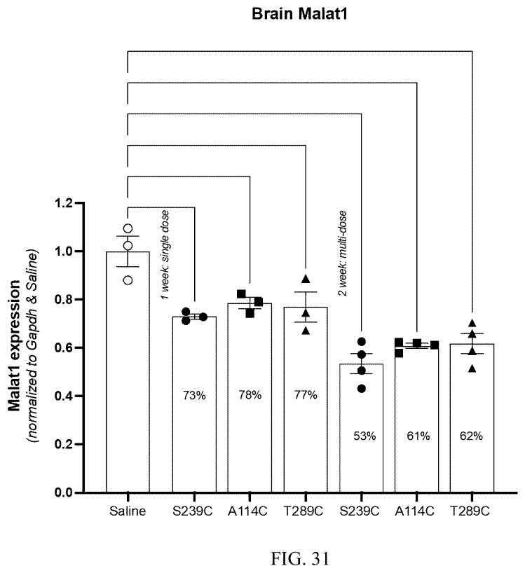

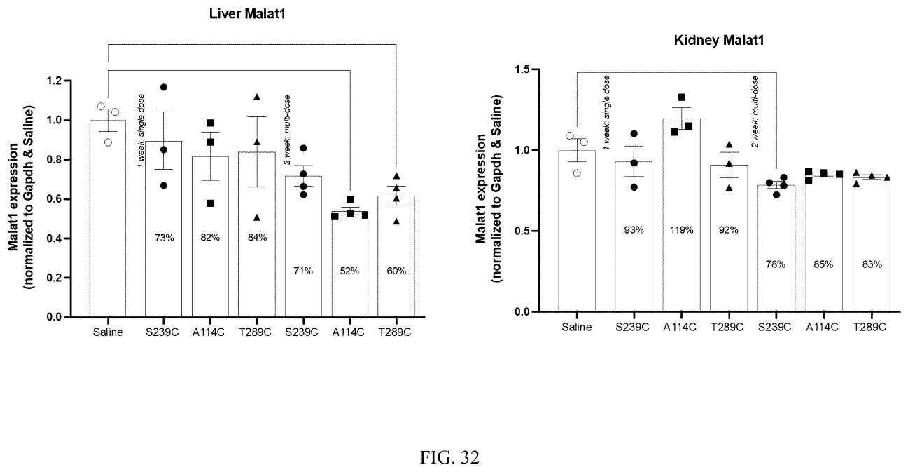

The results are shown in FIGS. 27-32. The data demonstrate that ASO conjugation at A114C or T289C does not significantly alter the plasma PK profile of OTV compared to the S239C site previously described (FIG. 27). The data also demonstrate that all three conjugation sites allow for brain, liver and kidney uptake of ASO (FIGS. 28-30) as well knockdown of ASO target RNA (Malat1)(FIGS. 31-32). The data also support that the S239C site provides the greatest plasma stability, brain uptake and brain knockdown of the three tested conjugation sites.

### Example 11: Construction of OTV:HPRT siRNA

5′-maleimide modified HPRT siRNA strand synthesis and duplexing. HPRT siRNA sense and antisense strands were synthesized according to the sequences in Table 7 below. The sense strand was modified with a maleimide linker on the 3′ end via C6 amino modifier and maleimidopropionic acid NHS ester coupling. The sense and antisense strands were annealed by shaking a 1:1 solution in water at rt for 5 min. Conversion rate was analyzed by SEC-HPLC.

The resulting 5′-maleimide modified HPRT siRNAs were conjugated to the Fab-Fc dimer fusions generated above containing the S239C cysteine modification for conjugation according to the bioconjugation method described below.

TABLE 7

| | | |
|---|---|---|
| HPRT | 5′-U_{fo}CₘₒC_{fo}UₘₒA_{fo}UₘₒG_{fo}AₘₒC_{fo}UₘₒG_{fo}UₘₒA_{fo}GₘₒA_{fo}UₘₒU_{fo}UₘₒU_{fo} | SEQ ID |
| sense | AₘₒU_{fo}-3′ | NO: 870 |
| HPRT | 5′-AₘₒU_{fo}AₘₒA_{foAmo}A_{fo}UₘₒC_{fo}UₘₒA_{fo}CₘₒA_{fo}GₘₒU_{fo}CₘₒA_{fo}UₘₒA_{fo}Gₘₒ | SEQ ID |
| antisense | G_{fo}AₘₛA_{fs}Uₘₒ-3′ | NO: 871 |

f= 2′-F RNA; m = 2′-methoxy RNA; o = phosphodiester bond; s = phosphorothioate bond

Bioconjugation of linker:HPRT siRNA to TV. The Fab-Fc dimer fusions generated above containing the S239C cysteine modification for conjugation was first reduced using 30 molar equivalents of TCEP and 2 mM EDTA, 37° C. for 1 hour. Reduction was confirmed by LC/MS. Post reduction, remaining TCEP was removed by dialysis using 1×PBS, pH 6.8 with 2 mM EDTA (purification by e.g. dialysis) and the Fab-Fc dimer fusions were reoxidized with 50 molar equivalents of dHAA at room temperature for 3 hours. Oxidation was confirmed by LC/MS of dHAA. For the bioconjugation, 1.2 molar equivalents of the 5′-maleimide modified HPRT siRNAs, generated above were added to the oxidized Fab-Fc dimer fusions at room temperature for 1 hour. The resulting conjugates were purified by anion exchange chromatography using Resource Q column (equilibration buffer: 50 mM Tris, pH 7.5, elution buffer: 50 mM Tris, pH 7.5+2M NaCl) to remove unwanted and unconjugated products. Purity of the conjugates were determined by LC/MS and SEC. The resulting conjugates are referred to as OTV:HPRT siRNA. Formulation buffer for OTV:HPRT siRNA was 40 mM PB, 40 mM Arginine, 100 mM NaCl, 6% Sucrose, pH 8.0.

### Example 12: OTV:HPRT siRNA Distribution and Level of Mouse HPRT Knockdown

The OTV:HPRT siRNA produced above were tested for delivering small interfering RNA (siRNA) through the blood-brain barrier (BBB) and into neurons and deep brain regions. siRNA are double-stranded, non-coding oligonucleotides that engage with the catalytic RNA-induced silencing complex (RISC) to degrade mRNA products after transcription, thereby preventing protein translation. Unlike antisense oligonucleotides (ASOs), synthetic siRNA molecules have poor cellular uptake, relying on conjugated ligands to enter the cell. In our study, an siRNA designed to target mouse hypoxanthine guanine phosphoribosyl transferase (HPRT), a common house-keeping gene expressed in cells. The OTV:siRNA molecules were also analyzed for their circulation stability, brain penetrance, selective/non-selective cellular uptake, gene silencing potency, and gene silencing duration.

TfR^{ms/hu} knock-in mice were administered either intravenously a single dose or intravenously and subcutaneously in the multi-dose arms: four doses (Day 0, 3, 7, 10). Anti-CD4 was administered prior to the initial doses in all arms to prevent anti-drug antibody responses in the mice. The OTV:siRNA conjugate was diluted in sterile saline and dosed at 25 mpk. Plasma was collected at 4 and 24 hours after the initial dose, and 48 hours after the third dose. Tissue samples (i.e., brain, spinal cord, kidney, liver and quadricep) and terminal blood was collected 72 hours after the final dose to determine siRNA distribution and level of mouse HPRT knockdown relative to saline dosed mice. All animals were perfused with sterile saline and blood was collected in EDTA plasma tubes, spun at 14,000 rpm for 5 minutes, and plasma was isolated for subsequent analysis.

huIgG Assay. The total huIgG concentrations in plasma were quantified using a generic anti-human IgG sandwich-format ELISA. Briefly, plates were coated overnight at 4° C. with donkey anti-human IgG (JR #709-006-098) at 1 μg/ml in sodium bicarbonate solution (Sigma #C3041-50CAP) with gentle agitation. Plates were then washed 3× with wash buffer (PBS+0.05% Tween 20). Assay standards and samples were diluted in PBS+0.05% Tween 20 and 1% BSA (10 mg/mL). Standard curve preparation ranged from 0.41 to 1,500 ng/mL or 0.003 to 10 nM (BLQ<0.03 nM). Standards and diluted samples were incubated with agitation for 2 hr at room temperature. After incubation, plates were washed 3× with wash buffer. Detection antibody, goat anti-human IgG (JIR #109-036-098), was diluted in blocking buffer (PBS+0.05% Tween 20+5% BSA (50 mg/mL)) to a final concentration of 0.02 μg/mL and plates were incubated with agitation for 1 hr at room temperature. After a final 3× wash, plates were developed by adding TMB substrate and incubated for 5-10 minutes. Reaction was quenched by adding 4N H₂SO₄ and read using 450 nM absorbance.

Total Antisense (4) and Sense Strand (SS) Assay. Quantification of either AS or SS (in conjugated and free forms) in mouse plasma and tissue homogenates were measured using a hybridization-based electrochemiluminescence immunoassay (ECLIA). Briefly, custom biotinylated and digoxigenin-conjugated antisense probes (synthesized by Integrated DNA Technologies, Coralville, IA) at working concentrations were combined with prepared test samples (with sample pre-dilution, where appropriate) and relevant standards in TE Buffer (10 mM Tris-HCL containing 1 mM EDTA). Prepared samples in TE buffer were added, in a 1:1 mix, into 1×SSC Buffer (Sigma-Aldrich, St. Louis, MO) containing a working concentration of recombinant proteinase K enzyme (Thermofisher, Waltham, MA). Hybridization/Enzyme mixture was then digested, denatured, annealed, and cooled in a thermal cycler instrument. Following hybrid product incubation, samples were added to the wells of an MSD GOLD 96-well streptavidin-coated microtiter plate (Meso Scale Discovery, Rockville, MD) and incubated for approximately 30 mins. Following incubation and a plate wash step, secondary ruthenylated (SULFO-TAG) sheep anti-digoxigenin antibody (Novus Biologicals, Littleton, CO, labelled in-house) at a working concentration in assay diluent was added to the plate and incubated for approximately 30 mins. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

Intact siRNA-OTV Assay. Quantification of Intact OTV (humanized anti-TfR antibody conjugated to an antisense oligonucleotide (ASO) in mouse plasma and tissue lysates were measured using a hybridization-based electrochemiluminescence immunoassay (ECLIA). Briefly, custom biotinylated antisense probes (synthesized by Integrated DNA Technologies, Coralville, IA) at a working concentration were incubated with prepared test samples (with sample pre-dilution, where appropriate) and relevant standards in TE Buffer (10 mM Tris-HCL containing 1 mM EDTA) and hybridized at an appropriate temperature for 45 mins. Following the incubation, hybridized product was added to the wells of an MSD GOLD 96-well streptavidin-coated microtiter plate (Meso Scale Discovery, Rockville, MD) and incubated for approximately 30 mins. Following hybrid product incubation and a plate wash step, secondary ruthenylated (SULFO-TAG) goat anti-human IgG antibody (Meso Scale Discovery, Rockville, MD) at a working concentration in assay diluent was added to the assay plate and incubated for approximately 1 hr. Following a plate wash, a 1×MSD Read Buffer T (Meso Scale Discovery, Rockville, MD) was then added to generate the electrochemiluminescence (ECL) assay signal, which was then expressed in ECL units (ECLU). All of the assay reaction steps were performed at ambient temperature with shaking on a plate shaker (where appropriate); and all test samples were pre-diluted at the assay MRD of 1:20 prior to analyzing in the assay plate. Sample ECLU signals generated in the assay subsequently were processed into concentrations by back-calculating off the assay calibration (CS) curve. The assay CS curve was fitted with a weighted four-parameter nonlinear logistic regression for use in calculating concentrations for unknown/test samples.

TABLE 8

| | | |
|---|---|---|
| HPRT AS Assay Probes | | |
| Capture | /5BioTEG/ATTC+CTAT+GACT | SEQ ID NO: 872 |
| Detection | GT+A+GA+TTT+TAT/3Dig_N/ | SEQ ID NO: 873 |
| HPRT SS Assay Probes | | |
| Capture | /5BioTEG/AT+A+AAAT+C+TAC | SEQ ID NO: 874 |
| Detection | AGT+CATA+G+GA/3Dig_N/ | SEQ ID NO: 875 |
| OTV: HPRT Assay | | |
| Probe | | |
| Capture | /5BioTEG/ATAAAAT+CTA+CA+GT+CATA+GGA | SEQ ID NO: 876 |

“/5Bio TEG/” is a biotin conjugation with TEG spacer
“/3Dig_N/” is a digoxygenin conjugation
“+” before the nucleotide signifies that it is a locked nucleic acid sugar modification

HPRT knockdown in brain, spinal cord, and quad was observed with OTV:HPRT siRNA, indicating functional delivery of the siRNA to these tissues using the OTV.

### Example 14: Analysis of TfR Binding Affinity, CNS Gene Knockdown, and Safety Profile

To assess the effect of TfR binding affinity on CNS gene knockdown and safety profile, the S239C (100 nM) and 500 nM TfR affinity variants described above were compared with a 10 nM TfR affinity binder. The 10 nM TfR affinity binder is a non-TV Fab-Fc dimer fusion (i.e., does not have a Tfr binding site in a constant domain) conjugated to Malat1 ASO where a monovalent Fab binds TfR with an affinity of 10 nM (and the other Fab is a non-binding RSV Fab). The 100 nM, 500 nM, and 10 nM variants were diluted in sterile saline before administration. As controls, saline, unconjugated ASO, and RSV-ASO groups were included.

For the single dose study, 2 mo old TfR^{ms/hu} female mice were administered doses intravenously according to the groups (n=4) in Table 9. Tissue was collected 24 hours after the single dose. In particular, brain, spinal cord, and peripheral organs (kidney, lung, liver, and quadricep muscle) were harvested. Terminal blood was also collected 24 hours after the single dose.

For the multi-dose study, 2 mo old TfR^{ms/hu} female mice were administered doses intravenously according to the groups (n=6) in Table 8 on Day 1, Day 7, and Day 14. Plasma collections were taken at 30 min, 4 hours, 24 hours, 48 hours, 72 hours, and 1 week. Tissue was collected 72 hours after the last dose. In particular, brain, spinal cord, and peripheral organs (kidney, lung, liver, and quadricep muscle) were harvested. Terminal blood was also collected 72 hours after the last dose.

TABLE 9

| | Group | Dose |
|---|---|---|
| 1 | Neg Control: Saline | |
| 2 | Neg Control: Naked ASO (unconjugated ASO) | 1 mg/kg |
| 3 | Neg Control: RSV-ASO | 25 mg/kg |
| 4 | S239C (100 nM) | 25 mg/kg |
| 7 | 500 nM | 25 mg/kg |
| 8 | 10 nM | 25 mg/kg |

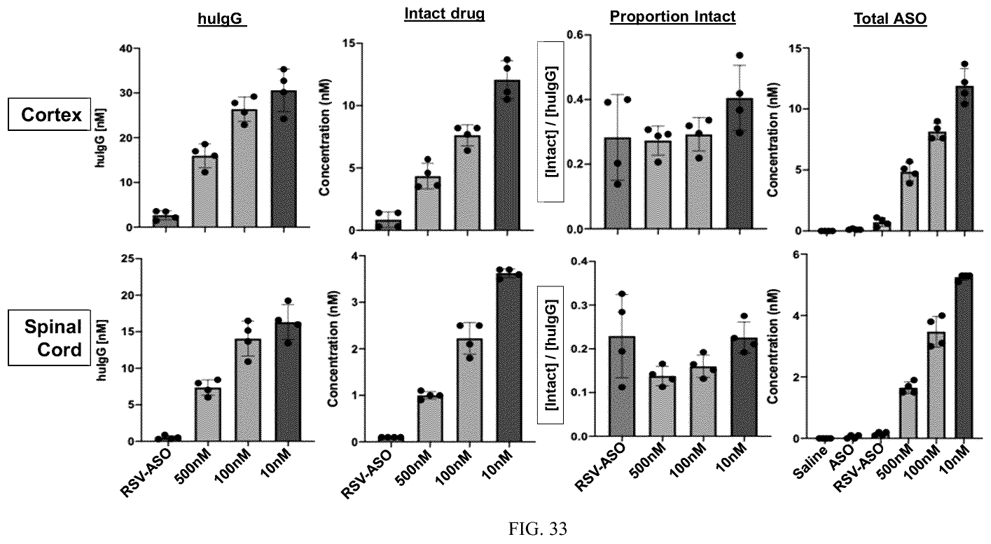

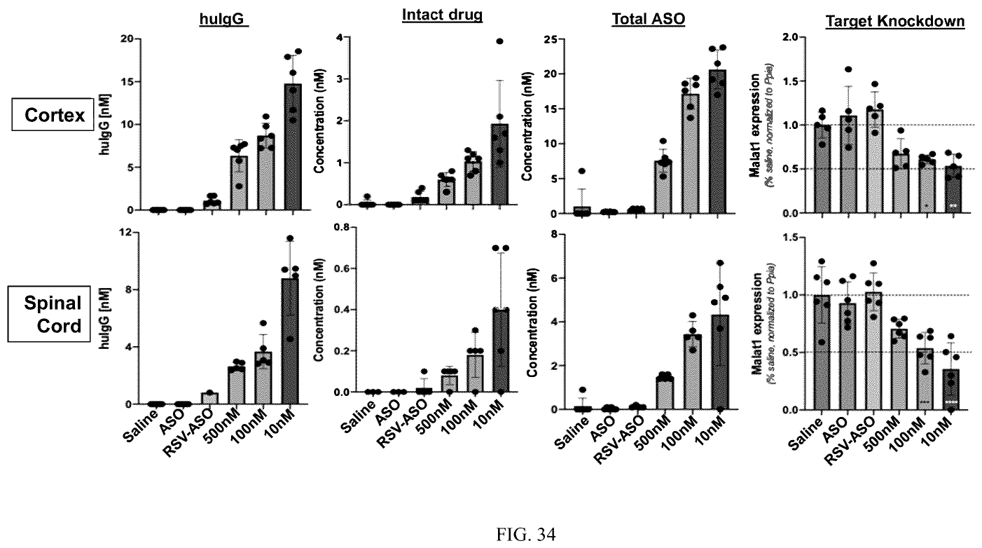

Intact drug and total ASO in the brain and spinal cord for the single- and multi-dose study were measured according to the methods described above. FIG. 33 shows increased delivery of intact drug and total ASO to the CNS increases with TfR affinity in the single-dose study, i.e., 10 nM TfR affinity delivering the most intact drug and total ASO to the CNS. A similar trend is seen in the multidose study (FIG. 34). Malat1 knockdown in the brain and spinal cord for the multi-dose study was quantified using methods described above. Results are shown in Slide (FIG. 34). Malat1 knockdown was seen at all 3 TfR binding affinities. Increasing TfR affinity increased Malat1 knockdown in the CNS, with the highest knockdown seen with 10 nM TfR affinity.

Plasma PK for the multi-dose study was quantified using methods described above. CMax (C₀), total exposure (AUC), and clearance results are shown in Table 10 below. The 500 nM TfR affinity molecule clears more slowly than the 100 nM and 10 nM TfR affinity molecules.

TABLE 10
Plasma PK

| | C₀ (uM) | AUC (uM*hr) | CL (mL/d/kg) |
|---|---|---|---|
| RSV-ASO | 2.04 | 84.7 | 35.5 |
| 500 nM affinity | 3.29 | 50.0 | 81.0 |
| S239C (100 nM affinity) | 2.53 | 36.2 | 113 |
| 10 nM affinity | 2.07 | 28.0 | 124 |

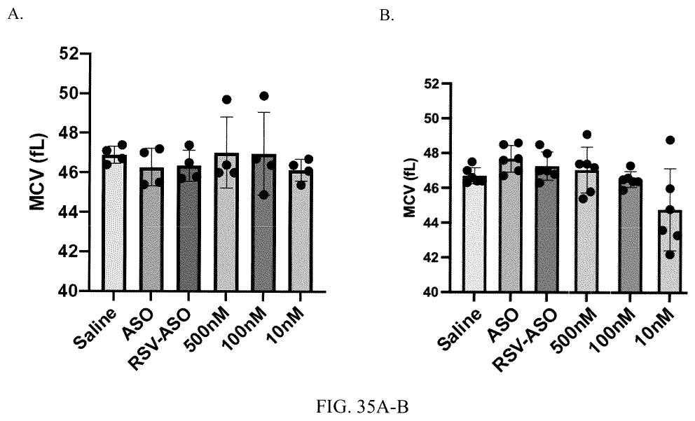

Additionally, MCV (Mean Cell Volume) was measured in plasma samples collected from the single and multidose studies. The mean cell volume (MCV) indicates the volume of the “average” red blood cell (RBC) in a sample and is an indication of iron uptake. Blood samples were collected into an anticoagulant, EDTA, and MCV was measured by an automated hematology analyzer. Results are shown in FIG. 35. MCV is lower with 10 nM TfR binding in the single dose study and MCV is further decreased in the multidose study. This suggests increasing impairment of iron uptake and erythropoiesis which may result in increasing iron-deficient anemia for TfR binding with higher affinity than 10 nM, especially with chronic dosing.

Furthermore, the S239 variant (described above) which binds TfR with an affinity of 100 nM was compared to an anti-TfR bivalent antibody that binds with an affinity of 0.12 nM conjugated to Malat1 ASO.

Each of these molecules was diluted in sterile saline and administered to TfRms/hu knock-in mice intravenously at a weekly dose of 50 mg/kg for 4 weeks. Two control groups of TfR^{ms/hu} mice were dosed with either sterile saline or unconjugated ASO intravenously. Three days after the fourth dose, tissues were collected and frozen for molecular and biochemical analysis. Tissues include the brain, spinal cord, liver, heart, quadricep, diaphragm, and sciatic nerve.

Four tissues with robust TfR expression (brain vasculature, brain parenchyma, liver, and heart) were homogenized with a bead homogenizer in 1% NP-40 buffer at a final concentration of 100 mg tissue per mL of buffer. Homogenized tissues were then centrifuged at 15,000×g for 15 minutes and the supernatant was prepared for western blot. Briefly, samples were mixed with a reducing agent and boiled at 95° C. prior to running on a gel and transferring to a nitrocellulose blot. The blot was blocked with 5% milk then incubated in an antibody solution containing anti-TfR at a dilution of 1:2000. A single piece of brain tissue was used for a capillary depletion in order to separate the brain vasculature from the brain parenchyma.

Malat1 expression was measured in the brain, spinal cord, liver, heart, quadricep, diaphragm, and sciatic nerve as follows. A <50 mg piece of tissue was homogenized with a bead homogenizer in Trizol for bulk RNA isolation. Homogenized tissues were incubated with chloroform for 3-5 minutes to allow for phase separation after centrifugation. The aqueous phase was then incubated with isopropanol for 10 minutes to allow for RNA precipitation followed by a 75% ethanol wash and resuspension in nuclease-free water. Malat1 expression was then measured by qPCR using the Express One-Step Superscript Kit and normalized to expression of the housekeeping gene Gapdh.

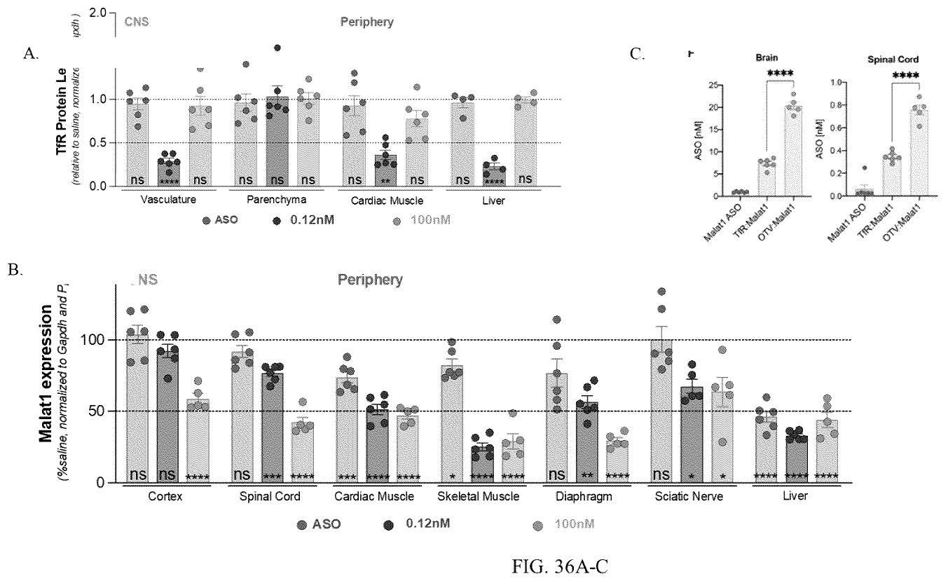

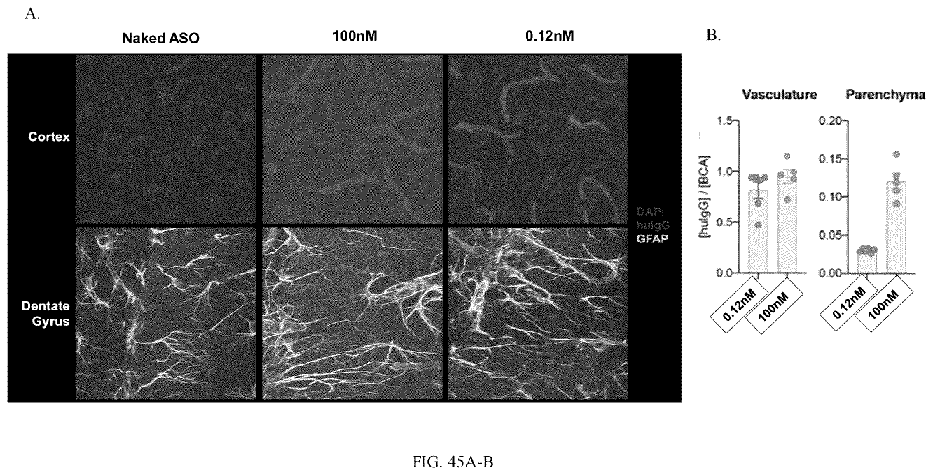

Results are shown in Slide 36A-C. The 100 nM variant deposited significantly more ASO in the brain and spinal cord (FIG. 36C) and heart and comparable levels in liver and quadricep muscle. Superior CNS knockdown with 100 nM TfR binding is seen compared to 0.12 nM TfR binding (FIG. 36B). Very little knockdown is observed in the cortex and spinal cord with 0.12 nM affinity suggesting that the molecule is not getting into the brain, i.e., crossing the endothelial cell layer at the BBB, and instead accumulating in the brain vasculature and failing to move into parenchymal tissue. Additionally, brain tissue samples were stained for huIgG 24 hours after administration of the 100 nM and 0.1 mM variant. Homogenous huIgG signal was observed throughout the brains of 100 nM variant-dosed mice, but very heterogenous distribution in 0.12 nM variant-dosed mice, with robust huIgG signal in vessels but minimal huIgG detectable in parenchymal tissue (FIG. 45A). This difference was confirmed by huIgG ELISA after brain capillary depletion, which showed significantly more huIgG in the parenchyma of mice dosed with the 100 nM variant, but comparable huIgG levels in the vasculature (FIG. 45B).

Furthermore, TfR degradation is observed in the liver, heart, and brain vasculature, but not the brain parenchyma with the 0.12 nM affinity variant further suggesting inefficient crossing of the BBB due to enhanced TfR binding in the vasculature. Additionally, the significant TfR degradation seen with 0.12 nM TfR binding compared with 100 nM TfR binding (FIG. 36A) is a safety concern. A lower number of TfR on the cell surface leads to decreased transferrin binding and impairs the cell's iron uptake mechanism.

Taken together, the data suggests that delivering the ASO via a TfR affinity in the range of 500 nM to 10 nM strikes a balance between clearance rate, pharmacodynamic/knockdown, and safety concerns.

### Example 15: Malat1 Knockdown in Intravenously Delivered Unconjugated ASO v. OTV:ASO

Unconjugated Malat1 ASO (“naked ASO”) and OTV conjugated to Malat1 ASO (“OTV”) were administered to 2-month old TfR^{ms/hu} KI mice intravenously at 2.5 mg/kg ASO (or molar equivalent) and ATV:BACE1 was administered to a control cohort of TfR^{ms/hu} KI mice at 57 mg/kg (protein molar equivalent to OTV) in a single dose administration. For the multi-dose arm naked ASO, OTV, and control were administered as above to mice once a week for 4 weeks. Each cohort size was n=5 mice.

The mice cohorts were sacrificed 72 hours after single dose administration or 72 hours after the fourth dose in the multi-dose study for analysis. Mice were perfused with chilled PBS, and the following organs were collected for RNA extraction: cortex, brain stem, hippocampus, striatum, cerebellum, thalamus, cervical spinal cord, lumbar spinal cord, retina, sciatic nerve, quadriceps, heart, diaphragm, spleen, intestine, lung, liver, and kidney.

RNA isolation and Malat1 mRNA measurement: RNA was extracted using the RNeasy Plus Mini Kit (Qiagen) and concentration determine on the nanodrop. Malat1 mRNA levels were measured using qRT-PCR (ThermoFisher EXPRESS One-Step) with Malat1 primers/probes (ThermoFisher Mm01227912) and normalized to Gapdh (ThermoFisher Mm99999915) and Ppia (ThermoFisher Mm02342430) mRNA levels. The delta delta Ct method of quantification was used.

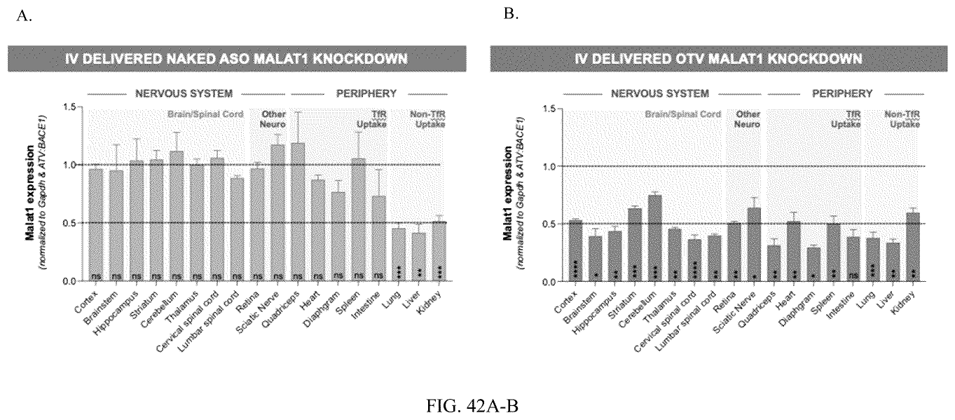

The results from the single-dose study are shown in FIG. 42. OTV provides uniform target RNA knockdown across not only multiple regions of the CNS, but also in peripheral nerves, muscle, and other peripheral organs. This enables OTV to be used for not only CNS disorders but neuromuscular disorders where there is both a CNS and muscle phenotype.

### Example 16: ASO PK/PD Kinetics after OTV Administration

To understand the rates of ASO degradation across different CNS and peripheral tissues and the impact of ASO degradation on target knockdown, TfRms/hu KI mice (n=5) were intravenously administered either a single dose of OTV or a loading window of 6×IV dose of OTV as follows:

Single dose: 1 mg/kg ASO molar equivalent OTV. Plasma, brain (frontal cortex), spinal cord, quadricep muscle, liver, and kidney were collected at 1 d, 4 d, 1 wk, 2 wk, 4 wk, and 8 wk post dose.

Multi-dose: 6×IV dose of OTV (1 mg/kg parent ASO) administered biweekly (twice a week); After the 6×1 mg/kg ASO molar equivalent OTV administrations over the course of 3 weeks, plasma, brain (frontal cortex), spinal cord, quadricep muscle, liver, and kidney were collected at 1 wk, 2 wk, 4 wk, 8 wk and 12 wk post dose.

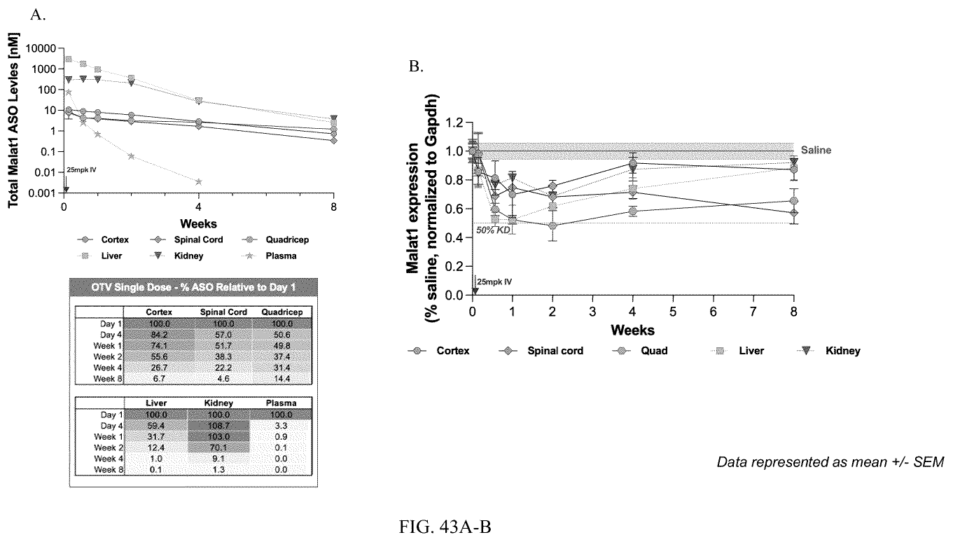

A single dose of OTV shows the highest starting concentration of ASO in liver and kidney, though the ASO half-life in those tissues is much shorter than TfR mediated uptake tissues, such as brain, spinal cord, and quadricep muscle (FIG. 43A). Despite the higher initial ASO concentration in liver and kidney, there is no added knockdown benefit compared to the TfR mediated uptake tissues (brain, spinal cord, quadricep muscle) (FIG. 43B). Notably, a single OTV dose provides sustained 8 week knockdown in the quadricep and spinal cord CNS tissues.

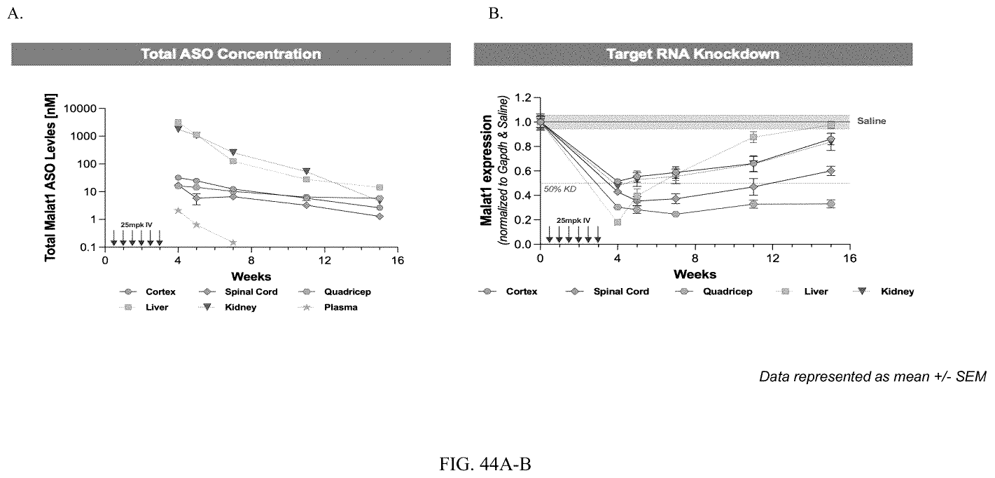

Similarly, a multi-dose loading window of OTV shows the highest starting concentration of ASO in liver and kidney, though the ASO half-life in those tissues is much shorter than TfR mediated uptake tissues, such as brain, spinal cord, and quadricep muscle (FIG. 44A). As with the single-dose study, despite the higher initial ASO concentration in liver and kidney, there is no added knockdown benefit compared to the TfR mediated uptake tissues (brain, spinal cord, quadricep muscle) (FIG. 44B). ASO delivered via OTV shows extended knockdown in CNS and muscle compared to liver. Liver Malat1 RNA levels rebound the quickest. In contrast, brain, spinal cord, and quadricep all still show Malat1 knockdown even after 12 weeks post-loading doses.

TABLE A
CH3B Register Positions and Mutations

| Sequence | Seq. | | | | | | | | | | |
|---|---|---|---|---|---|---|---|---|---|---|---|
| name | group | 118 | 119 | 120 | 121 | 122 | . . . | 210 | 211 | 212 | 213 |
| Wild-type | n/a | E | P | Q | V | Y | . . . | T | Q | K | S |
| CH3B.1 | 1 | F | D | Y | V | T | . . . | G | F | H | D |
| CH3B.2 | 1 | F | D | M | V | T | . . . | G | F | H | D |
| CH3B.3 | 1 | F | E | Y | V | T | . . . | G | F | H | D |
| CH3B.4 | 1 | F | E | M | V | T | . . . | G | F | H | D |
| CH3B.5 | 1 | F | E | L | V | T | . . . | G | F | H | D |
| CH3B.6 | 1 | F | E | I | V | T | . . . | G | F | H | D |
| CH3B.7 | 1 | F | D | I | V | T | . . . | G | F | H | D |
| CH3B.8 | 1 | F | D | Y | V | T | . . . | G | F | H | D |
| CH3B.9 | 1 | F | G | M | V | T | . . . | G | F | H | D |
| CH3B.10 | 1 | F | A | D | V | T | . . . | G | F | Y | D |
| CH3B.11 | 1 | F | G | L | V | T | . . . | G | F | H | D |
| CH3B.12 | 1 | F | D | Y | V | T | . . . | G | F | S | D |
| CH3B.13 | 1 | I | D | Y | V | T | . . . | G | F | S | D |
| CH3B.14 | 1 | F | K | D | V | T | . . . | G | F | F | D |
| CH3B.15 | 1 | F | D | L | V | T | . . . | G | F | Y | D |
| CH3B.16 | 1 | I | D | Y | V | T | . . . | G | F | S | D |
| CH3B.17 | 1 | F | E | L | V | A | . . . | G | F | H | D |

TABLE B
CH3C Register Positions and Mutations

| Sequence | Seq. | | | | | | | | | | | | | | | | | |
|---|---|---|---|---|---|---|---|---|---|---|---|---|---|---|---|---|---|---|
| name | group | 157 | 158 | 159 | 160 | 161 | 162 | 163 | 164 | 186 | 187 | 188 | 189 | 190 | 191 | 192 | 193 | 194 |
| Wild-type | n/a | N | G | Q | P | E | N | N | Y | D | K | S | R | W | Q | Q | G | N |
| CH3C.1 | | L | G | L | V | W | V | G | Y | A | K | S | T | W | Q | Q | G | W |
| CH3C.2 | | Y | G | T | V | W | S | H | Y | S | K | S | E | W | Q | Q | G | Y |
| CH3C.3 | | Y | G | T | E | W | S | Q | Y | E | K | S | D | W | Q | Q | G | H |
| CH3C.4 | | V | G | T | P | W | A | L | Y | L | K | S | E | W | Q | Q | G | W |
| CH3C.17 | 2 | Y | G | T | V | W | S | K | Y | S | K | S | E | W | Q | Q | G | F |
| CH3C.18 | 1 | L | G | H | V | W | A | V | Y | P | K | S | T | W | Q | Q | G | W |
| CH3C.21 | 1 | L | G | L | V | W | V | G | Y | P | K | S | T | W | Q | Q | G | W |
| CH3C.25 | 1 | M | G | H | V | W | V | G | Y | D | K | S | T | W | Q | Q | G | W |
| CH3C.34 | 1 | L | G | L | V | W | V | F | S | P | K | S | T | W | Q | Q | G | W |
| CH3C.35 | 2 | Y | G | T | E | W | S | S | Y | T | K | S | E | W | Q | Q | G | F |
| CH3C.44 | 2 | Y | G | T | E | W | S | N | Y | S | K | S | E | W | Q | Q | G | F |
| CH3C.51 | 1/2 | L | G | H | V | W | V | G | Y | S | K | S | E | W | Q | Q | G | W |
| CH3C.3.1-3 | 1 | L | G | H | V | W | V | A | T | P | K | S | T | W | Q | Q | G | W |
| CH3C.3.1-9 | 1 | L | G | P | V | W | V | H | T | P | K | S | T | W | Q | Q | G | W |
| CH3C.3.2-5 | 1 | L | G | H | V | W | V | D | Q | P | K | S | T | W | Q | Q | G | W |
| CH3C.3.2-19 | 1 | L | G | H | V | W | V | N | Q | P | K | S | T | W | Q | Q | G | W |
| CH3C.3.2-1 | 1 | L | G | H | V | W | V | N | F | P | K | S | T | W | Q | Q | G | W |
| CH3C.3.4-1 | | W | G | F | V | W | S | T | Y | P | K | S | N | W | Q | Q | G | F |
| CH3C.3.4-19 | | W | G | H | V | W | S | T | Y | P | K | S | N | W | Q | Q | G | Y |
| CH3C.3.2-3 | | L | G | H | V | W | V | E | Q | P | K | S | T | W | Q | Q | G | W |
| CH3C.3.2-14 | | L | G | H | V | W | V | G | V | P | K | S | T | W | Q | Q | G | W |
| CH3C.3.2-24 | | L | G | H | V | W | V | H | T | P | K | S | T | W | Q | Q | G | W |
| CH3C.3.4-26 | | W | G | T | V | W | G | T | Y | P | K | S | N | W | Q | Q | G | Y |
| CH3C.3.2-17 | | L | G | H | V | W | V | G | T | P | K | S | T | W | Q | Q | G | W |

TABLE C
Exploration of Acceptable Diversity Within Register and Hot Spot Positions for CH3C.35.21

| | 151 | 152 | 153 | 154 | 155 | 156 | 157 | 158 | 159 | 160 | 161 | 162 | 163 | 164 |
|---|---|---|---|---|---|---|---|---|---|---|---|---|---|---|
| Wild-type | A | V | E | W | E | S | N | G | Q | P | E | N | N | Y |
| CH3C.35.20.1 | . | . | . | . | . | . | F | . | T | E | W | S | S | . |
| CH3C.35.20.2 | . | . | . | . | . | . | Y | . | T | E | W | A | S | . |
| CH3C.35.20.3 | . | . | . | . | . | . | Y | . | T | E | W | V | S | . |
| CH3C.35.20.4 | . | . | . | . | . | . | Y | . | T | E | W | S | S | . |
| CH3C.35.20.5 | . | . | . | . | . | . | F | . | T | E | W | A | S | . |
| CH3C.35.20.6 | . | . | . | . | . | . | F | . | T | E | W | V | S | . |
| CH3C.35.21.a.1 | . | . | W | . | . | . | F | . | T | E | W | S | S | . |
| CH3C.35.21.a.2 | . | . | W | . | . | . | Y | . | T | E | W | A | S | . |
| CH3C.35.21.a.3 | . | . | W | . | . | . | Y | . | T | E | W | V | S | . |
| CH3C.35.21.a.4 | . | . | W | . | . | . | Y | . | T | E | W | S | S | . |
| CH3C.35.21.a.5 | . | . | W | . | . | . | F | . | T | E | W | A | S | . |
| CH3C.35.21.a.6 | . | . | W | . | . | . | F | . | T | E | W | V | S | . |
| CH3C.35.23.1 | . | . | . | . | . | . | F | . | T | E | W | S | . | . |
| CH3C.35.23.2 | . | . | . | . | . | . | Y | . | T | E | W | A | . | . |
| CH3C.35.23.3 | . | . | . | . | . | . | Y | . | T | E | W | V | . | . |
| CH3C.35.23.4 | . | . | . | . | . | . | Y | . | T | E | W | S | . | . |
| CH3C.35.23.5 | . | . | . | . | . | . | F | . | T | E | W | A | . | . |
| CH3C.35.23.6 | . | . | . | . | . | . | F | . | T | E | W | V | . | . |
| CH3C.35.24.1 | . | . | W | . | . | . | F | . | T | E | W | S | . | . |
| CH3C.35.24.2 | . | . | W | . | . | . | Y | . | T | E | W | A | . | . |
| CH3C.35.24.3 | . | . | W | . | . | . | Y | . | T | E | W | V | . | . |
| CH3C.35.24.4 | . | . | W | . | . | . | Y | . | T | E | W | S | . | . |
| CH3C.35.24.5 | . | . | W | . | . | . | F | . | T | E | W | A | . | . |
| CH3C.35.24.6 | . | . | W | . | . | . | F | . | T | E | W | V | . | . |
| CH3C.35.21.17.1 | . | . | L | . | . | . | F | . | T | E | W | S | S | . |
| CH3C.35.21.17.2 | . | . | L | . | . | . | Y | . | T | E | W | A | S | . |
| CH3C.35.21.17.3 | . | . | L | . | . | . | Y | . | T | E | W | V | S | . |
| CH3C.35.21.17.4 | . | . | L | . | . | . | Y | . | T | E | W | S | S | . |
| CH3C.35.21.17.5 | . | . | L | . | . | . | F | . | T | E | W | A | S | . |
| CH3C.35.21.17.6 | . | . | L | . | . | . | F | . | T | E | W | V | S | . |
| CH3C.35.20 | . | . | . | . | . | . | Y | . | T | E | W | S | S | . |
| CH3C.35.21 | . | . | W | . | . | . | Y | . | T | E | W | S | S | . |
| CH3C.35.22 | . | . | W | . | . | . | Y | . | T | E | W | S | . | . |
| CH3C.35.23 | . | . | . | . | . | . | Y | . | T | E | W | S | . | . |
| CH3C.35.24 | . | . | W | . | . | . | Y | . | T | E | W | S | . | . |
| CH3C.35.21.17 | . | . | L | . | . | . | Y | . | T | E | W | S | S | . |
| CH3C.35.N390 | . | . | . | . | . | . | Y | . | T | E | W | S | . | . |
| CH3C.35.20.1.1 | | | | | | | F | | T | E | W | S | S | |
| CH3C.35.23.2.1 | | | | | | | Y | | T | E | W | A | | |
| CH3C.35.23.1.1 | | | | | | | F | | T | E | W | S | | |
| CH3C.35.S413 | | | | | | | Y | | T | E | W | S | S | |
| CH3C.35.23.3.1 | | | | | | | Y | | T | E | W | V | | |
| CH3C.35.N390.1 | | | | | | | Y | | T | E | W | S | | |
| CH3C.35.23.6.1 | | | | | | | F | | T | E | W | V | | |
| | 165 | 184 | 185 | 186 | 187 | 188 | 189 | 190 | 191 | 192 | 193 | 194 | 195 | 196 |
| Wild-type | K | T | V | D | K | S | R | W | Q | Q | G | N | V | F |
| CH3C.35.20.1 | . | . | . | T | . | E | E | . | . | . | . | F | . | . |
| CH3C.35.20.2 | . | . | . | T | . | E | E | . | . | . | . | F | . | . |
| CH3C.35.20.3 | . | . | . | T | . | E | E | . | . | . | . | F | . | . |
| CH3C.35.20.4 | . | . | . | S | . | E | E | . | . | . | . | F | . | . |
| CH3C.35.20.5 | . | . | . | T | . | E | E | . | . | . | . | F | . | . |
| CH3C.35.20.6 | . | . | . | T | . | E | E | . | . | . | . | F | . | . |
| CH3C.35.21.a.1 | . | . | . | T | . | E | E | . | . | . | . | F | . | . |
| CH3C.35.21.a.2 | . | . | . | T | . | E | E | . | . | . | . | F | . | . |
| CH3C.35.21.a.3 | . | . | . | T | . | E | E | . | . | . | . | F | . | . |
| CH3C.35.21.a.4 | . | . | . | S | . | E | E | . | . | . | . | F | . | . |
| CH3C.35.21.a.5 | . | . | . | T | . | E | E | . | . | . | . | F | . | . |
| CH3C.35.21.a.6 | . | . | . | T | . | E | E | . | . | . | . | F | . | . |
| CH3C.35.23.1 | . | . | . | T | . | E | E | . | . | . | . | F | . | . |
| CH3C.35.23.2 | . | . | . | T | . | E | E | . | . | . | . | F | . | . |
| CH3C.35.23.3 | . | . | . | T | . | E | E | . | . | . | . | F | . | . |
| CH3C.35.23.4 | . | . | . | S | . | E | E | . | . | . | . | F | . | . |
| CH3C.35.23.5 | . | . | . | T | . | E | E | . | . | . | . | F | . | . |
| CH3C.35.23.6 | . | . | . | T | . | E | E | . | . | . | . | F | . | . |
| CH3C.35.24.1 | . | . | . | T | . | E | E | . | . | . | . | F | . | . |
| CH3C.35.24.2 | . | . | . | T | . | E | E | . | . | . | . | F | . | . |
| CH3C.35.24.3 | . | . | . | T | . | E | E | . | . | . | . | F | . | . |
| CH3C.35.24.4 | . | . | . | S | . | E | E | . | . | . | . | F | . | . |
| CH3C.35.24.5 | . | . | . | T | . | E | E | . | . | . | . | F | . | . |
| CH3C.35.24.6 | . | . | . | T | . | E | E | . | . | . | . | F | . | . |
| CH3C.35.21.17.1 | . | . | . | T | . | E | E | . | . | . | . | F | . | . |
| CH3C.35.21.17.2 | . | . | . | T | . | E | E | . | . | . | . | F | . | . |
| CH3C.35.21.17.3 | . | . | . | T | . | E | E | . | . | . | . | F | . | . |
| CH3C.35.21.17.4 | . | . | . | S | . | E | E | . | . | . | . | F | . | . |
| CH3C.35.21.17.5 | . | . | . | T | . | E | E | . | . | . | . | F | . | . |
| CH3C.35.21.17.6 | . | . | . | T | . | E | E | . | . | . | . | F | . | . |
| CH3C.35.20 | . | . | . | T | . | E | E | . | . | . | . | F | . | . |
| CH3C.35.21 | . | . | . | T | . | E | E | . | . | . | . | F | . | . |
| CH3C.35.22 | . | . | . | T | . | . | E | . | . | . | . | F | . | . |
| CH3C.35.23 | . | . | . | T | . | E | E | . | . | . | . | F | . | . |
| CH3C.35.24 | . | . | . | T | . | E | E | . | . | . | . | F | . | . |
| CH3C.35.21.17 | . | . | . | T | . | E | E | . | . | . | . | F | . | . |
| CH3C.35.N390 | . | . | . | T | . | . | E | . | . | . | . | F | . | . |
| CH3C.35.20.1.1 | | | | S | | E | E | | | | | F | | |
| CH3C.35.23.2.1 | | | | S | | | E | | | | | F | | |
| CH3C.35.23.1.1 | | | | S | | E | E | | | | | F | | |
| CH3C.35.S413 | | | | S | | | E | | | | | F | | |
| CH3C.35.23.3.1 | | | | S | | E | E | | | | | F | | |
| CH3C.35.N390.1 | | | | S | | | E | | | | | F | | |
| CH3C.35.23.6.1 | | | | S | | E | E | | | | | F | | |

Sequences

| SEQ ID | | |
|---|---|---|
| NO: | Sequence | Description |
| 1 | PCPAPELLGGPSVFLFPPKPKDTLMISRTPEVTCVVVDVSHEDPEVKFNWYVD | Wild-type human Fc |
| | GVEVHNAKTKPREEQYNSTYRVVSVLTVLHQDWLNGKEYKCKVSNKALPAPIE | sequence; |
| | KTISKAKGQPREPQVYTLPPSRDELTKNQVSLTCLVKGFYPSDIAVEWESNGQ | amino acids 1-3 |
| | PENNYKTTPPVLDSDGSFFLYSKLTVDKSRWQQGNVFSCSVMHEALHNHYTQK | (PCP) are from a |
| | SLSLSPGK | hinge region |
| 2 | PCPAPELLGGPSVFLFPPKPKDTLMISRTPEVTCVVVDVSHEDPEVKFNWYVD | CH2 domain |
| | GVEVHNAKTKPREEQYNSTYRVVSVLTVLHQDWLNGKEYKCKVSNKALPAPIE | sequence, including |
| | KTISKAK | three amino acids |
| | | (PCP) at the N- |
| | | terminus from the |
| | | hinge region |
| 3 | GQPREPQVYTLPPSRDELTKNQVSLTCLVKGFYPSDIAVEWESNGQPENNYKT | CH3 domain |
| | TPPVLDSDGSFFLYSKLTVDKSRWQQGNVFSCSVMHEALHNHYTQKSLSLSPG | sequence |
| | K | |
| 700 | ASTKGPSVFPLAPSSKSTSGGTAALGCLVKDYFPEPVTVSWNSGALTSGVHTF | CH1 domain |
| | PAVLQSSGLYSLSSVVTVPSSSLGTQTYICNVNHKPSNTKVDKKV | sequence |
| 232 | DKTHTCP | Portion of human |
| | | IgG1 hinge sequence |
| 233 | DKTHTCPPCP | Portion of human |
| | | IgG1 hinge sequence |
| 234 | EPKSCDKTHTCPPCP | Human IgG1 hinge |
| | | amino acid sequence |
| 623 | ASTKGPSVFPLAPSSKSTSGGTAALGCLVKDYFPEPVTVSWNSGALTSGVHTF | IGHG1_P01857 |
| | PAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYKCKVSNKALP | |
| | APIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNH | |
| | YTQKSLSLSPGK | |
| 624 | ASTKGPSVFPLAPCSRSTSESTAALGCLVKDYFPEPVTVSWNSGALTSGVHTF | IGHG2_P01859 |
| | PAVLQSSGLYSLSSVVTVPSSNFGTQTYTCNVDHKPSNTKVDKTVERKCCVEC | |
| | PPCPAPPVAGPSVFLFPPKPKDTLMISRTPEVTCVVVDVSHEDPEVQFNWYVD | |
| | GVEVHNAKTKPREEQFNSTFRVVSVLTVVHQDWLNGKEYKCKVSNKGLPAPIE | |
| | KTISKTKGQPREPQVYTLPPSREEMTKNQVSLTCLVKGFYPSDISVEWESNGQ | |
| | PENNYKTTPPMLDSDGSFFLYSKLTVDKSRWQQGNVFSCSVMHEALHNHYTQK | |
| | SLSLSPGK | |
| 625 | ASTKGPSVFPLAPCSRSTSGGTAALGCLVKDYFPEPVTVSWNSGALTSGVHTF | IGHG3_P01860 |
| | PAVLQSSGLYSLSSVVTVPSSSLGTQTYTCNVNHKPSNTKVDKRVELKTPLGD | |
| | TTHTCPRCPEPKSCDTPPPCPRCPEPKSCDTPPPCPRCPEPKSCDTPPPCPRC | |
| | PAPELLGGPSVFLFPPKPKDTLMISRTPEVTCVVVDVSHEDPEVQFKWYVDGV | |
| | EVHNAKTKPREEQYNSTFRVVSVLTVLHQDWLNGKEYKCKVSNKALPAPIEKT | |
| | ISKTKGQPREPQVYTLPPSREEMTKNQVSLTCLVKGFYPSDIAVEWESSGQPE | |
| | NNYNTTPPMLDSDGSFFLYSKLTVDKSRWQQGNIFSCSVMHEALHNRFTQKSL | |
| | SLSPGK | |
| 626 | ASTKGPSVFPLAPCSRSTSESTAALGCLVKDYFPEPVTVSWNSGALTSGVHTF | IGHG4_P01861 |
| | PAVLQSSGLYSLSSVVTVPSSSLGTKTYTCNVDHKPSNTKVDKRVESKYGPPC | |
| | PSCPAPEFLGGPSVFLFPPKPKDTLMISRTPEVTCVVVDVSQEDPEVQFNWYV | |
| | DGVEVHNAKTKPREEQFNSTYRVVSVLTVLHQDWLNGKEYKCKVSNKGLPSSI | |
| | EKTISKAKGQPREPQVYTLPPSQEEMTKNQVSLTCLVKGFYPSDIAVEWESNG | |
| | QPENNYKTTPPVLDSDGSFFLYSRLTVDKSRWQEGNVFSCSVMHEALHNHYTQ | |
| | KSLSLSLGK | |
| 701 | RTVAAPSVFIFPPSDEQLKSGTASVVCLINNFYPREAKVQWKVDNALQSG | Kappa constant |
| | NSQESVTEQDSKDSTYSLSSTLTLSKADYEKHKVYACEVTHQGLSSPVTK | region sequence |
| | SFNRGEC | |
| 107 | NSVIIVDKNGRLVYLVENPGGYVAYSKAATVTGKLVHANFGTKKDFEDLYTPV | Human TfR apical |
| | NGSIVIVRAGKITFAEKVANAESLNAIGVLIYMDQTKFPIVNAELSFFGHAHL | domain |
| | GTGDPYTPGFPSFNHTQFPPSRSSGLPNIPVQTISRAAAEKLFGNMEGDCPSD | |
| | WKTDSTCRMVTSESKNVKLTVS | |
| 235 | MMDQARSAFSNLFGGEPLSYTRESLARQVDGDNSHVEMKLAVDEEENADNNTK | Human transferrin |
| | ANVTKPKRCSGSICYGTIAVIVFFLIGFMIGYLGYCKGVEPKTECERLAGTES | receptor protein 1 |
| | PVREEPGEDFPAARRLYWDDLKRKLSEKLDSTDFTGTIKLLNENSYVPREAGS | (TFR1) |
| | QKDENLALYVENQFREFKLSKVWRDQHFVKIQVKDSAQNSVIIVDKNGRLVYL | |
| | VENPGGYVAYSKAATVTGKLVHANFGTKKDFEDLYTPVNGSIVIVRAGKITFA | |
| | EKVANAESLNAIGVLIYMDQTKFPIVNAELSFFGHAHLGTGDPYTPGFPSFNH | |
| | TQFPPSRSSGLPNIPVQTISRAAAEKLFGNMEGDCPSDWKTDSTCRMVTSESK | |
| | NVKLTVSNVLKEIKILNIFGVIKGFVEPDHYVVVGAQRDAWGPGAAKSGVGTA | |
| | LLLKLAQMFSDMVLKDGFQPSRSIIFASWSAGDFGSVGATEWLEGYLSSLHLK | |
| | AFTYINLDKAVLGTSNFKVSASPLLYTLIEKTMQNVKHPVTGQFLYQDSNWAS | |
| | KVEKLTLDNAAFPFLAYSGIPAVSFCFCEDTDYPYLGTTMDTYKELIERIPEL | |
| | NKVARAAAEVAGQFVIKLTHDVELNLDYERYNSQLLSFVRDLNQYRADIKEMG | |
| | LSLQWLYSARGDFFRATSRLTTDFGNAEKTDRFVMKKLNDRVMRVEYHFLSPY | |
| | VSPKESPFRHVFWGSGSHTLPALLENLKLRKONNGAFNETLERNQLALATWTI | |
| | QGAANALSGDVWDIDNEF | |
| 4 | PCPAPELLGGPSVFLFPPKPKDTLMISRTPEVTCVVVDVSHEDPEVKFNWYVD | Clone CH3C.1 |
| | GVEVHNAKTKPREEQYNSTYRVVSVLTVLHQDWLNGKEYKCKVSNKALPAPIE | (Clone CH3C.18.4) |
| | KTISKAKGQPREPQVYTLPPSRDELTKNQVSLTCLVKGFYPSDIAVEWESLGL | |
| | VWVGYKTTPPVLDSDGSFFLYSKLTVAKSTWQQGWVFSCSVMHEALHNHYTQK | |
| | SLSLSPGK | |
| 5 | PCPAPELLGGPSVFLFPPKPKDTLMISRTPEVTCVVVDVSHEDPEVKFNWYVD | Clone CH3C.2 |
| | GVEVHNAKTKPREEQYNSTYRVVSVLTVLHQDWLNGKEYKCKVSNKALPAPIE | (Clone CH3C.18.2) |
| | KTISKAKGQPREPQVYTLPPSRDELTKNQVSLTCLVKGFYPSDIAVEWESYGT | |
| | VWSHYKTTPPVLDSDGSFFLYSKLTVSKSEWQQGYVFSCSVMHEALHNHYTQK | |
| | SLSLSPGK | |
| 6 | PCPAPELLGGPSVFLFPPKPKDTLMISRTPEVTCVVVDVSHEDPEVKFNWYVD | Clone CH3C.3 |
| | GVEVHNAKTKPREEQYNSTYRVVSVLTVLHQDWLNGKEYKCKVSNKALPAPIE | (Clone CH3C.18.3) |
| | KTISKAKGQPREPQVYTLPPSRDELTKNQVSLTCLVKGFYPSDIAVEWESYGT | |
| | EWSQYKTTPPVLDSDGSFFLYSKLTVEKSDWQQGHVFSCSVMHEALHNHYTQK | |
| | SLSLSPGK | |
| 7 | PCPAPELLGGPSVFLFPPKPKDTLMISRTPEVTCVVVDVSHEDPEVKFNWYVD | Clone CH3C.4 |
| | GVEVHNAKTKPREEQYNSTYRVVSVLTVLHQDWLNGKEYKCKVSNKALPAPIE | (Clone CH3C.18.1) |
| | KTISKAKGQPREPQVYTLPPSRDELTKNQVSLTCLVKGFYPSDIAVEWESVGT | |
| | PWALYKTTPPVLDSDGSFFLYSKLTVLKSEWQQGWVFSCSVMHEALHNHYTQK | |
| | SLSLSPGK | |
| 8 | PCPAPELLGGPSVFLFPPKPKDTLMISRTPEVTCVVVDVSHEDPEVKFNWYVD | Clone CH3C.17 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | VWSKYKTTPPVLDSDGSFFLYSKLTVSKSEWQQGFVFSCSVMHEALHNHYTQK | |
| | SLSLSPGK | |
| 9 | PCPAPELLGGPSVFLFPPKPKDTLMISRTPEVTCVVVDVSHEDPEVKFNWYVD | Clone CH3C.18 |
| | GVEVHNAKTKPREEQYNSTYRVVSVLTVLHQDWLNGKEYKCKVSNKALPAPIE | (Clone |
| | KTISKAKGQPREPQVYTLPPSRDELTKNQVSLTCLVKGFYPSDIAVEWESLGH | CH3C.18.1.18) |
| | VWAVYKTTPPVLDSDGSFFLYSKLTVPKSTWQQGWVFSCSVMHEALHNHYTQK | |
| | SLSLSPGK | |
| 10 | PCPAPELLGGPSVFLFPPKPKDTLMISRTPEVTCVVVDVSHEDPEVKFNWYVD | Clone CH3C.21 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGL | |
| | VWVGYKTTPPVLDSDGSFFLYSKLTVPKSTWOQGWVFSCSVMHEALHNHYTQK | |
| | SLSLSPGK | |

| | | |
|---|---|---|
| 11 | PCPAPELLGGPSVFLFPPKPKDTLMISRTPEVTCVVVDVSHEDPEVKFNWYVD | Clone CH3C.25 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESMGH | |
| | VWVGYKTTPPVLDSDGSFFLYSKLTVDKSTWQQGWVFSCSVMHEALHNHYTQK | |
| | SLSLSPGK | |
| 12 | PCPAPELLGGPSVFLFPPKPKDTLMISRTPEVTCVVVDVSHEDPEVKFNWYVD | Clone CH3C.34 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGL | |
| | VWVFSKTTPPVLDSDGSFFLYSKLTVPKSTWQQGWVFSCSVMHEALHNHYTQK | |
| | SLSLSPGK | |
| 13 | PCPAPELLGGPSVFLFPPKPKDTLMISRTPEVTCVVVDVSHEDPEVKFNWYVD | Clone CH3C.35 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSSYKTTPPVLDSDGSFFLYSKLTVTKSEWQQGFVFSCSVMHEALHNHYTQK | |
| | SLSLSPGK | |
| 14 | PCPAPELLGGPSVFLFPPKPKDTLMISRTPEVTCVVVDVSHEDPEVKFNWYVD | Clone CH3C.44 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVSKSEWQQGFVFSCSVMHEALHNHYTQK | |
| | SLSLSPGK | |
| 15 | PCPAPELLGGPSVFLFPPKPKDTLMISRTPEVTCVVVDVSHEDPEVKFNWYVD | Clone CH3C.51 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGH | |
| | VWVGYKTTPPVLDSDGSFFLYSKLTVSKSEWQQGWVFSCSVMHEALHNHYTQK | |
| | SLSLSPGK | |
| 16 | PCPAPELLGGPSVFLFPPKPKDTLMISRTPEVTCVVVDVSHEDPEVKFNWYVD | Clone CH3C.3.1-3 |
| | GVEVHNAKTKPREEQYNSTYRVVSVLTVLHQDWLNGKEYKCKVSNKALPAPIE | (Clone CH3C.18.3.1- |
| | KTISKAKGQPREPQVYTLPPSRDELTKNQVSLTCLVKGFYPSDIAVEWESLGH | 3) |
| | VWVATKTTPPVLDSDGSFFLYSKLTVPKSTWQQGWVFSCSVMHEALHNHYTQK | |
| | SLSLSPGK | |
| 17 | PCPAPELLGGPSVFLFPPKPKDTLMISRTPEVTCVVVDVSHEDPEVKFNWYVD | Clone CH3C.3.1-9 |
| | GVEVHNAKTKPREEQYNSTYRVVSVLTVLHQDWLNGKEYKCKVSNKALPAPIE | (Clone CH3C.18.3.1- |
| | KTISKAKGQPREPQVYTLPPSRDELTKNQVSLTCLVKGFYPSDIAVEWESLGP | 9) |
| | VWVHTKTTPPVLDSDGSFFLYSKLTVPKSTWQQGWVFSCSVMHEALHNHYTQK | |
| | SLSLSPGK | |
| 18 | PCPAPELLGGPSVFLFPPKPKDTLMISRTPEVTCVVVDVSHEDPEVKFNWYVD | Clone CH3C.3.2-5 |
| | GVEVHNAKTKPREEQYNSTYRVVSVLTVLHQDWLNGKEYKCKVSNKALPAPIE | (Clone CH3C.18.3.2- |
| | KTISKAKGQPREPQVYTLPPSRDELTKNQVSLTCLVKGFYPSDIAVEWESLGH | 5) |
| | VWVDQKTTPPVLDSDGSFFLYSKLTVPKSTWQQGWVESCSVMHEALHNHYTQK | |
| | SLSLSPGK | |
| 19 | PCPAPELLGGPSVFLFPPKPKDTLMISRTPEVTCVVVDVSHEDPEVKFNWYVD | Clone CH3C.3.2-19 |
| | GVEVHNAKTKPREEQYNSTYRVVSVLTVLHQDWLNGKEYKCKVSNKALPAPIE | (Clone CH3C.18.3.2- |
| | KTISKAKGQPREPQVYTLPPSRDELTKNQVSLTCLVKGFYPSDIAVEWESLGH | 19) |
| | VWVNQKTTPPVLDSDGSFFLYSKLTVPKSTWQQGWVFSCSVMHEALHNHYTQK | |
| | SLSLSPGK | |
| 20 | PCPAPELLGGPSVFLFPPKPKDTLMISRTPEVTCVVVDVSHEDPEVKFNWYVD | Clone CH3C.3.2-1 |
| | GVEVHNAKTKPREEQYNSTYRVVSVLTVLHQDWLNGKEYKCKVSNKALPAPIE | (Clone CH3C.18.3.2- |
| | KTISKAKGQPREPQVYTLPPSRDELTKNQVSLTCLVKGFYPSDIAVEWESLGH | 1) |
| | VWVNFKTTPPVLDSDGSFFLYSKLTVPKSTWQQGWVFSCSVMHEALHNHYTQK | |
| | SLSLSPGK | |
| 21 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.18.E153W |
| | KTISKAKGQPREPQVYTLPPSRDELTKNQVSLTCLVKGFYPSDIAVWWESLGH | (CH3C.35.13) |
| | VWAVYKTTPPVLDSDGSFFLYSKLTVPKSTWQQGWVFSCSVMHEALHNHYTQK | |
| | SLSLSPGK | |
| 22 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.18.K165Q |
| | KTISKAKGQPREPQVYTLPPSRDELTKNQVSLTCLVKGFYPSDIAVEWESLGH | (CH3C.35.14) |
| | VWAVYQTTPPVLDSDGSFFLYSKLTVPKSTWQQGWVFSCSVMHEALHNHYTQK | |
| | SLSLSPGK | |
| 23 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.18.E153W. |
| | KTISKAKGQPREPQVYTLPPSRDELTKNQVSLTCLVKGFYPSDIAVWWESLGH | (CH3C.35.15) |
| | VWAVYQTTPPVLDSDGSFFLYSKLTVPKSTWQQGWVFSCSVMHEALHNHYTQK | |
| | SLSLSPGK | |
| 24 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.E153W |
| | KTISKAKGQPREPQVYTLPPSRDELTKNQVSLTCLVKGFYPSDIAVWWESYGT | K165Q(CH3C.35.19) |
| | EWSSYKTTPPVLDSDGSFFLYSKLTVTKSEWQQGFVFSCSVMHEALHNHYTQK | |
| | SLSLSPGK | |
| 25 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.S188E |
| | KTISKAKGQPREPQVYTLPPSRDELTKNQVSLTCLVKGFYPSDIAVEWESYGT | (CH3C.35.20) |
| | EWSSYKTTPPVLDSDGSFFLYSKLTVTKEEWQQGFVFSCSVMHEALHNHYTQK | |
| | SLSLSPGK | |
| 26 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.E153W. |
| | KTISKAKGQPREPQVYTLPPSRDELTKNQVSLTCLVKGFYPSDIAVWWESYGT | S188E (CH3C.35.21) |
| | EWSSYKTTPPVLDSDGSFFLYSKLTVTKEEWQQGFVFSCSVMHEALHNHYTQK | |
| | SLSLSPGK | |
| 27 | PCPAPELLGGPSVFLFPPKPKDTLMISRTPEVTCVVVDVSHEDPEVKFNWYVD | Clone CH3C.35.N163 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVTKSEWQQGFVFSCSVMHEALHNHYTQK | |
| | SLSLSPGK | |
| 28 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.K165Q |
| | KTISKAKGQPREPQVYTLPPSRDELTKNQVSLTCLVKGFYPSDIAVEWESYGT | |
| | EWSSYQTTPPVLDSDGSFFLYSKLTVTKSEWQQGFVFSCSVMHEALHNHYTQK | |
| | SLSLSPGK | |
| 29 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.N163. |
| | KTISKAKGQPREPQVYTLPPSRDELTKNQVSLTCLVKGFYPSDIAVEWESYGT | K165Q |
| | EWSNYQTTPPVLDSDGSFFLYSKLTVTKSEWQQGFVFSCSVMHEALHNHYTQK | |
| | SLSLSPGK | |
| 236 | PCPAPELLGGPSVFLFPPKPKDTLMISRTPEVTCVVVDVSHEDPEVKFNWYVD | Clone CH3C.35.19 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYKTTPPVLDSDGSFFLYSKLTVTKSEWQQGFVFSCSVMHEALHNHYTQK | |
| | SLSLSPGK | |
| 237 | PCPAPELLGGPSVFLFPPKPKDTLMISRTPEVTCVVVDVSHEDPEVKFNWYVD | Clone CH3C.35.20 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSSYKTTPPVLDSDGSFFLYSKLTVTKEEWQQGFVFSCSVMHEALHNHYTQK | |
| | SLSLSPGK | |
| 238 | PCPAPELLGGPSVFLFPPKPKDTLMISRTPEVTCVVVDVSHEDPEVKFNWYVD | Clone CH3C.35.21 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYKTTPPVLDSDGSFFLYSKLTVTKEEWQQGFVFSCSVMHEALHNHYTQK | |
| | SLSLSPGK | |
| 239 | PCPAPELLGGPSVFLFPPKPKDTLMISRTPEVTCVVVDVSHEDPEVKFNWYVD | Clone CH3C.35.22 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSNYKTTPPVLDSDGSFFLYSKLTVTKSEWQQGFVFSCSVMHEALHNHYTQK | |
| | SLSLSPGK | |
| 240 | PCPAPELLGGPSVFLFPPKPKDTLMISRTPEVTCVVVDVSHEDPEVKFNWYVD | Clone CH3C.35.23 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVTKEEWQQGFVFSCSVMHEALHNHYTQK | |
| | SLSLSPGK | |
| 241 | PCPAPELLGGPSVFLFPPKPKDTLMISRTPEVTCVVVDVSHEDPEVKFNWYVD | Clone CH3C.35.24 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSNYKTTPPVLDSDGSFFLYSKLTVTKEEWQQGFVFSCSVMHEALHNHYTQK | |
| | SLSLSPGK | |
| 242 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESLGH | |
| | VWAVYKTTPPVLDSDGSFFLYSKLTVPKSTWQQGWVFSCSVMHEALHNHYTQK | |
| | SLSLSPGK | |
| 243 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESLGH | |
| | VWAVYKTTPPVLDSDGSFFLYSKLTVPKSTWQQGWVFSCSVMHEALHNHYTQK | |
| | SLSLSPGK | |
| 244 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVYWESLGH | |
| | VWAVYKTTPPVLDSDGSFFLYSKLTVPKSTWQQGWVFSCSVMHEALHNHYTQK | |
| | SLSLSPGK | |
| 245 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGH | |
| | VWAVYQTTPPVLDSDGSFFLYSKLTVPKSTWQQGWVFSCSVMHEALHNHYTQK | |
| | SLSLSPGK | |
| 246 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGH | |
| | VWAVYFTTPPVLDSDGSFFLYSKLTVPKSTWOQGWVFSCSVMHEALHNHYTQK | |
| | SLSLSPGK | |
| 247 | PCPAPELLGGPSVFLFPPKPKDTLMISRTPEVTCVVVDVSHEDPEVKFNWYVD | CH3C.18 variant |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGH | |
| | VWAVYHTTPPVLDSDGSFFLYSKLTVPKSTWQQGWVFSCSVMHEALHNHYTQK | |
| | SLSLSPGK | |
| 248 | PCPAPELLGGPSVFLFPPKPKDTLMISRTPEVTCVVVDVSHEDPEVKFNWYVD | Clone CH3C.35.21.1 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYKTTPPVLDSDGSFFLYSKLTVTKSEWQQGFVFSCSVMHEALHNHYTQK | |
| | SLSLSPGK | |
| 249 | PCPAPELLGGPSVFLFPPKPKDTLMISRTPEVTCVVVDVSHEDPEVKFNWYVD | Clone CH3C.35.21.2 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTKSEWQQGFVFSCSVMHEALHNHYTQK | |
| | SLSLSPGK | |
| 250 | PCPAPELLGGPSVFLFPPKPKDTLMISRTPEVTCVVVDVSHEDPEVKFNWYVD | Clone CH3C.35.21.3 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFSCSVMHEALHNHYTQK | |
| | SLSLSPGK | |
| 251 | PCPAPELLGGPSVFLFPPKPKDTLMISRTPEVTCVVVDVSHEDPEVKFNWYVD | Clone CH3C.35.21.4 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTGEEWQQGFVFSCSVMHEALHNHYTQK | |
| | SLSLSPGK | |
| 252 | PCPAPELLGGPSVFLFPPKPKDTLMISRTPEVTCVVVDVSHEDPEVKFNWYVD | Clone CH3C.35.21.5 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFSCWVMHEALHNHYTQK | |
| | SLSLSPGK | |
| 253 | PCPAPELLGGPSVFLFPPKPKDTLMISRTPEVTCVVVDVSHEDPEVKFNWYVD | Clone CH3C.35.21.6 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTKEEWQQGFVFSCWVMHEALHNHYTQK | |
| | SLSLSPGK | |
| 254 | PCPAPELLGGPSVFLFPPKPKDTLMISRTPEVTCVVVDVSHEDPEVKFNWYVD | Clone CH3C.35.21.7 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFTCWVMHEALHNHYTQK | |
| | SLSLSPGK | |
| 255 | PCPAPELLGGPSVFLFPPKPKDTLMISRTPEVTCVVVDVSHEDPEVKFNWYVD | Clone CH3C.35.21.8 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFTCGVMHEALHNHYTQK | |
| | SLSLSPGK | |
| 256 | PCPAPELLGGPSVFLFPPKPKDTLMISRTPEVTCVVVDVSHEDPEVKFNWYVD | Clone CH3C.35.21.9 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFECWVMHEALHNHYTQK | |
| | SLSLSPGK | |
| 257 | PCPAPELLGGPSVFLFPPKPKDTLMISRTPEVTCVVVDVSHEDPEVKFNWYVD | Clone CH3C.35.21.10 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTREEWQQGFVFKCWVMHEALHNHYTQK | |
| | SLSLSPGK | |
| 258 | PCPAPELLGGPSVFLFPPKPKDTLMISRTPEVTCVVVDVSHEDPEVKFNWYVD | Clone CH3C.35.21.11 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTPEEWQQGFVFKCWVMHEALHNHYTQK | |
| | SLSLSPGK | |
| 259 | PCPAPELLGGPSVFLFPPKPKDTLMISRTPEVTCVVVDVSHEDPEVKFNWYVD | Clone CH3C.35.21.12 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYRTTPPVLDSDGSFFLYSKLTVTREEWQQGFVFSCSVMHEALHNHYTQK | |
| | SLSLSPGK | |
| 260 | PCPAPELLGGPSVFLFPPKPKDTLMISRTPEVTCVVVDVSHEDPEVKFNWYVD | Clone CH3C.35.21.13 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYRTTPPVLDSDGSFFLYSKLTVTGEEWQQGFVFSCSVMHEALHNHYTQK | |
| | SLSLSPGK | |
| 261 | PCPAPELLGGPSVFLFPPKPKDTLMISRTPEVTCVVVDVSHEDPEVKFNWYVD | Clone CH3C.35.21.14 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYRTTPPVLDSDGSFFLYSKLTVTREEWQQGFVFTCWVMHEALHNHYTQK | |
| | SLSLSPGK | |
| 262 | PCPAPELLGGPSVFLFPPKPKDTLMISRTPEVTCVVVDVSHEDPEVKFNWYVD | Clone CH3C.35.21.15 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYRTTPPVLDSDGSFFLYSKLTVTGEEWQQGFVFTCWVMHEALHNHYTQK | |
| | SLSLSPGK | |
| 263 | PCPAPELLGGPSVFLFPPKPKDTLMISRTPEVTCVVVDVSHEDPEVKFNWYVD | Clone CH3C.35.21.16 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSSYRTTPPVLDSDGSFFLYSKLTVTREEWQQGFVFTCGVMHEALHNHYTQK | |
| | SLSLSPGK | |
| 264 | PCPAPELLGGPSVFLFPPKPKDTLMISRTPEVTCVVVDVSHEDPEVKFNWYVD | Clone CH3C.35.21.17 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYKTTPPVLDSDGSFFLYSKLTVTKEEWQQGFVFSCSVMHEALHNHYTQK | |
| | SLSLSPGK | |
| 265 | PCPAPELLGGPSVFLFPPKPKDTLMISRTPEVTCVVVDVSHEDPEVKFNWYVD | Clone CH3C.35.21.18 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVLWESYGT | |
| | EWSSYRTTPPVLDSDGSFFLYSKLTVTKEEWQQGFVFSCSVMHEALHNHYTQK | |
| | SLSLSPGK | |
| 266 | PCPAPELLGGPSVFLFPPKPKDTLMISRTPEVTCVVVDVSHEDPEVKFNWYVD | Clone CH3C.35.20.1 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWSSYKTTPPVLDSDGSFFLYSKLTVTKEEWQQGFVFSCSVMHEALHNHYTQK | |
| | SLSLSPGK | |
| 267 | PCPAPELLGGPSVFLFPPKPKDTLMISRTPEVTCVVVDVSHEDPEVKFNWYVD | Clone CH3C.35.20.2 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWASYKTTPPVLDSDGSFFLYSKLTVTKEEWQQGFVFSCSVMHEALHNHYTQK | |
| | SLSLSPGK | |
| 268 | PCPAPELLGGPSVFLFPPKPKDTLMISRTPEVTCVVVDVSHEDPEVKFNWYVD | Clone CH3C.35.20.3 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWVSYKTTPPVLDSDGSFFLYSKLTVTKEEWQQGFVFSCSVMHEALHNHYTQK | |
| | SLSLSPGK | |
| 269 | PCPAPELLGGPSVFLFPPKPKDTLMISRTPEVTCVVVDVSHEDPEVKFNWYVD | Clone CH3C.35.20.4 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSSYKTTPPVLDSDGSFFLYSKLTVSKEEWQQGFVFSCSVMHEALHNHYTQK | |
| | SLSLSPGK | |
| 270 | PCPAPELLGGPSVFLFPPKPKDTLMISRTPEVTCVVVDVSHEDPEVKFNWYVD | Clone CH3C.35.20.5 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWASYKTTPPVLDSDGSFFLYSKLTVTKEEWQQGFVFSCSVMHEALHNHYTQK | |
| | SLSLSPGK | |
| 271 | PCPAPELLGGPSVFLFPPKPKDTLMISRTPEVTCVVVDVSHEDPEVKFNWYVD | Clone CH3C.35.20.6 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWVSYKTTPPVLDSDGSFFLYSKLTVTKEEWQQGFVFSCSVMHEALHNHYTQK | |
| | SLSLSPGK | |
| 272 | PCPAPELLGGPSVFLFPPKPKDTLMISRTPEVTCVVVDVSHEDPEVKFNWYVD | Clone CH3C35.21.a.1 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESFGT | |
| | EWSSYKTTPPVLDSDGSFFLYSKLTVTKEEWQQGFVFSCSVMHEALHNHYTQK | |
| | SLSLSPGK | |
| 273 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.a.2 |
| | KTISKAKGQPREPQVYTLPPSRDELTKNQVSLTCLVKGFYPSDIAVWWESYGT | |
| | EWASYKTTPPVLDSDGSFFLYSKLTVTKEEWQQGFVFSCSVMHEALHNHYTQK | |
| | SLSLSPGK | |
| 274 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.a.3 |
| | KTISKAKGQPREPQVYTLPPSRDELTKNQVSLTCLVKGFYPSDIAVWWESYGT | |
| | EWVSYKTTPPVLDSDGSFFLYSKLTVTKEEWQQGFVFSCSVMHEALHNHYTQK | |
| | SLSLSPGK | |
| 275 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.a.4 |
| | KTISKAKGQPREPQVYTLPPSRDELTKNQVSLTCLVKGFYPSDIAVWWESYGT | |
| | EWSSYKTTPPVLDSDGSFFLYSKLTVSKEEWQQGFVFSCSVMHEALHNHYTQK | |
| | SLSLSPGK | |
| 276 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.a.5 |
| | KTISKAKGQPREPQVYTLPPSRDELTKNQVSLTCLVKGFYPSDIAVWWESFGT | |
| | EWASYKTTPPVLDSDGSFFLYSKLTVTKEEWQQGFVFSCSVMHEALHNHYTQK | |
| | SLSLSPGK | |
| 277 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.a.6 |
| | KTISKAKGQPREPQVYTLPPSRDELTKNQVSLTCLVKGFYPSDIAVWWESFGT | |
| | EWVSYKTTPPVLDSDGSFFLYSKLTVTKEEWQQGFVFSCSVMHEALHNHYTQK | |
| | SLSLSPGK | |
| 278 | PCPAPELLGGPSVFLFPPKPKDTLMISRTPEVTCVVVDVSHEDPEVKFNWYVD | Clone CH3C.35.23.1 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWSNYKTTPPVLDSDGSFFLYSKLTVTKEEWQQGFVFSCSVMHEALHNHYTQK | |
| | SLSLSPGK | |
| 279 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWANYKTTPPVLDSDGSFFLYSKLTVTKEEWQQGFVFSCSVMHEALHNHYTQK | |
| | SLSLSPGK | |
| 280 | PCPAPELLGGPSVFLFPPKPKDTLMISRTPEVTCVVVDVSHEDPEVKFNWYVD | Clone CH3C.35.23.3 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWVNYKTTPPVLDSDGSFFLYSKLTVTKEEWQQGFVFSCSVMHEALHNHYTQK | |
| | SLSLSPGK | |
| 281 | PCPAPELLGGPSVFLFPPKPKDTLMISRTPEVTCVVVDVSHEDPEVKFNWYVD | Clone CH3C.35.23.4 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVSKEEWQQGFVFSCSVMHEALHNHYTQK | |
| | SLSLSPGK | |
| 282 | PCPAPELLGGPSVFLFPPKPKDTLMISRTPEVTCVVVDVSHEDPEVKFNWYVD | Clone CH3C.35.23.5 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWANYKTTPPVLDSDGSFFLYSKLTVTKEEWQQGFVFSCSVMHEALHNHYTQK | |
| | SLSLSPGK | |
| 283 | PCPAPELLGGPSVFLFPPKPKDTLMISRTPEVTCVVVDVSHEDPEVKFNWYVD | Clone CH3C.35.23.6 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESFGT | |
| | EWVNYKTTPPVLDSDGSFFLYSKLTVTKEEWQQGFVFSCSVMHEALHNHYTQK | |
| | SLSLSPGK | |
| 284 | PCPAPELLGGPSVFLFPPKPKDTLMISRTPEVTCVVVDVSHEDPEVKFNWYVD | Clone CH3C.35.24.1 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESFGT | |
| | EWSNYKTTPPVLDSDGSFFLYSKLTVTKEEWQQGFVFSCSVMHEALHNHYTQK | |
| | SLSLSPGK | |
| 285 | PCPAPELLGGPSVFLFPPKPKDTLMISRTPEVTCVVVDVSHEDPEVKFNWYVD | Clone CH3C.35.24.2 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWANYKTTPPVLDSDGSFFLYSKLTVTKEEWQQGFVFSCSVMHEALHNHYTQK | |
| | SLSLSPGK | |
| 286 | PCPAPELLGGPSVFLFPPKPKDTLMISRTPEVTCVVVDVSHEDPEVKFNWYVD | Clone CH3C.35.24.3 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWVNYKTTPPVLDSDGSFFLYSKLTVTKEEWQQGFVFSCSVMHEALHNHYTQK | |
| | SLSLSPGK | |
| 287 | PCPAPELLGGPSVFLFPPKPKDTLMISRTPEVTCVVVDVSHEDPEVKFNWYVD | Clone CH3C.35.24.4 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESYGT | |
| | EWSNYKTTPPVLDSDGSFFLYSKLTVSKEEWQQGFVFSCSVMHEALHNHYTQK | |
| | SLSLSPGK | |
| 288 | PCPAPELLGGPSVFLFPPKPKDTLMISRTPEVTCVVVDVSHEDPEVKFNWYVD | Clone CH3C.35.24.5 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESFGT | |
| | EWANYKTTPPVLDSDGSFFLYSKLTVTKEEWQQGFVFSCSVMHEALHNHYTQK | |
| | SLSLSPGK | |
| 289 | PCPAPELLGGPSVFLFPPKPKDTLMISRTPEVTCVVVDVSHEDPEVKFNWYVD | Clone CH3C.35.24.6 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESFGT | |
| | EWVNYKTTPPVLDSDGSFFLYSKLTVTKEEWQQGFVFSCSVMHEALHNHYTQK | |
| | SLSLSPGK | |
| 290 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.1 |
| | KTISKAKGQPREPQVYTLPPSRDELTKNQVSLTCLVKGFYPSDIAVLWESFGT | |
| | EWSSYKTTPPVLDSDGSFFLYSKLTVTKEEWQQGFVFSCSVMHEALHNHYTQK | |
| | SLSLSPGK | |
| 291 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLTCLVKGFYPSDIAVLWESYGT | |
| | EWASYKTTPPVLDSDGSFFLYSKLTVTKEEWQQGFVFSCSVMHEALHNHYTQK | |
| | SLSLSPGK | |
| 292 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.3 |
| | KTISKAKGQPREPQVYTLPPSRDELTKNQVSLTCLVKGFYPSDIAVLWESYGT | |
| | EWVSYKTTPPVLDSDGSFFLYSKLTVTKEEWQQGFVFSCSVMHEALHNHYTQK | |
| | SLSLSPGK | |
| 293 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.4 |
| | KTISKAKGQPREPQVYTLPPSRDELTKNQVSLTCLVKGFYPSDIAVLWESYGT | |
| | EWSSYKTTPPVLDSDGSFFLYSKLTVSKEEWQQGFVFSCSVMHEALHNHYTQK | |
| | SLSLSPGK | |
| 294 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.5 |
| | KTISKAKGQPREPQVYTLPPSRDELTKNQVSLTCLVKGFYPSDIAVLWESFGT | |
| | EWASYKTTPPVLDSDGSFFLYSKLTVTKEEWQQGFVFSCSVMHEALHNHYTQK | |
| | SLSLSPGK | |
| 295 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.6 |
| | KTISKAKGQPREPQVYTLPPSRDELTKNQVSLTCLVKGFYPSDIAVLWESFGT | |
| | EWVSYKTTPPVLDSDGSFFLYSKLTVTKEEWQQGFVFSCSVMHEALHNHYTQK | |
| | SLSLSPGK | |
| 296 | PCPAPELLGGPSVFLFPPKPKDTLMISRTPEVTCVVVDVSHEDPEVKFNWYVD | Clone CH3C.35.N390 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVTKSEWQQGFVFSCSVMHEALHNHYTQK | |
| | SLSLSPGK | |
| 297 | PCPAPELLGGPSVFLFPPKPKDTLMISRTPEVTCVVVDVSHEDPEVKFNWYVD | Clone CH3C.35.16 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESLGH | |
| | VWVNQKTTPPVLDSDGSFFLYSKLTVPKSTWOQGWVFSCSVMHEALHNHYTQK | |
| | SLSLSPGK | |
| 298 | PCPAPELLGGPSVFLFPPKPKDTLMISRTPEVTCVVVDVSHEDPEVKFNWYVD | Clone CH3C.35.17 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESLGH | |
| | VWVNQQTTPPVLDSDGSFFLYSKLTVPKSTWQQGWVFSCSVMHEALHNHYTQK | |
| | SLSLSPGK | |
| 299 | PCPAPELLGGPSVFLFPPKPKDTLMISRTPEVTCVVVDVSHEDPEVKFNWYVD | Clone CH3C.35.18 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVWWESLGH | |
| | VWVNQQTTPPVLDSDGSFFLYSKLTVPKSTWQQGWVFSCSVMHEALHNHYTQK | |
| | SLSLSPGK | |
| 302 | METDTLLLWVLLLWVPGSTGDKTHTCPPCPAPEAAGGPSVFLFPPKPKDTLYI | Expressed CH3C.18 |
| | TREPEVTCVVVDVSHEDPEVKFNWYVDGVEVHNAKTKPREEQYNSTYRVVSVL | Fc sequence |
| | TVLHQDWLNGKEYKCKVSNKALGAPIEKTISKAKGQPREPQVYTLPPSRDELT | |
| | KNQVSLTCLVKGFYPSDIAVEWESLGHVWAVYKTTPPVLDSDGSFFLYSKLTV | |
| | PKSTWQQGWVFSCSVMHEALHNHYTQKSLSLSPGK | |
| 421 | METDTLLLWVLLLWVPGSTGDKTHTCPPCPAPEAAGGPSVFLFPPKPKDTLYI | Expressed CH3C.35 |
| | TREPEVTCVVVDVSHEDPEVKFNWYVDGVEVHNAKTKPREEQYNSTYRVVSVL | Fc sequence |
| | TVLHQDWLNGKEYKCKVSNKALGAPIEKTISKAKGQPREPQVYTLPPSRDELT | |
| | KNQVSLTCLVKGFYPSDIAVEWESYGTEWSSYKTTPPVLDSDGSFFLYSKLTV | |
| | TKSEWQQGFVFSCSVMHEALHNHYTQKSLSLSPGK | |
| 422 | PCPAPELLGGPSVFLFPPKPKDTLMISRTPEVTCVVVDVSHEDPEVKFNWYVD | Clone CH3C. 18.3.4-1 |
| | GVEVHNAKTKPREEQYNSTYRVVSVLTVLHQDWLNGKEYKCKVSNKALPAPIE | (CH3C.3.4-1) |
| | KTISKAKGQPREPQVYTLPPSRDELTKNQVSLTCLVKGFYPSDIAVEWESWGF | |
| | VWSTYKTTPPVLDSDGSFFLYSKLTVPKSNWQQGFVFSCSVMHEALHNHYTQK | |
| | SLSLSPGK | |
| 423 | PCPAPELLGGPSVFLFPPKPKDTLMISRTPEVTCVVVDVSHEDPEVKFNWYVD | Clone CH3C.18.3.4- |
| | GVEVHNAKTKPREEQYNSTYRVVSVLTVLHQDWLNGKEYKCKVSNKALPAPIE | 19 |
| | KTISKAKGQPREPQVYTLPPSRDELTKNQVSLTCLVKGFYPSDIAVEWESWGH | (CH3C.3.4-19) |
| | VWSTYKTTPPVLDSDGSFFLYSKLTVPKSNWQQGYVFSCSVMHEALHNHYTQK | |
| | SLSLSPGK | |
| 424 | PCPAPELLGGPSVFLFPPKPKDTLMISRTPEVTCVVVDVSHEDPEVKFNWYVD | Clone CH3C.18.3.2-3 |
| | GVEVHNAKTKPREEQYNSTYRVVSVLTVLHQDWLNGKEYKCKVSNKALPAPIE | (CH3C.3.2-3) |
| | KTISKAKGQPREPQVYTLPPSRDELTKNQVSLTCLVKGFYPSDIAVEWESLGH | |
| | VWVEQKTTPPVLDSDGSFFLYSKLTVPKSTWQQGWVFSCSVMHEALHNHYTQK | |
| | SLSLSPGK | |
| 425 | PCPAPELLGGPSVFLFPPKPKDTLMISRTPEVTCVVVDVSHEDPEVKFNWYVD | Clone CH3C.18.3.2- |
| | GVEVHNAKTKPREEQYNSTYRVVSVLTVLHQDWLNGKEYKCKVSNKALPAPIE | 14 |
| | KTISKAKGQPREPQVYTLPPSRDELTKNQVSLTCLVKGFYPSDIAVEWESLGH | (CH3C.3.2-14) |
| | VWVGVKTTPPVLDSDGSFFLYSKLTVPKSTWOQGWVFSCSVMHEALHNHYTQK | |
| | SLSLSPGK | |
| 426 | PCPAPELLGGPSVFLFPPKPKDTLMISRTPEVTCVVVDVSHEDPEVKFNWYVD | Clone CH3C.18.3.2- |
| | GVEVHNAKTKPREEQYNSTYRVVSVLTVLHQDWLNGKEYKCKVSNKALPAPIE | 24 |
| | KTISKAKGQPREPQVYTLPPSRDELTKNQVSLTCLVKGFYPSDIAVEWESLGH | (CH3C.3.2-24) |
| | VWVHTKTTPPVLDSDGSFFLYSKLTVPKSTWQQGWVFSCSVMHEALHNHYTQK | |
| | SLSLSPGK | |
| 427 | PCPAPELLGGPSVFLFPPKPKDTLMISRTPEVTCVVVDVSHEDPEVKFNWYVD | Clone CH3C.18.3.4- |
| | GVEVHNAKTKPREEQYNSTYRVVSVLTVLHQDWLNGKEYKCKVSNKALPAPIE | 26 |
| | KTISKAKGQPREPQVYTLPPSRDELTKNQVSLTCLVKGFYPSDIAVEWESWGT | (CH3C.3.4-26) |
| | VWGTYKTTPPVLDSDGSFFLYSKLTVPKSNWQQGYVFSCSVMHEALHNHYTQK | |
| | SLSLSPGK | |
| 428 | PCPAPELLGGPSVFLFPPKPKDTLMISRTPEVTCVVVDVSHEDPEVKFNWYVD | Clone CH3C.18.3.2- |
| | GVEVHNAKTKPREEQYNSTYRVVSVLTVLHQDWLNGKEYKCKVSNKALPAPIE | 17 |
| | KTISKAKGQPREPQVYTLPPSRDELTKNQVSLTCLVKGFYPSDIAVEWESLGH | (CH3C.3.2-17) |
| | VWVGTKTTPPVLDSDGSFFLYSKLTVPKSTWQQGWVFSCSVMHEALHNHYTQK | |
| | SLSLSPGK | |
| 429 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.20.1.1 |
| | KTISKAKGQPREPQVYTLPPSRDELTKNQVSLTCLVKGFYPSDIAVEWESFGT | |
| | EWSSYKTTPPVLDSDGSFFLYSKLTVSKEEWQQGFVFSCSVMHEALHNHYTQK | |
| | SLSLSPGK | |
| 430 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.23.2.1 |
| | KTISKAKGQPREPQVYTLPPSRDELTKNQVSLTCLVKGFYPSDIAVEWESYGT | |
| | EWANYKTTPPVLDSDGSFFLYSKLTVSKSEWQQGFVFSCSVMHEALHNHYTQK | |
| | SLSLSPGK | |
| 431 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.23.1.1 |
| | KTISKAKGQPREPQVYTLPPSRDELTKNQVSLTCLVKGFYPSDIAVEWESFGT | |
| | EWSNYKTTPPVLDSDGSFFLYSKLTVSKEEWQQGFVFSCSVMHEALHNHYTQK | |
| | SLSLSPGK | |
| 432 | PCPAPELLGGPSVFLFPPKPKDTLMISRTPEVTCVVVDVSHEDPEVKFNWYVD | Clone CH3C.35.S413 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSSYKTTPPVLDSDGSFFLYSKLTVSKSEWQQGFVFSCSVMHEALHNHYTQK | |
| | SLSLSPGK | |
| 433 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.23.3.1 |
| | KTISKAKGQPREPQVYTLPPSRDELTKNQVSLTCLVKGFYPSDIAVEWESYGT | |
| | EWVNYKTTPPVLDSDGSFFLYSKLTVSKEEWQQGFVFSCSVMHEALHNHYTQK | |
| | SLSLSPGK | |
| 434 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.N390.1 |
| | KTISKAKGQPREPQVYTLPPSRDELTKNQVSLTCLVKGFYPSDIAVEWESYGT | |
| | EWSNYKTTPPVLDSDGSFFLYSKLTVSKSEWQQGFVFSCSVMHEALHNHYTQK | |
| | SLSLSPGK | |
| 435 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.23.6.1 |
| | KTISKAKGQPREPQVYTLPPSRDELTKNQVSLTCLVKGFYPSDIAVEWESFGT | |
| | EWVNYKTTPPVLDSDGSFFLYSKLTVSKEEWQQGFVFSCSVMHEALHNHYTQK | |
| | SLSLSPGK | |
| 554 | X₁WESX₂GX₃X₄WX₅X₆ | CH3C.35_consensus |
| | X₁ is E, L, S, V, W, or Y; X₂ is an aromatic amino | sequence_1 |
| | acid (e.g., Y, F, or W), M, P, or V; X₃ is T, N, or V; | |
| | X₄ is E, I, P, or V; X₅ is an aliphatic amino acid | |
| | (e.g., A, I, or V), S, or T; and X₆ is S, N, R, or T | |
| 555 | X₁KX₂X₃WQQGX₄VFX₅CX₆ | CH3C.35_consensus |
| | X₁ is T, H, or S; X₂ is E, S, D, G, T, P, Q, or R; | sequence_2 |
| | X₃ is E or R; X₄ is F, H, K, Y, or W; X₅ is S, T, or W; | |
| | and X₆ is S, C, P, M, or W | |
| 556 | GQPREPQVYTLPPSRDELTKNQVSLTCLVKGFYPSDIAVX₁WESX₂GX₃X₄WX₅ | CH3C.35_consensus |
| | X₆YKTTPPVLDSDGSFFLYSKLTVX₇KX₈X₉WQQGX₁₀VFX₁₁CX₁₂VMHEALHNH | sequence_3 |
| | YTQKSLSLSPGK | |
| | X₁ is E, L, S, V, W, or Y; X₂ is an aromatic amino | |
| | acid (e.g., Y, F, or W), M, P, or V; X₃ is T, N, or | |
| | V; X₄ is E, I, P, or V; X₅ is an aliphatic amino acid | |
| | (e.g., A, I, or V), S, or T; X₆ is S, N, R, or T; | |
| | X₇ is T, H, or S; X₈ is E, S, D, G, T, P, Q, or R; | |
| | X₉ is E or R; X₁₀ is F, H, K, Y, or W; X₁₁ is S, T, or | |
| | W; and X₁₂ is S, C, P, M, or W | |
| 361 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob mutation |
| | KTISKAKGQPREPQVYTLPPSRDELTKNQVSLWCLVKGFYPSDIAVEWESYGT | |
| | EWANYKTTPPVLDSDGSFFLYSKLTVTKEEWQQGFVFSCSVMHEALHNHYTQK | |
| | SLSLSPGK | |
| 362 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWANYKTTPPVLDSDGSFFLYSKLTVTKEEWQQGFVFSCSVMHEALHNHYTQK | |
| | SLSLSPGK | |
| 363 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESYGT | LALAPG mutations |
| | EWANYKTTPPVLDSDGSFFLYSKLTVTKEEWQQGFVFSCSVMHEALHNHYTQK | |
| | SLSLSPGK | |
| 632 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALSAPIE | with knob and |
| | KTISKAKGQPREPQVYTLPPSRDELTKNQVSLWCLVKGFYPSDIAVEWESYGT | LALAPS mutations |
| | EWANYKTTPPVLDSDGSFFLYSKLTVTKEEWQQGFVFSCSVMHEALHNHYTQK | |
| | SLSLSPGK | |
| 364 | PCPAPELLGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob and YTE |
| | KTISKAKGQPREPQVYTLPPSRDELTKNQVSLWCLVKGFYPSDIAVEWESYGT | mutations |
| | EWANYKTTPPVLDSDGSFFLYSKLTVTKEEWQQGFVFSCSVMHEALHNHYTQK | |
| | SLSLSPGK | |
| 365 | PCPAPEAAGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and YTE mutations |
| | EWANYKTTPPVLDSDGSFFLYSKLTVTKEEWQQGFVFSCSVMHEALHNHYTQK | |
| | SLSLSPGK | |
| 366 | PCPAPEAAGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and YTE mutations |
| | EWANYKTTPPVLDSDGSFFLYSKLTVTKEEWQQGFVFSCSVMHEALHNHYTQK | |
| | SLSLSPGK | |
| 367 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole mutations |
| | KTISKAKGQPREPQVYTLPPSRDELTKNQVSLSCAVKGFYPSDIAVEWESYGT | |
| | EWANYKTTPPVLDSDGSFFLVSKLTVTKEEWQQGFVFSCSVMHEALHNHYTQK | |
| | SLSLSPGK | |
| 368 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole and LALA |
| | KTISKAKGQPREPQVYTLPPSRDELTKNQVSLSCAVKGFYPSDIAVEWESYGT | mutations |
| | EWANYKTTPPVLDSDGSFFLVSKLTVTKEEWQQGFVFSCSVMHEALHNHYTQK | |
| | SLSLSPGK | |
| 369 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with hole and |
| | KTISKAKGQPREPQVYTLPPSRDELTKNQVSLSCAVKGFYPSDIAVEWESYGT | LALAPG mutations |
| | EWANYKTTPPVLDSDGSFFLVSKLTVTKEEWQQGFVFSCSVMHEALHNHYTQK | |
| | SLSLSPGK | |
| 370 | PCPAPELLGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole and YTE |
| | KTISKAKGQPREPQVYTLPPSRDELTKNQVSLSCAVKGFYPSDIAVEWESYGT | mutations |
| | EWANYKTTPPVLDSDGSFFLVSKLTVTKEEWQQGFVFSCSVMHEALHNHYTQK | |
| | SLSLSPGK | |
| 371 | PCPAPEAAGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | YTE mutations |
| | EWANYKTTPPVLDSDGSFFLVSKLTVTKEEWQQGFVFSCSVMHEALHNHYTQK | |
| | SLSLSPGK | |
| 372 | PCPAPEAAGGPSVFLFPPKPKDTLYITREPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and YTE mutations |
| | EWANYKTTPPVLDSDGSFFLVSKLTVTKEEWQQGFVFSCSVMHEALHNHYTQK | |
| | SLSLSPGK | |
| 491 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with M201L and |
| | KTISKAKGQPREPQVYTLPPSRDELTKNQVSLTCLVKGFYPSDIAVEWESYGT | N207S mutations |
| | EWANYKTTPPVLDSDGSFFLYSKLTVTKEEWQQGFVFSCSVLHEALHSHYTQK | |
| | SLSLSPGK | |
| 492 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob and |
| | KTISKAKGQPREPQVYTLPPSRDELTKNQVSLWCLVKGFYPSDIAVEWESYGT | M201L and N207S |
| | EWANYKTTPPVLDSDGSFFLYSKLTVTKEEWQQGFVFSCSVLHEALHSHYTQK | mutations |
| | SLSLSPGK | |
| 493 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and M201L and |
| | EWANYKTTPPVLDSDGSFFLYSKLTVTKEEWQQGFVESCSVLHEALHSHYTQK | N207S mutations |
| | SLSLSPGK | |
| 494 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and M201L and |
| | EWANYKTTPPVLDSDGSFFLYSKLTVTKEEWQQGFVFSCSVLHEALHSHYTQK | N207S mutations |
| | SLSLSPGK | |
| 495 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole and M201L |
| | KTISKAKGQPREPQVYTLPPSRDELTKNQVSLSCAVKGFYPSDIAVEWESYGT | and N207S mutations |
| | EWANYKTTPPVLDSDGSFFLVSKLTVTKEEWQQGFVFSCSVLHEALHSHYTQK | |
| | SLSLSPGK | |
| 496 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | M201L and N207S |
| | EWANYKTTPPVLDSDGSFFLVSKLTVTKEEWQQGFVFSCSVLHEALHSHYTQK | mutations |
| | SLSLSPGK | |
| 497 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and M201L and |
| | EWANYKTTPPVLDSDGSFFLVSKLTVTKEEWQQGFVFSCSVLHEALHSHYTQK | N207S mutations |
| | SLSLSPGK | |
| 702 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and S239C mutations |
| | EWANYKTTPPVLDSDGSFFLYSKLTVTKEEWQQGFVFSCSVMHEALHNHYTQK | |
| | SLSLSPGK | |
| 703 | PCPAPEAAGGPSVFLFPPKPKDTLMISRTPEVTCVVVDVSHEDPEVKFNWYVD | Clone CH3C.35.23.2 |
| | GVEVHNAKCKPREEQYNSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and T289C mutations |
| | EWANYKTTPPVLDSDGSFFLYSKLTVTKEEWQQGFVFSCSVMHEALHNHYTQK | |
| | SLSLSPGK | |
| 704 | PCPAPEAAGGPSVFLFPPKPKDTLMISRTPEVTCVVVDVSHEDPEVKFNWYVD | Clone CH3C.35.23.2 |
| | GVEVHNAKTKPREEQYASTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and N297A mutations |
| | EWANYKTTPPVLDSDGSFFLYSKLTVTKEEWQQGFVFSCSVMHEALHNHYTQK | |
| | SLSLSPGK | |
| 705 | PCPAPEAAGGPSVFLFPPKPKDTLMISRTPEVTCVVVDVSHEDPEVKFNWYVD | Clone CH3C.35.23.2 |
| | GVEVHNAKTKPREEQYGSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and N297G mutations |
| | EWANYKTTPPVLDSDGSFFLYSKLTVTKEEWQQGFVFSCSVMHEALHNHYTQK | |
| | SLSLSPGK | |
| 706 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPCPIE | with knob, LALA, |
| | KTISKAKGQPREPQVYTLPPSRDELTKNQVSLWCLVKGFYPSDIAVEWESYGT | and A330C mutations |
| | EWANYKTTPPVLDSDGSFFLYSKLTVTKEEWQQGFVFSCSVMHEALHNHYTQK | |
| | SLSLSPGK | |
| 707 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | and S442C mutations |
| | KTISKAKGQPREPQVYTLPPSRDELTKNQVSLWCLVKGFYPSDIAVEWESYGT | with knob, LALA, |
| | EWANYKTTPPVLDSDGSFFLYSKLTVTKEEWQQGFVFSCSVMHEALHNHYTQK | |
| | SLCLSPGK | |
| 708 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPCPIE | with knob, LALA, |
| | KTISKAKGQPREPQVYTLPPSRDELTKNQVSLWCLVKGFYPSDIAVEWESYGT | S239C, A330C |
| | EWANYKTTPPVLDSDGSFFLYSKLTVTKEEWQQGFVFSCSVMHEALHNHYTQK | mutations |
| | SLSLSPGK | |
| 709 | CSTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | 35.23.2, knob, LALA, |
| | WYVDGVEVHNAKTKPREEQYNSTYRVVSVLTVLHQDWLNGKEYKCKVSNKALP | and A114C mutations |
| | APIEKTISKAKGQPREPQVYTLPPSRDELTKNQVSLWCLVKGFYPSDIAVEWE | |
| | SYGTEWANYKTTPPVLDSDGSFFLYSKLTVTKEEWQQGFVFSCSVMHEALHNH | |
| | YTQKSLSLSPGK | |
| 710 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and S239C mutations |
| | EWANYKTTPPVLDSDGSFFLYSKLTVTKEEWQQGFVFSCSVMHEALHNHYTQK | |
| | SLSLSPGK | |
| 711 | PCPAPEAAGGPSVFLFPPKPKDTLMISRTPEVTCVVVDVSHEDPEVKFNWYVD | Clone CH3C.35.23.2 |
| | GVEVHNAKCKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and T289C mutations |
| | EWANYKTTPPVLDSDGSFFLYSKLTVTKEEWQQGFVFSCSVMHEALHNHYTQK | |
| | SLSLSPGK | |
| 712 | PCPAPEAAGGPSVFLFPPKPKDTLMISRTPEVTCVVVDVSHEDPEVKFNWYVD | Clone CH3C.35.23.2 |
| | GVEVHNAKTKPREEQYASTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and N297A mutations |
| | EWANYKTTPPVLDSDGSFFLYSKLTVTKEEWQQGFVFSCSVMHEALHNHYTQK | |
| | SLSLSPGK | |
| 713 | PCPAPEAAGGPSVFLFPPKPKDTLMISRTPEVTCVVVDVSHEDPEVKFNWYVD | Clone CH3C.35.23.2 |
| | GVEVHNAKTKPREEQYGSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and N297G mutations |
| | EWANYKTTPPVLDSDGSFFLYSKLTVTKEEWQQGFVFSCSVMHEALHNHYTQK | |
| | SLSLSPGK | |
| 714 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGCPIE | with knob, LALAPG, |
| | KTISKAKGQPREPQVYTLPPSRDELTKNQVSLWCLVKGFYPSDIAVEWESYGT | and A330C mutations |
| | EWANYKTTPPVLDSDGSFFLYSKLTVTKEEWQQGFVFSCSVMHEALHNHYTQK | |
| | SLSLSPGK | |
| 715 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and S442C mutations |
| | EWANYKTTPPVLDSDGSFFLYSKLTVTKEEWQQGFVFSCSVMHEALHNHYTQK | |
| | SLCLSPGK | |
| 716 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGCPIE | with knob, LALAPG, |
| | KTISKAKGQPREPQVYTLPPSRDELTKNQVSLWCLVKGFYPSDIAVEWESYGT | S239C, A330C |
| | EWANYKTTPPVLDSDGSFFLYSKLTVTKEEWQQGFVFSCSVMHEALHNHYTQK | mutations |
| | SLSLSPGK | |
| 717 | CSTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | 35.23.2, knob, |
| | WYVDGVEVHNAKTKPREEQYNSTYRVVSVLTVLHQDWLNGKEYKCKVSNKALG | LALAPG, and |
| | APIEKTISKAKGQPREPQVYTLPPSRDELTKNQVSLWCLVKGFYPSDIAVEWE | A114C mutations |
| | SYGTEWANYKTTPPVLDSDGSFFLYSKLTVTKEEWQQGFVFSCSVMHEALHNH | |
| | YTQKSLSLSPGK | |
| 743 | ASTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | 35.23.2, knob, and |
| | WYVDGVEVHNAKTKPREEQYNSTYRVVSVLTVLHQDWLNGKEYKCKVSNKALP | LALA, mutations |
| | APIEKTISKAKGQPREPQVYTLPPSRDELTKNQVSLWCLVKGFYPSDIAVEWE | |
| | SYGTEWANYKTTPPVLDSDGSFFLYSKLTVTKEEWQQGFVFSCSVMHEALHNH | |
| | YTQKSLSLSPGK | |
| 744 | ASTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | 35.23.2, knob, and |
| | WYVDGVEVHNAKTKPREEQYNSTYRVVSVLTVLHQDWLNGKEYKCKVSNKALG | LALAPG, mutations |
| | APIEKTISKAKGQPREPQVYTLPPSRDELTKNQVSLWCLVKGFYPSDIAVEWE | |
| | SYGTEWANYKTTPPVLDSDGSFFLYSKLTVTKEEWQQGFVFSCSVMHEALHNH | |
| | YTQKSLSLSPGK | |
| 718 | PCPAPEAAGGPSVFLFPPKPKDTLMISRTPEVTCVVVDVSHEDPEVKFNWYVD | Clone CH3C.35.23.4 |
| | GVEVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWSNYKTTPPVLDSDGSFFLYSKLTVSKEEWQQGFVFSCSVMHEALHNHYTQK | |
| | SLSLSPGK | |
| 397 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob mutation |
| | EWASYKTTPPVLDSDGSFFLYSKLTVTKEEWQQGFVFSCSVMHEALHNHYTQK | |
| | SLSLSPGK | |
| 398 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob and LALA |
| | EWASYKTTPPVLDSDGSFFLYSKLTVTKEEWQQGFVFSCSVMHEALHNHYTQK | mutations |
| | SLSLSPGK | |
| 399 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLWCLVKGFYPSDIAVLWESYGT | with knob and |
| | EWASYKTTPPVLDSDGSFFLYSKLTVTKEEWQQGFVFSCSVMHEALHNHYTQK | LALAPG mutations |
| | SLSLSPGK | |
| 633 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALSAPIE | CH3C.35.21.17.2 |
| | KTISKAKGQPREPQVYTLPPSRDELTKNQVSLWCLVKGFYPSDIAVLWESYGT | with knob and |
| | EWASYKTTPPVLDSDGSFFLYSKLTVTKEEWQQGFVFSCSVMHEALHNHYTQK | LALAPS mutations |
| | SLSLSPGK | |
| 400 | PCPAPELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob and YTE |
| | EWASYKTTPPVLDSDGSFFLYSKLTVTKEEWQQGFVFSCSVMHEALHNHYTQK | mutations |
| | SLSLSPGK | |
| 401 | PCPAPEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob, LALA, |
| | EWASYKTTPPVLDSDGSFFLYSKLTVTKEEWQQGFVFSCSVMHEALHNHYTQK | and YTE mutations |
| | SLSLSPGK | |
| 402 | PCPAPEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLWCLVKGFYPSDIAVLWESYGT | with knob, LALAPG, |
| | EWASYKTTPPVLDSDGSFFLYSKLTVTKEEWQQGFVFSCSVMHEALHNHYTQK | and YTE mutations |
| | SLSLSPGK | |
| 403 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole mutations |
| | EWASYKTTPPVLDSDGSFFLVSKLTVTKEEWQQGFVFSCSVMHEALHNHYTQK | |
| | SLSLSPGK | |
| 404 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole and LALA |
| | EWASYKTTPPVLDSDGSFFLVSKLTVTKEEWQQGFVFSCSVMHEALHNHYTQK | mutations |
| | SLSLSPGK | |
| 405 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLSCAVKGFYPSDIAVLWESYGT | with hole and |
| | EWASYKTTPPVLDSDGSFFLVSKLTVTKEEWQQGFVFSCSVMHEALHNHYTQK | LALAPG mutations |
| | SLSLSPGK | |
| 406 | PCPAPELLGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole and YTE |
| | EWASYKTTPPVLDSDGSFFLVSKLTVTKEEWQQGFVFSCSVMHEALHNHYTQK | mutations |
| | SLSLSPGK | |
| 407 | PCPAPEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole, LALA, and |
| | EWASYKTTPPVLDSDGSFFLVSKLTVTKEEWQQGFVFSCSVMHEALHNHYTQK | YTE mutations |
| | SLSLSPGK | |
| 408 | PCPAPEAAGGPSVFLFPPKPKDTLYITREPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLSCAVKGFYPSDIAVLWESYGT | with hole, LALAPG, |
| | EWASYKTTPPVLDSDGSFFLVSKLTVTKEEWQQGFVFSCSVMHEALHNHYTQK | and YTE mutations |
| | SLSLSPGK | |
| 512 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLTCLVKGFYPSDIAVLWESYGT | with M201L and |
| | EWASYKTTPPVLDSDGSFFLYSKLTVTKEEWQQGFVFSCSVLHEALHSHYTQK | N207S mutations |
| | SLSLSPGK | |
| 513 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob and |
| | EWASYKTTPPVLDSDGSFFLYSKLTVTKEEWQQGFVFSCSVLHEALHSHYTQK | M201L and N207S |
| | SLSLSPGK | mutations |
| 514 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob, LALA, |
| | EWASYKTTPPVLDSDGSFFLYSKLTVTKEEWQQGFVFSCSVLHEALHSHYTQK | and M201L and |
| | SLSLSPGK | N207S mutations |
| 515 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLWCLVKGFYPSDIAVLWESYGT | with knob, LALAPG, |
| | EWASYKTTPPVLDSDGSFFLYSKLTVTKEEWQQGFVFSCSVLHEALHSHYTQK | and M201L and |
| | SLSLSPGK | N207S mutations |
| 516 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole and M201L |
| | EWASYKTTPPVLDSDGSFFLVSKLTVTKEEWQQGFVFSCSVLHEALHSHYTQK | and N207S mutations |
| | SLSLSPGK | |
| 517 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLSCAVKGFYPSDIAVLWESYGT | with hole, LALA, and |
| | EWASYKTTPPVLDSDGSFFLVSKLTVTKEEWQQGFVESCSVLHEALHSHYTQK | M201L and N207S |
| | SLSLSPGK | mutations |
| 518 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLSCAVKGFYPSDIAVLWESYGT | with hole, LALAPG, |
| | EWASYKTTPPVLDSDGSFFLVSKLTVTKEEWQQGFVFSCSVLHEALHSHYTQK | and M201L and |
| | SLSLSPGK | N207S mutations |
| 655 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPAPIE | knob and LALA |
| | KTISKAKGQPREPQVYTLPPSRDELTKNQVSLWCLVKGFYPSDIAVEWESNGQ | mutations |
| | PENNYKTTPPVLDSDGSFFLYSKLTVDKSRWQQGNVFSCSVMHEALHNHYTQK | |
| | SLSLSPGK | |
| 557 | PCPAPELLGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALPAPIE | mutations |
| | KTISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQK | |
| | SLSLSPGK | |
| 558 | PCPAPEAAGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALPAPIE | and LALA mutations |
| | KTISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQK | |
| | SLSLSPGK | |
| 559 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPAPIE | hole, LALA, and |
| | KTISKAKGQPREPQVYTLPPSRDELTKNQVSLSCAVKGFYPSDIAVEWESNGQ | S239C mutations |
| | PENNYKTTPPVLDSDGSFFLVSKLTVDKSRWQQGNVFSCSVMHEALHNHYTQK | |
| | SLSLSPGK | |
| 560 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPCPIE | hole, LALA and |
| | KTISKAKGQPREPQVYTLPPSRDELTKNQVSLSCAVKGFYPSDIAVEWESNGQ | A330C mutations |
| | PENNYKTTPPVLDSDGSFFLVSKLTVDKSRWQQGNVFSCSVMHEALHNHYTQK | |
| | SLSLSPGK | |
| 561 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPAPIE | hole, LALA and |
| | KTISKAKGQPREPQVYTLPPSRDELTKNQVSLSCAVKGFYPSDIAVEWESNGQ | S442C mutations |
| | PENNYKTTPPVLDSDGSFFLVSKLTVDKSRWQQGNVFSCSVMHEALHNHYTQK | |
| | SLCLSPGK | |
| 719 | PCPAPEAAGGPSVFLFPPKPKDTLMISRTPEVTCVVVDVSHEDPEVKFNWYVD | Fc sequence with |
| | GVEVHNAKCKPREEQYNSTYRVVSVLTVLHQDWLNGKEYKCKVSNKALPAPIE | hole, LALA, and |
| | KTISKAKGQPREPQVYTLPPSRDELTKNQVSLSCAVKGFYPSDIAVEWESNGQ | T289C mutations |
| | PENNYKTTPPVLDSDGSFFLVSKLTVDKSRWQQGNVFSCSVMHEALHNHYTQK | |
| | SLSLSPGK | |
| 720 | PCPAPEAAGGPSVFLFPPKPKDTLMISRTPEVTCVVVDVSHEDPEVKFNWYVD | Fc sequence with |
| | GVEVHNAKTKPREEQYASTYRVVSVLTVLHQDWLNGKEYKCKVSNKALPAPIE | hole, LALA, and |
| | KTISKAKGQPREPQVYTLPPSRDELTKNQVSLSCAVKGFYPSDIAVEWESNGQ | N297A mutations |
| | PENNYKTTPPVLDSDGSFFLVSKLTVDKSRWQQGNVFSCSVMHEALHNHYTQK | |
| | SLSLSPGK | |
| 721 | PCPAPEAAGGPSVFLFPPKPKDTLMISRTPEVTCVVVDVSHEDPEVKFNWYVD | Fc sequence with |
| | GVEVHNAKTKPREEQYGSTYRVVSVLTVLHQDWLNGKEYKCKVSNKALPAPIE | hole, LALA, and |
| | KTISKAKGQPREPQVYTLPPSRDELTKNQVSLSCAVKGFYPSDIAVEWESNGQ | N297G mutations |
| | PENNYKTTPPVLDSDGSFFLVSKLTVDKSRWQQGNVFSCSVMHEALHNHYTQK | |
| | SLSLSPGK | |
| 722 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPCPIE | hole, LALA, S239C, |
| | KTISKAKGQPREPQVYTLPPSRDELTKNQVSLSCAVKGFYPSDIAVEWESNGQ | and A330C mutations |
| | PENNYKTTPPVLDSDGSFFLVSKLTVDKSRWQQGNVFSCSVMHEALHNHYTQK | |
| | SLSLSPGK | |
| 723 | CSTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | hole, LALA, and |
| | WYVDGVEVHNAKTKPREEQYNSTYRVVSVLTVLHQDWLNGKEYKCKVSNKALP | A114C mutations |
| | APIEKTISKAKGQPREPQVYTLPPSRDELTKNQVSLSCAVKGFYPSDIAVEWE | |
| | SNGQPENNYKTTPPVLDSDGSFFLVSKLTVDKSRWQQGNVFSCSVMHEALHNH | |
| | YTQKSLSLSPGK | |
| 627 | PCPAPEAAGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALGAPIE | and LALAPG |
| | KTISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQK | |
| | SLSLSPGK | |
| 628 | PCPAPEAAGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALSAPIE | and LALAPS |
| | KTISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQK | |
| | SLSLSPGK | |
| 629 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSAPIE | hole, LALAPS, and |
| | KTISKAKGQPREPQVYTLPPSRDELTKNQVSLSCAVKGFYPSDIAVEWESNGQ | S239C mutations |
| | PENNYKTTPPVLDSDGSFFLVSKLTVDKSRWQQGNVFSCSVMHEALHNHYTQK | |
| | SLSLSPGK | |
| 630 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSCPIE | hole, LALAPS and |
| | KTISKAKGQPREPQVYTLPPSRDELTKNQVSLSCAVKGFYPSDIAVEWESNGQ | A330C mutations |
| | PENNYKTTPPVLDSDGSFFLVSKLTVDKSRWQQGNVFSCSVMHEALHNHYTQK | |
| | SLSLSPGK | |
| 631 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSAPIE | hole, LALAPS and |
| | KTISKAKGQPREPQVYTLPPSRDELTKNQVSLSCAVKGFYPSDIAVEWESNGQ | S442C mutations |
| | PENNYKTTPPVLDSDGSFFLVSKLTVDKSRWQQGNVFSCSVMHEALHNHYTQK | |
| | SLCLSPGK | |
| 724 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGAPIE | hole, LALAPG, and |
| | KTISKAKGQPREPQVYTLPPSRDELTKNQVSLSCAVKGFYPSDIAVEWESNGQ | S239C mutations |
| | PENNYKTTPPVLDSDGSFFLVSKLTVDKSRWQQGNVFSCSVMHEALHNHYTQK | |
| | SLSLSPGK | |
| 725 | PCPAPEAAGGPSVFLFPPKPKDTLMISRTPEVTCVVVDVSHEDPEVKFNWYVD | Fc sequence with hole |
| | GVEVHNAKCKPREEQYNSTYRVVSVLTVLHQDWLNGKEYKCKVSNKALGAPIE | and LALAPG, and |
| | KTISKAKGQPREPQVYTLPPSRDELTKNQVSLSCAVKGFYPSDIAVEWESNGQ | T289C mutations |
| | PENNYKTTPPVLDSDGSFFLVSKLTVDKSRWQQGNVFSCSVMHEALHNHYTQK | |
| | SLSLSPGK | |
| 726 | PCPAPEAAGGPSVFLFPPKPKDTLMISRTPEVTCVVVDVSHEDPEVKFNWYVD | Fc sequence with hole |
| | GVEVHNAKTKPREEQYGSTYRVVSVLTVLHQDWLNGKEYKCKVSNKALGAPIE | and LALAPG, and |
| | KTISKAKGQPREPQVYTLPPSRDELTKNQVSLSCAVKGFYPSDIAVEWESNGQ | N297G mutations |
| | PENNYKTTPPVLDSDGSFFLVSKLTVDKSRWQQGNVFSCSVMHEALHNHYTQK | |
| | SLSLSPGK | |
| 727 | PCPAPEAAGGPSVFLFPPKPKDTLMISRTPEVTCVVVDVSHEDPEVKFNWYVD | Fc sequence with hole |
| | GVEVHNAKTKPREEQYASTYRVVSVLTVLHQDWLNGKEYKCKVSNKALGAPIE | and LALAPG, and |
| | KTISKAKGQPREPQVYTLPPSRDELTKNQVSLSCAVKGFYPSDIAVEWESNGQ | N297A mutations |
| | PENNYKTTPPVLDSDGSFFLVSKLTVDKSRWQQGNVFSCSVMHEALHNHYTQK | |
| | SLSLSPGK | |
| 728 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGCPIE | hole, LALAPG, and |
| | KTISKAKGQPREPQVYTLPPSRDELTKNQVSLSCAVKGFYPSDIAVEWESNGQ | A330C mutations |
| | PENNYKTTPPVLDSDGSFFLVSKLTVDKSRWQQGNVFSCSVMHEALHNHYTQK | |
| | SLSLSPGK | |
| 729 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGAPIE | hole, LALAPG, and |
| | KTISKAKGQPREPQVYTLPPSRDELTKNQVSLSCAVKGFYPSDIAVEWESNGQ | S442C mutations |
| | PENNYKTTPPVLDSDGSFFLVSKLTVDKSRWQQGNVFSCSVMHEALHNHYTQK | |
| | SLCLSPGK | |
| 730 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALGCPIE | hole, LALAPG, |
| | KTISKAKGQPREPQVYTLPPSRDELTKNQVSLSCAVKGFYPSDIAVEWESNGQ | S239C, and A330C |
| | PENNYKTTPPVLDSDGSFFLVSKLTVDKSRWQQGNVFSCSVMHEALHNHYTQK | mutations |
| | SLSLSPGK | |
| 731 | CSTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | hole, LALAPG, and |
| | WYVDGVEVHNAKTKPREEQYNSTYRVVSVLTVLHQDWLNGKEYKCKVSNKALG | A114C mutations |
| | APIEKTISKAKGQPREPQVYTLPPSRDELTKNQVSLSCAVKGFYPSDIAVEWE | |
| | SNGQPENNYKTTPPVLDSDGSFFLVSKLTVDKSRWQQGNVFSCSVMHEALHNH | |
| | YTQKSLSLSPGK | |
| 732 | RTVAAPSVFIFPPSDEQLKSGTASVVCLLNNFYPREAKVQWCVDNALQSGNSQ | Kappa constant |
| | ESVTEQDSKDSTYSLSSTLTLSKADYEKHKVYACEVTHQGLSSPVTKSFNRGE | region sequence with |
| | C | K149C mutation |
| 800 | DIQMTQSPSTLSASVGDRVTITCKCQLSVGYMHWYQQKPGKAPKLLIYDTSKL | RSV Light Chain |
| | ASGVPSRESGSGSGTEFTLTISSLQPDDFATYYCFQGSGYPFTFGGGTKLEIK | |
| | RTVAAPSVFIFPPSDEQLKSGTASVVCLLNNFYPREAKVQWKVDNALQSGNSQ | |
| | ESVTEQDSKDSTYSLSSTLTLSKADYEKHKVYACEVTHQGLSSPVTKSFNRGE | |
| | C | |
| 801 | QVTLRESGPALVKPTQTLTLTCTFSGFSLSTSGMSVGWIRQPPGKALEWLADI | RSV Heavy Chain: |
| | WWDDKKDYNPSLKSRLTISKDTSKNQVVLKVINMEPADTATYYCARSMITNWY | 35.23.2 with knob |
| | FDVWGAGTTVTVSSASTKGPSVFPLAPSSKSTSGGTAALGCLVKDYFPEPVTV | and LALA mutations |
| | SWNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLWCL | |
| | VKGFYPSDIAVEWESYGTEWANYKTTPPVLDSDGSFFLYSKLTVTKEEWQQGF | |
| | VFSCSVMHEALHNHYTQKSLSLSPGK | |
| 802 | QVTLRESGPALVKPTQTLTLTCTFSGFSLSTSGMSVGWIRQPPGKALEWLADI | RSV Heavy Chain: |
| | WWDDKKDYNPSLKSRLTISKDTSKNQVVLKVTNMEPADTATYYCARSMITNWY | 35.23.2 with knob, |
| | FDVWGAGTTVTVSSASTKGPSVFPLAPSSKSTSGGTAALGCLVKDYFPEPVTV | LALA and S239C |
| | SWNSGALTSGVHTFPAVLQSSGLYSLSSVVTVPSSSLGTQTYICNVNHKPSNT | mutations |
| | KVDKKVEPKSCDKTHTCPPCPAPEAAGGPCVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLWCL | |
| | VKGFYPSDIAVEWESYGTEWANYKTTPPVLDSDGSFFLYSKLTVTKEEWQQGF | |
| | VFSCSVMHEALHNHYTQKSLSLSPGK | |
| 803 | QVTLRESGPALVKPTQTLTLTCTESGFSLSTSGMSVGWIRQPPGKALEWLADI | RSV Heavy Chain: |
| | WWDDKKDYNPSLKSRLTISKDTSKNQVVLKVINMEPADTATYYCARSMITNWY | 35.23.2 with knob, |
| | FDVWGAGTTVTVSSASTKGPSVFPLAPSSKSTSGGTAALGCLVKDYFPEPVTV | LALA, S239C, and |
| | SWNSGALTSGVHTFPAVLQSSGLYSLSSVVTVPSSSLGTQTYICNVNHKPSNT | A330C mutations |
| | KVDKKVEPKSCDKTHTCPPCPAPEAAGGPCVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPCPIEKTISKAKGQPREPQVYTLPPSRDELTKNQVSLWCL | |
| | VKGFYPSDIAVEWESYGTEWANYKTTPPVLDSDGSFFLYSKLTVTKEEWQQGF | |
| | VFSCSVMHEALHNHYTQKSLSLSPGK | |
| 804 | DKTHTCPPCPAPEAAGGPSVFLFPPKPKDTLMISRTPEVTCVVVDVSHEDPEV | Clone 35.23.2 with |
| | KFNWYVDGVEVHNAKTKPREEQYNSTYRVVSVLTVLHQDWLNGKEYKCKVSNK | portion of human |
| | ALPAPIEKTISKAKGQPREPQVYTLPPSRDELTKNQVSLWCLVKGFYPSDIAV | IgGI hinge sequence |
| | EWESYGTEWANYKTTPPVLDSDGSFFLYSKLTVTKEEWQQGFVFSCSVMHEAL | and knob and LALA |
| | HNHYTQKSLSLSPGK | mutations |
| 805 | QVTLRESGPALVKPTQTLTLTCTFSGFSLSTSGMSVGWIRQPPGKALEWLADI | RSV Heavy Chain: |
| | WWDDKKDYNPSLKSRLTISKDTSKNQVVLKVTNMEPADTATYYCARSMITNWY | 35.23.4 with knob, |
| | FDVWGAGTTVTVSSASTKGPSVFPLAPSSKSTSGGTAALGCLVKDYFPEPVTV | and LALA mutations |
| | SWNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVSKEEWQQGF | |
| | VFSCSVMHEALHNHYTQKSLSLSPGK | |
| 806 | QVTLRESGPALVKPTQTLTLTCTESGFSLSTSGMSVGWIRQPPGKALEWLADI | RSV Heavy chain: Fc |
| | WWDDKKDYNPSLKSRLTISKDTSKNQVVLKVINMEPADTATYYCARSMITNWY | with hole, LALA, and |
| | FDVWGAGTTVTVSSASTKGPSVFPLAPSSKSTSGGTAALGCLVKDYFPEPVTV | S239C mutations |
| | SWNSGALTSGVHTFPAVLQSSGLYSLSSVVTVPSSSLGTQTYICNVNHKPSNT | |
| | KVDKKVEPKSCDKTHTCPPCPAPEAAGGPCVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQGN | |
| | VFSCSVMHEALHNHYTQKSLSLSPGK | |
| 807 | QVTLRESGPALVKPTQTLTLTCTFSGFSLSTSGMSVGWIRQPPGKALEWLADI | RSV Heavy chain: Fc |
| | WWDDKKDYNPSLKSRLTISKDTSKNQVVLKVTNMEPADTATYYCARSMITNWY | with hole, LALA, |
| | FDVWGAGTTVTVSSASTKGPSVFPLAPSSKSTSGGTAALGCLVKDYFPEPVTV | S239C, and A330C |
| | SWNSGALTSGVHTFPAVLQSSGLYSLSSVVTVPSSSLGTQTYICNVNHKPSNT | mutations |
| | KVDKKVEPKSCDKTHTCPPCPAPEAAGGPCVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPCPIEKTISKAKGQPREPQVYTLPPSRDELTKNQVSLSCA | |
| | VKGFYPSDIAVEWESNGQPENNYKTTPPVLDSDGSFFLVSKLTVDKSRWQQGN | |
| | VFSCSVMHEALHNHYTQKSLSLSPGK | |
| 808 | QVTLRESGPALVKPTQTLTLTCTFSGFSLSTSGMSVGWIRQPPGKALEWLADI | RSV Heavy chain: Fc |
| | WWDDKKDYNPSLKSRLTISKDTSKNQVVLKVTNMEPADTATYYCARSMITNWY | with hole, LALA, and |
| | FDVWGAGTTVTVSSASTKGPSVFPLAPSSKSTSGGTAALGCLVKDYFPEPVTV | A330C mutations |
| | SWNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPCPIEKTISKAKGQPREPQVYTLPPSRDELTKNQVSLSCA | |
| | VKGFYPSDIAVEWESNGQPENNYKTTPPVLDSDGSFFLVSKLTVDKSRWQQGN | |
| | VFSCSVMHEALHNHYTQKSLSLSPGK | |
| 809 | QVTLRESGPALVKPTQTLTLTCTFSGFSLSTSGMSVGWIRQPPGKALEWLADI | RSV Heavy chain: Fc |
| | WWDDKKDYNPSLKSRLTISKDTSKNQVVLKVTNMEPADTATYYCARSMITNWY | with hole, LALA, and |
| | FDVWGAGTTVTVSSASTKGPSVFPLAPSSKSTSGGTAALGCLVKDYFPEPVTV | S442C mutations |
| | SWNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTCV | |
| | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQGN | |
| | VFSCSVMHEALHNHYTQKSLCLSPGK | |
| 810 | KFNWYVDGVEVHNAKTKPREEQYNSTYRVVSVLTVLHQDWLNGKEYKCKVSNK | Fc with portion of |
| | ALPAPIEKTISKAKGQPREPQVYTLPPSRDELTKNQVSLSCAVKGFYPSDIAV | human IgG1 hinge |
| | EWESNGQPENNYKTTPPVLDSDGSFFLVSKLTVDKSRWQQGNVFSCSVMHEAL | sequence and hole, |
| | HNHYTQKSLSLSPGK | LALA, and S239C |
| | | mutations |
| 811 | EIVMTQSPATLSLSPGERATLSCSASSSVSYMYWYQQKPGQAPRLLIYDTSNL | DNP02 Light Chain |
| | ASGIPARFSGSGSGTDFTLTISSLQPEDFAVYYCQQWSSYPPITFGQGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| 812 | EIVMTQSPATLSLSPGERATLSCSASSSVSYMYWYQQKPGQAPRLLIYDTSNL | DNP02 Light Chain |
| | ASGIPARFSGSGSGTDFTLTISSLQPEDFAVYYCQQWSSYPPITFGQGTKVEI | with K149C mutation |
| | KRTVAAPSVFIFPPSDEQLKSGTASVVCLLNNFYPREAKVQWCVDNALQSGNS | |
| | QESVTEQDSKDSTYSLSSTLTLSKADYEKHKVYACEVTHQGLSSPVTKSFNRG | |
| | EC | |
| 813 | QVQLQESGPGLVKPSETLSLTCAVSGYSITSDYAWNWIRQPPGKGLEWIGYMS | DNP02 Heavy Chain |
| | YSGSTRYNPSLRSRVTISVDTSKNQFSLKLSSVTAADTAVYYCARGWPLAYWG | 35.23.2 with knob, |
| | QGTLVTVSSASTKGPSVFPLAPSSKSTSGGTAALGCLVKDYFPEPVTVSWNSG | and LALAPG |
| | ALTSGVHTFPAVLQSSGLYSLSSVVTVPSSSLGTQTYICNVNHKPSNTKVDKK | mutations |
| | VEPKSCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALGAPIEKTISKAKGQPREPQVYTLPPSRDELTKNQVSLWCLVKGFY | |
| | PSDIAVEWESYGTEWANYKTTPPVLDSDGSFFLYSKLTVTKEEWQQGFVFSCS | |
| | VMHEALHNHYTQKSLSLSPGK | |
| 814 | QVQLQESGPGLVKPSETLSLTCAVSGYSITSDYAWNWIRQPPGKGLEWIGYMS | DNP02 Heavy Chain: |
| | YSGSTRYNPSLRSRVTISVDTSKNQFSLKLSSVTAADTAVYYCARGWPLAYWG | 35.23.2 with knob, |
| | QGTLVTVSSASTKGPSVFPLAPSSKSTSGGTAALGCLVKDYFPEPVTVSWNSG | LALAPG,and |
| | ALTSGVHTFPAVLQSSGLYSLSSVVTVPSSSLGTQTYICNVNHKPSNTKVDKK | N297G mutations |
| | VEPKSCDKTHTCPPCPAPEAAGGPSVFLFPPKPKDTLMISRTPEVTCVVVDVS | |
| | HEDPEVKFNWYVDGVEVHNAKTKPREEQYGSTYRVVSVLTVLHQDWLNGKEYK | |
| | CKVSNKALGAPIEKTISKAKGQPREPQVYTLPPSRDELTKNQVSLWCLVKGFY | |
| | PSDIAVEWESYGTEWANYKTTPPVLDSDGSFFLYSKLTVTKEEWQQGFVESCS | |
| | VMHEALHNHYTQKSLSLSPGK | |
| 815 | QVQLQESGPGLVKPSETLSLTCAVSGYSITSDYAWNWIRQPPGKGLEWIGYMS | DNP02 Heavy Chain: |
| | YSGSTRYNPSLRSRVTISVDTSKNQFSLKLSSVTAADTAVYYCARGWPLAYWG | Fc with hole, and |
| | QGTLVTVSSASTKGPSVFPLAPSSKSTSGGTAALGCLVKDYFPEPVTVSWNSG | LALAPG, mutations |
| | ALTSGVHTFPAVLQSSGLYSLSSVVTVPSSSLGTQTYICNVNHKPSNTKVDKK | |
| | VEPKSCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALGAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGNVFSCS | |
| | VMHEALHNHYTQKSLSLSPGK | |
| 816 | QVQLQESGPGLVKPSETLSLTCAVSGYSITSDYAWNWIRQPPGKGLEWIGYMS | DNP02 Heavy Chain: |
| | YSGSTRYNPSLRSRVTISVDTSKNQFSLKLSSVTAADTAVYYCARGWPLAYWG | Fc with hole, |
| | QGTLVTVSSASTKGPSVFPLAPSSKSTSGGTAALGCLVKDYFPEPVTVSWNSG | LALAPG, and |
| | ALTSGVHTFPAVLQSSGLYSLSSVVTVPSSSLGTQTYICNVNHKPSNTKVDKK | N297G mutations |
| | VEPKSCDKTHTCPPCPAPEAAGGPSVFLFPPKPKDTLMISRTPEVTCVVVDVS | |
| | HEDPEVKFNWYVDGVEVHNAKTKPREEQYGSTYRVVSVLTVLHQDWLNGKEYK | |
| | CKVSNKALGAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGNVESCS | |
| | VMHEALHNHYTQKSLSLSPGK | |
| 817 | QVQLQESGPGLVKPSETLSLTCAVSGYSITSDYAWNWIROPPGKGLEWIGYMS | DNP02 Heavy Chain: |
| | YSGSTRYNPSLRSRVTISVDTSKNQFSLKLSSVTAADTAVYYCARGWPLAYWG | Fc with hole, |
| | QGTLVTVSSCSTKGPSVFPLAPSSKSTSGGTAALGCLVKDYFPEPVTVSWNSG | LALAPG, and |
| | ALTSGVHTFPAVLQSSGLYSLSSVVTVPSSSLGTQTYICNVNHKPSNTKVDKK | A114C mutations |
| | VEPKSCDKTHTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQDWINGKEYK | |
| | CKVSNKALGAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGNVESCS | |
| | VMHEALHNHYTQKSLSLSPGK | |
| 818 | QVQLQESGPGLVKPSETLSLTCAVSGYSITSDYAWNWIRQPPGKGLEWIGYMS | DNP02 Heavy Chain: |
| | YSGSTRYNPSLRSRVTISVDTSKNQFSLKLSSVTAADTAVYYCARGWPLAYWG | Fc with hole, |
| | QGTLVTVSSASTKGPSVFPLAPSSKSTSGGTAALGCLVKDYFPEPVTVSWNSG | LALAPG, and T289C |
| | ALTSGVHTFPAVLQSSGLYSLSSVVTVPSSSLGTQTYICNVNHKPSNTKVDKK | mutations |
| | VEPKSCDKTHTCPPCPAPEAAGGPSVFLFPPKPKDTLMISRTPEVTCVVVDVS | |
| | HEDPEVKFNWYVDGVEVHNAKCKPREEQYNSTYRVVSVLTVLHQDWLNGKEYK | |
| | CKVSNKALGAPIEKTISKAKGQPREPQVYTLPPSRDELTKNQVSLSCAVKGFY | |
| | PSDIAVEWESNGQPENNYKTTPPVLDSDGSFFLVSKLTVDKSRWQQGNVFSCS | |
| | VMHEALHNHYTQKSLSLSPGK | |
| 634 | PCPAPELLGGPSVFLFPPKPKDTLMISRTPEVTCVVVDVSHEDPEVKFNWYVD | Wild-type human Fc |
| | GVEVHNAKTKPREEQYNSTYRVVSVLTVLHQDWLNGKEYKCKVSNKALPAPIE | sequence |
| | KTISKAKGQPREPQVYTLPPSRDELTKNQVSLTCLVKGFYPSDIAVEWESNGQ | amino acids 1-3 |
| | PENNYKTTPPVLDSDGSFFLYSKLTVDKSRWQQGNVFSCSVMHEALHNHYTQK | (PCP) are from a |
| | SLSLSPG | hinge region |
| 635 | PCPAPELLGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALPAPIE | mutations |
| | KTISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQK | |
| | SLSLSPG | |
| 636 | PCPAPEAAGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALPAPIE | and LALA mutations |
| | KTISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQK | |
| | SLSLSPG | |
| 637 | PCPAPEAAGGPSVFLFPPKPKDTLMISRTPEVTCVVVDVSHEDPEVKFNWYVD | Fc |
| | GVEVHNAKTKPREEQYNSTYRVVSVLTVLHQDWLNGKEYKCKVSNKALGAPIE | sequence with hole |
| | KTISKAKGQPREPQVYTLPPSRDELTKNQVSLSCAVKGFYPSDIAVEWESNGQ | and LALAPG |
| | PENNYKTTPPVLDSDGSFFLVSKLTVDKSRWQQGNVFSCSVMHEALHNHYTQK | mutations |
| | SLSLSPG | |
| 638 | PCPAPEAAGGPSVFLFPPKPKDTLMISRTPEVTCVVVDVSHEDPEVKFNWYVD | Fc sequence with hole |
| | GVEVHNAKTKPREEQYNSTYRVVSVLTVLHQDWLNGKEYKCKVSNKALSAPIE | and LALAPS |
| | KTISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQK | |
| | SLSLSPG | |
| 639 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPAPIE | hole, LALA, and |
| | KTISKAKGQPREPQVYTLPPSRDELTKNQVSLSCAVKGFYPSDIAVEWESNGQ | S239C mutations |
| | PENNYKTTPPVLDSDGSFFLVSKLTVDKSRWQQGNVFSCSVMHEALHNHYTQK | |
| | SLSLSPG | |
| 640 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPCPIE | hole, LALA and |
| | KTISKAKGQPREPQVYTLPPSRDELTKNQVSLSCAVKGFYPSDIAVEWESNGQ | A330C mutations |
| | PENNYKTTPPVLDSDGSFFLVSKLTVDKSRWQQGNVFSCSVMHEALHNHYTQK | |
| | SLSLSPG | |
| 641 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPAPIE | hole, LALA and |
| | KTISKAKGQPREPQVYTLPPSRDELTKNQVSLSCAVKGFYPSDIAVEWESNGQ | S442C mutations |
| | PENNYKTTPPVLDSDGSFFLVSKLTVDKSRWQQGNVFSCSVMHEALHNHYTQK | |
| | SLCLSPG | |
| 642 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSAPIE | hole, LALAPS, and |
| | KTISKAKGQPREPQVYTLPPSRDELTKNQVSLSCAVKGFYPSDIAVEWESNGQ | S239C mutations |
| | PENNYKTTPPVLDSDGSFFLVSKLTVDKSRWQQGNVFSCSVMHEALHNHYTQK | |
| | SLSLSPG | |
| 643 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSCPIE | hole, LALAPS and |
| | KTISKAKGQPREPQVYTLPPSRDELTKNQVSLSCAVKGFYPSDIAVEWESNGQ | A330C mutations |
| | PENNYKTTPPVLDSDGSFFLVSKLTVDKSRWQQGNVFSCSVMHEALHNHYTQK | |
| | SLSLSPG | |
| 644 | PCPAPEAAGGPSVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALSAPIE | hole, LALAPS and |
| | KTISKAKGQPREPQVYTLPPSRDELTKNQVSLSCAVKGFYPSDIAVEWESNGQ | S442C mutations |
| | PENNYKTTPPVLDSDGSFFLVSKLTVDKSRWQQGNVFSCSVMHEALHNHYTQK | |
| | SLCLSPG | |
| 733 | PCPAPEAAGGPCVFLFPPKPKDTLMISRTPEVTCVVVDVSHEDPEVKFNWYVD | Fc sequence with |
| | GVEVHNAKTKPREEQYNSTYRVVSVLTVLHQDWLNGKEYKCKVSNKALPCPIE | hole, LALA, S239C, |
| | KTISKAKGQPREPQVYTLPPSRDELTKNQVSLSCAVKGFYPSDIAVEWESNGQ | and A330C mutations |
| | PENNYKTTPPVLDSDGSFFLVSKLTVDKSRWQQGNVFSCSVMHEALHNHYTQK | |
| | SLSLSPG | |
| 734 | PCPAPEAAGGPSVFLFPPKPKDTLMISRTPEVTCVVVDVSHEDPEVKFNWYVD | Fc sequence with hole |
| | GVEVHNAKCKPREEQYNSTYRVVSVLTVLHQDWLNGKEYKCKVSNKALGAPIE | and LALAPG, and |
| | KTISKAKGQPREPQVYTLPPSRDELTKNQVSLSCAVKGFYPSDIAVEWESNGQ | T289C mutations |
| | PENNYKTTPPVLDSDGSFFLVSKLTVDKSRWQQGNVFSCSVMHEALHNHYTQK | |
| | SLSLSPG | |
| 735 | PCPAPEAAGGPSVFLFPPKPKDTLMISRTPEVTCVVVDVSHEDPEVKFNWYVD | Fc sequence with hole |
| | GVEVHNAKTKPREEQYGSTYRVVSVLTVLHQDWLNGKEYKCKVSNKALGAPIE | and LALAPG, and |
| | KTISKAKGQPREPQVYTLPPSRDELTKNQVSLSCAVKGFYPSDIAVEWESNGQ | N297G mutations |
| | PENNYKTTPPVLDSDGSFFLVSKLTVDKSRWQQGNVFSCSVMHEALHNHYTQK | |
| | SLSLSPG | |
| 736 | DKTHTCPPCPAPEAAGGPCVFLFPPKPKDTLMISRTPEVTCVVVDVSHEDPEV | Fc with portion of |
| | KFNWYVDGVEVHNAKTKPREEQYNSTYRVVSVLTVLHQDWLNGKEYKCKVSNK | human IgG1 hinge |
| | ALPAPIEKTISKAKGQPREPQVYTLPPSRDELTKNQVSLSCAVKGFYPSDIAV | sequence and hole, |
| | EWESNGQPENNYKTTPPVLDSDGSFFLVSKLTVDKSRWQQGNVFSCSVMHEAL | LALA, and S239C |
| | HNHYTQKSLSLSPG | mutations |
| 737 | CSTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | hole, LALAPG, and |
| | WYVDGVEVHNAKTKPREEQYNSTYRVVSVLTVLHQDWLNGKEYKCKVSNKALG | A114C mutations |
| | APIEKTISKAKGQPREPQVYTLPPSRDELTKNQVSLSCAVKGFYPSDIAVEWE | |
| | SNGQPENNYKTTPPVLDSDGSFFLVSKLTVDKSRWQQGNVFSCSVMHEALHNH | |
| | YTQKSLSLSPG | |
| 645 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWANYKTTPPVLDSDGSFFLYSKLTVTKEEWQQGFVFSCSVMHEALHNHYTQK | |
| | SLSLSPG | |
| 646 | PCPAPELLGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob mutation |
| | KTISKAKGQPREPQVYTLPPSRDELTKNQVSLWCLVKGFYPSDIAVEWESYGT | |
| | EWANYKTTPPVLDSDGSFFLYSKLTVTKEEWQQGFVFSCSVMHEALHNHYTQK | |
| | SLSLSPG | |
| 647 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWANYKTTPPVLDSDGSFFLYSKLTVTKEEWQQGFVFSCSVMHEALHNHYTQK | |
| | SLSLSPG | |
| 648 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESYGT | LALAPG mutations |
| | EWANYKTTPPVLDSDGSFFLYSKLTVTKEEWQQGFVFSCSVMHEALHNHYTQK | |
| | SLSLSPG | |
| 649 | PCPAPEAAGGPSVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALSAPIE | with knob and |
| | KTISKAKGQPREPQVYTLPPSRDELTKNQVSLWCLVKGFYPSDIAVEWESYGT | LALAPS mutations |
| | EWANYKTTPPVLDSDGSFFLYSKLTVTKEEWQQGFVFSCSVMHEALHNHYTQK | |
| | SLSLSPG | |
| 738 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPAPIE | with knob, LALA, |
| | KTISKAKGQPREPQVYTLPPSRDELTKNQVSLWCLVKGFYPSDIAVEWESYGT | and S239C mutations |
| | EWANYKTTPPVLDSDGSFFLYSKLTVTKEEWQQGFVFSCSVMHEALHNHYTQK | |
| | SLSLSPG | |
| 739 | PCPAPEAAGGPCVFLFPPKPKDTLMISRTPEVTCVVVDVSHEDPEVKFNWYVD | Clone CH3C.35.23.2 |
| | GVEVHNAKTKPREEQYNSTYRVVSVLTVLHQDWLNGKEYKCKVSNKALPCPIE | with knob, LALA, |
| | KTISKAKGQPREPQVYTLPPSRDELTKNQVSLWCLVKGFYPSDIAVEWESYGT | S239C, A330C |
| | EWANYKTTPPVLDSDGSFFLYSKLTVTKEEWQQGFVFSCSVMHEALHNHYTQK | mutations |
| | SLSLSPG | |
| 740 | PCPAPEAAGGPSVFLFPPKPKDTLMISRTPEVTCVVVDVSHEDPEVKFNWYVD | Clone CH3C.35.23.2 |
| | GVEVHNAKTKPREEQYGSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and N297G mutations |
| | EWANYKTTPPVLDSDGSFFLYSKLTVTKEEWQQGFVFSCSVMHEALHNHYTQK | |
| | SLSLSPG | |
| 741 | DKTHTCPPCPAPEAAGGPSVFLFPPKPKDTLMISRTPEVTCVVVDVSHEDPEV | Clone 35.23.2 with |
| | KFNWYVDGVEVHNAKTKPREEQYNSTYRVVSVLTVLHQDWLNGKEYKCKVSNK | portion of human |
| | ALPAPIEKTISKAKGQPREPQVYTLPPSRDELTKNQVSLWCLVKGFYPSDIAV | IgG1 hinge sequence |
| | EWESYGTEWANYKTTPPVLDSDGSFFLYSKLTVTKEEWQQGFVFSCSVMHEAL | and knob and LALA |
| | HNHYTQKSLSLSPG | mutations |
| 745 | ASTKGPSVFPLAPSSKSTSGGTAALGCLVKDYFPEPVTVSWNSGALTSGVHTF | Heavy constant |
| | PAVLQSSGLYSLSSVVTVPSSSLGTQTYICNVNHKPSNTKVDKKVEPKSCDKT | region sequence with |
| | HTCPPCPAPEAAGGPSVFLFPPKPKDTLMISRTPEVTCVVVDVSHEDPEVKFN | 35.23.2, knob, and |
| | WYVDGVEVHNAKTKPREEQYNSTYRVVSVLTVLHQDWLNGKEYKCKVSNKALG | LALAPG, mutations |
| | APIEKTISKAKGQPREPQVYTLPPSRDELTKNQVSLWCLVKGFYPSDIAVEWE | |
| | SYGTEWANYKTTPPVLDSDGSFFLYSKLTVTKEEWQQGFVFSCSVMHEALHNH | |
| | YTQKSLSLSPG | |
| 746 | PCPAPELLGGPSVFLFPPKPKDTLMISRTPEVTCVVVDVSHEDPEVKFNWYVD | Clone CH3C.35.23.4 |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESYGT | |
| | EWSNYKTTPPVLDSDGSFFLYSKLTVSKEEWQQGFVFSCSVMHEALHNHYTQK | |
| | SLSLSPG | |
| 742 | PCPAPEAAGGPSVFLFPPKPKDTLMISRTPEVTCVVVDVSHEDPEVKFNWYVD | Clone CH3C.35.23.4 |
| | GVEVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWSNYKTTPPVLDSDGSFFLYSKLTVSKEEWQQGFVFSCSVMHEALHNHYTQK | |
| | SLSLSPG | |
| 650 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLTCLVKGFYPSDIAVLWESYGT | |
| | EWASYKTTPPVLDSDGSFFLYSKLTVTKEEWQQGFVFSCSVMHEALHNHYTQK | |
| | SLSLSPG | |
| 651 | PCPAPELLGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob mutation |
| | EWASYKTTPPVLDSDGSFFLYSKLTVTKEEWQQGFVFSCSVMHEALHNHYTQK | |
| | SLSLSPG | |
| 652 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALPAPIE | CH3C.35.21.17.2 |
| | KTISKAKGQPREPQVYTLPPSRDELTKNQVSLWCLVKGFYPSDIAVLWESYGT | with knob and LALA |
| | EWASYKTTPPVLDSDGSFFLYSKLTVTKEEWQQGFVFSCSVMHEALHNHYTQK | mutations |
| | SLSLSPG | |
| 653 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALGAPIE | CH3C.35.21.17.2 |
| | KTISKAKGQPREPQVYTLPPSRDELTKNQVSLWCLVKGFYPSDIAVLWESYGT | with knob and |
| | EWASYKTTPPVLDSDGSFFLYSKLTVTKEEWQQGFVFSCSVMHEALHNHYTQK | LALAPG mutations |
| | SLSLSPG | |
| 654 | PCPAPEAAGGPSVFLFPPKPKDTLMISRTPEVTCVVVDVSHEDPEVKFNWYVD | Clone |
| | GVEVHNAKTKPREEQYNSTYRVVSVLTVLHQDWLNGKEYKCKVSNKALSAPIE | CH3C.35.21.17.2 |
| | KTISKAKGQPREPQVYTLPPSRDELTKNQVSLWCLVKGFYPSDIAVLWESYGT | with knob and |
| | EWASYKTTPPVLDSDGSFFLYSKLTVTKEEWQQGFVFSCSVMHEALHNHYTQK | LALAPS mutations |
| | SLSLSPG | |
| 656 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | Exemplary ASO1 |
| 657 | L'-Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | Exemplary ASO1 |
| | | linked to “L′” |
| 819 | SASSSVSMYWY | NBVR1 CDR-L1 |
| 820 | RASQSVSSYLAWY | NBVR2 CDR-L1 |
| 821 | DTSNLAS | NBVR1 CDR-L2 |
| 822 | DASNLAT | NBVR2 CDR-L2 |
| 823 | QQWSSYPPIT | NBVR1 CDR-L3 |
| 824 | QQRSNWPPIT | NBVR2 CDR-L3 |
| 825 | GYSITSDYAWN | NBVR1 CDR-H1 |
| 826 | GYSITSDYAWG | NBVR2 CDR-H1 |
| 827 | YMSYSGSTRYNPSLRS | NBVR1 CDR-H2 |
| 828 | SMSYSGSTYYNPSLKS | NBVR2 CDR-H2 |
| 829 | ARGWPLAY | NBVR1 CDR-H3 |
| 829 | ARGWPLAY | NBVR2 CDR-H3 |
| 830 | MDFQVQIFSFLLISASVILSRGQIVLTQSPAIMSASPGEKVTMTCSASSSVYY | AN02 light chain |
| | MYWYQQKPGSSPRLLIYDTSNLASGVPVRFSGSGSGTSYSLTISRMEAEDAAT | variable domain |
| | YYCQQWSSYPPITFGVGTKLELKRA | |
| 831 | MRVILLWLFTAFPGILSDVQLQESGPGLVKPSQSQSLTCTVTGYSITSDYAWN | AN02 heavy chain |
| | WIRQFPGNKLEWMGYMSYSGSTRYNPSLRSRISITRDTSKNQFFLQLKSVTTE | variable domain |
| | DTATYFCARGWPLAYWGQGTQVSVSE | |
| 832 | EIVMTQSPATLSLSPGERATLSCSASSSVSYMYWYQQKPGQAPRLLIYDTSNL | NBVR1 Light chain |
| | ASGIPARFSGSGSGTDFTLTISSLQPEDFAVYYCQQWSSYPPITFGQGTKVEI | variable domain |
| | K | |
| 833 | EIVMTQSPATLSLSPGERATLSCSASSSVSYMYWYQQKPGQAPRLLIYDTSNL | NBVR1 light chain |
| | ASGIPARFSGSGSGTDFTLTISSLQPEDFAVYYCQQWSSYPPITFGQGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| 834 | RTVAAPSVFIFPPSDEQLKSGTASVVCLLNNFYPREAKVQWKVDNALQSGNSQ | NBVR1 light chain |
| | ESVTEQDSKDSTYSLSSTLTLSKADYEKHKVYACEVTHQGLSSPVTKSFNRGE | constant domain |
| | C | |
| 835 | EIVMTQSPATLSLSPGERATLSCSASSSVSYMYWYQQKPGQAPRLLIYDTSNL | NBVR1 K149C light |
| | ASGIPARFSGSGSGTDFTLTISSLQPEDFAVYYCQQWSSYPPITFGQGTKVEI | chain |
| | KRTVAAPSVFIFPPSDEQLKSGTASVVCLLNNFYPREAKVQWCVDNALQSGNS | |
| | QESVTEQDSKDSTYSLSSTLTLSKADYEKHKVYACEVTHQGLSSPVTKSFNRG | |
| | EC | |
| 836 | RTVAAPSVFIFPPSDEQLKSGTASVVCLLNNFYPREAKVQWCVDNALQSGNSQ | NBVR1 light chain |
| | ESVTEQDSKDSTYSLSSTLTLSKADYEKHKVYACEVTHQGLSSPVTKSFNRGE | constant domain |
| | C | |
| 837 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | variable domain |
| | QGTLVTVSS | |
| 838 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDK | |
| | VEPK | |
| 839 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSC | |
| 840 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCP | |
| 841 | QVQLQESGPGLVKPSETLSLTCAVSGYSITSDYAWNWIRQPPGKGLEWIGYMS | NBVR1 heavy chain |
| | YSGSTRYNPSLRSRVTI SVDTSKNQFSLKLSSVTAADTAVYYCARGWPLAYWG | Fab' |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCP | |
| 842 | ASTKGPSVFPLAPSSKSTSGGTAALGCLVKDYFPEPVTVSWNSGALTSGVHTF | NBVR1 heavy chain |
| | PAVLQSSGLYSLSSVVTVPSSSLGTQTYICNVNHKPSNTKVDKKVEPK | CH1 domain |
| 843 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | Fc-conjugate |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVFSCS | |
| | VMHEALHNHYTQKSLSLSPGK | |
| 844 | QVQLQESGPGLVKPSETLSLTCAVSGYSITSDYAWNWIRQPPGKGLEWIGYMS | NBVR1 A114C |
| | YSGSTRYNPSLRSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPK | |
| 845 | QVQLQESGPGLVKPSETLSLTCAVSGYSITSDYAWNWIRQPPGKGLEWIGYMS | NBVR1 A114C |
| | YSGSTRYNPSLRSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSC | |
| 846 | QVQLQESGPGLVKPSETLSLTCAVSGYSITSDYAWNWIRQPPGKGLEWIGYMS | NBVR1 A114C |
| | YSGSTRYNPSLRSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCP | |
| 847 | QVQLQESGPGLVKPSETLSLTCAVSGYSITSDYAWNWIRQPPGKGLEWIGYMS | NBVR1 heavy chain |
| | YSGSTRYNPSLRSRVTISVDTSKNQFSLKLSSVTAADTAVYYCARGWPLAYWG | Fab' |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCP | |
| 848 | CSTKGPSVFPLAPSSKSTSGGTAALGCLVKDYFPEPVTVSWNSGALTSGVHTF | NBVR1 A114C |
| | PAVLQSSGLYSLSSVVTVPSSSLGTQTYICNVNHKPSNTKVDKKVEPK | heavy chain CH1 |
| | | domain |
| 849 | QVQLQESGPGLVKPSETLSLTCAVSGYSITSDYAWNWIRQPPGKGLEWIGYMS | NBVR1 A114C |
| | YSGSTRYNPSLRSRVTISVDTSKNQFSLKLSSVTAADTAVYYCARGWPLAYWG | heavy chain Fc- |
| | QGTLVTVSSCSTKGPSVFPLAPSSKSTSGGTAALGCLVKDYFPEPVTVSWNSG | conjugate |
| | ALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVESCS | |
| | VMHEALHNHYTQKSLSLSPGK | |
| 850 | EIVLTQSPATLSLSPGERATLSCRASQSVSSYLAWYQQKPGQAPRLLIYDASN | NBVR2 light chain |
| | LATGIPARFSGSGSGTDFTLTISSLEPEDFAVYYCQQRSNWPPITFGQGTKVE | |
| | IKRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFNR | |
| | GEC | |
| 851 | EIVLTQSPATLSLSPGERATLSCRASQSVSSYLAWYQQKPGQAPRLLIYDASN | NBVR2 light chain |
| | LATGIPARFSGSGSGTDFTLTISSLEPEDFAVYYCOORSNWPPITFGQGTKVE | variable domain |
| | IK | |
| 852 | EIVLTQSPATLSLSPGERATLSCRASQSVSSYLAWYQQKPGQAPRLLIYDASN | NBVR2 |
| | LATGIPARFSGSGSGTDFTLTISSLEPEDFAVYYCQQRSNWPPITFGQGTKVE | K149C light |
| | IKRTVAAPSVFIFPPSDEQLKSGTASVVCLLNNFYPREAKVQWCVDNALQSGN | chain |
| | SQESVTEQDSKDSTYSLSSTLTLSKADYEKHKVYACEVTHQGLSSPVTKSFNR | |
| | GEC | |
| 853 | QVQLQESGPGLVKPSETLSLTCAVSGYSITSDYAWGWIRQPPGKGLEWIGSMS | NBVR2 heavy chain |
| | YSGSTYYNPSLKSRVTISVDTSKNQFSLKLSSVTAADTAVYYCARGWPLAYWG | variable domain |
| | QGTLVTVSS | |
| 854 | QVQLQESGPGLVKPSETLSLTCAVSGYSITSDYAWGWIRQPPGKGLEWIGSMS | NBVR2 heavy chain |
| | YSGSTYYNPSLKSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPK | |
| 855 | QVQLQESGPGLVKPSETLSLTCAVSGYSITSDYAWGWIROPPGKGLEWIGSMS | NBVR2 heavy chain |
| | YSGSTYYNPSLKSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSC | |
| 856 | QVQLQESGPGLVKPSETLSLTCAVSGYSITSDYAWGWIRQPPGKGLEWIGSMS | NBVR2 heavy chain |
| | YSGSTYYNPSLKSRVTISVDTSKNQFSLKLSSVTAADTAVYYCARGWPLAYWG | Fab |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCP | |
| 857 | QVQLQESGPGLVKPSETLSLTCAVSGYSITSDYAWGWIRQPPGKGLEWIGSMS | NBVR2 heavy chain |
| | YSGSTYYNPSLKSRVTISVDTSKNQFSLKLSSVTAADTAVYYCARGWPLAYWG | Fab′ |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCP | |
| 858 | QVQLQESGPGLVKPSETLSLTCAVSGYSITSDYAWGWIRQPPGKGLEWIGSMS | NBVR2 heavy chain |
| | YSGSTYYNPSLKSRVTISVDTSKNOFSLKLSSVTAADTAVYYCARGWPLAYWG | Fc-conjugate |
| | QGTLVTVSSASTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVESCS | |
| | VMHEALHNHYTQKSLSLSPGK | |
| 859 | QVQLQESGPGLVKPSETLSLTCAVSGYSITSDYAWGWIRQPPGKGLEWIGSMS | NBVR2 A114C |
| | YSGSTYYNPSLKSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPK | |
| 860 | QVQLQESGPGLVKPSETLSLTCAVSGYSITSDYAWGWIRQPPGKGLEWIGSMS | NBVR2 A114C |
| | YSGSTYYNPSLKSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSC | |
| 861 | QVQLQESGPGLVKPSETLSLTCAVSGYSITSDYAWGWIRQPPGKGLEWIGSMS | NBVR2 A114C |
| | YSGSTYYNPSLKSRVTISVDTSKNQFSLKLSSVTAADTAVYYCARGWPLAYWG | heavy chain Fab |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCP | |
| 862 | QVQLQESGPGLVKPSETLSLTCAVSGYSITSDYAWGWIRQPPGKGLEWIGSMS | NBVR2 A114C |
| | YSGSTYYNPSLKSRVTISVDTSKNQFSLKLSSVTAADTAVYYCARGWPLAYWG | heavy chain Fab′ |
| | QGTLVTVSSCSTKGPSVFPLAPSSKSTSGGTAALGCLVKDYFPEPVTVSWNSG | |
| | ALTSGVHTFPAVLQSSGLYSLSSVVTVPSSSLGTQTYICNVNHKPSNTKVDKK | |
| | VEPKSCDKTHTCPPCP | |
| 863 | QVQLQESGPGLVKPSETLSLTCAVSGYSITSDYAWGWIRQPPGKGLEWIGSMS | NBVR2 A114C |
| | YSGSTYYNPSLKSRVTISVDTSKNQFSLKLSSVTAADTAVYYCARGWPLAYWG | heavy chain-Fc |
| | QGTLVTVSSCSTKGPSVFPLAPSSKSTSGGTAALGCLVKDYFPEPVTVSWNSG | conjugate |
| | ALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVFSCS | |
| | VMHEALHNHYTQKSLSLSPGK | |
| 864 | gaaattgtcctcacccagagcccagccactctttccctctctccaggggaaag | NBVR2 light chain |
| | agcaacactgagttgccgggcctcccagagtgtaagttcctacctcgcatggt | variable domain |
| | atcaacagaagcctggtcaagcaccccggcttctgatttacgatgccagcaat | coding sequence |
| | ctcgccaccggaatccccgcaaggttttcagggagcggttcagggactgactt | |
| | taccctgaccataagcagtcttgagcctgaggattttgcagtgtattactgcc | |
| | aacagagaagtaactggccccccataacctttggacaggggactaaggtggag | |
| | attaaa | |
| 865 | caagtgcaactgcaggaaagtgggcctggtttggtcaaacccagtgaaacact | NBVR2 heavy chain |
| | tagtcttacttgcgctgtatctggatactctattacaagcgattatgcctggg | variable domain |
| | ggtggattcggcagccccctggaaaaggcttggagtggatcggttcaatgagt | coding sequence |
| | tatagtggtagtacatactacaacccttccctgaagtctcgcgtcacaataag | |
| | cgtagatacaagtaagaatcaattttccctgaaacttagcagtgtaactgccg | |
| | ctgataccgcagtctactattgtgccagaggatggcccctggcctactggggt | |
| | cagggcaccctcgtgaccgtatcatca | |
| 866 | gagatcgtactcactcaaagtccagccaccctgtcactttctccaggagaacg | NBVR2 light chain |
| | cgccactctctcttgtcgggctagtcagagcgttagtagctatctcgcatggt | variable domain |
| | atcaacaaaagcccggtcaggccccacgcctgttgatctatgatgccagtaac | coding sequence |
| | ctggccaccggtattccagcccgtttctctggcagtggaagtggaactgactt | |
| | cacactgaccatcagctccctcgagcccgaagattttgctgtgtattactgcc | |
| | agcaaagatccaactggcctccaatcactttcggccaaggaacaaaagtagag | |
| | attaag | |
| 867 | caggttcaacttcaagagtcaggcccaggcttggtgaagccctccgaaacttt | NBVR2 heavy chain |
| | gagcttgacttgcgctgtgtccggttacagtataacctctgactatgcctggg | variable domain |
| | ggtggataaggcaacctcctggcaaaggattggaatggatcggttcaatgtcc | coding sequence |
| | tactccggttctacatactataacccctccttgaagtcccgagtaactatcag | |
| | tgtggacacttcaaagaatcaatttagtctgaaattgtcttcagtcaccgctg | |
| | cagacactgctgtctattattgtgccaggggttggccccttgcttattggggc | |
| | caggggaccctggtgacagtatcaagc | |
| 868 | EPKSCDKTHTCPPCP | Antibody hinge |
| | | region sequence |
| 869 | X₁MSYSGSTX₂YNPSLX₃S | NBVR CDR-H2 |
| | Wherein: X₁ = Y or S; X₂ = R or Y; and X₃ = R or K | consensus |
| 870 | UcCuAuGaCuGuAgAuUuUaU | HPRT siRNA sense |
| | | strand sequence |
| 871 | aUaAaAuCuAcAgUcAuAgGaAu | HPRT siRNA |
| | | antisense strand |
| | | sequence |
| 872 | ATTCCTATGACT | HPRT siRNA AS |
| | | Assay Capture Probe |
| 873 | GTAGATTTTAT | HPRT siRNA AS |
| | | Assay Detection |
| | | Probe |
| 874 | ATAAAATCTAC | HPRT siRNA SS |
| | | Assay Capture Probe |
| 875 | AGTCATAGGA | HPRT siRNA SS |
| | | Assay Detection |
| | | Probe |
| 876 | ATAAAATCTACAGTCATAGGA | OTV: HPRT Assay |
| | | Capture Probe |

The abbreviations refer to the components as follows: d: DNA; k: LNA; ^{m}C: 5-methylcytidine (methylated cytosine); s: phosphorothioate backbone (PS); and L′ is: 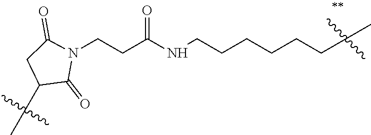 [SMILES: *CCCCCCNC(=O)CCN1C(=O)CC(*)C1=O]
wherein valence marked ** is attached through a phosphate (—O—P(═O)₂—O—) at the 5′ end of the ASO.

What is claimed is:

1. A conjugate of formula I:
P-(L-(X)_{y})ₙ (I)
wherein,
each X is independently an oligonucleotide;
each L is independently a linking group;
P is a protein comprising an engineered binding site that specifically binds to a transferrin receptor (TfR) with an affinity of about 3 nM to about 600 nM;
each y is independently at least 1; and
n is at least 1.

2. The conjugate of claim 1, wherein the engineered binding site binds TfR with an affinity of about 500 nM to about 3 nM.

3. The conjugate of claim 1 or 2, wherein the engineered binding site binds TfR with an affinity of about 400 nM to about 20 nM.

4. The conjugate of claim 1 or 2, wherein the engineered binding site binds TfR with an affinity of about 300 nM to about 30 nM.

5. The conjugate of claim 1 or 2, wherein the engineered binding site binds TfR with an affinity of about 200 nM to about 40 nM.

6. The conjugate of claim 1 or 2, wherein the engineered binding site binds TfR with an affinity of about 150 nM to about 50 nM.

7. The conjugate of claim 1 or 2, wherein the engineered binding site binds TfR with an affinity of about 130 nM to about 80 nM.

8. The conjugate of any one of claims 1-7, wherein the engineered binding site is a non-native binding site.

9. The conjugate of claim 8, wherein the non-native binding site is in a constant domain of P.

10. The conjugate of any one of claim 1-9, wherein P is a protein comprising 1) a modified constant domain that specifically binds to a transferrin receptor; and 2) one or more modified sites, which facilitate the attachment of P to each L.

11. The conjugate of claim 10, wherein P comprises an Fc polypeptide comprising the modified constant domain.

12. The conjugate of claim 11, wherein P comprises an Fc polypeptide dimer, comprising a first Fc polypeptide comprising the modified constant domain; and a second Fc polypeptide dimerized to the first Fc polypeptide.

13. The conjugate of any one of claims 10-12, wherein the modified site is an amino acid substitution.

14. The conjugate of claim 13, wherein the modified site is a cysteine substitution.

15. The conjugate of claim 14, wherein the cysteine substitution is selected from the group consisting of: S239C, S442C, A330C, K149C, and T289C, according to EU numbering and A114C according to Kabat numbering.

16. The conjugate of any one of claims 1-15, wherein the oligonucleotide is an antisense oligonucleotide (ASO) or a siRNA.

17. The conjugate of any one of claims 1-16, wherein the linking group L comprises a moiety having the structure: 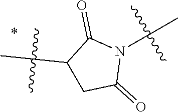
wherein, * denotes the attachment point to a sulfur atom of a modified site within P.

18. The conjugate of any one of claims 1-17, wherein y is an integer from 1 to 4.

19. The conjugate of any claim 18, wherein y is 1.

20. The conjugate of any one of claims 1-19, wherein n is an integer from 1 to 6.

21. The conjugate of claim 20, wherein n is 1.

22. An Fc polypeptide dimer, or a Fab-Fc dimer fusion thereof, comprising:
(a) a first Fc polypeptide comprising a modified constant domain that specifically binds to a transferrin receptor; and
(b) a second Fc polypeptide dimerized to the first Fc polypeptide of (a);
wherein the Fab-Fc dimer fusion further comprises a first Fab and a second Fab; and,
wherein the Fc polypeptide dimer, or a Fab-Fc dimer fusion comprises one or more cysteine substitutions.

23. The Fc polypeptide dimer of claim 22, wherein the Fab-Fc dimer fusion is a non-targeting Fab-Fc dimer fusion.

24. The Fc polypeptide dimer of claim 22 or 23, wherein the cysteine substitution(s) is selected from the group consisting of: S239C, S442C, A330C and T289C according to EU numbering, and A114C according to Kabat numbering.

25. The Fc polypeptide dimer of any one of claims 22-24, wherein the first and second Fc polypeptides each comprise a cysteine substitution; or wherein the first and second Fab-Fc fusions each comprise a cysteine substitution.

26. The Fc polypeptide dimer of claim 25, wherein the first and second Fc polypeptides each comprise a cysteine substitution at S239C; or wherein the first and second Fab-Fc fusions each comprise a cysteine substitution at S239C.

27. The Fc polypeptide dimer of any one of claims 22-26, wherein the first and second Fc polypeptides each comprise two cysteine substitutions; or wherein the first and second Fab-Fc fusions each comprise two cysteine substitutions.

28. The Fc polypeptide dimer of claim 27, wherein the first and second Fc polypeptides each comprise a cysteine substitution at S239C and A330C; or wherein the first and second Fab-Fc fusions each comprise a cysteine substitution at S239C and A330C.

29. The Fc polypeptide dimer of any one of claims 22-24, wherein the second Fc polypeptide comprises a cysteine substitution; or wherein the second Fab-Fc fusion comprises a cysteine substitution.

30. The Fc polypeptide dimer of claim 29, wherein the cysteine substitution is S239C.

31. An Fc polypeptide dimer comprising:
(a) a first Fc polypeptide comprising a modified constant domain that specifically binds to a transferrin receptor; and
(b) a second Fc polypeptide dimerized to the first Fc polypeptide of (a);
wherein the first and/or the second Fc polypeptide comprises one or more substitutions selected from the group consisting of N297A and N297G.

32. The Fc polypeptide dimer of claim 31, wherein the first and the second Fc polypeptides are each joined to a non-targeting Fab fragment or a portion thereof, to produce a Fab-Fc dimer fusion.

33. The Fc polypeptide dimer of any one of claims 22-33, wherein the modified constant domain is a modified CH3 domain comprising two, three, four, five, six, seven, eight, nine, ten, eleven, twelve, thirteen, fourteen, fifteen or sixteen positions selected from the following: position 153 is Trp, Tyr, Leu, Gln, or Glu; position 157 is Leu, Tyr, Met, Val, Phe or Trp; position 159 is Leu, Thr, His, Pro or Phe; position 160 is Val, Pro, or an acidic amino acid; position 161 is Trp; position 162 is Val, Ser, Ala or Gly; position 163 is Asn, Gly, His, Gln, Leu, Lys, Val, Phe, Ser, Ala, Asp, Thr or Glu; position 164 is Ser, Thr, Gln, Phe, Tyr or Val; position 165 is Gln, Phe, or His; position 186 is Glu, Ala, Ser, Leu, Thr, Pro or Asp; position 187 is Lys, Arg, Gly, or Pro; position 188 is Glu or Ser; position 189 is Thr, Asn or an acidic amino acid; position 194 is Trp, Tyr, His, or Phe; position 197 is Ser, Thr, Glu, Lys or Trp; and position 199 is Ser, Trp, Gly, Cys, Pro or Met, as numbered with reference to SEQ ID NO:1.

34. The Fc polypeptide dimer of any one of claims 22-33,
wherein the first Fc polypeptide or Fab-Fc dimer fusion comprises a sequence having at least 85%, 90%, 95%, 96%, 97%, 98% or 99% sequence identity to the sequence of any one of SEQ ID NOS:279, 281, 361-366, 491-494, 702-718, 632, 645-649, 738-746, 804, and
wherein the second Fc polypeptide or Fab-Fc dimer fusion comprises a sequence having at least 85%, 90%, 95%, 96%, 97%, 98% or 99% sequence identity to the sequence of any one of SEQ ID NOS: 557-561, 627-631, 635-644, 719-723, 724-731, 733-737, and 810.

35. The Fc polypeptide dimer of claim 34, wherein the first Fc polypeptide or Fab-Fc dimer fusion comprises a Glu at position 153, Tyr at position 157, Thr at position 159, Glu at position 160, Trp at position 161, Ser, or Ala at position 162, Asn at position 163, Thr or Ser at position 186, Glu at position 188, Glu at position 189, and Phe at position 194, as numbered with reference to SEQ ID NO:1.

36. The Fc polypeptide dimer of claim 35, wherein:
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:702 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:708 and the second Fc polypeptide or Fab-Fc dimer fusion comprises the amino acid sequence of SEQ ID NO:722;
the first Fc polypeptide or Fab-Fc dimer fusion comprises the amino acid sequence of SEQ ID NO:718 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:560;
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:561; or
the first Fc polypeptide comprises the amino acid sequence of SEQ ID NO:804 and the second Fc polypeptide comprises the amino acid sequence of SEQ ID NO:810.

37. A conjugate of formula I:
P-(L-(X)_{y})ₙ (I)
wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1.

38. The conjugate of claim 37, wherein P comprises an Fc polypeptide comprising the modified constant domain.

39. The conjugate of claim 38, wherein the protein comprises an Fc polypeptide dimer, comprising a first Fc polypeptide comprising the modified constant domain; and a second Fc polypeptide dimerized to the first Fc polypeptide.

40. The conjugate of claim 38 or 39, wherein the Fc polypeptide is joined to a non-targeting Fab fragment or a portion thereof, to produce a Fab-Fc dimer fusion; or the first and second Fc polypeptides of the Fc polypeptide dimer are each joined to a non-targeting Fab fragment or a portion thereof, to produce a Fab-Fc dimer fusion.

41. The conjugate of any one of claims 37-40, wherein the modified site is an amino acid substitution.

42. The conjugate of claim 41, wherein the modified site is a cysteine substitution.

43. The conjugate of claim 42, wherein the cysteine substitution is selected from the group consisting of: S239C, S442C, A330C, K149C, and T289C, according to EU numbering and A114C according to Kabat numbering.

44. The conjugate of any one of claims 37-43, wherein the linking group L comprises a moiety having the structure: 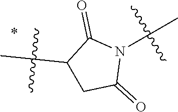
wherein, * denotes the attachment point to a sulfur atom of a modified site within P.

45. The conjugate of claim 41, wherein the modified site is an alanine or glycine substitution.

46. The conjugate of claim 45, wherein the alanine or glycine substitution is N297A or N297G, according to EU numbering.

47. The conjugate of any one of claims 37-41 and 45-46, wherein L is conjugated to P at Q295.

48. The conjugate of claim 47, wherein L is conjugate to P by enzymatic conjugation.

49. The conjugate of claim 48, wherein the enzymatic conjugation uses a bacterial transglutaminase (BTG).

50. The conjugate of any one of claims 37-49, wherein the modified constant domain is a modified CH3 domain comprising two, three, four, five, six, seven, eight, nine, ten, eleven, twelve, thirteen, fourteen, fifteen or sixteen positions selected from the following: position 153 is Trp, Tyr, Leu, Gln, or Glu; position 157 is Leu, Tyr, Met, Val, Phe or Trp; position 159 is Leu, Thr, His, Pro or Phe; position 160 is Val, Pro, or an acidic amino acid; position 161 is Trp; position 162 is Val, Ser, Ala or Gly; position 163 is Asn, Gly, His, Gln, Leu, Lys, Val, Phe, Ser, Ala, Asp, Thr or Glu; position 164 is Ser, Thr, Gln, Phe, Tyr or Val; position 165 is Gln, Phe, or His; position 186 is Glu, Ala, Ser, Leu, Thr, Pro or Asp; position 187 is Lys, Arg, Gly, or Pro; position 188 is Glu or Ser; position 189 is Thr, Asn or an acidic amino acid; position 194 is Trp, Tyr, His, or Phe; position 197 is Ser, Thr, Glu, Lys or Trp; and position 199 is Ser, Trp, Gly, Cys, Pro or Met, as numbered with reference to SEQ ID NO:1.

51. The conjugate of any one of claims 37-50,
wherein the first Fc polypeptide or Fab-Fc dimer fusion comprises a sequence having at least 85%, 90%, 95%, 96%, 97%, 98% or 99% sequence identity to the sequence of any one of SEQ ID NOS:279, 281, 361-366, 491-494, 702-718, 632, 645-649, 738-746, 804; and
wherein the second Fc polypeptide or Fab-Fc dimer fusion comprises a sequence having at least 85%, 90%, 95%, 96%, 97%, 98% or 99% sequence identity to the sequence of any one of SEQ ID NOS: 557-561, 627-631, 635-644, 719-723, 724-731, 733-737, and 810.

52. The conjugate of claim 51, wherein the first Fc polypeptide or Fab-Fc dimer fusion comprises a Glu at position 153, Tyr at position 157, Thr at position 159, Glu at position 160, Trp at position 161, Ser, or Ala at position 162, Asn at position 163, Thr or Ser at position 186, Glu at position 188, Glu at position 189, and Phe at position 194, as numbered with reference to SEQ ID NO:1.

53. The conjugate of claim 51, wherein:
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:702 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:708 and the second Fc polypeptide or Fab-Fc dimer fusion comprises the amino acid sequence of SEQ ID NO:722;
the first Fc polypeptide or Fab-Fc dimer fusion comprises the amino acid sequence of SEQ ID NO:718 and the second Fc polypeptide or Fab-Fc dimer fusion comprises the amino acid sequence of SEQ ID NO:559;
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:560;
the first Fc polypeptide or Fab-Fc dimer fusion comprises the amino acid sequence of SEQ ID NO:362 and the second Fc polypeptide or Fab-Fc dimer fusion comprises the amino acid sequence of SEQ ID NO:561; or
the first Fc polypeptide comprises the amino acid sequence of SEQ ID NO:804 and the second Fc polypeptide comprises the amino acid sequence of SEQ ID NO:810.

54. The conjugate of any one of claims 37-53, wherein the oligonucleotide is an antisense oligonucleotide (ASO) or a siRNA.

55. The conjugate of claim 54, wherein each oligonucleotide is independently an ASO.

56. The conjugate of claim 55, wherein the ASO is a gapmer having a modification pattern of formula 5′-Xₐ-Yₐ-Zₐ-3′, wherein Xₐ and Zₐ each comprise 3 LNA modified nucleotides, and wherein the gap region Yₐ comprises PS linkages.

57. The conjugate of claim 55 or 56, wherein each L is linked to the 5′ end of an ASO.

58. The conjugate of claim 55 or 56, wherein each L is linked to the 3′ end of an ASO.

59. The conjugate of any one of claims 37-58, wherein L comprises a C₆ amine group having the formula —(CH₂)₆—NH—.

60. The conjugate of any one of claims 37-59, wherein P binds TfR with an affinity of about 500 nM to about 10 nM.

61. The conjugate of claim 60, wherein P binds TfR with an affinity of about 400 nM to about 20 nM.

62. The conjugate of claim 61, wherein P binds TfR with an affinity of about 300 nM to about 30 nM.

63. The conjugate of claim 62, wherein P binds TfR with an affinity of about 200 nM to about 40 nM.

64. The conjugate of claim 63, wherein P binds TfR with an affinity of about 150 nM to about 50 nM.

65. The conjugate of claim 64, wherein P binds TfR with an affinity of about 100 nM.

66. The conjugate of any one of claims 37-65, wherein y is an integer from 1 to 4.

67. The conjugate of claim 66, wherein y is 1.

68. The conjugate of any one of claims 37-67, wherein n is an integer from 1 to 6.

69. The conjugate of claim 68, wherein n is 1.

70. The conjugate of any one of claims 37-39 and 41-69, wherein P does not comprise a Fab fragment or an antigen-binding portion or variable domain portion a portion thereof.

71. A conjugate of formula I that comprises more than one oligonucleotide:
P-(L-(X)_{y})ₙ (I)
wherein,
each X is independently an oligonucleotide;
each L is independently a linking group;
P is a protein comprising a modified constant domain that specifically binds to a transferrin receptor;
each y is independently at least 1; and
n is 2 or more.

72. The conjugate of claim 71, wherein n is 2 or 4.

73. The conjugate of claim 71 or 72, wherein y is 1.

74. The conjugate of claim 71 or 73, wherein P comprises an Fc polypeptide comprising the modified constant domain.

75. The conjugate of any one of claims 71-74, wherein P comprises an Fc polypeptide dimer, comprising a first Fc polypeptide comprising the modified constant domain; and a second Fc polypeptide capable of dimerizing to the first Fc polypeptide.

76. The conjugate of claim 74 or 75, wherein the Fc polypeptide is joined to a non-targeting Fab fragment or a portion thereof to produce a Fab-Fc dimer fusion; or the first and second Fc polypeptides of the Fc polypeptide dimer are each joined to a non-targeting Fab fragment or a portion thereof to produce a Fab-Fc dimer fusion.

77. The conjugate of any one of claims 71-76, wherein P comprises one or more modified sites, which facilitate the attachment of P to each L.

78. The conjugate of claim 77, wherein the modified site is an amino acid substitution.

79. The conjugate of claim 78, wherein the modified site is a cysteine substitution.

80. The conjugate of claim 79, wherein the cysteine substitution is selected from the group consisting of: S239C, S442C, A330C, K149C, and T289C, according to EU numbering and A114C according to Kabat numbering.

81. The conjugate of any one of claims 75-80, wherein n is 2, and wherein the first Fc polypeptide and the second Fc polypeptide are each attached to an L through a cysteine residue at position 239.

82. The conjugate of any one of claims 75-80, wherein n is 4, and wherein the first Fc polypeptide and the second Fc polypeptide are each attached to two Ls through cysteine residues at positions 239 and 330, respectively.

83. The conjugate of any one of claims 71-82, wherein the oligonucleotide is an antisense oligonucleotide (ASO) or a siRNA.

84. A conjugate of formula I that comprises more than one oligonucleotide:
P-(L-(X)_{y})ₙ (I)
wherein,
each X is independently an oligonucleotide;
each L is independently a linking group;
P is a protein comprising a modified constant domain that specifically binds to a transferrin receptor;
each y is independently at least 1, wherein at least one y is 2 or more; and
n is at least one.

85. The conjugate of claim 84, wherein y is 2.

86. The conjugate of claim 85, wherein at least one L is attached at the 5′ end of a first oligonucleotide and a second oligonucleotide is linked to the 3′end of the first oligonucleotide.

87. The conjugate of claim 86, wherein the first and second oligonucleotide are linked by a nucleic acid linker or a non-oligonucleotide cleavable linker.

88. The conjugate of any one of claims 84-87, wherein P comprises an Fc polypeptide comprising the modified constant domain.

89. The conjugate of any one of claims 84-87, wherein P comprises an Fc polypeptide dimer, comprising a first Fc polypeptide comprising the modified constant domain; and a second Fc polypeptide dimerized to the first Fc polypeptide.

90. The conjugate of claim 88 or 89, wherein the Fc polypeptide is joined to a non-targeting Fab fragment or a portion thereof to produce a Fab-Fc dimer fusion; or the first and second Fc polypeptides of the Fc polypeptide dimer are each joined to a non-targeting Fab fragment or a portion thereof to produce a Fab-Fc dimer fusion.

91. The conjugate of any one of claims 84-90, wherein P further comprises one or more modified sites, which facilitate the attachment of P to each L.

92. The conjugate of claim 91, wherein the modified site is an amino acid substitution.

93. The conjugate of claim 92, wherein the modified site is a cysteine substitution.

94. The conjugate of claim 93, wherein the cysteine substitution is selected from the group consisting of: S239C, S442C, A330C, K149C, and T289C, according to EU numbering and A114C according to Kabat numbering.

95. The conjugate of any one of claims 84-94, wherein the oligonucleotide is an antisense oligonucleotide (ASO) or a siRNA.

96. A conjugate of formula I:
P-(L-(X)_{y})ₙ (I)
wherein,
each X is independently an oligonucleotide;
each L is a linking group independently selected from the group consisting of: 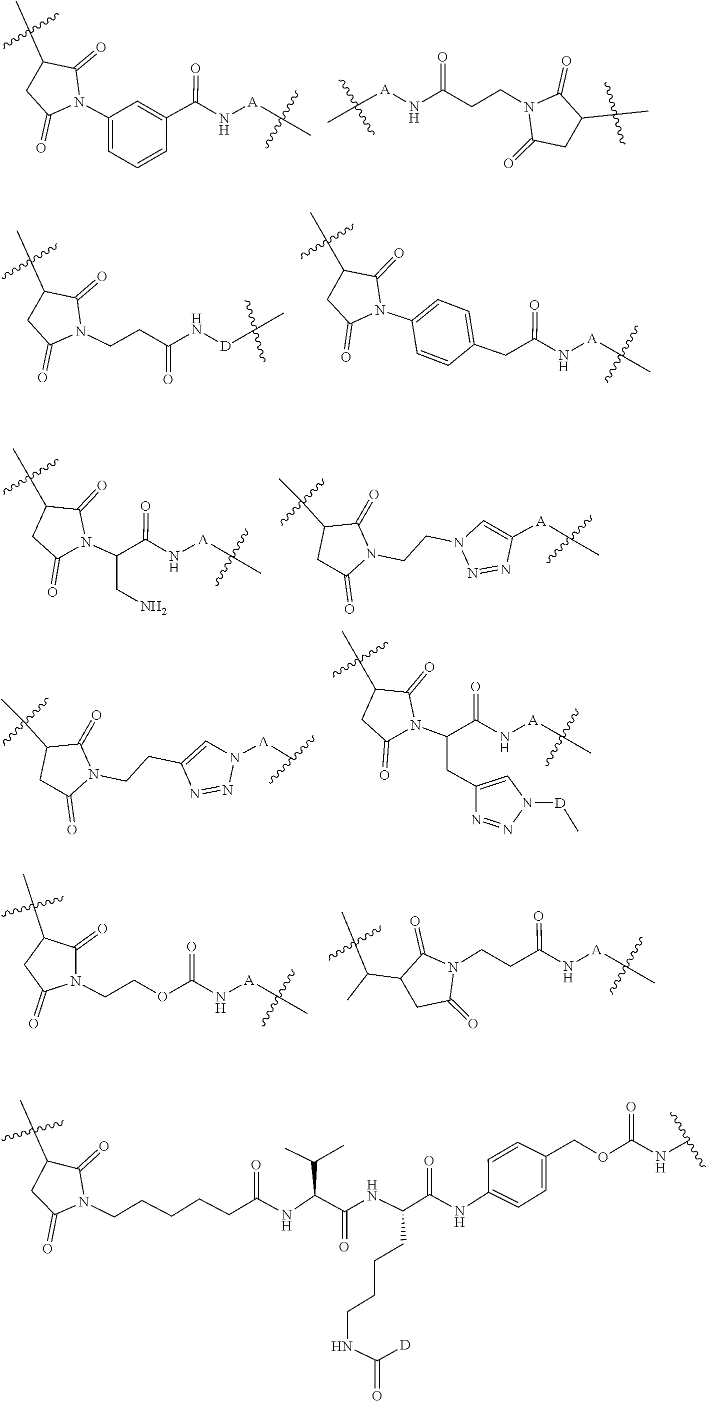 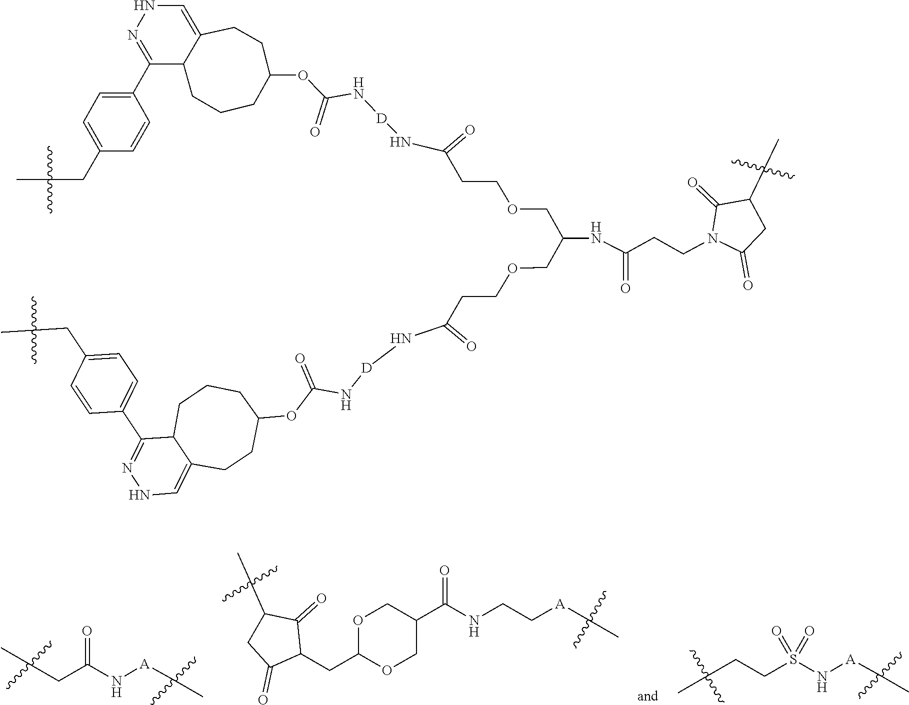
wherein,
each A is independently (C₁-C₁₅)alkylene;
each D is —(CH₂—CH₂—O)ₘ—; and
each m is 1, 2, 3, 4, 5, 6, 7, 8, 9, 10, 11, 12, 13, 14, 15, 16, 17, 18, 19, 20, 21, 22, 23, or 24;
P is a protein comprising a modified constant domain that specifically binds to a transferrin receptor;
each y is independently at least 1; and
n is at least 1.

97. The conjugate of claim 96, wherein each L is a linking group independently selected from the group consisting of:

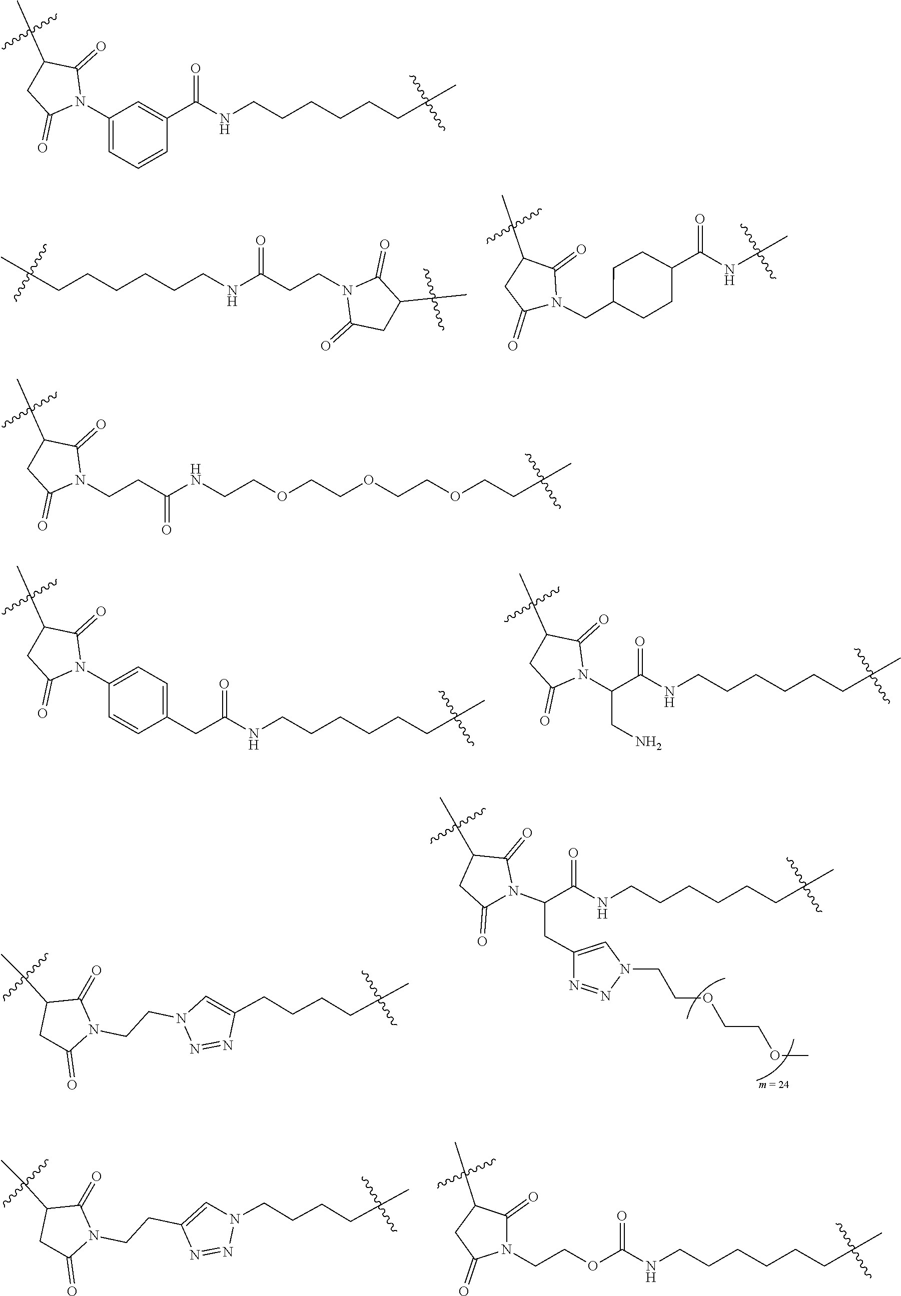

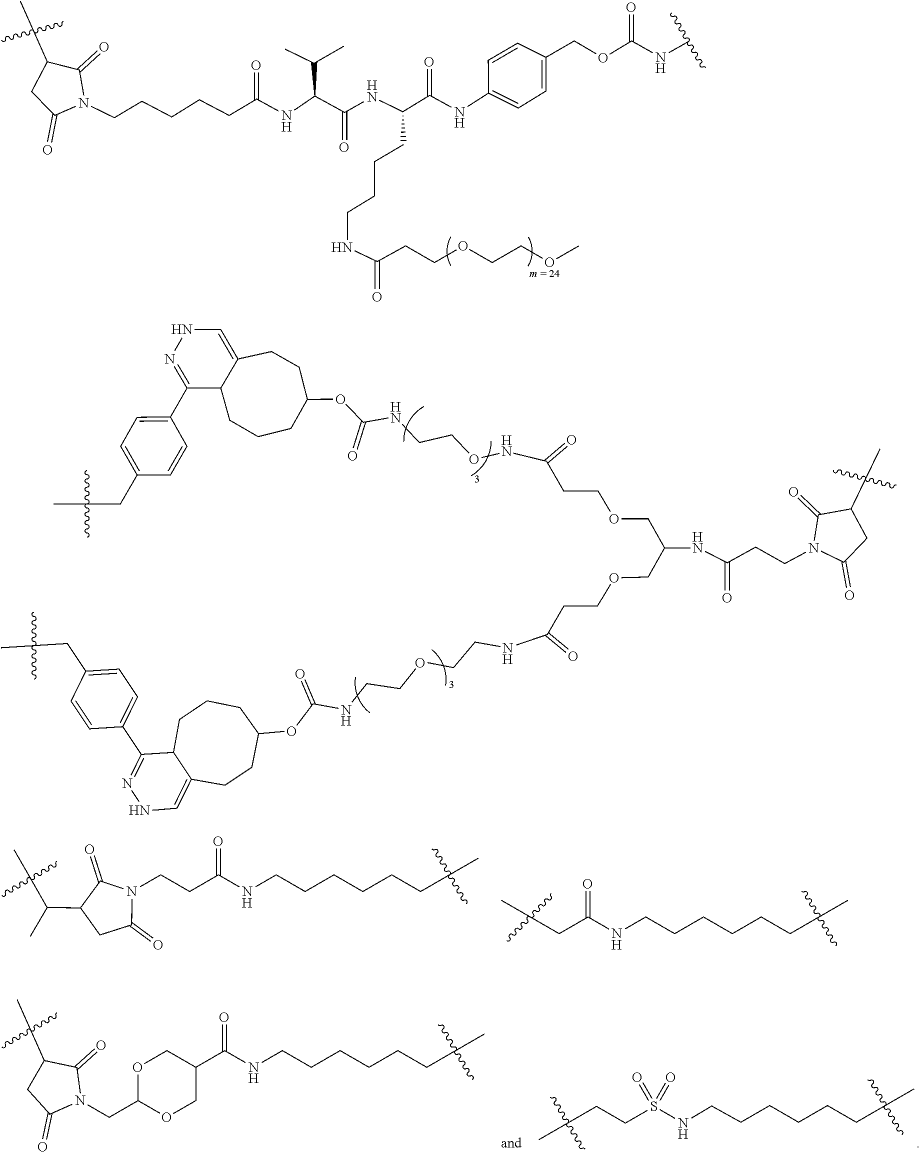

98. The conjugate of any one of claims 96-97, wherein P comprises an Fc polypeptide comprising the modified constant domain.

99. The conjugate of any one of claims 96-97, wherein P comprises an Fc polypeptide dimer, comprising a first Fc polypeptide comprising the modified constant domain; and a second Fc polypeptide dimerized to the first Fc polypeptide.

100. The conjugate of claim 98 or 99, wherein the Fc polypeptide is joined to a non-targeting Fab fragment or a portion thereof to produce a Fab-Fc dimer fusion; or the first and second Fc polypeptides of the Fc polypeptide dimer are each joined to a non-targeting Fab fragment or a portion thereof to produce a Fab-Fc dimer fusion.

101. The conjugate of any one of claims 96-100, wherein P further comprises one or more modified sites, which facilitate the attachment of P to each L.

102. The conjugate of claim 101, wherein the modified site is an amino acid substitution.

103. The conjugate of claim 102, wherein the modified site is a cysteine substitution.

104. The conjugate of claim 103, wherein the cysteine substitution is selected from the group consisting of: S239C, S442C, A330C, K149C, A114C, and T289C.

105. A conjugate of formula I:
P-(L-(X)_{y})ₙ (I)
wherein,
each X is independently an oligonucleotide;
each L is independently a linking group;
P is a protein comprising a modified constant domain that specifically binds to a transferrin receptor, wherein P does not comprise a Fab fragment or a portion thereof;
each y is independently 1 or more; and
n is 1 or more.

106. The conjugate of claim 105, wherein the oligonucleotide is an antisense oligonucleotide (ASO) or a siRNA.

107. The conjugate of claim 105, wherein the conjugate is greater than 50 kDa in size.

108. The conjugate of any one of claims 105-107, wherein P comprises an Fc polypeptide comprising the modified constant domain.

109. The conjugate of any one of claims 105-107, wherein P comprises an Fc polypeptide dimer, comprising a first Fc polypeptide comprising the modified constant domain; and a second Fc polypeptide dimerized to the first Fc polypeptide.

110. The conjugate of any one of claims 105-110, wherein P further comprises one or more modified sites, which facilitate the attachment of P to each L.

111. The conjugate of claim 110, wherein the modified site is an amino acid substitution.

112. The conjugate of claim 111, wherein the modified site is a cysteine substitution.

113. The conjugate of claim 112, wherein the cysteine substitution is selected from the group consisting of: S239C, S442C, A330C, and T289C.

114. A conjugate of formula I:
P-(L-(X)_{y})ₙ (I)
wherein,
each X is independently an oligonucleotide;
each L is independently a linking group;
P is a protein comprising 1) a modified constant domain that specifically binds to a transferrin receptor; and 2) one or more non-targeting Fab fragments or a portion thereof;
each y is independently 1 or more; and
n is 1 or more.

115. The conjugate of claim 114, wherein the oligonucleotide is an antisense oligonucleotide (ASO) or a siRNA.

116. The conjugate of any one of claims 114-115, wherein P comprises an Fc polypeptide comprising the modified constant domain.

117. The conjugate of any one of claims 114-115, wherein P comprises an Fc polypeptide dimer, comprising a first Fc polypeptide comprising the modified constant domain; and a second Fc polypeptide dimerized to the first Fc polypeptide.

118. The conjugate of claim 116 or 117, wherein the Fc polypeptide is joined to the non-targeting Fab fragment or a portion thereof to produce a Fab-Fc dimer fusion; or the first and second Fc polypeptides of the Fc polypeptide dimer are each joined to a non-targeting Fab fragment or a portion thereof to produce a Fab-Fc dimer fusion.

119. The conjugate of any one of claims 114-118, wherein P further comprises one or more modified sites, which facilitate the attachment of P to each L.

120. The conjugate of claim 119, wherein the modified site is an amino acid substitution.

121. The conjugate of claim 120, wherein the modified site is a cysteine substitution.

122. The conjugate of claim 121, wherein the cysteine substitution is selected from the group consisting of: S239C, S442C, A330C, K149C, A114C, and T289C.

123. A conjugate of formula I:
P-(L-(X)_{y})ₙ (I)
wherein,
each X is independently an oligonucleotide;
each L is independently 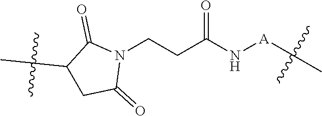
wherein A is (C₁-C₁₅)alkylene;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, and wherein the one or more modified sites are selected from the group consisting of S239C, S442C, A330C, K149C, and T289C, according to EU numbering and A114C according to Kabat numbering;
each y is independently at least 1; and
n is at least 1.

124. The conjugate of claim 123, wherein y is an integer from 1 to 4.

125. The conjugate of claim 124, wherein y is 1.

126. The conjugate of any one of claims 123-125, wherein n is an integer from 1 to 6.

127. The conjugate of claim 126, wherein n is 1.

128. A pharmaceutical composition comprising a conjugate of formula (I) of any one of claims 1-21 and 37-127 and a pharmaceutically acceptable excipient.

129. The composition of claim 128, comprising a plurality of conjugates of formula (I).

130. The composition of claim 129, wherein the ratio of oligonucleotide to protein in the composition is about 1:1 to about 4:1.

131. The composition of claim 129, wherein the ratio of oligonucleotide to protein in the composition is about 1:1 to about 2:1.

132. The composition of claim 129, wherein the ratio of oligonucleotide to protein in the composition is about 1.23.

133. The composition of claim 129, wherein the ratio of oligonucleotide to protein in the composition is about 2:1 to about 3:1.

134. The composition of claim 133, wherein the ratio of oligonucleotide to protein in the composition is about 2.5.

135. A method of targeting delivery of an oligonucleotide to muscle tissue and/or brain tissue in a patient comprising,
administering to the subject the conjugate of any one of claims 1-21 and 37-127 or the pharmaceutical composition of claim 128.

136. A method of modulating gene expression in a brain cell or a plurality of brain cells comprising administering to a patient a conjugate comprising:
(i) a protein that specifically binds TfR and;
(ii) an oligonucleotide,
wherein the protein binds TfR with an affinity of about 3 nM to about 600 nM and the conjugate is transported across the BBB, and wherein in the cell, the oligonucleotide modulates the expression of a target gene.

137. The method of claim 136, wherein the affinity is about 500 nM to about 3 nM, or about 400 nM to about 20 nM, or about 300 nM to about 30 nM, or about 200 nM to about 40 nM, or about 150 nM to about 50 nM.

138. The method of any one of claims 136-137, wherein the modulation of target gene expression is gene knockdown or gene knockout.

139. The method of claim 138, wherein the gene knockdown or gene knockout is at least 20%, at least 25%, at least 30%, at least 40%, at least 50% as compared to the expression without administering the oligonucleotide.

140. The method of any one of claims 136-139, wherein administration of the conjugate decreases MCV volume by less than about 2 femtoliters.

141. The method of any one of claims 136-140, wherein administration of the conjugate decreases TfR protein levels on the brain vasculature by less than about 10%, about 15%, about 20%, about 25%, about 30%, about 40%, about 50%, or about 60%.

142. The method of any one of claims 136-141, wherein the brain cell is a neuron, an endothelial cell, an oligodendrocyte, an astrocyte, or a microglia.

143. The method of any one of claims 136-141, wherein the plurality of cells comprise at least two, three, four, or five cell types selected from the group consisting of: a neuron, an endothelial cell, an oligodendrocyte, an astrocyte, and a microglia.

144. The method of claim 142 or 143, wherein the neuron is an excitatory neuron or inhibitory neuron.

145. The method of any one of claims 136-144, wherein the conjugate is administered to the patient intravenously.

146. A method of delivering an oligonucleotide to deep brain regions, comprising administering to a patient a conjugate comprising:
(i) a protein that specifically binds TfR and;
(ii) an oligonucleotide,
wherein the protein binds TfR with an affinity of about 3 nM to about 600 nM and the conjugate is transported across the BBB, and wherein in a brain cell in the deep brain regions, the oligonucleotide modulates the expression of a target gene.

147. The method of claim 146, wherein the affinity is about 500 nM to about 3 nM, or about 400 nM to about 20 nM, or about 300 nM to about 30 nM, or about 200 nM to about 40 nM, or about 150 nM to about 50 nM.

148. The method of any one of claims 146-147, wherein the deep brain regions comprise at least one of cortex, brainstem, hippocampus, striatum, cerebellum, or thalamus.

149. The method of any one of claims 146-148, further comprising delivering the oligonucleotide to spinal cord.

150. The method of claim 149, wherein the spinal cord is cervical spinal cord and/or lumbar spinal cord.

151. A method of delivering an antisense oligonucleotide across brain regions, comprising administering to a patient a conjugate comprising:
(i) a protein that specifically binds TfR and;
(ii) an oligonucleotide,
wherein the protein binds TfR with an affinity of about 3 nM to about 600 nM and the conjugate is transported across the BBB, and wherein in a cell in a brain region, the oligonucleotide modulates the expression of a target gene.

152. A method of generating a neuron cell with decreased target gene expression, comprising delivering to the neuron cell an oligonucleotide, wherein the oligonucleotide is transported across the BBB as part of a conjugate with a protein that binds TfR with a an affinity of about 3 nm to about 600 nM, and wherein said oligonucleotide decreases the expression level of the target gene.

153. A method of modifying a neuron cell to decrease target gene expression, comprising delivering to the neuron cell an oligonucleotide, wherein the oligonucleotide is transported across the BBB as part of a conjugate with a protein that binds TfR with an affinity of about 3 nM to about 600 mM, and wherein said oligonucleotide decreases the expression level of the target gene.

154. A method of transporting an oligonucleotide across the blood brain barrier (BBB) of a patient, comprising administering to the patient a conjugate of formula I:
P-(L-(X)_{y})ₙ (I)
wherein,
each X is independently an oligonucleotide;
each L is independently 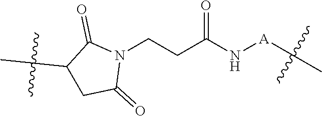
wherein A is (C₁-C₁₅)alkylene;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, and wherein the one or more modified sites are selected from the group consisting of S239C, S442C, A330C, K149C, and T289C, according to EU numbering and A114C according to Kabat numbering;
each y is independently at least 1; and
n is at least 1.

155. The method of claim 154, wherein the conjugate is administered to the patient intravenously.

156. A method of modulating the expression of a target gene in a cell within the brain of a patient, comprising administering to the patient a conjugate of formula I:
P-(L-(X)_{y})ₙ (I)
wherein,
each X is independently an oligonucleotide;
each L is independently 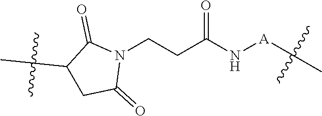
wherein A is (C₁-C₁₅)alkylene;
P is a protein comprising a Fab-Fc dimer fusion, comprising 1) a modified constant domain that specifically binds to a transferrin receptor with an affinity of about 100 nm; and 2) one or more modified sites, which facilitate the attachment of P to each L, selected from the group consisting of S239C, S442C, A330C, K149C, and T289C, according to EU numbering and A114C according to Kabat numbering;
each y is independently at least 1; and
n is at least 1.

157. The method of claim 156, wherein the modulation of target gene expression is gene knockdown or gene knockout.

158. The method of any one of claim 156 or 157, wherein the conjugate is administered to the patient intravenously.

159. A method of modulating the expression of a target gene in a cell within the brain of a patient, comprising administering to patient a conjugate of formula I:
P-(L-(X)_{y})ₙ (I)
wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1.

160. The method of claim 159, wherein the cell is a neuron, an endothelial cell, an oligodendrocyte, an astrocyte, or a microglia.

161. The method of claim 159, which modulates the expression of a target gene in a plurality of cells within the brain of the patient.

162. The method of claim 161, wherein the plurality of cells comprise at least two, three, four, or five cell types selected from the group consisting of: a neuron, an endothelial cell, an oligodendrocyte, an astrocyte, and a microglia.

163. The method of any one of claims 160-162, wherein the neuron is an excitatory neuron or inhibitory neuron.

164. The method of any one of claims 160-163, wherein the modulation of target gene expression is gene knockdown or gene knockout.

165. The method of any one of claims 160-164, wherein the conjugate is administered to the patient intravenously.

166. A method of transporting an oligonucleotide across the blood brain barrier (BBB) of a patient, comprising administering to the patient a conjugate of formula I:
P-(L-(X)_{y})ₙ (I)
wherein,
each X is independently an oligonucleotide;
each L is independently a linking group;
P is a protein comprising 1) a modified constant domain that specifically binds to a transferrin receptor; and 2) one or more modified sites, which facilitate the attachment of P to each L;
each y is independently at least 1; and
n is at least 1,
and,
wherein the oligonucleotide is distributed throughout brain regions or the central nervous system.

167. The method of claim 166, wherein brain regions comprises at least one deep brain region.

168. The method of any one of claims 166-167, wherein the conjugate is administered to the patient intravenously.

169. The method of any one of claims 166-168, wherein the oligonucleotide modulates the expression of a target gene.

170. The method of claim 169, wherein the modulation of target gene expression is gene knockdown or gene knockout.

171. A method of delivering or administering an effective amount of an oligonucleotide to the CNS of a patient, comprising administering to the patient a conjugate of formula I:
P-(L-(X)_{y})ₙ (I)
wherein,
each X is independently an oligonucleotide;
each L is independently a linking group;
P is a protein comprising a modified constant domain that specifically binds to a transferrin receptor;
each y is independently at least 1; and
n is at least 1,
and,
wherein the administered oligonucleotide modulates gene expression throughout brain regions or the central nervous system.

172. The method of claim 171, wherein the brain regions comprises at least one deep brain region.

173. The method of claim 172, wherein the at least one deep brain region is selected from: striatum, thalamus, caudate putamen, substantia nigra, and brainstem.

174. The method of any one of 171-173, wherein the brain regions comprise cells within the brain regions.

175. The method of claim 174, wherein the cells within the brain regions comprise endothelial cells, neurons, astrocytes, oligodendrocytes, and microglia.

176. The method of claim 175, wherein the oligonucleotide modulates gene expression in the endothelial cells, neurons, astrocytes, oligodendrocytes, and/or microglia.

177. The method of any one of claims 171-176, wherein the effective amount reduces gene expression by at least about 10%, 20%, 30%, 40%, 50%, 60%, 70%, 80%, 90%, 95%, 99% or 100%, as compared to the expression without administering the oligonucleotide.

178. The method of any one of claims 171-176, wherein the gene expression is reduced by at least about 50%.

179. The method of any one of claims 171-178, wherein the gene expression is reduced by at least about 70%.

180. The method of any one of claims 171-179, wherein the conjugate is administered to the patient intravenously.

181. The Fab-Fc dimer of any one of claims 22-30 or 32-36 or the conjugate of any one of claims 40-70, 76-83, 90-95, 100-104, or 119-134, wherein each of the Fab of the Fab-Fc dimer and the non-targeting Fab (NTF) of the conjugate comprises a light chain variable region and a heavy change variable region, does not specifically bind to a naturally occurring epitope in a subject, and comprises three heavy chain CDRs and three light chain CDRs, wherein
CDR-H1 comprises an amino acid sequence having at least 90% sequence identity to SEQ ID NO: 825 or 826,
CDR-H2 comprises an amino acid sequence having at least 90% sequence identity to SEQ ID NO: 827, 828, or 869,
CDR-H3 comprises an amino acid sequence having at least 90% sequence identity to SEQ ID NO: 829,
CDR-L1 comprises an amino acid sequence having at least 90% sequence identity to SEQ ID NO: 819 or 820,
CDR-L2 comprises an amino acid sequence having at least 90% sequence identity to SEQ ID NO: 821 or 822, and
CDR-L3 comprises an amino acid sequence having at least 90% sequence identity to SEQ ID NO: 823 or 824.

182. The Fab-Fc dimer or the conjugate of claim 181, wherein
(a) the heavy chain variable region comprises an amino acid sequence having at least 70%, at least 75%, at least 80%, at least 85%, at least 90%, or at least 95% identity to the amino acid sequence of SEQ ID NO: 837 or 853; and
(b) the light chain variable region comprises an amino acid sequence having at least 70%, at least 75%, at least 80%, at least 85%, at least 90%, or at least 95% identity to the amino acid sequence of SEQ ID NO: 832 or 851.

183. The Fab-Fc dimer or the conjugate of claim 182, wherein
(a) the heavy chain variable region comprises an amino acid sequence having at least 70%, at least 75%, at least 80%, at least 85%, at least 90%, or at least 95% identity to the amino acid sequence of SEQ ID NO: 837 and the light chain variable region comprises an amino acid sequence having at least 70%, at least 75%, at least 80%, at least 85%, at least 90%, or at least 95% identity to the amino acid sequence of SEQ ID NO: 832; or
(a) the heavy chain variable region comprises an amino acid sequence having at least 70%, at least 75%, at least 80%, at least 85%, at least 90%, or at least 95% identity to the amino acid sequence of SEQ ID NO: 853 and the light chain variable region comprises an amino acid sequence having at least 70%, at least 75%, at least 80%, at least 85%, at least 90%, or at least 95% identity to the amino acid sequence of SEQ ID NO: 851.

184. The Fab-Fc dimer or the conjugate of claim 182, wherein
(a) the heavy chain variable region comprises the amino acid sequence of SEQ ID NO: 837 or 853; and
(b) the light chain variable region comprises the amino acid sequence of SEQ ID NO: 832 or 851.

185. The Fab-Fc dimer or the conjugate of claim 1847, wherein
(a) the heavy chain variable region comprises the amino acid sequence of SEQ ID NO: 837 and the light chain variable region comprises to the amino acid sequence of SEQ ID NO: 832; or
(a) the heavy chain variable region comprises the amino acid sequence of SEQ ID NO: 853 and the light chain variable region comprises the amino acid sequence of SEQ ID NO: 851.

186. The Fab-Fc dimer or the conjugate of claim 181, wherein the NTF comprises
(a) a heavy chain comprising an amino acid sequence having at least 70%, at least 75%, at least 80%, at least 85%, at least 90%, or at least 95% identity to the amino acid sequence of any one of SEQ ID NO: 838, 839, 840, 841, 844, 845, 846, 847, 854, 855, 856, 857, 859, 860, 861, and 862; and
(b) a light chain comprising an amino acid sequence having at least 70%, at least 75%, at least 80%, at least 85%, at least 90%, or at least 95% identity to the amino acid sequence of any one of SEQ ID NO: 833, 835, 850, and 852.

187. The Fab-Fc dimer or the conjugate of claim 186, wherein
(a) the heavy chain comprises the amino acid sequence of any one of SEQ ID NO: 838, 839, 840, 841, 844, 845, 846, 847, 854, 855, 856, 857, 859, 860, 861, and 862; and
(b) the light chain comprises the amino acid sequence of any one of SEQ ID NO: 833, 835, 850, and 852.

188. The Fab-Fc dimer or the conjugate of claim 187, wherein
(a) the heavy chain comprises the amino acid sequence of SEQ ID NO: 838, 839, 840, or 841, and the light chain comprises the amino acid sequence of SEQ ID NO: 833;
(b) the heavy chain comprises the amino acid sequence of SEQ ID NO: 844, 845, 846, or 847, and the light chain comprises the amino acid sequence of SEQ ID NO: 833;
(c) the heavy chain comprises the amino acid sequence of SEQ ID NO: 838, 839, 840, or 841, and the light chain comprises the amino acid sequence of SEQ ID NO: 835;
(d) the heavy chain comprises the amino acid sequence of SEQ ID NO: 844, 845, 846, or 847, and the light chain comprises the amino acid sequence of SEQ ID NO: 835;
(e) the heavy chain comprises the amino acid sequence of SEQ ID NO: 854, 855, 856, or 857, and the light chain comprises the amino acid sequence of SEQ ID NO: 850;
(f) the heavy chain comprises the amino acid sequence of SEQ ID NO: 859, 860, 861, or 862, and the light chain comprises the amino acid sequence of SEQ ID NO: 850;
(g) the heavy chain comprises the amino acid sequence of SEQ ID NO: 854, 855, 856, or 857, and the light chain comprises the amino acid sequence of SEQ ID NO: 852; and,
(h) the heavy chain comprises the amino acid sequence of SEQ ID NO: 859, 860, 861, or 862, and the light chain comprises the amino acid sequence of SEQ ID NO: 852.

189. The Fab-Fc dimer of any one of claims 23-30 or 32-36 or the conjugate of any one of claims 40-70, 76-83, 90-95, 100-104, or 119-134, wherein each of the Fab of the Fab-Fc dimer and the non-targeting Fab (NTF) of the conjugate comprises a light chain variable region and a heavy change variable region, does not specifically bind to a naturally occurring epitope in a subject, and comprises three light chain CDRs and three heavy chain CDRs, wherein
CDR-H1 comprises SEQ ID NO: 825 or 826,
CDR-H2 comprises SEQ ID NO: 827, 828, or 869,
CDR-H3 comprises SEQ ID NO: 829,
CDR-L1 comprises SEQ ID NO: 819 or 820,
CDR-L2 comprises SEQ ID NO: 821 or 822, and
CDR-L3 comprises SEQ ID NO: 823 or 824.


SEQUENCE LISTING (876 sequences)

SEQ ID NO: 1 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 113 aa, synthetic construct
  REGION 1..113: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 107 aa, synthetic construct
  REGION 1..107: note = Synthetic sequence
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGLVWVGYKTTPPVLDSDGSFFLYSKLTVAKSTWQQGWVFSCSVMHEALHNHYTQKSLSLSPGK

SEQ ID NO: 5 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTVWSHYKTTPPVLDSDGSFFLYSKLTVSKSEWQQGYVFSCSVMHEALHNHYTQKSLSLSPGK

SEQ ID NO: 6 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSQYKTTPPVLDSDGSFFLYSKLTVEKSDWQQGHVFSCSVMHEALHNHYTQKSLSLSPGK

SEQ ID NO: 7 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESVGTPWALYKTTPPVLDSDGSFFLYSKLTVLKSEWQQGWVFSCSVMHEALHNHYTQKSLSLSPGK

SEQ ID NO: 8 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTVWSKYKTTPPVLDSDGSFFLYSKLTVSKSEWQQGFVFSCSVMHEALHNHYTQKSLSLSPGK

SEQ ID NO: 9 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWAVYKTTPPVLDSDGSFFLYSKLTVPKSTWQQGWVFSCSVMHEALHNHYTQKSLSLSPGK

SEQ ID NO: 10 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGLVWVGYKTTPPVLDSDGSFFLYSKLTVPKSTWQQGWVFSCSVMHEALHNHYTQKSLSLSPGK

SEQ ID NO: 11 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESMGHVWVGYKTTPPVLDSDGSFFLYSKLTVDKSTWQQGWVFSCSVMHEALHNHYTQKSLSLSPGK

SEQ ID NO: 12 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGLVWVFSKTTPPVLDSDGSFFLYSKLTVPKSTWQQGWVFSCSVMHEALHNHYTQKSLSLSPGK

SEQ ID NO: 13 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKSEWQQGFVFSCSVMHEALHNHYTQKSLSLSPGK

SEQ ID NO: 14 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKSEWQQGFVFSCSVMHEALHNHYTQKSLSLSPGK

SEQ ID NO: 15 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVGYKTTPPVLDSDGSFFLYSKLTVSKSEWQQGWVFSCSVMHEALHNHYTQKSLSLSPGK

SEQ ID NO: 16 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVATKTTPPVLDSDGSFFLYSKLTVPKSTWQQGWVFSCSVMHEALHNHYTQKSLSLSPGK

SEQ ID NO: 17 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGPVWVHTKTTPPVLDSDGSFFLYSKLTVPKSTWQQGWVFSCSVMHEALHNHYTQKSLSLSPGK

SEQ ID NO: 18 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVDQKTTPPVLDSDGSFFLYSKLTVPKSTWQQGWVFSCSVMHEALHNHYTQKSLSLSPGK

SEQ ID NO: 19 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVNQKTTPPVLDSDGSFFLYSKLTVPKSTWQQGWVFSCSVMHEALHNHYTQKSLSLSPGK

SEQ ID NO: 20 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVNFKTTPPVLDSDGSFFLYSKLTVPKSTWQQGWVFSCSVMHEALHNHYTQKSLSLSPGK

SEQ ID NO: 21 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWAVYKTTPPVLDSDGSFFLYSKLTVPKSTWQQGWVFSCSVMHEALHNHYTQKSLSLSPGK

SEQ ID NO: 22 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWAVYQTTPPVLDSDGSFFLYSKLTVPKSTWQQGWVFSCSVMHEALHNHYTQKSLSLSPGK

SEQ ID NO: 23 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWAVYQTTPPVLDSDGSFFLYSKLTVPKSTWQQGWVFSCSVMHEALHNHYTQKSLSLSPGK

SEQ ID NO: 24 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKSEWQQGFVFSCSVMHEALHNHYTQKSLSLSPGK

SEQ ID NO: 25 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKEEWQQGFVFSCSVMHEALHNHYTQKSLSLSPGK

SEQ ID NO: 26 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 27 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVTKSEWQQGFVFSCSVMHEALHNHYTQKSLSLSPGK

SEQ ID NO: 28 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYQTTPPVLDSDGSFFLYSKLTVTKSEWQQGFVFSCSVMHEALHNHYTQKSLSLSPGK

SEQ ID NO: 29 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYQTTPPVLDSDGSFFLYSKLTVTKSEWQQGFVFSCSVMHEALHNHYTQKSLSLSPGK

SEQ ID NO: 30-106 - skipped (77 entries)

SEQ ID NO: 107 - protein, 181 aa, synthetic construct
  REGION 1..181: note = Synthetic sequence
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 108-231 - skipped (124 entries)

SEQ ID NO: 232 - protein, 7 aa, synthetic construct
  REGION 1..7: note = Synthetic sequence
DKTHTCP

SEQ ID NO: 233 - protein, 10 aa, synthetic construct
  REGION 1..10: note = Synthetic sequence
DKTHTCPPCP

SEQ ID NO: 234 - protein, 15 aa, synthetic construct
  REGION 1..15: note = Synthetic sequence
EPKSCDKTHTCPPCP

SEQ ID NO: 235 - protein, 760 aa, synthetic construct
  REGION 1..760: note = Synthetic sequence
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 236 - protein, 220 aa, synthetic construct; identical to SEQ ID NO: 24
  REGION 1..220: note = Synthetic sequence

SEQ ID NO: 237 - protein, 220 aa, synthetic construct; identical to SEQ ID NO: 25
  REGION 1..220: note = Synthetic sequence

SEQ ID NO: 238 - protein, 220 aa, synthetic construct; identical to SEQ ID NO: 26
  REGION 1..220: note = Synthetic sequence

SEQ ID NO: 239 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVTKSEWQQGFVFSCSVMHEALHNHYTQKSLSLSPGK

SEQ ID NO: 240 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVTKEEWQQGFVFSCSVMHEALHNHYTQKSLSLSPGK

SEQ ID NO: 241 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVTKEEWQQGFVFSCSVMHEALHNHYTQKSLSLSPGK

SEQ ID NO: 242 - protein, 220 aa, synthetic construct; identical to SEQ ID NO: 21
  REGION 1..220: note = Synthetic sequence

SEQ ID NO: 243 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESLGHVWAVYKTTPPVLDSDGSFFLYSKLTVPKSTWQQGWVFSCSVMHEALHNHYTQKSLSLSPGK

SEQ ID NO: 244 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVYWESLGHVWAVYKTTPPVLDSDGSFFLYSKLTVPKSTWQQGWVFSCSVMHEALHNHYTQKSLSLSPGK

SEQ ID NO: 245 - protein, 220 aa, synthetic construct; identical to SEQ ID NO: 22
  REGION 1..220: note = Synthetic sequence

SEQ ID NO: 246 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWAVYFTTPPVLDSDGSFFLYSKLTVPKSTWQQGWVFSCSVMHEALHNHYTQKSLSLSPGK

SEQ ID NO: 247 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWAVYHTTPPVLDSDGSFFLYSKLTVPKSTWQQGWVFSCSVMHEALHNHYTQKSLSLSPGK

SEQ ID NO: 248 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKSEWQQGFVFSCSVMHEALHNHYTQKSLSLSPGK

SEQ ID NO: 249 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKSEWQQGFVFSCSVMHEALHNHYTQKSLSLSPGK

SEQ ID NO: 250 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFSCSVMHEALHNHYTQKSLSLSPGK

SEQ ID NO: 251 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTGEEWQQGFVFSCSVMHEALHNHYTQKSLSLSPGK

SEQ ID NO: 252 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFSCWVMHEALHNHYTQKSLSLSPGK

SEQ ID NO: 253 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKEEWQQGFVFSCWVMHEALHNHYTQKSLSLSPGK

SEQ ID NO: 254 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFTCWVMHEALHNHYTQKSLSLSPGK

SEQ ID NO: 255 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFTCGVMHEALHNHYTQKSLSLSPGK

SEQ ID NO: 256 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFECWVMHEALHNHYTQKSLSLSPGK

SEQ ID NO: 257 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTREEWQQGFVFKCWVMHEALHNHYTQKSLSLSPGK

SEQ ID NO: 258 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTPEEWQQGFVFKCWVMHEALHNHYTQKSLSLSPGK

SEQ ID NO: 259 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFSCSVMHEALHNHYTQKSLSLSPGK

SEQ ID NO: 260 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTGEEWQQGFVFSCSVMHEALHNHYTQKSLSLSPGK

SEQ ID NO: 261 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFTCWVMHEALHNHYTQKSLSLSPGK

SEQ ID NO: 262 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTGEEWQQGFVFTCWVMHEALHNHYTQKSLSLSPGK

SEQ ID NO: 263 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYRTTPPVLDSDGSFFLYSKLTVTREEWQQGFVFTCGVMHEALHNHYTQKSLSLSPGK

SEQ ID NO: 264 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVTKEEWQQGFVFSCSVMHEALHNHYTQKSLSLSPGK

SEQ ID NO: 265 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYRTTPPVLDSDGSFFLYSKLTVTKEEWQQGFVFSCSVMHEALHNHYTQKSLSLSPGK

SEQ ID NO: 266 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 267 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWASYKTTPPVLDSDGSFFLYSKLTVTKEEWQQGFVFSCSVMHEALHNHYTQKSLSLSPGK

SEQ ID NO: 268 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWVSYKTTPPVLDSDGSFFLYSKLTVTKEEWQQGFVFSCSVMHEALHNHYTQKSLSLSPGK

SEQ ID NO: 269 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVSKEEWQQGFVFSCSVMHEALHNHYTQKSLSLSPGK

SEQ ID NO: 270 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWASYKTTPPVLDSDGSFFLYSKLTVTKEEWQQGFVFSCSVMHEALHNHYTQKSLSLSPGK

SEQ ID NO: 271 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWVSYKTTPPVLDSDGSFFLYSKLTVTKEEWQQGFVFSCSVMHEALHNHYTQKSLSLSPGK

SEQ ID NO: 272 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWSSYKTTPPVLDSDGSFFLYSKLTVTKEEWQQGFVFSCSVMHEALHNHYTQKSLSLSPGK

SEQ ID NO: 273 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWASYKTTPPVLDSDGSFFLYSKLTVTKEEWQQGFVFSCSVMHEALHNHYTQKSLSLSPGK

SEQ ID NO: 274 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWVSYKTTPPVLDSDGSFFLYSKLTVTKEEWQQGFVFSCSVMHEALHNHYTQKSLSLSPGK

SEQ ID NO: 275 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVSKEEWQQGFVFSCSVMHEALHNHYTQKSLSLSPGK

SEQ ID NO: 276 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWASYKTTPPVLDSDGSFFLYSKLTVTKEEWQQGFVFSCSVMHEALHNHYTQKSLSLSPGK

SEQ ID NO: 277 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWVSYKTTPPVLDSDGSFFLYSKLTVTKEEWQQGFVFSCSVMHEALHNHYTQKSLSLSPGK

SEQ ID NO: 278 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSNYKTTPPVLDSDGSFFLYSKLTVTKEEWQQGFVFSCSVMHEALHNHYTQKSLSLSPGK

SEQ ID NO: 279 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 280 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 281 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 282 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWANYKTTPPVLDSDGSFFLYSKLTVTKEEWQQGFVFSCSVMHEALHNHYTQKSLSLSPGK

SEQ ID NO: 283 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWVNYKTTPPVLDSDGSFFLYSKLTVTKEEWQQGFVFSCSVMHEALHNHYTQKSLSLSPGK

SEQ ID NO: 284 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWSNYKTTPPVLDSDGSFFLYSKLTVTKEEWQQGFVFSCSVMHEALHNHYTQKSLSLSPGK

SEQ ID NO: 285 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWANYKTTPPVLDSDGSFFLYSKLTVTKEEWQQGFVFSCSVMHEALHNHYTQKSLSLSPGK

SEQ ID NO: 286 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWVNYKTTPPVLDSDGSFFLYSKLTVTKEEWQQGFVFSCSVMHEALHNHYTQKSLSLSPGK

SEQ ID NO: 287 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSNYKTTPPVLDSDGSFFLYSKLTVSKEEWQQGFVFSCSVMHEALHNHYTQKSLSLSPGK

SEQ ID NO: 288 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWANYKTTPPVLDSDGSFFLYSKLTVTKEEWQQGFVFSCSVMHEALHNHYTQKSLSLSPGK

SEQ ID NO: 289 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESFGTEWVNYKTTPPVLDSDGSFFLYSKLTVTKEEWQQGFVFSCSVMHEALHNHYTQKSLSLSPGK

SEQ ID NO: 290 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWSSYKTTPPVLDSDGSFFLYSKLTVTKEEWQQGFVFSCSVMHEALHNHYTQKSLSLSPGK

SEQ ID NO: 291 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPGK

SEQ ID NO: 292 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWVSYKTTPPVLDSDGSFFLYSKLTVTKEEWQQGFVFSCSVMHEALHNHYTQKSLSLSPGK

SEQ ID NO: 293 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWSSYKTTPPVLDSDGSFFLYSKLTVSKEEWQQGFVFSCSVMHEALHNHYTQKSLSLSPGK

SEQ ID NO: 294 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWASYKTTPPVLDSDGSFFLYSKLTVTKEEWQQGFVFSCSVMHEALHNHYTQKSLSLSPGK

SEQ ID NO: 295 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESFGTEWVSYKTTPPVLDSDGSFFLYSKLTVTKEEWQQGFVFSCSVMHEALHNHYTQKSLSLSPGK

SEQ ID NO: 296 - protein, 220 aa, synthetic construct; identical to SEQ ID NO: 27
  REGION 1..220: note = Synthetic sequence

SEQ ID NO: 297 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWVNQKTTPPVLDSDGSFFLYSKLTVPKSTWQQGWVFSCSVMHEALHNHYTQKSLSLSPGK

SEQ ID NO: 298 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVNQQTTPPVLDSDGSFFLYSKLTVPKSTWQQGWVFSCSVMHEALHNHYTQKSLSLSPGK

SEQ ID NO: 299 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESLGHVWVNQQTTPPVLDSDGSFFLYSKLTVPKSTWQQGWVFSCSVMHEALHNHYTQKSLSLSPGK

SEQ ID NO: 300-301 - skipped (2 entries)

SEQ ID NO: 302 - protein, 247 aa, synthetic construct
  REGION 1..247: note = Synthetic sequence
METDTLLLWVLLLWVPGSTGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESLGHVWAVYKTTPPVLDSDGSFFLYSKLTVPKSTWQQGWVFSCSVMHEALHNHYTQKSLSLSPGK

SEQ ID NO: 303-360 - skipped (58 entries)

SEQ ID NO: 361 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 362 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 363 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 364 - protein, 220 aa, synthetic construct
  REGION 1..220: note = Synthetic sequence
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 365 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 366 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 367 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 368 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 369 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 370 - protein, 220 aa, synthetic construct
  REGION 1..220: note = Synthetic sequence
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 371 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 372 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 373-396 - skipped (24 entries)

SEQ ID NO: 397 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 398 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 399 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 400 - protein, 220 aa, synthetic construct
  REGION 1..220: note = Synthetic sequence
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 401 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 402 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 403 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 404 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 405 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 406 - protein, 220 aa, synthetic construct
  REGION 1..220: note = Synthetic sequence
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 407 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 408 - protein, 220 aa, synthetic construct
  REGION 1..220: note = Synthetic sequence
PCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVMHEALHNHYTQKSLSLSPGK

SEQ ID NO: 409-420 - skipped (12 entries)

SEQ ID NO: 421 - protein, 247 aa, synthetic construct
  REGION 1..247: note = Synthetic sequence
METDTLLLWVLLLWVPGSTGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESYGTEWSSYKTTPPVLDSDGSFFLYSKLTVTKSEWQQGFVFSCSVMHEALHNHYTQKSLSLSPGK

SEQ ID NO: 422 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESWGFVWSTYKTTPPVLDSDGSFFLYSKLTVPKSNWQQGFVFSCSVMHEALHNHYTQKSLSLSPGK

SEQ ID NO: 423 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESWGHVWSTYKTTPPVLDSDGSFFLYSKLTVPKSNWQQGYVFSCSVMHEALHNHYTQKSLSLSPGK

SEQ ID NO: 424 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVEQKTTPPVLDSDGSFFLYSKLTVPKSTWQQGWVFSCSVMHEALHNHYTQKSLSLSPGK

SEQ ID NO: 425 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVGVKTTPPVLDSDGSFFLYSKLTVPKSTWQQGWVFSCSVMHEALHNHYTQKSLSLSPGK

SEQ ID NO: 426 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVHTKTTPPVLDSDGSFFLYSKLTVPKSTWQQGWVFSCSVMHEALHNHYTQKSLSLSPGK

SEQ ID NO: 427 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESWGTVWGTYKTTPPVLDSDGSFFLYSKLTVPKSNWQQGYVFSCSVMHEALHNHYTQKSLSLSPGK

SEQ ID NO: 428 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESLGHVWVGTKTTPPVLDSDGSFFLYSKLTVPKSTWQQGWVFSCSVMHEALHNHYTQKSLSLSPGK

SEQ ID NO: 429 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSSYKTTPPVLDSDGSFFLYSKLTVSKEEWQQGFVFSCSVMHEALHNHYTQKSLSLSPGK

SEQ ID NO: 430 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVSKSEWQQGFVFSCSVMHEALHNHYTQKSLSLSPGK

SEQ ID NO: 431 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSNYKTTPPVLDSDGSFFLYSKLTVSKEEWQQGFVFSCSVMHEALHNHYTQKSLSLSPGK

SEQ ID NO: 432 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVSKSEWQQGFVFSCSVMHEALHNHYTQKSLSLSPGK

SEQ ID NO: 433 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWVNYKTTPPVLDSDGSFFLYSKLTVSKEEWQQGFVFSCSVMHEALHNHYTQKSLSLSPGK

SEQ ID NO: 434 - protein, 220 aa, synthetic construct; identical to SEQ ID NO: 14
  REGION 1..220: note = Synthetic sequence

SEQ ID NO: 435 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWVNYKTTPPVLDSDGSFFLYSKLTVSKEEWQQGFVFSCSVMHEALHNHYTQKSLSLSPGK

SEQ ID NO: 436-490 - skipped (55 entries)

SEQ ID NO: 491 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVLHEALHSHYTQKSLSLSPGK

SEQ ID NO: 492 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVLHEALHSHYTQKSLSLSPGK

SEQ ID NO: 493 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVLHEALHSHYTQKSLSLSPGK

SEQ ID NO: 494 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVLHEALHSHYTQKSLSLSPGK

SEQ ID NO: 495 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVLHEALHSHYTQKSLSLSPGK

SEQ ID NO: 496 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVLHEALHSHYTQKSLSLSPGK

SEQ ID NO: 497 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVLHEALHSHYTQKSLSLSPGK

SEQ ID NO: 498-511 - skipped (14 entries)

SEQ ID NO: 512 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVLHEALHSHYTQKSLSLSPGK

SEQ ID NO: 513 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVLHEALHSHYTQKSLSLSPGK

SEQ ID NO: 514 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVLHEALHSHYTQKSLSLSPGK

SEQ ID NO: 515 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVLHEALHSHYTQKSLSLSPGK

SEQ ID NO: 516 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVLHEALHSHYTQKSLSLSPGK

SEQ ID NO: 517 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVLHEALHSHYTQKSLSLSPGK

SEQ ID NO: 518 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVLWESYGTEWASYKTTPPVLDSDGSFFLVSKLTVTKEEWQQGFVFSCSVLHEALHSHYTQKSLSLSPGK

SEQ ID NO: 519-553 - skipped (35 entries)

SEQ ID NO: 554 - protein, 11 aa, synthetic construct
  REGION 1..11: note = Synthetic sequence
  VAR_SEQ 1: note = MISC_FEATURE - X is E, L, S, V, W, or Y
  VAR_SEQ 5: note = MISC_FEATURE - X is Y, F, W, M, P, or V
  VAR_SEQ 7: note = MISC_FEATURE - X is T, N, or V
  VAR_SEQ 8: note = MISC_FEATURE - X is E, I, P, or V
  VAR_SEQ 10: note = MISC_FEATURE - X is A, I, V, S, or T
  VAR_SEQ 11: note = MISC_FEATURE - X is S, N, R, or T
XWESXGXXWXX

SEQ ID NO: 555 - protein, 14 aa, synthetic construct
  REGION 1..14: note = Synthetic sequence
  VAR_SEQ 1: note = MISC_FEATURE - X is T, H, or S
  VAR_SEQ 3: note = MISC_FEATURE - X is E, S, D, G, T, P, Q, or R
  VAR_SEQ 4: note = MISC_FEATURE - X is E or R
  VAR_SEQ 9: note = MISC_FEATURE - X is F, H, K, Y, or W
  VAR_SEQ 12: note = MISC_FEATURE - X is S, T, or W
  VAR_SEQ 14: note = MISC_FEATURE - X is S, C, P, M, or W
XKXXWQQGXVFXCX

SEQ ID NO: 556 - protein, 107 aa, synthetic construct
  REGION 1..107: note = Synthetic sequence
  VAR_SEQ 40: note = MISC_FEATURE - X is E, L, S, V, W, or Y
  VAR_SEQ 44: note = MISC_FEATURE - X is Y, F, W, M, P, or V
  VAR_SEQ 46: note = MISC_FEATURE - X is T, N, or V
  VAR_SEQ 47: note = MISC_FEATURE - X is E, I, P, or V
  VAR_SEQ 49: note = MISC_FEATURE - X is A, I, V, S, or T
  VAR_SEQ 50: note = MISC_FEATURE - X is S, N, R, or T
  VAR_SEQ 73: note = MISC_FEATURE - X is T, H, or S
  VAR_SEQ 75: note = MISC_FEATURE - X is E, S, D, G, T, P, Q, or R
  VAR_SEQ 76: note = MISC_FEATURE - X is E or R
  VAR_SEQ 81: note = MISC_FEATURE - X is F, H, K, Y, or W
  VAR_SEQ 84: note = MISC_FEATURE - X is S, T, or W
  VAR_SEQ 86: note = MISC_FEATURE - X is S, C, P, M, or W
GQPREPQVYTLPPSRDELTKNQVSLTCLVKGFYPSDIAVXWESXGXXWXXYKTTPPVLDSDGSFFLYSKLTVXKXXWQQGXVFXCXVMHEALHNHYTQKSLSLSPGK

SEQ ID NO: 557 - protein, 220 aa, synthetic construct
  REGION 1..220: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 558 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 559 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 560 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 561 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 562-622 - skipped (61 entries)

SEQ ID NO: 623 - protein, 330 aa, synthetic construct [sequence omitted]

SEQ ID NO: 624 - protein, 326 aa, synthetic construct
  REGION 1..326: note = Synthetic sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 625 - protein, 377 aa, synthetic construct
  REGION 1..377: note = Synthetic sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 626 - protein, 327 aa, synthetic construct
  REGION 1..327: note = Synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 627 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 628 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 629 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 630 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 631 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 632 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 633 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPGK

SEQ ID NO: 634 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 635 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 636 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 637 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 638 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 639 - protein, 219 aa, synthetic construct
  REGION 1..219: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 640 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 641 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPG

SEQ ID NO: 642 - protein, 219 aa, synthetic construct
  REGION 1..219: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 643 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 644 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPG

SEQ ID NO: 645 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPG

SEQ ID NO: 646 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 647 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 648 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 649 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 650 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWESYGTEWASYKTTPPVLDSDGSFFLYSKLTVTKEEWQQGFVFSCSVMHEALHNHYTQKSLSLSPG

SEQ ID NO: 651 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 652 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 653 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 654 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVLWESYGTEWASYKTTPPVLDSDGSFFLYSKLTVTKEEWQQGFVFSCSVMHEALHNHYTQKSLSLSPG

SEQ ID NO: 655 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 656 - DNA, 16 bp, synthetic construct
  misc_feature 1..16: note = Synthetic sequence
  source 1..16: mol_type = other DNA
gcattctaatagcagc

SEQ ID NO: 657 - DNA, 16 bp, synthetic construct
  misc_feature 1..16: note = Synthetic sequence
  source 1..16: mol_type = other DNA
gcattctaatagcagc

SEQ ID NO: 658-699 - skipped (42 entries)

SEQ ID NO: 700 - protein, 98 aa, synthetic construct
  REGION 1..98: note = Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 701 - protein, 107 aa, synthetic construct [sequence omitted]

SEQ ID NO: 702 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 703 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 704 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 705 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 706 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 707 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLCLSPGK

SEQ ID NO: 708 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 709 - protein, 330 aa, synthetic construct
  REGION 1..330: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 710 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 711 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 712 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 713 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 714 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 715 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLCLSPGK

SEQ ID NO: 716 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 717 - protein, 330 aa, synthetic construct
  REGION 1..330: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 718 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 719 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 720 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 721 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 722 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 723 - protein, 330 aa, synthetic construct
  REGION 1..330: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 724 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 725 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 726 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 727 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 728 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 729 - protein, 220 aa, synthetic construct
  REGION 1..220: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 730 - protein, 220 aa, synthetic construct
  REGION 1..220: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALGCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 731 - protein, 330 aa, synthetic construct
  REGION 1..330: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 732 - protein, 107 aa, synthetic construct
  REGION 1..107: note = Synthetic sequence
RTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 733 - protein, 219 aa, synthetic construct
  REGION 1..219: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 734 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 735 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 736 - protein, 226 aa, synthetic construct
  REGION 1..226: note = Synthetic sequence
DKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 737 - protein, 329 aa, synthetic construct
  REGION 1..329: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 738 - protein, 219 aa, synthetic construct
  REGION 1..219: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 739 - protein, 219 aa, synthetic construct
  REGION 1..219: note = Synthetic sequence
PCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 740 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 741 - protein, 226 aa, synthetic construct
  REGION 1..226: note = Synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 742 - protein, 219 aa, synthetic construct
  REGION 1..219: note = Synthetic sequence
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPG

SEQ ID NO: 743 - protein, 330 aa, synthetic construct
  REGION 1..330: note = Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 744 - protein, 330 aa, synthetic construct
  REGION 1..330: note = Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 745 - protein, 329 aa, synthetic construct
  REGION 1..329: note = Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 746 - protein, 219 aa, synthetic construct
  REGION 1..219: note = Synthetic sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKEEWQQGFVFSCSVMHEALHNHYTQKSLSLSPG

SEQ ID NO: 747-799 - skipped (53 entries)

SEQ ID NO: 800 - protein, 213 aa, synthetic construct
  REGION 1..213: note = Synthetic sequence
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 801 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 802 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 803 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 804 - protein, 227 aa, synthetic construct
  REGION 1..227: note = Synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 805 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 806 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 807 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 808 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPCPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 809 - protein, 450 aa, synthetic construct
  REGION 1..450: note = Synthetic sequence
QVTLRESGPALVKPTQTLTLTCTFSGFSLSTSGMSVGWIRQPPGKALEWLADIWWDDKKDYNPSLKSRLTISKDTSKNQVVLKVTNMEPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLCLSPGK

SEQ ID NO: 810 - protein, 227 aa, synthetic construct
  REGION 1..227: note = Synthetic sequence
DKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 811 - protein, 214 aa, synthetic construct
  REGION 1..214: note = Synthetic sequence
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 812 - protein, 214 aa, synthetic construct
  REGION 1..214: note = Synthetic sequence
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 813 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 814 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 815 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 816 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 817 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 818 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 819 - protein, 11 aa, synthetic construct
  REGION 1..11: note = Synthetic sequence
SASSSVSMYWY

SEQ ID NO: 820 - protein, 13 aa, synthetic construct
  REGION 1..13: note = Synthetic sequence
RASQSVSSYLAWY

SEQ ID NO: 821 - protein, 7 aa, synthetic construct
  REGION 1..7: note = Synthetic sequence
DTSNLAS

SEQ ID NO: 822 - protein, 7 aa, synthetic construct
  REGION 1..7: note = Synthetic sequence
DASNLAT

SEQ ID NO: 823 - protein, 10 aa, synthetic construct
  REGION 1..10: note = Synthetic sequence
QQWSSYPPIT

SEQ ID NO: 824 - protein, 10 aa, synthetic construct
  REGION 1..10: note = Synthetic sequence
QQRSNWPPIT

SEQ ID NO: 825 - protein, 11 aa, synthetic construct
  REGION 1..11: note = Synthetic sequence
GYSITSDYAWN

SEQ ID NO: 826 - protein, 11 aa, synthetic construct
  REGION 1..11: note = Synthetic sequence
GYSITSDYAWG

SEQ ID NO: 827 - protein, 16 aa, synthetic construct
  REGION 1..16: note = Synthetic sequence
YMSYSGSTRYNPSLRS

SEQ ID NO: 828 - protein, 16 aa, synthetic construct
  REGION 1..16: note = Synthetic sequence
SMSYSGSTYYNPSLKS

SEQ ID NO: 829 - protein, 8 aa, synthetic construct
  REGION 1..8: note = Synthetic sequence
ARGWPLAY

SEQ ID NO: 830 - protein, 131 aa, synthetic construct
  REGION 1..131: note = Synthetic sequence
MDFQVQIFSFLLISASVILSRGQIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRA

SEQ ID NO: 831 - protein, 132 aa, synthetic construct
  REGION 1..132: note = Synthetic sequence
MRVILLWLFTAFPGILSDVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSE

SEQ ID NO: 832 - protein, 107 aa, synthetic construct
  REGION 1..107: note = Synthetic sequence
EIVMTQSPATLSLSPGERATLSCSASSSVSYMYWYQQKPGQAPRLLIYDTSNLASGIPARFSGSGSGTDFTLTISSLQPEDFAVYYCQQWSSYPPITFGQGTKVEIK

SEQ ID NO: 833 - protein, 214 aa, synthetic construct; identical to SEQ ID NO: 811
  REGION 1..214: note = Synthetic sequence

SEQ ID NO: 834 - protein, 107 aa, synthetic construct [sequence omitted]

SEQ ID NO: 835 - protein, 214 aa, synthetic construct; identical to SEQ ID NO: 812
  REGION 1..214: note = Synthetic sequence

SEQ ID NO: 836 - protein, 107 aa, synthetic construct; identical to SEQ ID NO: 732
  REGION 1..107: note = Synthetic sequence

SEQ ID NO: 837 - protein, 115 aa, synthetic construct
  REGION 1..115: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSS

SEQ ID NO: 838 - protein, 216 aa, synthetic construct
  REGION 1..216: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 839 - protein, 218 aa, synthetic construct
  REGION 1..218: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 840 - protein, 225 aa, synthetic construct
  REGION 1..225: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 841 - protein, 228 aa, synthetic construct
  REGION 1..228: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 842 - protein, 101 aa, synthetic construct
  REGION 1..101: note = Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 843 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 844 - protein, 216 aa, synthetic construct
  REGION 1..216: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 845 - protein, 218 aa, synthetic construct
  REGION 1..218: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 846 - protein, 225 aa, synthetic construct
  REGION 1..225: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 847 - protein, 228 aa, synthetic construct
  REGION 1..228: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 848 - protein, 101 aa, synthetic construct
  REGION 1..101: note = Synthetic sequence
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 849 - protein, 445 aa, synthetic construct
  REGION 1..445: note = Synthetic sequence
QVQLQESGPGLVKPSETLSLTCAVSGYSITSDYAWNWIRQPPGKGLEWIGYMSYSGSTRYNPSLRSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 850 - protein, 215 aa, synthetic construct
  REGION 1..215: note = Synthetic sequence
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 851 - protein, 108 aa, synthetic construct
  REGION 1..108: note = Synthetic sequence
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIK

SEQ ID NO: 852 - protein, 215 aa, synthetic construct
  REGION 1..215: note = Synthetic sequence
EIVLTQSPATLSLSPGERATLSCRASQSVSSYLAWYQQKPGQAPRLLIYDASNLATGIPARFSGSGSGTDFTLTISSLEPEDFAVYYCQQRSNWPPITFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 853 - protein, 115 aa, synthetic construct
  REGION 1..115: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSS

SEQ ID NO: 854 - protein, 216 aa, synthetic construct
  REGION 1..216: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 855 - protein, 218 aa, synthetic construct
  REGION 1..218: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 856 - protein, 225 aa, synthetic construct
  REGION 1..225: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 857 - protein, 228 aa, synthetic construct
  REGION 1..228: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 858 - protein, 445 aa, synthetic construct
  REGION 1..445: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 859 - protein, 216 aa, synthetic construct
  REGION 1..216: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 860 - protein, 218 aa, synthetic construct
  REGION 1..218: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 861 - protein, 225 aa, synthetic construct
  REGION 1..225: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 862 - protein, 228 aa, synthetic construct
  REGION 1..228: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 863 - protein, 445 aa, synthetic construct
  REGION 1..445: note = Synthetic sequence
QVQLQESGPGLVKPSETLSLTCAVSGYSITSDYAWGWIRQPPGKGLEWIGSMSYSGSTYYNPSLKSRVTISVDTSKNQFSLKLSSVTAADTAVYYCARGWPLAYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 864 - DNA, 324 bp, synthetic construct
  misc_feature 1..324: note = Synthetic sequence
  source 1..324: mol_type = other DNA
gaaattgtcctcacccagagcccagccactctttccctctctccaggggaaagagcaacactgagttgccgggcctcccagagtgtaagttcctacctcgcatggtatcaacagaagcctggtcaagcaccccggcttctgatttacgatgccagcaatctcgccaccggaatccccgcaaggttttcagggagcggttcagggactgactttaccctgaccataagcagtcttgagcctgaggattttgcagtgtattactgccaacagagaagtaactggccccccataacctttggacaggggactaaggtggagattaaa

SEQ ID NO: 865 - DNA, 345 bp, synthetic construct
  misc_feature 1..345: note = Synthetic sequence
  source 1..345: mol_type = other DNA
caagtgcaactgcaggaaagtgggcctggtttggtcaaacccagtgaaacacttagtcttacttgcgctgtatctggatactctattacaagcgattatgcctgggggtggattcggcagccccctggaaaaggcttggagtggatcggttcaatgagttatagtggtagtacatactacaacccttccctgaagtctcgcgtcacaataagcgtagatacaagtaagaatcaattttccctgaaacttagcagtgtaactgccgctgataccgcagtctactattgtgccagaggatggcccctggcctactggggtcagggcaccctcgtgaccgtatcatca

SEQ ID NO: 866 - DNA, 324 bp, synthetic construct
  misc_feature 1..324: note = Synthetic sequence
  source 1..324: mol_type = other DNA
gagatcgtactcactcaaagtccagccaccctgtcactttctccaggagaacgcgccactctctcttgtcgggctagtcagagcgttagtagctatctcgcatggtatcaacaaaagcccggtcaggccccacgcctgttgatctatgatgccagtaacctggccaccggtattccagcccgtttctctggcagtggaagtggaactgacttcacactgaccatcagctccctcgagcccgaagattttgctgtgtattactgccagcaaagatccaactggcctccaatcactttcggccaaggaacaaaagtagagattaag

SEQ ID NO: 867 - DNA, 345 bp, synthetic construct
  misc_feature 1..345: note = Synthetic sequence
  source 1..345: mol_type = other DNA
caggttcaacttcaagagtcaggcccaggcttggtgaagccctccgaaactttgagcttgacttgcgctgtgtccggttacagtataacctctgactatgcctgggggtggataaggcaacctcctggcaaaggattggaatggatcggttcaatgtcctactccggttctacatactataacccctccttgaagtcccgagtaactatcagtgtggacacttcaaagaatcaatttagtctgaaattgtcttcagtcaccgctgcagacactgctgtctattattgtgccaggggttggccccttgcttattggggccaggggaccctggtgacagtatcaagc

SEQ ID NO: 868 - protein, 15 aa, synthetic construct
  REGION 1..15: note = Synthetic sequence
EPKSCDKTHTCPPCP

SEQ ID NO: 869 - protein, 16 aa, synthetic construct
  REGION 1..16: note = Synthetic sequence
  VAR_SEQ 1: note = MISC_FEATURE - X is Y or S
  VAR_SEQ 9: note = MISC_FEATURE - X is R or Y
  VAR_SEQ 15: note = MISC_FEATURE - X is R or K
XMSYSGSTXYNPSLXS

SEQ ID NO: 870 - RNA, 21 nt, synthetic construct
  misc_feature 1..21: note = Synthetic sequence
  source 1..21: mol_type = other RNA
tcctatgactgtagattttat

SEQ ID NO: 871 - RNA, 23 nt, synthetic construct
  misc_feature 1..23: note = Synthetic sequence
  source 1..23: mol_type = other RNA
ataaaatctacagtcataggaat

SEQ ID NO: 872 - DNA, 12 bp, synthetic construct
  misc_feature 1..12: note = Synthetic sequence
  source 1..12: mol_type = other DNA
attcctatgact

SEQ ID NO: 873 - DNA, 11 bp, synthetic construct
  misc_feature 1..11: note = Synthetic sequence
  source 1..11: mol_type = other DNA
gtagattttat

SEQ ID NO: 874 - DNA, 11 bp, synthetic construct
  misc_feature 1..11: note = Synthetic sequence
  source 1..11: mol_type = other DNA
ataaaatctac

SEQ ID NO: 875 - DNA, 10 bp, synthetic construct
  misc_feature 1..10: note = Synthetic sequence
  source 1..10: mol_type = other DNA
agtcatagga

SEQ ID NO: 876 - DNA, 21 bp, synthetic construct
  misc_feature 1..21: note = Synthetic sequence
  source 1..21: mol_type = other DNA
ataaaatctacagtcatagga